SEQUENCE LISTING (44 sequences)

SEQ ID NO: 1 - DNA, 20 bp, synthetic construct
  misc_feature 1..20: note = Synthetic: NT1
  source 1..20: mol_type = other DNA
aaaaagcttccgcctgatgg

SEQ ID NO: 2 - DNA, 20 bp, synthetic construct
  misc_feature 1..20: note = Synthetic: NT2
  source 1..20: mol_type = other DNA
cataggtccctagcaactcc

SEQ ID NO: 3 - DNA, 20 bp, synthetic construct
  misc_feature 1..20: note = Synthetic: BRD1#1
  source 1..20: mol_type = other DNA
aaataggattgcgaatcagg

SEQ ID NO: 4 - DNA, 20 bp, synthetic construct
  misc_feature 1..20: note = Synthetic: BRD1#2
  source 1..20: mol_type = other DNA
aaactgcttcttccgctgaa

SEQ ID NO: 5 - DNA, 20 bp, synthetic construct
  misc_feature 1..20: note = Synthetic: CBX5#1
  source 1..20: mol_type = other DNA
tacccagggagcacaatact

SEQ ID NO: 6 - DNA, 20 bp, synthetic construct
  misc_feature 1..20: note = Synthetic: CBX5#2
  source 1..20: mol_type = other DNA
taaattcagaaattagctca

SEQ ID NO: 7 - DNA, 20 bp, synthetic construct
  misc_feature 1..20: note = Synthetic: CHD3#1
  source 1..20: mol_type = other DNA
gcaactctgcttctgacctg

SEQ ID NO: 8 - DNA, 20 bp, synthetic construct
  misc_feature 1..20: note = Synthetic: CHD3#2
  source 1..20: mol_type = other DNA
ggagcatgtgttctctgagg

SEQ ID NO: 9 - DNA, 20 bp, synthetic construct
  misc_feature 1..20: note = Synthetic: EPC1#1
  source 1..20: mol_type = other DNA
tttaggaacatcatcttcag

SEQ ID NO: 10 - DNA, 20 bp, synthetic construct
  misc_feature 1..20: note = Synthetic: EPC1#2
  source 1..20: mol_type = other DNA
gcgggctatttcagcacagc

SEQ ID NO: 11 - DNA, 20 bp, synthetic construct
  misc_feature 1..20: note = Synthetic: EPC2#1
  source 1..20: mol_type = other DNA
ttgacgttgctctctgcctc

SEQ ID NO: 12 - DNA, 20 bp, synthetic construct
  misc_feature 1..20: note = Synthetic: EPC2#2
  source 1..20: mol_type = other DNA
tattacaatcgcttgtacaa

SEQ ID NO: 13 - DNA, 20 bp, synthetic construct
  misc_feature 1..20: note = Synthetic: EZH2#1
  source 1..20: mol_type = other DNA
cggaaatcttaaaccaagaa

SEQ ID NO: 14 - DNA, 20 bp, synthetic construct
  misc_feature 1..20: note = Synthetic: EZH2#2
  source 1..20: mol_type = other DNA
gcaatgagctcacagaagtc

SEQ ID NO: 15 - DNA, 20 bp, synthetic construct
  misc_feature 1..20: note = Synthetic: HDAC2#1
  source 1..20: mol_type = other DNA
tgggtcatgcggattctatg

SEQ ID NO: 16 - DNA, 20 bp, synthetic construct
  misc_feature 1..20: note = Synthetic: HDAC2#2
  source 1..20: mol_type = other DNA
acagcaagttatgggtcatg

SEQ ID NO: 17 - DNA, 20 bp, synthetic construct
  misc_feature 1..20: note = Synthetic: KDM1A#1
  source 1..20: mol_type = other DNA
atactcatcttctgagaggt

SEQ ID NO: 18 - DNA, 20 bp, synthetic construct
  misc_feature 1..20: note = Synthetic: KDM1A#2
  source 1..20: mol_type = other DNA
accccctcaagccccacctg

SEQ ID NO: 19 - DNA, 20 bp, synthetic construct
  misc_feature 1..20: note = Synthetic: KDM5B#1
  source 1..20: mol_type = other DNA
actcccagtactttgcaatc

SEQ ID NO: 20 - DNA, 20 bp, synthetic construct
  misc_feature 1..20: note = Synthetic: KDM5B#2
  source 1..20: mol_type = other DNA
gcaaagtactgggagttaca

SEQ ID NO: 21 - DNA, 20 bp, synthetic construct
  misc_feature 1..20: note = Synthetic: KMT5B#1
  source 1..20: mol_type = other DNA
tttcacaggtaatggtaact

SEQ ID NO: 22 - DNA, 20 bp, synthetic construct
  misc_feature 1..20: note = Synthetic: KMT5B#2
  source 1..20: mol_type = other DNA
agtcgctatgtaccatcctc

SEQ ID NO: 23 - DNA, 20 bp, synthetic construct
  misc_feature 1..20: note = Synthetic: MTA2#1
  source 1..20: mol_type = other DNA
gcgccatcaactgaagcacc

SEQ ID NO: 24 - DNA, 20 bp, synthetic construct
  misc_feature 1..20: note = Synthetic: MTA2#2
  source 1..20: mol_type = other DNA
gaagaggaatcaaagcagcc

SEQ ID NO: 25 - DNA, 20 bp, synthetic construct
  misc_feature 1..20: note = Synthetic: MTF2#1
  source 1..20: mol_type = other DNA
tgattgattcagatgaaaaa

SEQ ID NO: 26 - DNA, 20 bp, synthetic construct
  misc_feature 1..20: note = Synthetic: MTF2#2
  source 1..20: mol_type = other DNA
cagatgaaaaatggctctgt

SEQ ID NO: 27 - DNA, 20 bp, synthetic construct
  misc_feature 1..20: note = Synthetic: RBBP4#1
  source 1..20: mol_type = other DNA
tcatgtcacctggttacatc

SEQ ID NO: 28 - DNA, 20 bp, synthetic construct
  misc_feature 1..20: note = Synthetic: RBBP4#2
  source 1..20: mol_type = other DNA
attgccgttacagaccagaa

SEQ ID NO: 29 - DNA, 20 bp, synthetic construct
  misc_feature 1..20: note = Synthetic: RNF2#1
  source 1..20: mol_type = other DNA
atcatcacagcccttagaag

SEQ ID NO: 30 - DNA, 20 bp, synthetic construct
  misc_feature 1..20: note = Synthetic: RNF2#2
  source 1..20: mol_type = other DNA
aattcactgtgtagacttcg

SEQ ID NO: 31 - DNA, 20 bp, synthetic construct
  misc_feature 1..20: note = Synthetic: SMARCA4#1
  source 1..20: mol_type = other DNA
atggtccctctcgcagccca

SEQ ID NO: 32 - DNA, 20 bp, synthetic construct
  misc_feature 1..20: note = Synthetic: SMARCA4#2
  source 1..20: mol_type = other DNA
ctttcatctggttgtagcgc

SEQ ID NO: 33 - DNA, 20 bp, synthetic construct
  misc_feature 1..20: note = Synthetic: SMARCE1#1
  source 1..20: mol_type = other DNA
cagcaaatgcccagcacacc

SEQ ID NO: 34 - DNA, 20 bp, synthetic construct
  misc_feature 1..20: note = Synthetic: SMARCE1#2
  source 1..20: mol_type = other DNA
tcctaccgtgacccggctgt

SEQ ID NO: 35 - DNA, 20 bp, synthetic construct
  misc_feature 1..20: note = Synthetic: SMARCAD1#1
  source 1..20: mol_type = other DNA
aactgtattggagcaatttg

SEQ ID NO: 36 - DNA, 20 bp, synthetic construct
  misc_feature 1..20: note = Synthetic: SMARCAD1#2
  source 1..20: mol_type = other DNA
gaactgtattggagcaattt

SEQ ID NO: 37 - DNA, 20 bp, synthetic construct
  misc_feature 1..20: note = Synthetic: RCOR2#1
  source 1..20: mol_type = other DNA
tctgtggcataagcacgatg

SEQ ID NO: 38 - DNA, 20 bp, synthetic construct
  misc_feature 1..20: note = Synthetic: RCOR2#2
  source 1..20: mol_type = other DNA
gcatttgccatggaagccaa

SEQ ID NO: 39 - DNA, 20 bp, synthetic construct
  misc_feature 1..20: note = Synthetic: SOX4#1
  source 1..20: mol_type = other DNA
gcccgagtccgagctctcgc

SEQ ID NO: 40 - DNA, 20 bp, synthetic construct
  misc_feature 1..20: note = Synthetic: SOX4#2
  source 1..20: mol_type = other DNA
attgttggtttgctgcacca

SEQ ID NO: 41 - DNA, 20 bp, synthetic construct
  misc_feature 1..20: note = Synthetic: SOX11#1
  source 1..20: mol_type = other DNA
gcggatcatggtgcagcagg

SEQ ID NO: 42 - DNA, 20 bp, synthetic construct
  misc_feature 1..20: note = Synthetic: SOX11#2
  source 1..20: mol_type = other DNA
attcatggcttgcagcccgg

SEQ ID NO: 43 - DNA, 20 bp, synthetic construct
  misc_feature 1..20: note = Synthetic: KLF12#1
  source 1..20: mol_type = other DNA
cgtccacaactatcccgata

SEQ ID NO: 44 - DNA, 20 bp, synthetic construct
  misc_feature 1..20: note = Synthetic: KLF12#2
  source 1..20: mol_type = other DNA
acagataacgagtcctccgg

(19) United States
(12) Patent Application Publication
(10) Pub. No.: US 2024/0279600 A1
(43) Pub. Date: Aug. 22, 2024

(54) COMPOSITIONS AND METHODS FOR PROMOTING IN VITRO MATURATION OF CELLS

(71) Applicant: MEMORIAL SLOAN-KETTERING CANCER CENTER, New York, NY (US)

(72) Inventors: Lorenz Studer, New York, NY (US); Emiliano Hergenreder, New York, NY (US); Gabriele Clceri, New York, NY (US)

(73) Assignee: MEMORIAL SLOAN-KETTERING CANCER CENTER, New York, NY (US)

(21) Appl. No.: 18/647,342

(22) Filed: Apr. 26, 2024

Related U.S. Application Data
(63) Continuation of application No. PCT/US2022/048161, filed on Oct. 28, 2022.
(60) Provisional application No. 63/272,946, filed on Oct. 28, 2021.

(51) Int. Cl. C12N 5/079

(52) U.S. Cl. CPC C12N 5/0618; C12N 2501/999

(57) ABSTRACT

The present disclosure provides compositions, kits, and methods for promoting in vitro maturation of cells. The present disclosure also provides methods of screening compounds that are suitable for promoting in vitro maturation of cells.

### CROSS-REFERENCE TO RELATED APPLICATIONS

This application is a continuation of International Patent Application No. PCT/US2022/048161, filed Oct. 28, 2022, which claims priority to U.S. Provisional Patent Application No. 63/272,946, filed on Oct. 28, 2021, the content of each which is incorporated by reference in its entirety herein, and to each of which priority is claimed.

### GRANT INFORMATION

The present disclosure was made with government support under Grant No. CA008748 awarded by the National Cancer Institute and Grant No. AG054720 awarded by the National Institute of Aging. The government has certain rights in the disclosure.

### SEQUENCE LISTING

The instant application contains a Sequence Listing which has been submitted electronically in XML format and is hereby incorporated by reference in its entirety. Said XML copy, created on Apr. 26, 2024, is named 072734.1619.xml and is 56,442 bytes in size.

### 1. TECHNICAL FIELD

The present disclosure provides compositions, kits, and methods for promoting in vitro maturation of cells. The present disclosure also provides methods of screening compounds that are suitable for promoting in vitro maturation of cells.

### 2. BACKGROUND

Recent advances in human pluripotent stem cell (hPSC) differentiation have enabled the derivation of various specific subtypes of neurons on demand. However, the application of this technology remains hampered by the slow maturation rates of human cells, resulting in prolonged culture periods for the emergence of disease-relevant phenotypes. Most neurological and psychiatric disorders manifest as impairments in postnatal or adult neuron functions such as synaptic connectivity, dendritic arborization, and electrophysiological function. Developing strategies to accelerate the maturation of hPSC-derived cells is critical to realize their full potential in modeling and treating diseases. Therefore, there remain needs for compositions and methods of promoting in vitro maturation of cells.

### 3. SUMMARY OF THE INVENTION

The present disclosure relates to compositions, kits, and methods for promoting in vitro maturation of cells. The present disclosure also provides methods of screening compounds that are suitable for promoting in vitro maturation of cells.

In certain embodiments, the present disclosure provides a composition for promoting in vitro maturation of cells, comprising at least one inhibitor of an epigenetic regulator, and at least one agonist of a calcium channel.

In certain embodiments, the at least one inhibitor of the epigenetic regulator comprises a lysine-specific demethylase 1 (LSD1) inhibitor, a disruptor of telomerase-like 1 (DOT1L) inhibitor, or a combination thereof. In certain embodiments, the at least one agonist of the calcium channel comprises a glutamate receptor agonist, an L-type calcium channel (LTCC) agonist, or a combination thereof.

In certain embodiments, the LSD1 inhibitor is selected from the group consisting of GSK2879552, OG-L002, GSK-LSD1, derivatives thereof, and combinations thereof. In certain embodiments, the DOT1L inhibitor is selected from the group consisting of EPZ-5676, EPZ004777, SYC-522, SGC0946, Dot1L-IN-2, Dot1L-IN-4, Dot1L-IN-5, Dot1L-IN-6, CN-SAH, derivatives thereof, and combinations thereof. In certain embodiments, the glutamate receptor agonist is selected from the group consisting of NMDA, (RS)-(tetratazol-5-yl)glycine, ibotenic acid, derivatives thereof, and combinations thereof. In certain embodiments, the LTCC agonist is selected from the group consisting of Bay K 8644, FPL 64176, derivatives thereof, and combinations thereof.

In certain embodiments, the composition comprises an LSD1 inhibitor, a DOT1L inhibitor, a glutamate receptor agonist, and an LTCC agonist. In certain embodiments, the composition comprises GSK2879552, EPZ-5676, NMDA, and Bay K 8644.

In certain embodiments, the concentration of the LSD1 inhibitor is between about 0.1 μM and about 10 μM. In certain embodiments, the concentration of the LSD1 inhibitor is about 1 μM. In certain embodiments, the concentration of the DOT1L inhibitor is between about 0.1 μM and about 10 μM. In certain embodiments, the concentration of the DOT1L inhibitor is about 1 μM. In certain embodiments, the concentration of the glutamate receptor agonist is between about 0.1 μM and about 10 μM. In certain embodiments, the concentration of the glutamate receptor agonist is about 1 μM. In certain embodiments, the concentration of the LTCC agonist is between about 0.1 μM and about 10 μM. In certain embodiments, the concentration of the LTCC agonist is about 1 μM.

In certain embodiments, the present disclosure provides a composition for promoting in vitro maturation of cells, comprising at least one inhibitor of an epigenetic regulator.

In certain embodiments, the at least one inhibitor of the epigenetic regulator comprises a disruptor of telomerase-like 1 (DOT1L) inhibitor, an enhancer of zeste homolog 2 (EZH2) inhibitor, an euchromatic histone-lysine-N-methyltransferases 1 and 2 (EHMT1/2) inhibitor, or a combination thereof.

In certain embodiments, the EZH2 inhibitor is selected from the group consisting of 3-deazaneplanocin A (DZNep), GSK343, GSK126, EPZ-6438, EPZ005687, GSK926, EPZ6438, EPZ011989, CPI-1205, CPI-169, ZLD1039, PF-06821497, UNC1999, PR-S1/OR-S2, DS-3201b, A-395, EBI-2511, EED226, EEDi-5285, EI1, EZH2-IN-2, EZH2-IN-3, EZH2-IN-4, EZH2-IN-5, GNA002, GSK503, JQEZ5, MAK683, MS1943, PF-06726304, UNC 1999, UNC6852, UNC6852, AM41-44A, BR-001, CPI-1328, CPI-905, DCE_254, EBI-2511, YM181, YM181, ZLD1039, ZLD10A, derivatives thereof, and combinations thereof.

In certain embodiments, the EHMT1/2 inhibitor is selected from the group consisting of UNC0638, UNC0224, UNC0321, UNC0642, UNC0646, UNC0642, UNC0631, A-366, BIX-01294, BRD4770, BRD9539, CM-272, CM-579, CPUY074020, CSV0C018875, EHMT2-IN-1, EHMT2-IN-2, EML741, derivatives thereof, and combinations thereof.

In certain embodiments, the DOT1L inhibitor is selected from the group consisting of EPZ-5676, EPZ004777, SYC-522, SGC0946, Dot1L-IN-2, Dot1L-IN-4, Dot1L-IN-5, Dot1L-IN-6, CN-SAH, derivatives thereof, and combinations thereof.

In certain embodiments, the composition comprises GSK343, EPZ004777, UNC0638, or a combination thereof.

In certain embodiments, the concentration of the at least one inhibitor of the epigenetic regulator is between about 0.1 μM and about 10 μM.

In certain embodiments, the concentration of the at least one inhibitor of the epigenetic regulator is about 2 μM or about 4 μM.

In certain embodiments, the present disclosure provides an in vitro method for promoting the maturation of cells, comprising contacting the cells with at least one inhibitor of an epigenetic regulator, and at least one agonist of a calcium channel.

In certain embodiments, the at least one inhibitor of the epigenetic regulator comprises a lysine-specific demethylase 1 (LSD1) inhibitor, a disruptor of telomerase-like 1 (DOT1L) inhibitor, or a combination thereof. In certain embodiments, the at least one agonist of the calcium channel comprises a glutamate receptor agonist, an L-type calcium channel (LTCC) agonist, or a combination thereof.

In certain embodiments, the LSD1 inhibitor is selected from the group consisting of GSK2879552, OG-L002, GSK-LSD1, derivatives thereof, and combinations thereof. In certain embodiments, the DOT1L inhibitor is selected from the group consisting of EPZ-5676, EPZ004777, SYC-522, SGC0946, Dot1L-IN-2, Dot1L-IN-4, Dot1L-IN-5, Dot 1L-IN-6, CN-SAH, derivatives thereof, and combinations thereof. In certain embodiments, the glutamate receptor agonist is selected from the group consisting of NMDA, (RS)-(tetratazol-5-yl)glycine, ibotenic acid, derivatives thereof, and combinations thereof. In certain embodiments, the LTCC agonist is selected from the group consisting of Bay K 8644, FPL 64176, derivatives thereof, and combinations thereof.

In certain embodiments, the method comprises contacting the cells with an LSD1 inhibitor, a DOT1L inhibitor, a glutamate receptor agonist, and an LTCC agonist. In certain embodiments, the method comprises contacting the cells with GSK2879552, EPZ-5676, NMDA, and Bay K 8644.

In certain embodiments, the concentration of the LSD1 inhibitor is between about 0.1 μM and about 10 μM. In certain embodiments, the concentration of the LSD1 inhibitor is about 1 μM. In certain embodiments, the concentration of the DOT1L inhibitor is between about 0.1 μM and about 10 μM. In certain embodiments, the concentration of the DOT1L inhibitor is about 1 μM. In certain embodiments, the concentration of the glutamate receptor agonist is between about 0.1 μM and about 10 μM. In certain embodiments, the concentration of the glutamate receptor agonist is about 1 μM. In certain embodiments, the concentration of the LTCC agonist is between about 0.1 μM and about 10 μM. In certain embodiments, the concentration of the LTCC agonist is about 1 μM.

In certain embodiments, the cells are contacted with the at least one inhibitor of the epigenetic regulator and the at least one agonist of the calcium channel for at least about 3 days and/or for up to about 30 days.

In certain embodiments, the present disclosure provides an in vitro method for promoting the maturation of cells, comprising contacting the cells with at least one inhibitor of an epigenetic regulator.

In certain embodiments, the at least one inhibitor of the epigenetic regulator comprises a disruptor of telomerase-like 1 (DOT1L) inhibitor, an enhancer of zeste homolog 2 (EZH2) inhibitor, an euchromatic histone-lysine-N-methyltransferases 1 and 2 (EHMT1/2) inhibitor, or a combination thereof.

In certain embodiments, the EZH2 inhibitor is selected from the group consisting of 3-deazaneplanocin A (DZNep), GSK343, GSK126, EPZ-6438, EPZ005687, GSK926, EPZ6438, EPZ011989, CPI-1205, CPI-169, ZLD1039, PF-06821497, UNC1999, PR-S1/OR-S2, DS-3201b, A-395, EBI-2511, EED226, EEDi-5285, EI1, EZH2-IN-2, EZH2-IN-3, EZH2-IN-4, EZH2-IN-5, GNA002, GSK503, JQEZ5, MAK683, MS1943, PF-06726304, UNC 1999, UNC6852, UNC6852, AM41-44A, BR-001, CPI-1328, CPI-905, DCE_254, EBI-2511, YM181, YM181, ZLD1039, ZLD10A, derivatives thereof, and combinations thereof.

In certain embodiments, the EHMT1/2 inhibitor is selected from the group consisting of UNC0638, UNC0224, UNC0321, UNC0642, UNC0646, UNC0642, UNC0631, A-366, BIX-01294, BRD4770, BRD9539, CM-272, CM-579, CPUY074020, CSV0C018875, EHMT2-IN-1, EHMT2-IN-2, EML741, derivatives thereof, and combinations thereof.

In certain embodiments, the DOT1L inhibitor is selected from the group consisting of EPZ-5676, EPZ004777, SYC-522, SGC0946, Dot1L-IN-2, Dot1L-IN-4, Dot1L-IN-5, Dot1L-IN-6, CN-SAH, derivatives thereof, and combinations thereof.

In certain embodiments, the method comprises contacting the cells with GSK343, EPZ004777, UNC0638, or a combination thereof.

In certain embodiments, the concentration of the at least one inhibitor of the epigenetic regulator is between about 0.1 μM and about 10 μM.

In certain embodiments, the concentration of the at least one inhibitor of the epigenetic regulator is about 2 μM or about 4 μM.

In certain embodiments, the cells are immature neuronal cells, precursors thereof, progenitors thereof, or a combination thereof. In certain embodiments, the neuronal cells are selected from the group consisting of cortical neurons, spinal motor neurons, and combinations thereof. In certain embodiments, the cells form a brain organoid. In certain embodiments, the brain organoid is a dorsal forebrain organoid. In certain embodiments, the cells are immature non-neuronal cells, precursors thereof, progenitors thereof, or a combination thereof. In certain embodiments, the cells are selected from the group consisting of pancreatic beta cells, melanocytes, and combinations thereof.

In certain embodiments, the cells are in vitro differentiated from stem cells. In certain embodiments, the stem cells are selected from the group consisting of embryonic stem cells, induced pluripotent stem cells, parthenogenetic stem cells, primordial germ cell-like pluripotent stem cells, epiblast stem cells, and F-class pluripotent stem cells, embryonic neural stem cells, adult neural stem cells, long-term self-renewing neural stem cells, and combinations thereof.

In certain embodiments, the present disclosure provides an in vitro method for promoting the maturation of cells, comprising contacting the cells with the presently disclosed composition.

In certain embodiments, the present disclosure provides use of the presently disclosed composition for promoting the maturation of cells.

In certain embodiments, the present disclosure provides a kit for promoting in vitro maturation of cells, comprising at least one inhibitor of an epigenetic regulator, and at least one agonist of a calcium channel.

In certain embodiments, the at least one inhibitor of the epigenetic regulator comprises a lysine-specific demethylase 1 (LSD1) inhibitor, a disruptor of telomerase-like 1 (DOT1L) inhibitor, or a combination thereof. In certain embodiments, the at least one agonist of the calcium channel comprises a glutamate receptor agonist, an L-type calcium channel (LTCC) agonist, or a combination thereof.

In certain embodiments, the LSD1 inhibitor is selected from the group consisting of GSK2879552, OG-L002, GSK-LSD1, derivatives thereof, and combinations thereof. In certain embodiments, the DOT1L inhibitor is selected from the group consisting of EPZ-5676, EPZ004777, SYC-522, SGC0946, Dot1L-IN-2, Dot1L-IN-4, Dot1L-IN-5, Dot1L-IN-6, CN-SAH, derivatives thereof, and combinations thereof. In certain embodiments, the glutamate receptor agonist is selected from the group consisting of NMDA, (RS)-(tetratazol-5-yl)glycine, ibotenic acid, derivatives thereof, and combinations thereof. In certain embodiments, the LTCC agonist is selected from the group consisting of Bay K 8644, FPL 64176, derivatives thereof, and combinations thereof.

In certain embodiments, the kit comprises an LSD1 inhibitor, a DOT1L inhibitor, a glutamate receptor agonist, and an LTCC agonist. In certain embodiments, the kit comprises GSK2879552, EPZ-5676, NMDA, and Bay K 8644.

In certain embodiments, the present disclosure provides a kit for promoting in vitro maturation of cells, comprising at least one inhibitor of an epigenetic regulator.

In certain embodiments, the at least one inhibitor of the epigenetic regulator comprises a disruptor of telomerase-like 1 (DOT1L) inhibitor, an enhancer of zeste homolog 2 (EZH2) inhibitor, an euchromatic histone-lysine-N-methyltransferases 1 and 2 (EHMT1/2) inhibitor, or a combination thereof.

In certain embodiments, the EZH2 inhibitor is selected from the group consisting of 3-deazaneplanocin A (DZNep), GSK343, GSK126, EPZ-6438, EPZ005687, GSK926, EPZ6438, EPZ011989, CPI-1205, CPI-169, ZLD1039, PF-06821497, UNC1999, PR-S1/OR-S2, DS-3201b, A-395, EBI-2511, EED226, EEDi-5285, EI1, EZH2-IN-2, EZH2-IN-3, EZH2-IN-4, EZH2-IN-5, GNA002, GSK503, JQEZ5, MAK683, MS1943, PF-06726304, UNC 1999, UNC6852, UNC6852, AM41-44A, BR-001, CPI-1328, CPI-905, DCE_254, EBI-2511, YM181, YM181, ZLD1039, ZLD10A, derivatives thereof, and combinations thereof.

In certain embodiments, the EHMT1/2 inhibitor is selected from the group consisting of UNC0638, UNC0224, UNC0321, UNC0642, UNC0646, UNC0642, UNC0631, A-366, BIX-01294, BRD4770, BRD9539, CM-272, CM-579, CPUY074020, CSV0C018875, EHMT2-IN-1, EHMT2-IN-2, EML741, derivatives thereof, and combinations thereof.

In certain embodiments, the DOT1L inhibitor is selected from the group consisting of EPZ-5676, EPZ004777, SYC-522, SGC0946, Dot1L-IN-2, Dot1L-IN-4, Dot1L-IN-5, Dot1L-IN-6, CN-SAH derivatives thereof, and combinations thereof.

In certain embodiments, the kit comprises GSK343, EPZ004777, UNC0638, or a combination thereof.

In certain embodiments, the kit further comprises instructions for promoting in vitro maturation of cells.

In certain embodiments, the present disclosure provides an in vitro method of screening a compound that is suitable for promoting in vitro maturation of cells, comprising: (a) contacting a population of immature neuronal cells to a test compound; (b) withdrawing the test compound; (c) contacting the cells with potassium chloride between about 3 days and about 20 days after the withdrawal of the test compound; (d) measuring nuclear morphology, neurite growth and membrane excitability of the cells; (e) performing principal component analysis on the nuclear morphology, neurite growth and membrane excitability measured in step (d); and (f) identifying a test compound that is suitable for promoting in vitro maturation of neuronal cells based on the principal component analysis performed in (e).

In certain embodiments, the cells are contacted with potassium chloride about 7 days after the withdrawal of the test compound.

In certain embodiments, the concentration of potassium chloride is between about 10 mM and about 100 mM. In certain embodiments, the concentration of potassium chloride is about 50 mM.

In certain embodiments, measuring the nuclear morphology comprises measuring nuclear area and nuclear roundness. In certain embodiments, the nuclear morphology is determined by DAPI counterstaining.

In certain embodiments, measuring the neurite growth comprises measuring neurite length and neurite branching. In certain embodiments, the neurite growth is determined by microtubule-associated protein 2 (MAP2) immunostaining.

In certain embodiments, measuring the membrane excitability comprises measuring percentage of cells expressing an immediate early gene (IEG) product. In certain embodiments, measuring the membrane excitability comprises subtracting the percentage of cells expressing the IEG product with percentage of control cells expressing the IEG product, wherein the control cells are not subject to the contact of potassium chloride. In certain embodiments, the IEG product comprises FOS, EGR1, and a combination thereof.

In certain embodiments, the neuronal cells are cortical neurons.

### 4. BRIEF DESCRIPTION OF THE DRAWINGS

FIGS. 1A-1E show high-content chemical screen for drivers of neuron maturation. FIG. 1A depicts the outline of screening protocol in hPSC-derived cortical neurons. 2SMAD-i, dual SMAD inhibition. FIG. 1B shows an example of input immunofluorescent images. Top: unstimulated neurons at day 21 post plating. Bottom, neurons received 50 mM of KCl 2 hours before fixation. FIG. 1C shows automated analysis of neuron morphology. Left, nuclei detection mask from DAPI channel. Right, automated neurite tracing from MAP2 channel. FIG. 1D shows quantification of neuron excitability by applying an intensity threshold to FOS and EGR1 channels within the nuclear mask. FIG. 1E shows principal component analysis of screened compound library computed from 6 maturity parameters (z-scores averaged from n=2 independent screen runs). Left, PCA plot of 2343 non-toxic library compounds (out of 2688 total compounds tested) with phenotypic clustering of maturation enhancing (orange), maturation inhibiting (blue), and non-neuronal proliferation enhancing (grey) compounds. Right, representative screen images and 10 representative hit compounds within each cluster. Scale bars are 50 μm.

FIGS. 2A-2E show that validation and combination of screen hits identified maturation-promoting cocktail GENtoniK. FIG. 2A shows ranking of primary hits by the mean of 4 maturity parameters (nuclear size and roundness, neurite length, and KCl-induced double FOS/EGR1+ cells) normalized to DMSO (n=3 microplate wells). 22 top-ranked compounds were selected for validation. FIG. 2B shows dose-response validation of 22 screen hits comparing the mean of 4 maturity parameters normalized to DMSO (n=15 microplate wells from 3 independent experiments). FIG. 2C shows comparison of confirmed hits GSK2879552, EPZ-5676, Bay K 8644, and a combination of the 3 (G+E+K) across maturity parameters (n=8 microplate wells from 2 independent experiments). FIG. 2D shows comparison of 3-hit drug combination (G+E+K) to the same with the addition of NMDA across maturity parameters (n=8 microplate wells from 2 independent experiments). FIG. 2E shows formulation of GENtoniK, a small molecule cocktail that promoted neuron maturation and representative images of DMSO and GENtoniK-treated cortical neurons. For FIGS. 2B-2D, two-tailed Welch's t-test; asterisks indicate statistical significance. Mean values are represented by a bar graph (FIG. 2A) or a line (FIGS. 2C-2D). Error bars represent S.E.M. Scale bars are 50 μm.

FIGS. 3A-3M show validation of small molecule maturation strategy with orthogonal readouts. FIG. 3A shows representative images for synaptic marker detection in day 35 hPSC-derived cortical neurons that received DMSO versus GENtoniK treatment from days 7 to 21. Orange dots represent instances of SYN1 and PSD95 apposition. Inset, input immunofluorescent images used for quantification, with examples of pre- and post-synaptic marker apposition highlighted by arrows. FIGS. 3B-3D show that GENtoniK increased density of SYN1, PSD-95, and their apposition expressed as punctate per neurite length (n=16 wells from n=2 independent experiments). FIGS. 3E-3H show that GENtoniK promoted excitability and mature resting properties in day 28 hPSC-cortical neurons. FIG. 3E shows that >90% of treated neurons fired evoked action potentials in contrast to <40% of DMSO controls. Traces show representative responses for each group. FIGS. 3F-3H show quantification of electrophysiology parameter AP frequency (FIG. 3F), AP threshold (FIG. 3G), and resting membrane potential (FIG. 3H) (n=11 neurons per group from 4-6 dishes and 3 independent experiments). FIGS. 3I-3M show that RNA-seq and CUT&RUN (3 biological replicates) revealed that GENtoniK induced shift from immature to mature transcriptional programs. FIG. 3I depicts gene ontology analysis showing enrichment for mature neuron function in genes upregulated by the cocktail; and enrichment for immature function and transcriptional regulation in genes downregulated by the cocktail or occupied by DOT1L-target H3K79 2-methylation. FIG. 3J shows that in the BrainSpan Atlas of the Developing Human Brain (www.brainspan.org), genes downregulated by GENtoniK displayed higher average expression during early development and decreased over time (left), genes upregulated by GENtoniK displayed an average expression that increased from early development to gestation and after birth (right). Top panels show smoothed means curves with confidence intervals, bottom panels show heatmaps of normalized expression (FIGS. 3K-3M), CUT&RUN peak profiles of LSD1 and DOT1L targets H3K4 and H3K79 2-methylation in immature, untreated d7 hPSC-cortical neurons across the whole genome (FIG. 3K) and in genes downregulated (FIG. 3L) or upregulated (FIG. 3M) by GENtoniK in RNA-seq. For FIGS. 3B-3D and 3F-3H, Two-tailed Welch's t-test; asterisks indicate statistical significance. Mean values are represented by a black line (FIGS. 3B-3D) or a bar graph (FIGS. 3F-3H). Error bars represent S.E.M. Scale bars are 50 μm.

FIGS. 4A-4R show validation of maturation strategy across neuronal and non-neuronal hPSC-derived cells. FIGS. 4A-4D show that GENtoniK treatment induced synaptogenesis and spontaneous activity in cortical organoids. FIG. 4A shows representative images of immunofluorescent staining for SYN1 and MAP2 in day 60 organoids. FIG. 4B shows quantification of total SYN1 puncta per field (n=8 cryosections randomly sampled from n=20 organoids). FIG. 4C shows representative images of immunofluorescence staining for EGR1 and MAP2 in unstimulated day 60 organoids. FIG. 4D shows quantification of EGR1⁺ cells per field (n=8 cryosections randomly sampled from n=20 organoids). FIGS. 4E-4H show that GENtoniK promoted maturation of hPSC-derived spinal motor neurons. FIG. 4E shows representative high-content maturation assay images of ISL1/2⁺ spinal motor neurons (day 40 of hPSC differentiation). FIGS. 4F-4H show quantification showing GENtoniK-improved KCl-induction of FOS⁺ cells (FIG. 4F), total neurite length (FIG. 4G), and nuclear area (FIG. 4H) in SMNs (n=12 microplate wells from 2 independent differentiations). FIGS. 4I-4L show that GENtoniK treatment increased firing rates and induced spontaneous bursting activity on SMNs plated on high-density multielectrode arrays. FIG. 4I shows sample single channel trace of GENtoniK-treated SMNs illustrating spike detection. FIG. 4J shows time-course analysis of average firing rates in SMNs plated on HD-MEAs, calculated from 60 s of activity in the 1/64 most active electrodes (n=128 electrodes from 2 MEA probes). FIG. 4K shows representative 60-second spike rastergrams (top) and average firing rates (bottom) of SMNs plated on a HD-MEAs. Only GENtoniK-treated SMNs displayed spontaneous bursting events (orange bars). FIG. 4L shows whole array heatmap of a 4-second bursting event. FIGS. 4M-4N shows that GENtoniK treatment induced early pigmentation in hPSC-melanocytes. FIG. 4M shows brightfield images of melanocytes (day 33 of hPSC differentiation) that received GENtoniK or DMSO from day 11. FIG. 4N shows a dot blot analysis of PBS or cell extract of melanocytes treated with GENtoniK or DMSO (n=3 biological replicates). FIGS. 4O-4R show that GENtoniK promoted maturation of hESC-derived beta-like cells. Representative flow cytometry analysis (FIG. 4O) and quantification (FIG. 4P) of the percentage of GCG⁺ cells in INS-GFP⁺ cells after 7 days treatment with GENtoniK or control followed by 2 days treatment-free (n=4 biological replicates). FIG. 4Q shows the total insulin content of INS-GFP⁺ cells after 7 days treatment with GENtoniK or control followed by 2 days treatment-free (n=6-7 biological replicates). FIG. 4R shows static KCL-stimulated human insulin secretion and fold change in beta-like cells after 7 days treatment with GENtoniK or control followed by 2 days treatment-free. The assay was performed in the presence of 2 mM D-glucose (n=8-9 biological replicates). For FIGS. 4B, 4D, 4F-4H, 4J and 4P-4R, two-tailed Welch's t-test; asterisks indicate statistical significance. Mean values are represented by a black line (FIGS. 4B, 4D, 4F-H) or a bar graph (FIGS. 4P-4R). Error represent S.E.M. Scale bars are 50 μm.

FIGS. 5A-5M show design and optimization of high-content maturation assay. FIGS. 5A-5C show immunofluorescent staining of day 10 hPSC-cortical neurons for pan-neuronal marker MAP2 (FIGS. 5A, 5B), forebrain marker FOXG1 (FIG. 5A), and deep-layer cortex marker TBR1 (FIG. 5B). FIG. 5C shows quantification of immunofluorescent staining (n=12 microplate wells). FIG. 5D shows time-course quantification of cell number in post-mitotic hPSC-cortical neurons (DAPI⁺ cells per field, n=24 microplate wells). FIG. 5E shows immunofluorescent staining of primary embryonic rat cortex neurons (E18) using high-content markers. FIGS. 5F-5I show quantification of maturation parameters primary rat neurons that demonstrated mature values for nuclear size (FIG. 5F), nuclear roundness (FIG. 5G), neurite length (FIG. 5H), and KCL-induced IEG expression (FIG. 5I) (n=12 microplate wells). FIGS. 5J-5M show time course quantification of maturation parameters in hPSC-derived cortical neurons showing time-dependent increases in nuclear size (FIG. 5J), nuclear roundness (FIG. 5K), neurite length (FIG. 5L), and KCL-induced IEG expression (FIG. 5M) (n=24 microplate wells). Mean values are represented by a black line. Error bars represent S.E.M. Scale bars are 50 μm.

FIGS. 6A-6C show high-content screen data preparation and analysis. FIG. 6A depicts the pipeline of analysis of high-content screen using a 2688-compound bioactive library. Normalization scores (z-scores) of 2 independent screens were averaged and used for selection of hits via PCA or single-parameter scores. FIG. 6B shows exclusion of toxic compounds with a mean z-score of total cell number below −2. Note that increases in total cell number were only observed for compounds inducing non-neural cells (FIG. 1E). FIG. 6C shows correlation of mean maturation z-scores from 2 screen runs among non-toxic compounds.

FIGS. 7A & 7B show single parameter hit selection. FIG. 7A shows representative high-content screen image of a DMSO control well (left) and library compounds (excluding the PCA hits already selected) plotted against individual maturation parameter (right). Selected compounds are highlighted in bold, non-highlighted compounds were not included due to phenotype and/or known molecular target unrelated to neuronal maturation. Screen images are representative of high-scoring compounds for each parameter. FIG. 7B shows the ranking of 42 primary hits (PCA and single parameter) in individual maturation parameters (n=3 microplate wells). Mean values are represented by bar graph. Error bars represent S.E.M. Scale bars are 50 μm.

FIGS. 8A-8C show that maturation-promoting small molecules did not significantly affect neuron survival. FIG. 8A shows representative staining images from hit combination experiments (FIGS. 2C-2E), showing day 21 neurons that received the specified treatment from days 7-14. FIG. 8B shows quantification of number of cells per well in neurons treated with screen hits GSK2879552, EPZ-5676, Bay K 8644, and a combination of the 3 (G+E+K). FIG. 8C shows quantification of number of cells per well in neurons treated with 3-hit drug combination (G+E+K) and the same with the addition of NMDA. n=8 microplate wells from 2 independent experiments. Error bars represent S.E.M. Scale bars are 50 μm.

FIGS. 9A-9F show RNA-seq results of day 21 neurons treated with maturation promoting small molecules from day 7-14. FIG. 9A shows principal component analysis of RNA-seq results from neurons treated with DMSO, two epigenetic drugs (G+E), two calcium influx driving compounds (N+K), or complete GENtoniK. FIGS. 9B-9D show volcano plots of RNA-seq differential expression analysis vs DMSO of calcium influx agonist NMDA and Bay K 8644 (FIG. 9B), epigenetic drugs GSK2879552 and EPZ-5676 (FIG. 9C), or complete GENtoniK (FIG. 9D). FIGS. 9E & 9F show heatmaps of genes within overrepresented biological process ontology categories among GENtoniK-downregulated (FIG. 9E), and upregulated (FIG. 9F) genes. RNA-seq results from 3 biological replicates. Heatmaps show expression normalized by row, calculated from mean TPM values. Displayed p-values are for enrichment of stated gene ontology categories among differentially expressed transcripts.

FIGS. 10A-10C show that GENtoniK induced transcriptional activation of diverse metabolic pathways in cortical neurons. Gene set enrichment analysis (GSEA) of RNA-seq results showing enrichment for oxidative phosphorylation (FIG. 10A), canonical glycolysis (FIG. 10B), and fatty acid metabolism (FIG. 10C) gene ontology categories enriched in GENtoniK-treated neurons. N=3 biological replicates.

FIGS. 11A-11E show CUT&RUN analysis of LSD1 and DOT1L-targeted histone marks in untreated day 10 immature neurons. FIG. 11A (left) shows normalized genome enrichment profile of H3K4me2 over IGG control along 12 Kb region surrounding the transcription start site (TSS). FIG. 11A (right) shows genome-wide distribution of gene features among H3K4me2 peaks. FIG. 11B (left) shows normalized genome enrichment profile of H3K79me2 over IGG control along 24 Kb region surrounding the transcription start site (TSS). FIG. 11B (right) shows genome-wide distribution of gene features among H3K79me2 peaks. FIGS. 11C-11E show that enrichment of H3K79me2 vs IGG control in gene ontology categories significantly overrepresented among H3K79me2 peaks with representative tracks for genes within each category: GO:0001764-neuron migration and GO:0007411-axon guidance (FIG. 11C), GO:0016569-covalent chromatin modification (FIG. 11D), and GO:0006397-mRNA processing (FIG. 11E). Displayed p-values are for enrichment of stated ontology categories among genes within H3K79me2 peaks.

FIGS. 12A-12H show that GENtoniK promoted maturation of cortical neurons derived from induced pluripotent stem cells (iPSCs). FIGS. 12A-12D show results for neurons derived from reprogrammed normal lung fibroblast line MRC5 (n=16 microplate wells): representative high-content maturation assay images (FIG. 12A), and quantification of maturation parameters nuclear size (FIG. 12B), neurite length (FIG. 12C) and IEG induction by KCl (FIG. 12D). FIGS. 12E-12H show results for neurons derived from reprogrammed skin fibroblasts of 10-year-old male (n=16 microplate wells): representative high-content maturation assay images (FIG. 12E), and quantification of maturation parameters nuclear size (FIG. 12F), neurite length (FIG. 12G) and IEG induction by KCL (FIG. 12H). Two-tailed Welch's t-test; asterisks indicate statistical significance. Mean values are represented by a black line. Error bars represent S.E.M. Scale bars are 50 μm.

FIGS. 13A-13E show that GENtoniK improves upon and complemented alternative neuron maturation strategies. FIG. 13A shows immunofluorescent stain for MAP2, FOS, and SYN1 of day 35 hPSC-derived cortical neurons in plain Neurobasal medium, BrainPhys medium+BDNF, Neurobasal with GENtoniK, and BrainPhys+BDNF with GENtoniK. FIGS. 13B-13E show time-course quantification of the maturity parameters: FOS induction by KCl (FIG. 13B), neurite length (FIG. 13C), nuclear size (FIG. 13D), and SYN1 puncta density (FIG. 13E) in neurons that received GENtoniK versus DMSO from day 7 from plating. Plates were collected for analysis every 7 days, beginning 7 days after the start of DMSO/GENtoniK treatment. n=12 microplate wells. Error bars represent S.E.M. Scale bars are 50 μm.

FIG. 14 shows that GENtoniK decreased migratory marker expression and increases neuronal activity marker expression in forebrain organoids. Representative images of immunofluorescent staining for FOS, DCX, and MAP2 in day 60 forebrain organoids that received DMSO (top) or GENtoniK (bottom) from days 15 to 50. Scale bars are 50 μm.

FIGS. 15A-15E show that GENtoniK increased dynamic insulin secretion and insulin+ granules in hPSC-derived beta-like cells. FIG. 15A depicts a schematic representation of the stepwise differentiation protocol. hESC-derived immature beta-like cells were treated with GENtoniK or DMSO from days 20 to 27. FIGS. 15B-15C show dynamic KCl stimulated human insulin secretion (FIG. 15B) and area under curve (AUC, FIG. 15C) in hESC derived cells after 7 days treatment with GENtoniK or control followed by 2 days treatment-free culture. The assay was performed in the presence of 2 mM D-glucose. Fold change was calculated by dividing the amount of secreted insulin at each time point by the average amount of secreted insulin at 2 mM D-glucose. N=5 biological replicates. Fig. D shows representative electron micrographs showing immunogold labelling of insulin in beta-like cells. Circles indicate insulin+ granules (10 nm gold particles). Magnification=50,000×. FIG. 15E shows percentage of insulin+ granules in control and GENtoniK-treated beta-like cells (N=16). For FIGS. 15C and 15E, Two-tailed Student's t-test; asterisks indicate statistical significance. Error bars represent S.E.M.

FIGS. 16A-16L show synchronized generation of cortical neurons from hPSCs. FIG. 16A shows a schematic of the experimental paradigm. FIGS. 16B-16C show expression of pluripotency (FIG. 16B) and cortical (FIG. 16C) specific markers by qRT-PCR throughout the differentiation. FIGS. 16D-16E show representative images (FIG. 16D) and quantification (FIG. 16E) of the fraction of cells that expressed Pax6, FoxG1 and Nestin cortical NPC markers at d20 of differentiation. FIGS. 16F-16G show representative images (FIG. 16F) and quantification (FIG. 16G) of the percentage of Ki67⁺ NPC and MAP2⁺ neurons after induction of synchronized neurogenesis at d20. FIGS. 16H-16I show representative images (FIG. 16H) and quantification (FIG. 16I) of the fraction of d40 MAP2 neurons that were labelled by EdU pulses of progenitor cells at the indicated days. FIG. 16J shows qRT-PCR expression of Ki67 and MAP2 throughout differentiation. Neurons generated under synchronized conditions were maintained for 100 days in vitro without new proliferative events. FIG. 16K shows representative images of neurons stained with antibodies against Tbr1. FIG. 16L show quantification of the fraction of neurons expressing Tbr1, Ctip2 and Satb2 cortical neuron markers. FIG. 16B, FIG. 16C; n=3 independent experiments. FIG. 16E, FIG. 16G, FIG. 16I, FIG. 16J, FIG. 16L; n=2 independent experiments. Histograms depict mean±s.e.m. Scale bars: (FIG. 16D, FIG. 16F) 100 μm; (FIG. 16H, FIG. 16K) 50 μm.

FIGS. 17A-17L show morphological, functional and maturation of synchronized cortical neurons. FIGS. 17A-17C show representative reconstructions of neuronal morphology (FIG. 17A) and quantification of neurites length (FIG. 17B) and complexity (Sholl analysis, FIG. 17C) during maturation (n=minimum 15 neurons per time point from 2 independent experiments). FIG. 17D shows representative traces of evoked action potentials. FIG. 17E shows electrophysiological measurements of action potential amplitude and rise slope of cortical neurons over time (n=25-43 neurons per time point from 10 independent experiments). FIG. 17F shows representative traces of mEPSCs at d75. FIG. 17G shows representative maximal intensity projection of time-lapse Ca²⁺ imaging at d70. FIG. 17H shows representative traces of normalized GCaMP6m intensity in d40 (left) and d70 (right) neurons during 1 min of imaging in one FOW. Colored lines indicate Ca²⁺ traces of individual neurons while black lines represent the averaged GCaMP6m signal. FIGS. 17I-17J show quantification of amplitude and frequency of spontaneous individual Ca²⁺ spikes (FIG. 17I) and rate of synchronous firing per min of imaging in each FOW (FIG. 17J) (n=10 FOW per time point from 2 independent experiment). FIG. 17K shows representative images of neurons stained with antibodies against SynI and MAP2. FIG. 17L shows a heatmap for the normalized temporal expression of transcripts important for neuronal excitability and connectivity from RNAseq experiments (n=3 independent experiments). Data are represented as mean±s.e.m. Dots represent individual neurons in (FIG. 17B, FIG. 17E, FIG. 17F, FIG. 17I) and FOW in (FIG. 17J). Scale bars are 100 μm (FIG. 17A, FIG. 17G); (FIG. 17K) 50 μm (right) and 20 μm (left). ****p<0.0001; ***p<0.001; **p<0.01; *p<0.05. Two-tailed unpaired t-test (FIG. 17B, FIG. 17D, FIG. 17J). Welch's one-way ANOVA with Games-Howell's multiple comparisons test (FIG. 17I).

FIGS. 18A-18H show molecular staging of neuronal maturation. FIG. 18A shows a PCA plot of a RNAseq dataset showing distribution of samples according to their time of differentiation based on 1000 differentially expressed transcripts with Variance Stabilized Normalization (VST) (n=3 independent experiments). FIG. 18B shows a waterfall plot of the top 150 enriched pathways in GSEA that are positively correlated with more mature neurons in d50 vs. d25 comparison. Color codes indicate neuronal excitability/synaptic connectivity, metabolism, second messenger signaling, extracellular matrix (ECM) and immunity-related pathways. FIG. 18C shows a heatmap for the VST normalized temporal expression of strict monotonically upregulated transcripts (maximum log FC>1, maximum RPKM>5 and s.e.m. at d100<1). FIG. 18D shows representative images of neurons at indicated time-points stained with antibodies for indicated maturation markers. FIG. 18E shows a PCA plot of ATACseq dataset showing segregation of samples according to their maturation stage (n=2 independent experiments). FIG. 18F shows agglomerative hierarchical clustering by Ward linkage of differentially accessible ATACseq peaks in neurons identified 9 groups of peaks with stage-specific accessibility. FIG. 18G shows the top 15 statistically enriched transcription factor motifs at late-opening ATACseq peaks (top, group 2; bottom, group 3). Odds ratio indicate the normalized enrichment of transcription factor motifs in the cluster compared to the background. FIG. 18H show GO for genes linked at late-opening group 2 (top) and 3 (bottom) peaks show enrichment for synaptic-related pathways.

FIGS. 19A-19E shows epigenetic switch drove neuronal maturation. FIG. 19A shows a waterfall plot of GSEA enriched pathways that are negatively correlated with neuronal maturation in d50 vs. d25 comparison. Red dots indicate epigenetic-related pathways. FIG. 19B shows a heatmap for VST normalized temporal expression of chromatin regulators that are monotonically downregulated during maturation (maximum log FC>1, s.e.m. at d100<1). Gene labelled in the heatmap were selected for perturbation studies. FIG. 19C shows a schematic of experimental paradigm for gene-KO in postmitotic hPSCs-derived neurons: Cas9 expressing neurons at d25 were infected with lentiviral vectors encoding gene-specific gRNAs. Induction of preconscious molecular and functional maturation was assessed by western blot and Ca²⁺ imaging respectively. FIG. 19D shows western blot analysis for the expression of Nefh and Stx1a maturation markers upon gene-KO in neurons (2 gRNA/gene). Histograms depict average log₂ Fold change±s.e.m. over neurons transduced with non-targeting gRNA. Dots represent replicates (n=3 independent experiments). FIG. 19E shows amplitude of spontaneous Ca²⁺ spikes of individual neurons in gene-KO experiments. Dotted line represents the average amplitude of the 2 non-targeting gRNA conditions and dots represent individual neurons (n=3-6 FOW from 2 independent experiments). ****p<0.0001; ***p<0.001; **p<0.01; *p<0.05. Welch's one-way ANOVA with Games-Howell's multiple comparisons test.

FIGS. 20A-20G show transient inhibition of epigenetic factors in NPCs drove faster maturation in neurons. FIG. 20A shows temporal expression of chromatin regulators hits from gene-KO studies at hPSCs, NPCs and neuron stages. FIG. 20B shows a schematic of experimental paradigm for transient inhibition of chromatin regulators at progenitor cell stage. NPCs were treated with small molecule from d12 to d20. Control and treated NPCs were induced for synchronized neurogenesis and neurons derived from all the treatments were maintained in the same conditions. Induction of preconscious molecular and functional maturation was assessed by western blot and Ca²⁺ imaging respectively. FIG. 20C shows western blot analysis for the expression of Nefh and Stx1a maturation markers. Histograms depict average fold change±s.e.m. over DMSO controls. Dotted line represents DMSO controls. Dots represent replicates (n=2-5 independent experiments). FIGS. 20D-20E show amplitude and frequency of spontaneous Ca²⁺ spikes of individual neurons (FIG. 20D) and synchronicity rate of spontaneous network activity (FIG. 20E) in treated vs. control conditions. Dots represent individual neurons in (FIG. 20D) and FOW in (FIG. 20E) (n=3-6 FOW from 2 independent experiments). FIG. 20F shows representative traces of normalized GCaMP6m intensity in DMSO control (left) and EZH2i (right) conditions during 1 min of imaging in one FOW. Colored lines indicate Ca²⁺ traces of individual neurons while black lines represent the averaged GCaMP6m signal. FIG. 20G shows K-means clustering of differentially expressed transcripts for RNAseq studies (n=3 independent experiments). ****p<0.0001; ***p<0.001; **p<0.01; *p<0.05. Welch's one-way ANOVA with Games-Howell's multiple comparisons test (FIG. 20E); ordinary one-way ANOVA (FIG. 20F).

FIGS. 21A-21F show a novel platform for the synchronized generation of cortical neurons from hPSCs. FIG. 21A shows a schematic of the differentiation protocol based on dual-SMAD and WNT inhibition. Top panel indicate differentiation days, basal media and small molecules treatments. Bottom panel indicate cell stages/types found at transition points. The red arrow indicates cell-passaging at low density in presence of notch pathway inhibitor DAPT. FIG. 21B shows a genome browser traces of ATACseq peaks at hPSCs, NPCs and neuron stages in Pluripotency (Nanog, Oct4) and cortical (Pax6, FoxG1) loci. FIG. 21C shows cell passaging at low density and DAPT treatment rapidly depleted the pool of progenitor cells. Cells were cultured in presence or absence of DAPT from d20, pulse labelled with EdU for 24 h at d25 and analyzed at d26 by immunostaining for EdU, Ki67 and MAP2. FIGS. 21D-21E shows representative images of cortical neurons generated through synchronized neurogenesis (FIG. 21D) and spontaneous neurogenesis (FIG. 21E, cortical organoids) and stained with antibodies against cortical neurons markers. FIG. 21F show synchronized cortical neurons maintained at high viability in long-term cultures. Representative images of cortical neurons stained with antibody against MAP2 at day 25, 50, 75 and 100 of differentiation. Scale bars are 100 μm (FIG. 21C), 50 μm (FIG. 21D, FIG. 21F) and 200 μm (FIG. 21E).

FIGS. 22A & 22B show gene ontology and BrainSpan comparison for maturation dependent transcripts. FIG. 22A shows GSEA plots for some of the GO terms related that positively correlate with neuronal maturation in d50 vs. d25 and d100 vs. d50 pairwise comparisons. FIG. 22B shows a heatmap for the normalized temporal expression of the corresponding monotonically upregulated transcripts in the BrainSpan atlas of the developing human brain (primary visual cortex) shown in FIG. 18C.

FIGS. 23A & 23B show pairwise comparisons of chromatin accessibility during maturation. FIG. 23A shows MA (left) and tornado plots (right) for differential accessible ATACseq peaks in d25 vs. d50 and d50 vs. d100 pairwise comparisons. FIG. 23B shows top transcription factor motifs enriched in differentially accessible ATACseq peaks in d50 vs. d25 and d100 vs. d50 pairwise comparisons.

FIGS. 24A & 24B show motif analysis for unbiassed ATACseq clusters. FIG. 24A shows pie charts of ATACseq peaks mapped to gene promoters, introns, exons and intergenic genomic regions for each of the cluster in FIG. 18G. FIG. 24B shows the top 15 transcription factor motifs enriched in the indicated groups of ATACseq peaks. Odds ratio indicates the normalized enrichment of transcription factor motifs in the cluster compared to the background.

FIGS. 25A & 25B show chromatin regulators are progressively downregulated during neuronal maturation. FIG. 25A shows GSEA plots for GO terms related to chromatin remodeling in d50 vs. d25 and d100 vs. d50 pairwise comparisons. FIG. 25B shows a heatmap for the normalized temporal expression of the corresponding monotonically downregulated chromatin regulators in the BrainSpan atlas of the developing human brain (primary visual cortex) shown in FIG. 19B.

FIGS. 26A-26E show strategy for gene knock-out in hPSCs-derived neurons. FIG. 26A show expression of GPI gene throughout differentiation. FIG. 26B shows targeting construct for the generation on the knock-in GPI::Cas9 hPSCs line. Cas9 was linked to the GPI gene via 2A self-cleaving peptide sequence. FIG. 26C shows karyotypic analysis of the GPI::Cas9 hPSCs clonal cell line used for the study. FIG. 26D shows expression of Cas9 mRNA in the GPI::Cas9 line at hPSC, NPC and neuron stages compared to wild type hPSCs. FIG. 26E shows western blot analysis for CRISPR/Cas9-based gene KO for Chd3 and Kdm5b in neurons using the same strategy shown in FIG. 19C. Cas9 expressing neurons at d25 were infected with lentiviral vectors encoding non-targeting and gene-specific gRNAs and analyzed at day 35 of differentiation. Histograms depict mean±s.e.m.

FIGS. 27A-27D shows loss-of-function of epigenetic factors in neurons induced preconscious maturation. FIG. 27A shows a schematic of experimental paradigm for gene KO in hPSCs-derived neurons. FIG. 27B shows gene expression from RNAseq for Nefh and Stx1a maturation markers throughout differentiation. FIG. 27C shows representative western blots for the loss-of-function genetic screen of chromatin regulators. FIG. 27D shows frequency of spontaneous Ca²⁺ spikes of individual neurons (top) and synchronicity rate of spontaneous network activity (bottom) in loss-of-function experiments. Dots represent individual neurons and FOW respectively (n=3-6 FOW from 2 independent experiments). Histograms and lines depict mean±s.e.m.

FIGS. 28A-28D show transient inhibition of epigenetic factors in NPCs did not alter cortical patterning and neurogenesis. FIG. 28A shows a schematic of experimental paradigm for transient inhibition of chromatin regulators at progenitor cell stage. NPCs were treated with small molecule from d12 to d20. FIG. 28B shows small molecules and relative intracellular targets used in the study. FIG. 28C shows representative images of d20 NPCs treated with small molecule before the induction of synchronized neurogenesis and stained with antibodies against cortical markers Pax6 and FoxG1, the proliferation marker Ki67 and the neuron marker MAP2. FIG. 28D shows quantification of the fraction of cells expressing each marker in treated vs. control conditions (n=2 independent experiments). Histograms depict mean±s.e.m. Scale bars are 50 μm.

FIG. 29 shows a small molecule Mini screen identified PRC2 inhibition in NPC as a maturation driver in neurons. FIG. 29 shows representative western blots for the expression of Nefh and Stx1a maturation markers in the transient inhibition of epigenetic factors in NPC experiments. NPC were treated with small molecule from d12 to d20 and neurons derived from each condition were analyzed at d35.

FIGS. 30A-30C show EZH2, EHMT1/2 and DOT1L inhibition in NPC drove molecular maturation in neurons. FIG. 30A shows a PCA plot of RNAseq dataset showing samples distributed according to the pharmacological treatments performed at NPC stage (n=3 independent experiments). FIG. 30B shows a volcano plot for the indicated pairwise comparisons from RNAseq studies. Red dots represent differentially expressed significant transcript (FDR 0.05) that show Fold Change>=2. FIG. 30C shows GO analysis for pathways enriched in upregulated (top) and downregulated (bottom) transcripts in the indicated pairwise comparisons.

FIGS. 31A-31B show that an epigenetic switch drives neuronal maturation. FIG. 31A shows branching tree from single-cell RNAseq from Di Bella et al. (Nature 595, 554-559, (2021)) showing expression of Dcx transcripts in the mouse neocortex. FIG. 31B shows temporal expression of chromatin regulators from perturbation studies in hPSC-derived neurons (FIG. 19D) in multiple neuronal identities in the mouse neocortex. UP, upper layer; DL, lower layer; CPN, callosal projection neurons; SCPN, subcerebral projection neurons; NP, near projecting; CThPN, cortico-thalamic projection neurons.

FIGS. 32A-32F show patterns of histone post translational modifications drive the maturation of hPSC-derived neurons. FIG. 32A shows unsupervised clustering of CUT&RUN peaks with differential density in H3K27ac, H3K4me4, H3K27me3 and H3K9me3 signal among NPC and neurons (n=2 replicates/condition). FIG. 32B shows pie charts of CUT&RUN peaks mapped to gene promoters, introns, exons, and intergenic genomic regions for each of the cluster. FIG. 32C shows GO for genes linked at each cluster. FIG. 32D shows top selected statistically significant enriched transcription factor motifs at peaks in each cluster. FIG. 32E shows mean normalized expression (z-transform) of differentially expressed genes during the maturation time course intersected with genes linked to each CUT&RUN cluster. FIG. 32F shows expression of differentially expressed transcripts from (FIG. 32E) in neurons derived from NPC treated with the indicated inhibitors respect to DMSO controls. Pink area in (FIG. 32E) is S.E.M. and whiskers in (FIG. 32F) depict 1.5*interquartile range beyond the 25th and 75th percentiles.

FIGS. 33A-33H show that an epigenetic barrier in NPCs controls the onset of maturation programs. FIG. 33A shows heatmap for cluster 1 from FIGS. 32A-32F representing bivalent peaks in NPCs decorated by H3K27me3 and H3K4me3 that lose H3K27me3 at neuron stage (n=2 replicate for each condition). FIG. 33B shows heatmap for the normalized expression of representative transcripts within the bivalent genes in d35 neurons derived from NPC treated with DOT1l, EHMT1/2 and EZH2 inhibitors at 2 and 4 μM (n=3 replicate for each condition). FIGS. 33C-33D show Representative Integrative Genome Viewer density tracks for H3K27me, H3K4mc3, and H3K27ac at indicated genomic loci in NPC and neurons (n=2 replicate for each condition). FIG. 33E shows temporal expression of CHD5 and JADE2 transcripts through the maturation time-course (n=3 independent experiments). FIG. 33F shows expression of CHD5 and JADE2 transcripts in d35 neurons derived from NPC treated with DOT1l, EHMT1/2 and EZH2 inhibitors at 2 and 4 μM (n=3 replicate for each condition). Histograms depict mean±s.e.m. FIGS. 33G-33H show the schematic of the main conclusion of the study. FIG. 33G shows the temporal unfolding of maturation signatures in hPSC-derived neurons proceed gradually and is marked by the retention of multiple epigenetic pathways that establish an epigenetic barrier at progenitor cell stage that gets inherited in neurons. FIG. 33H shows key members of the epigenetic barrier, including EZH2, maintain maturation programs in a poised state through deposition of repressive histone marks.

FIGS. 34A-34E illustrate characterization of the effect of EZH2 transient inhibition at progenitor cell stage on the electrophysiological properties of hPSC-derived cortical neurons. FIGS. 34A and 24B show intrinsic firing properties. FIG. 34A shows representative traces at d50 (+20 pA injected current). FIG. 34B shows quantification of the number of spikes per injected current (Control d30 n=7, d50 n=11; EZH2i d30 n=8, d50 n=12; two-way ANOVA), total number of spikes and amplitude (Control d30 n=7, d50 n=11; EZH2i d30 n=8, d50 n=12; unpaired t-tests). FIG. 34C shows mEPSC quantification recorded at +40 mV. Representative traces of the mEPSC; average of mEPSCs of 5 cells (control) vs 8 cells (EZH2i) and quantification of the frequency and amplitude. The quantification was performed taking all the events together (cumulative probability plots depicted in FIGS. 34D and 34E; Kolmogorov-Smirnov) or with the averaged frequency or amplitude for each cell (insets, unpaired t-tests).

FIGS. 35A-35C illustrate characterization of the effect of EZH2 transient inhibition on neuronal activity in hPSC-derived brain cortical organoids. FIG. 35A shows representative image of GCAMP6m signal by light-sheet microscopy in intact brain cortical organoids at day 55 of differentiation. FIGS. 35B and 35C show quantification of amplitude and frequency of spontaneous individual Ca2+ spikes in WA09 hESC-derived (FIG. 35B) and MSK-SRF001 iPSC-derived cortical brain organoids (FIG. 35C) treated transiently (day 17-day 26) with the EZH2 inhibitor GSK343. Data are represented as mean±s.e.m. Dots represent individual neurons from 2 independent batches of organoid differentiation. 2-3 organoids/batch for each treatment. Unpaired t-test with Welch's correction.

FIG. 36 depicts characterization of the effect of EZH2 transient inhibition at progenitor cell stage in hPSC-derived cortical neurons co-cultured with rat astrocytes. Quantification of amplitude, frequency and synchronicity of spontaneous individual Ca2+ spikes in cortical neurons derived from progenitor cells treated transiently with the EZH2 inhibitor GSK343. Neurons were plated on rat cortical astrocytes at day 25 of differentiation. Dots represent individual neurons (Amplitude and frequency) and FOW (synchronicity) from 2 independent differentiations. Mann-Whitney test.

FIG. 37 shows validation of maturation and epigenetic signatures across neurons derived from multiple human Pluripotent Stem Cell lines. qRT-PCR z-scored normalized expression for indicated transcripts at the indicated time points of differentiation in WA09 and WA01 hESC-derived cortical neurons and in MSK-SRF001 iPSC-derived cortical neurons (2 independent differentiations/each line). Data are represented as mean±s.e.m.

FIG. 38 shows validation of functional phenotypes in hPSC-derived cortical neurons derived from transient epigenetic inhibition at progenitor cell stage across human Pluripotent Stem Cell lines. Quantification of amplitude, frequency and synchronicity of spontaneous individual Ca2+ spikes in WA01 hESC-derived and MSK-SRF001 iPSC-derived cortical neurons. Neurons were derived from progenitor cells treated with epigenetic inhibitors as indicated (C). Data are represented as mean±s.e.m. Dots represent individual neurons (Amplitude and frequency) and FOW (synchronicity) from 2 independent differentiations. Data are represented as mean±s.e.m. Welch's one-way ANOVA with Games-Howell's multiple comparisons test.

### 5. DETAILED DESCRIPTION

The present disclosure relates to compositions, kits, and methods for promoting in vitro maturation of cells, for example, cells in vitro differentiated from stem cells. The present disclosure is partly based on the discovery that among thousands of compounds screened, inhibitors of epigenetic regulators and agonists of calcium channels were identified as compounds that can drive neuron maturation. The present disclosure further discovered that a combination of four compounds, including GSK2879552, EPZ-5676, NMDA and Bay K 8644, triggered cortical neuron maturation across all initial and additional orthogonal assays including synaptic density, electrophysiology, and transcriptomics. Surprisingly, the combination of the 4 compounds was effective in maturing cortical neurons, 3D cortical organoids, spinal motoneurons, and non-neural cell types, such as melanocytes and pancreatic beta cells.

Non-limiting embodiments of the present disclosure are described by the present specification and Examples.

For purposes of clarity of disclosure and not by way of limitation, the detailed description is divided into the following subsections:
5.1. Definitions;
5.2. Compositions for promoting in vitro maturation of cells;
5.3. Methods of promoting in vitro maturation of cells;
5.4. Cell populations and compositions;
5.5. Methods of screening maturation-promoting compounds;
5.6. Kits; and
5.7. Exemplary Embodiments.

### 5.1. Definitions

The terms used in this specification generally have their ordinary meanings in the art, within the context of the present disclosure and in the specific context where each term is used. Certain terms are discussed below, or elsewhere in the specification, to provide additional guidance to the practitioner in describing the compositions and methods of the present disclosure and how to make and use them.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold, or within 2-fold, of a value.

“Inhibitor” as used herein, refers to a compound or molecule (e.g., small molecule, peptide, peptidomimetic, natural compound, siRNA, anti-sense nucleic acid, aptamer, or antibody) that interferes with (e.g., reduces, decreases, suppresses, eliminates, or blocks) the function and/or activity of a molecule (e.g., lysine-specific demethylase 1 (LSD1) inhibitor and disruptor of telomerase-like 1 (DOT1L)). For example, an inhibitor of LSD1 can function, for example, via directly contacting LSD1, contacting LSD1 mRNA, causing conformational changes of LSD1, decreasing the LSD1 protein level, or interfering with LSD1's interactions with its target molecules (e.g., a monomethylated or dimethylated lysine), and affecting the expression of LSD1 target genes.

“Agonists,” as used herein, refer to compounds that increase, induce, stimulate, activate, facilitate, or enhance activation the function of a molecule, e.g., glutamate receptors, and L-type calcium channel (LTCC)).

As used herein, the term “derivative” refers to a chemical compound with a similar core structure.

As used herein, the term “stem cell” refers to a cell with the ability to divide for indefinite periods in culture and to give rise to specialized cells.

As used herein, the term “embryonic stem cell” and “ESC” refer to a primitive (undifferentiated) cell that is derived from preimplantation-stage embryo, capable of dividing without differentiating for a prolonged period in culture, and are known to develop into cells and tissues of the three primary germ layers. A human embryonic stem cell refers to an embryonic stem cell that is from a human embryo. As used herein, the term “human embryonic stem cell” or “hESC” refers to a type of pluripotent stem cells derived from early stage human embryos, up to and including the blastocyst stage, that is capable of dividing without differentiating for a prolonged period in culture, and are known to develop into cells and tissues of the three primary germ layers.

As used herein, the term “embryonic stem cell line” refers to a population of embryonic stem cells which have been cultured under in vitro conditions that allow proliferation without differentiation for up to days, months to years.

As used herein, the term “pluripotent” refers to an ability to develop into the three developmental germ layers of the organism including endoderm, mesoderm, and ectoderm.

As used herein, the term “multipotent” refers to an ability to develop into more than one cell type of the body.

As used herein, the term “induced pluripotent stem cell” or “iPSC” refers to a type of pluripotent stem cell formed by the introduction of certain embryonic genes (such as but not limited to OCT4, SOX2, and KLF4 transgenes) (see, for example, Takahashi and Yamanaka Cell 126, 663-676 (2006), herein incorporated by reference) into a somatic cell.

As used herein, the term “neuron” refers to a nerve cell, the principal functional units of the nervous system. A neuron consists of a cell body and its processes—an axon and at least one dendrite. Neurons transmit information to other neurons or cells by releasing neurotransmitters at synapses.

As used herein, the term “differentiation” refers to a process whereby an unspecialized embryonic cell acquires the features of a specialized cell such as a neuron, heart, liver, or muscle cell. Differentiation is controlled by the interaction of a cell's genes with the physical and chemical conditions outside the cell, usually through signaling pathways involving proteins embedded in the cell surface.

As used herein, the term “directed differentiation” refers to a manipulation of stem cell culture conditions to induce differentiation into a particular (for example, desired) cell type, such as midbrain dopamine neurons or precursors thereof. In references to a stem cell, “directed differentiation” refers to the use of small molecules, growth factor proteins, and other growth conditions to promote the transition of a stem cell from the pluripotent state into a more mature or specialized cell fate.

As used herein, the term “inducing differentiation” in reference to a cell refers to changing the default cell type (genotype and/or phenotype) to a non-default cell type (genotype and/or phenotype). Thus, “inducing differentiation in a stem cell” refers to inducing the stem cell (e.g., human stem cell) to divide into progeny cells with characteristics that are different from the stem cell, such as genotype (e.g., change in gene expression as determined by genetic analysis such as a microarray) and/or phenotype (e.g., change in expression of a protein marker)

As used herein, the term “cell culture” refers to a growth of cells in vitro in an artificial medium for research or medical treatment.

As used herein, the term “culture medium” refers to a liquid that covers cells in a culture vessel, such as a Petri plate, a multi-well plate, and the like, and contains nutrients to nourish and support the cells. Culture medium may also include growth factors added to produce desired changes in the cells.

As used herein, the term “contacting” a cell or cells with a compound (e.g., at least one inhibitor, activator, and/or inducer) refers to providing the compound in a location that permits the cell or cells access to the compound. The contacting may be accomplished using any suitable method. For example, contacting can be accomplished by adding the compound, in concentrated form, to a cell or population of cells, for example in the context of a cell culture, to achieve the desired concentration. Contacting may also be accomplished by including the compound as a component of a formulated culture medium.

As used herein, the term “in vitro” refers to an artificial environment and to processes or reactions that occur within an artificial environment. in vitro environments exemplified, but are not limited to, test tubes and cell cultures.

As used herein, the term “in vivo” refers to the natural environment (e.g., an animal or a cell) and to processes or reactions that occur within a natural environment, such as embryonic development, cell differentiation, neural tube formation, etc.

As used herein, the term “derived from” or “established from” or “differentiated from” when made in reference to any cell disclosed herein refers to a cell that was obtained from (e.g., isolated, purified, etc.) an ultimate parent cell in a cell line, tissue (such as a dissociated embryo, or fluids using any manipulation, such as, without limitation, single cell isolation, culture in vitro, treatment and/or mutagenesis using for example proteins, chemicals, radiation, infection with virus, transfection with DNA sequences, such as with a morphogen, etc., selection (such as by serial culture) of any cell that is contained in cultured parent cells. A derived cell can be selected from a mixed population by virtue of response to a growth factor, cytokine, selected progression of cytokine treatments, adhesiveness, lack of adhesiveness, sorting procedure, and the like.

An “individual” or “subject” herein is a vertebrate, such as a human or non-human animal, for example, a mammal. Mammals include, but are not limited to, humans, non-human primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, the term “immature cells” refers to fully differentiated cells that have acquired the identity of an adult cell type, but do not yet display the full range of characteristics and functionality of the adult form.

As used herein, the term “progenitor cells” refers to partially differentiated cells that can give rise to several types of adult cells.

As used herein, the term “precursor cells” refers to partially differentiated cells that can give rise to one type of adult cell.

As used herein, the term “adult-like function” refers to the set of activities and behaviors that enable a cell to fulfill its role in the adult body.

As used herein, the term “disease-relevant phenotype” refers to cellular properties and functions that are necessary for the manifestation of a particular disease.

### 5.2. Compositions for Promoting In Vitro Maturation of Cells

The present disclosure provides compositions for promoting in vitro maturation of cells (e.g., immature cells, precursors or progenitors disclosed in Section 5.3 of the present disclosure). In certain embodiments, the composition comprises at least one inhibitor of an epigenetic regulator. In certain embodiments, the composition comprises at least one inhibitor of an epigenetic regulator, and at least one agonist of a calcium channel.

In certain embodiments, the epigenetic regulator is lysine-specific demethylase 1 (LSD1), disruptor of telomerase-like 1 (DOT1L), REST corepressor (CoREST), enhancer of zeste homolog 2 (EZH2), euchromatic histone-lysine-N-methyltransferases 1 and 2 (EHMT1/2), or a combination thereof. In certain embodiments, the at least one inhibitor of an epigenetic regulator comprises an LSD1 inhibitor, a DOT1L inhibitor, a CoREST inhibitor, an EZH2 inhibitor, an EHMT1/2 inhibitor, or a combination thereof.

Lysine-specific demethylase 1 (LSD1) (also known as KDM1A, KIAA0601, BHC110, and AOF2) is a flavin-dependent monoamine oxidase (MAO) protein. LSD1 can specifically demethylates histone lysine residues H3K4me1/2 or H3K9me1/2, and thus repress or activates gene expression respectively. Non-limiting examples of LSD1 inhibitor that can be used with the present invention include GSK2879552, OG-L002, GSK-LSD1, derivatives thereof, and combinations thereof. In certain embodiments, the LSD1 inhibitor is GSK2879552.

GSK2879552 (also known as GT77Z6Y09Z) has the IUPAC name 4-[[4-[[[(1R,2S)-2-phenylcyclopropyl]amino]methyl]piperidin-1-yl]methyl]benzoic acid with the following chemical structure:

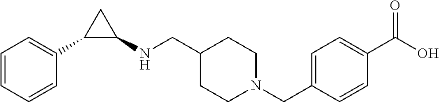 [SMILES: O=C(O)c1ccc(CN2CCC(CN[C@@H]3C[C@H]3c3ccccc3)CC2)cc1]

GSK2879552 can selectively and irreversibly inhibits LSD1.

Disruptor of telomerase-like 1 (DOT1L), also known as DOT1, KMT4, and DOT1 like histone lysine methyltransferase, is a histone H3K79-specific methyltransferase and catalyzes the mono-, di-, and trimethylation of H3K79. Non-limiting examples of DOT1L inhibitor that can be used with the present invention include EPZ-5676, EPZ004777, SYC-522, SGC0946, Dot1L-IN-2, Dot1L-IN-4, Dot1L-IN-5, Dot1L-IN-6, CN-SAH, derivatives thereof, and combinations thereof. In certain embodiments, the inhibitor of DOT1L is EPZ-5676.

EPZ-5676 (also known as pinometostat) has the IUPAC name (2R,3R,4S,5R)-2-(6-aminopurin-9-yl)-5-[[[3-[2-(6-tert-butyl-1H-benzimidazol-2-yl)ethyl]cyclobutyl]-propan-2-ylamino]methyl]oxolane-3,4-dioland, with the following chemical structure:

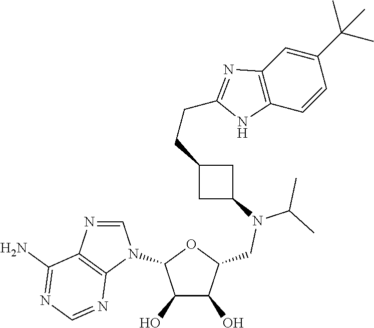 [SMILES: CC(C)N(C[C@H]1O[C@@H](n2cnc3c(N)ncnc32)[C@H](O)[C@@H]1O)[C@H]1C[C@@H](CCc2nc3cc(C(C)(C)C)ccc3[nH]2)C1]

EPZ5676 is a potent inhibitor of DOT1L that occupies the S-adenosyl methionine (SAM) binding pocket of DOT1L and induces conformational changes in DOT1L resulting in the opening of a hydrophobic pocket beyond the amino acid portion of SAM.

EPZ004777 has the IUPAC name 1-[3-[[(2R,3S,4R,5R)-5-(4-aminopyrrolo[2,3-d]pyrimidin-7-yl)-3,4-dihydroxyoxolan-2-yl]methyl-propan-2-ylamino]propyl]-3-(4-tert-butylphenyl)urea, with the following chemical structure:

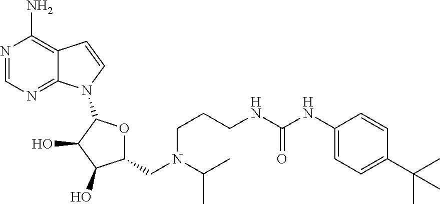

EPZ004777 is a potent, selective DOT1L inhibitor.

Enhancer of zeste homolog 2 (EZH2) is a histone-lysine N-methyltransferase enzyme that participates in histone methylation and transcriptional repression. EZH2 catalyzes the addition of methyl groups to histone H3 at lysine 27, by using the cofactor S-adenosyl-L-methionine. Non-limiting examples of EZH2 inhibitors that can be used with the present invention include 3-deazaneplanocin A (DZNep), GSK343, GSK126, EPZ-6438, EPZ005687, GSK926, EPZ6438, EPZ011989, CPI-1205, CPI-169, ZLD1039, PF-06821497, UNC1999, PR-S1/OR-S2, DS-3201b, A-395, EBI-2511, EED226, EEDi-5285, EI1, EZH2-IN-2, EZH2-IN-3, EZH2-IN-4, EZH2-IN-5, GNA002, GSK503, JQEZ5, MAK683, MS1943, PF-06726304, UNC 1999, UNC6852, UNC6852, AM41-44A, BR-001, CPI-1328, CPI-905, DCE_254, EBI-2511, YM181, YM181, ZLD1039, ZLD10A, derivatives thereof, and combinations thereof. In certain embodiments, the EZH2 inhibitor is GSK343.

GSK343 has the IUPAC name N-[(6-methyl-2-oxo-4-propyl-1H-pyridin-3-yl)methyl]-6-[2-(4-methylpiperazin-1-yl)pyridin-4-yl]-1-propan-2-ylindazole-4-carboxamide, with the following chemical structure:

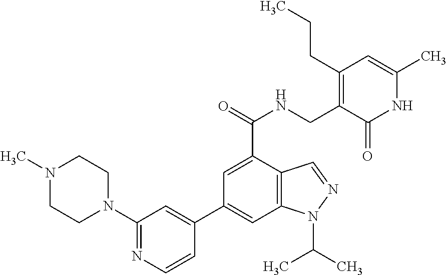

GSK343 is a highly potent and selective EZH2 inhibitor.

Euchromatic histone-lysine-N-methyltransferases 1 and 2 (EHMT1/2) catalyze dimethylation of histone H3 lysine 9 (H3K9me2) and have roles in epigenetic silencing of gene expression. Non-limiting examples of EHMT1/2 inhibitors that can be used with the present invention include UNC0638, UNC0224, UNC0321, UNC0642, UNC0646, UNC0642, UNC0631, A-366, BIX-01294, BRD4770, BRD9539, CM-272, CM-579, CPUY074020, CSV0C018875, EHMT2-IN-1, EHMT2-IN-2, EML741, derivatives thereof, and combinations thereof. In certain embodiments, the EHMT1/2 inhibitor is UNC0638.

UNC0638 has the IUPAC name 2-cyclohexyl-6-methoxy-N-(1-propan-2-ylpiperidin-4-yl)-7-(3-pyrrolidin-1-ylpropoxy)quinazolin-4-amine, with the following chemical structure:

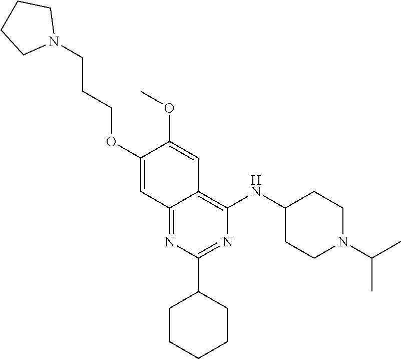 [SMILES: COc1cc2c(NC3CCN(C(C)C)CC3)nc(C3CCCCC3)nc2cc1OCCCN1CCCC1]

UNC0638 is a potent, selective and cell-penetrant chemical probe for G9a and GLP histone methyltransferase.

REST corepressor (CoREST) is known to be a corepressor of the neuronal-specific genes silencer, REST (RE1 silencing transcription factor/neural restrictive silencing factor). The repression function of the REST/CoREST complex is carried out through CoREST, stimulates demethylation on core histones and promotes demethylation of nucleosomal substrates through enhancing the association among histone demethylase and nucleosomes.

In certain embodiments, the agonist of a calcium channel comprises a glutamate receptor agonist, an L-type calcium channel (LTCC) agonist, a ryanodine receptor (RYR) agonist, an inositol trisphosphate receptor (InsP3R) agonist, or a combination thereof.

Non-limiting examples of glutamate receptor agonists that can be used with the present invention include NMDA, (RS)-(Tetratazol-5-yl)glycine, ibotenic acid, derivatives thereof, and combinations thereof.

NMDA, also known as N-methyl-d-aspartic acid or N-methyl-d-aspartate, has the IUPAC name (2R)-2-(methylamino)butanedioic acid, with the following chemical structure:

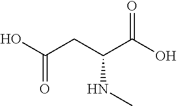

NMDA is an agonist of NMDA receptor (NMDAR), which a subtype of the ionotropic glutamate receptor. Activated NMDAR allows the influx of Ca2+ into the cell.

Non-limiting examples of LTCC agonist that can be used with the present invention include Bay K 8644, FPL 64176, derivatives thereof, and combinations thereof.

Bay K 8644 has the IUPAC name methyl 2,6-dimethyl-5-nitro-4-[2-(trifluoromethyl)phenyl]-1,4-dihydropyridine-3-carboxylate, with the following chemical structure:

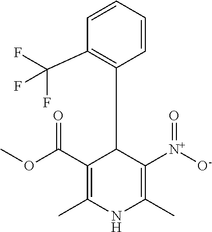 [SMILES: COC(=O)C1=C(C)NC(C)=C([N+](=O)[O-])C1c1ccccc1C(F)(F)F]

Non-limiting examples of RYR agonist that can be used with the present invention include BAYK 8644, S107, Chlorantraniliprole, Lomifylline, Ryanodol, MBED, derivatives thereof, and combinations thereof.

In certain embodiments, the composition comprises at least two inhibitors of an epigenetic regulator and at least two agonists of a calcium channel. In certain embodiments, the composition comprises an LSD1 inhibitor, a DOT1L inhibitor, a glutamate receptor agonist, and an LTCC agonist. In certain embodiments, the composition comprises GSK2879552, EPZ-5676, NMDA, and Bay K 8644.

In certain embodiments, the composition comprises at least one inhibitor of an epigenetic regulator. In certain embodiments, the composition comprises an EZH2 inhibitor, an EHMT1/2 inhibitor, a DOT1L inhibitor, or a combination thereof. In certain embodiments, the composition comprises GSK343, UNC0638, EPZ004777, or a combination thereof.

In certain embodiments, the composition comprises at least one inhibitor of an epigenetic regulator. In certain embodiments, the concentration of each inhibitor of the epigenetic regulator in the composition is between about 0.1 μM and about 10 μM, between about 0.1 μM and about 5 μM, between about 0.1 μM and about 2.5 μM, between about 0.1 μM and about 1.5 μM, between about 0.5 μM and about 10 μM, between about 0.5 μM and about 5 μM, between about 0.5 μM and about 2.5 μM, between about 0.5 μM and about 1.5 μM, between about 1 μM and about 10 μM, between about 1 μM and about 5 μM, between about 1 μM and about 2.5 μM, between about 1 μM and about 2 μM, between about 2 μM and about 5 μM, between about 2 μM and about 4 μM, between about 3 μM and about 5 μM, between about 3 μM and about 4 μM, or between about 5 μM and about 10 μM. In certain embodiments, the concentration of each inhibitor of the epigenetic regulator in the composition is between about 0.5 μM and about 1.5 μM. In certain embodiments, the concentration of each inhibitor of the epigenetic regulator in the composition is between about 0.5 μM and about 1 μM. In certain embodiments, the concentration of each inhibitor of the epigenetic regulator in the composition is between about 1 μM and about 2 μM. In certain embodiments, the concentration of each inhibitor of the epigenetic regulator in the composition is between about 2 μM and about 4 μM. In certain embodiments, the concentration of each inhibitor of the epigenetic regulator in the composition is about 1 μM. In certain embodiments, the concentration of each inhibitor of the epigenetic regulator in the composition is about 2 μM. In certain embodiments, the concentration of each inhibitor of the epigenetic regulator in the composition is about 4 μM. In certain embodiments, the at least one inhibitor of the epigenetic regulator comprises an LSD1 inhibitor, a DOT1L inhibitor, an EZH2 inhibitor, an EHMT1/2 inhibitor, or a combination thereof. In certain embodiments, the at least one inhibitor of the epigenetic regulator comprises GSK2879552, EPZ-5676, GSK343, UNC0638, EPZ004777, a derivative thereof, or a combination thereof. In certain embodiments, the at least one inhibitor of the epigenetic regulator comprises GSK2879552 and EPZ-5676. In certain embodiments, the at least one inhibitor of the epigenetic regulator comprises GSK343. In certain embodiments, the at least one inhibitor of the epigenetic regulator comprises UNC0638. In certain embodiments, the at least one inhibitor of the epigenetic regulator comprises EPZ004777.

In certain embodiments, the composition comprises at least one agonist of a calcium channel. In certain embodiments, the concentration of each agonist of the calcium channel in the composition is between about 0.1 μM and about 10 μM, between about 0.1 μM and about 5 μM, between about 0.1 μM and about 2.5 μM, between about 0.1 μM and about 1.5 μM, between about 0.5 μM and about 10 μM, between about 0.5 μM and about 5 μM, between about 0.5 μM and about 2.5 μM, between about 0.5 μM and about 1.5 μM, between about 1 μM and about 10 μM, between about 1 μM and about 5 μM, between about 1 μM and about 2.5 μM, or between about 5 μM and about 10 μM. In certain embodiments, the concentration of each agonist of the calcium channel in the composition is between about 0.5 μM and about 1.5 μM. In certain embodiments, the concentration of each agonist of the calcium channel in the composition is between about 0.5 μM and about 1 μM. In certain embodiments, the concentration of each agonist of the calcium channel in the composition is about 1 μM. In certain embodiments, the at least one agonist of the calcium channel comprises a glutamate receptor agonist, an LTCC agonist, or a combination thereof. In certain embodiments, the at least one agonist of the calcium channel comprises NMDA, Bay K 8644, a derivative thereof, or a combination thereof. In certain embodiments, the at least one agonist of the calcium channel comprises NMDA and Bay K 8644.

In certain embodiments, the composition is prepared from a stock composition, where the concentration of each component in the stock composition is at least about 2 times (e.g., about 5 times, about 10 times, about 50 times, about 100 times, about 500 times, about 1000 times) of the concentration of each component in the composition. In certain embodiments, the concentration of each component in the stock composition is at about 1000 times of the concentration of each component in the composition.

### 5.3. Methods of Promoting In Vitro Maturation of Cells

The present disclosure provides in vitro methods for promoting the maturation of cells. In certain embodiments, the method comprises contacting the cells with at least one inhibitor of an epigenetic regulator, and at least one agonist of a calcium channel (e.g., the inhibitors of an epigenetic regulator and the agonists of a calcium channel disclosed in Section 5.2. of the present disclosure). In certain embodiments, the method comprises contacting the cells with at least two inhibitors of an epigenetic regulator and at least two agonists of a calcium channel (e.g., the inhibitors of an epigenetic regulator and the agonists of a calcium channel disclosed in Section 5.2. of the present disclosure). In certain embodiments, the method comprises contacting the cells with an LSD1 inhibitor, a DOT1L inhibitor, a glutamate receptor agonist, and an LTCC agonist (e.g., the LSD1 inhibitors, DOT1L inhibitors, glutamate receptor agonist, and LTCC agonist disclosed in Section 5.2. of the present disclosure). In certain embodiments, the method comprises contacting the cells with GSK2879552, EPZ-5676, NMDA, and Bay K 8644.

In certain embodiments, the method comprises contacting the cells with at least one inhibitor of an epigenetic regulator (e.g., the inhibitors of an epigenetic regulator disclosed in Section 5.2. of the present disclosure). In certain embodiments, the method comprises contacting the cells with a DOT1L inhibitor (e.g., the DOT1L inhibitors disclosed in Section 5.2. of the present disclosure). In certain embodiments, the method comprises contacting the cells with an EZH2 inhibitor (e.g., the EZH2 inhibitors disclosed in Section 5.2. of the present disclosure). In certain embodiments, the method comprises contacting the cells with an EHMT1/2 inhibitor (e.g., the EHMT1/2 inhibitors disclosed in Section 5.2. of the present disclosure). In certain embodiments, the method comprises contacting the cells with GSK343, or a derivative thereof. In certain embodiments, the method comprises contacting the cells with UNC0638, or a derivative thereof. In certain embodiments, the method comprises contacting the cells with EPZ004777, or a derivative thereof.

In certain embodiments, the method comprises contacting the cells with a presently disclosed composition (e.g., the compositions disclosed in Section 5.2. of the present disclosure).

In certain embodiments, the cells are immature cells, progenitor cells, precursor cells, or a combination thereof. In certain embodiments, the presently disclosed methods promotes, accelerates, or induces the maturation or differentiation of the cells (e.g., immature cells, progenitor cells, precursor cells, or a combination thereof) into cells that have adult-like function or disease-relevant phenotype.

In certain embodiments, the cells comprise neuronal cells. In certain embodiments, the neuronal cells are immature neuronal cells, precursors thereof, progenitors thereof, or a combination thereof. In certain embodiments, the neuronal cells are selected from the group consisting of cortical neurons, spinal motor neurons, midbrain dopamine neurons, medium spiny neurons, interneurons, sensory neurons, enteric neurons, and combinations thereof.

In certain embodiments, the cells form a brain organoid, where the methods promote the maturation of the brain organoid. In certain embodiments, the brain organoid is a dorsal forebrain organoid, ventral forebrain organoid, midbrain organoid, spinal organoid, neuromuscular assembloid, or a combination thereof.

In certain embodiments, the cells comprise non-neuronal cells. In certain embodiments, the neuronal cells are immature non-neuronal cells, precursors thereof, progenitors thereof, or a combination thereof. In certain embodiments, the non-neuronal cells are selected from the group consisting of pancreatic beta cells, melanocytes, glial cells, myocytes, and combinations thereof.

In certain embodiments, the cells are obtained from a tissue of a subject (e.g., embryos, fetuses, developing tissues). In certain embodiments, the tissue of origin is embryonic rodent brain.

In certain embodiments, the cells are in vitro differentiated from stem cells (e.g., human stem cells). In certain embodiments, the stem cells are pluripotent stem cells. In certain embodiments, the stem cells are multipotent stem cells. Non-limiting examples of stem cells that can be used with the presently disclosed methods include nonembryonic stem cells, embryonic stem cells, induced pluripotent stem cells, engineered pluripotent stem cells, parthenogenetic stem cells, primordial germ cell-like pluripotent stem cells, epiblast stem cells, F-class pluripotent stem cells, embryonic neural stem cells, adult neural stem cells, and long-term self-renewing neural stem cell. In certain embodiments, the stem cells are human stem cells. Non-limiting examples of human stem cells include human embryonic stem cells (hESC), human pluripotent stem cell (hPSC), human induced pluripotent stem cells (hiPSC), human parthenogenetic stem cells, primordial germ cell-like pluripotent stem cells, epiblast stem cells, F-class pluripotent stem cells, somatic stem cells, cancer stem cells, or any other cell capable of lineage specific differentiation. In certain embodiments, the stem cells are non-human stem cells. In certain embodiments, the stem cell is a nonhuman primate stem cell. In certain embodiments, the stem cell is a rodent stem cell.

In certain embodiments, the concentration of each of the at least one inhibitor of an epigenetic regulator contacted with or exposed to the cells is between about 0.1 μM and about 10 μM, between about 0.1 μM and about 5 μM, between about 0.1 μM and about 2.5 μM, between about 0.1 μM and about 1.5 μM, between about 0.5 μM and about 10 μM, between about 0.5 μM and about 5 μM, between about 0.5 μM and about 2.5 μM, between about 0.5 μM and about 1.5 μM, between about 1 μM and about 10 μM, between about 1 μM and about 5 μM, between about 1 μM and about 2.5 μM, between about 1 μM and about 2 μM, between about 2 μM and about 5 μM, between about 2 μM and about 4 μM, between about 3 μM and about 5 μM, between about 3 μM and about 4 μM, or between about 5 μM and about 10 μM. In certain embodiments, the concentration of each of the at least one inhibitor of the epigenetic regulator contacted with or exposed to the cells is between about 0.5 μM and about 1.5 μM. In certain embodiments, the concentration of each of the at least one inhibitor of the epigenetic regulator contacted with or exposed to the cells is between about 0.5 μM and about 1 μM. In certain embodiments, the concentration of each of the at least one inhibitor of the epigenetic regulator contacted with or exposed to the cells is between about 1 μM and about 2 μM. In certain embodiments, the concentration of each of the at least one inhibitor of the epigenetic regulator contacted with or exposed to the cells is between about 2 μM and about 4 μM. In certain embodiments, the concentration of the at least one inhibitor of the epigenetic regulator contacted with or exposed to the cells is about 1 μM. In certain embodiments, the concentration of the at least one inhibitor of the epigenetic regulator contacted with or exposed to the cells is about 2 μM. In certain embodiments, the concentration of the at least one inhibitor of the epigenetic regulator contacted with or exposed to the cells is about 4 μM. In certain embodiments, the at least one inhibitor of the epigenetic regulator comprises an LSD1 inhibitor, a DOT1L inhibitor, an EZH2 inhibitor, an EHMT1/2 inhibitor, or a combination thereof. In certain embodiments, the at least one inhibitor of the epigenetic regulator comprises GSK2879552, EPZ-5676, GSK343, UNC0638, EPZ004777, or a combination thereof. In certain embodiments, the at least one inhibitor of the epigenetic regulator comprises GSK2879552 and EPZ-5676. In certain embodiments, the at least one inhibitor of the epigenetic regulator comprises GSK343. In certain embodiments, the at least one inhibitor of the epigenetic regulator comprises UNC0638. In certain embodiments, the at least one inhibitor of the epigenetic regulator comprises EPZ004777.

In certain embodiments, the concentration of each of the agonist of the calcium channel contacted with or exposed to the cells is between about 0.1 μM and about 10 μM, between about 0.1 μM and about 5 μM, between about 0.1 μM and about 2.5 μM, between about 0.1 μM and about 1.5 μM, between about 0.5 μM and about 10 μM, between about 0.5 μM and about 5 μM, between about 0.5 μM and about 2.5 μM, between about 0.5 μM and about 1.5 μM, between about 1 μM and about 10 μM, between about 1 μM and about 5 μM, between about 1 μM and about 2.5 μM, or between about 5 μM and about 10 μM. In certain embodiments, the concentration of each of the agonist of the calcium channel contacted with or exposed to the cells is between about 0.5 μM and about 1.5 μM. In certain embodiments, the concentration of each of the at least one inhibitor of the epigenetic regulator contacted with or exposed to the cells is between about 0.5 μM and about 1 μM. In certain embodiments, the concentration of each of the agonist of the calcium channel contacted with or exposed to the cells is about 1 μM. In certain embodiments, the at least one agonist of the calcium channel comprises a glutamate receptor agonist, an LTCC agonist, or a combination thereof. In certain embodiments, the at least one agonist of the calcium channel comprises NMDA, Bay K 8644, or a combination thereof. In certain embodiments, the at least one agonist of the calcium channel comprises NMDA and Bay K 8644.

In certain embodiments, the cells are contacted with the at least one inhibitor of the epigenetic regulator and the at least one agonist of the calcium channel for at least about 3 days and/or for up to about 30 days. In certain embodiments, the cells are contacted with the at least one inhibitor of the epigenetic regulator and the at least one agonist of the calcium channel for about 3 days, about 5 days, about 8 days, about 10 days, about 15 days, about 20 days, about 25 days, or about 30 days.

### 5.4. Cell Populations and Compositions

The presently disclosure provides a cell population of in vitro maturated cells obtained by the methods disclosed herein, for example, in Section 5.3. In addition, the present disclosure provides compositions comprising any of the in vitro maturated cells disclosed herein.

In certain embodiments, the cells are comprised in a composition that further comprises a biocompatible scaffold or matrix, for example, a biocompatible three-dimensional scaffold that facilitates tissue regeneration when the cells are implanted or grafted to a subject. In certain embodiments, the biocompatible scaffold comprises extracellular matrix material, synthetic polymers, cytokines, collagen, polypeptides or proteins, polysaccharides including fibronectin, laminin, keratin, fibrin, fibrinogen, hyaluronic acid, heparin sulfate, chondroitin sulfate, agarose or gelatin, and/or hydrogel. (See, e.g., U.S. Publication Nos. 2015/0159135, 2011/0296542, 2009/0123433, and 2008/0268019, the contents of each of which are incorporated by reference in their entireties).

In certain embodiments, the composition comprises a cell population of from about 1×10⁴ to about 1×10¹⁰, from about 1×10⁴ to about 1×10⁵, from about 1×10⁵ to about 1×10⁹, from about 1×10⁵ to about 1×10⁶, from about 1×10⁵ to about 1×10⁷, from about 1×10⁶ to about 1×10⁷, from about 1×10⁶ to about 1×10⁸, from about 1×10⁷ to about 1×10⁸, from about 1×10⁸ to about 1×10⁹, from about 1×10⁸ to about 1×10¹⁰, or from about 1×10⁹ to about 1×10¹⁰ of the presently disclosed in vitro maturated cells.

In certain embodiments, said composition is frozen. In certain embodiments, said composition further comprises at least one cryoprotectant, for example, but not limited to, dimethylsulfoxide (DMSO), glycerol, polyethylene glycol, sucrose, trehalose, dextrose, or a combination thereof.

In certain embodiments, the composition is a pharmaceutical composition that comprises a pharmaceutically acceptable carrier, excipient, diluent or a combination thereof.

The present disclosure also provides a device comprising the maturated cells or the composition comprising thereof, as disclosed herein. Non-limiting examples of devices include syringes, fine glass tubes, stereotactic needles and cannulas.

### 5.5. Methods of Screening Maturation-Promoting Compounds

The present disclosure provides an in vitro method of screening a compound that is suitable for promoting in vitro maturation of cells. In certain embodiments, the method comprises: (a) contacting a population of neuronal cells to a test compound; (b) withdrawing the test compound; (c) contacting the cells with potassium chloride between about 3 days and about 20 days after the withdrawal of the test compound; (d) measuring nuclear morphology, neurite growth and membrane excitability of the cells; (e) performing a computational analysis on the nuclear morphology, neurite growth and membrane excitability measured in step (d); and (f) identifying a test compound that is suitable for promoting in vitro maturation of neuronal cells based on the computational analysis performed in (e). In certain embodiments, the computational analysis performed in (e) comprises principal component analysis (PCA). In certain embodiments, the computational analysis performed in (e) comprises applying a machine learning classifier algorithm to predict neuron maturity.

In certain embodiments, the neuronal cells comprise immature neuronal cells, precursors thereof, progenitors thereof, or a combination thereof. In certain embodiments, the neuronal cells are selected from the group consisting of cortical neurons, spinal motor neurons, midbrain dopamine neurons, medium spiny neurons, interneurons, sensory neurons, enteric neurons, and combinations thereof. In certain embodiments, the neuronal cells comprise cortical neurons. In certain embodiments, the neuronal cells are in vitro differentiated from stem cells (e.g., human stem cells).

In certain embodiments, the cells are contacted with potassium chloride between about 3 days and about 20 days, between about 3 days and about 15 days, between about 3 days and about 10 days, between about 5 days and about 20 days, between about 5 days and about 15 days, between about 5 days and about 10 days, after the withdrawal of the test compound. In certain embodiments, the cells are contacted with potassium chloride between about 5 days and about 8 days after the withdrawal of the test compound. In certain embodiments, the cells are contacted with potassium chloride about 7 days after the withdrawal of the test compound.

In certain embodiments, the concentration of potassium chloride contacted with or exposed to the cells is between about 10 mM and about 150 mM, between about 30 mM and about 150 mM, between about 60 mM and about 150 mM, between about 100 mM and about 150 mM, between about 10 mM and about 100 mM, between about 30 mM and about 100 mM, between about 60 mM and about 100 mM. In certain embodiments, the concentration of potassium chloride contacted with or exposed to the cells is between about 40 mM and about 60 mM. In certain embodiments, the concentration of potassium chloride contacted with or exposed to the cells is about 50 mM.

In certain embodiments, measuring the nuclear morphology comprises measuring nuclear area, nuclear roundness (circularity), nuclear aspect ratio, nuclear perimeter, or a combination thereof. In certain embodiments, measuring the nuclear morphology comprises measuring nuclear area and nuclear roundness.

Any suitable methods known in the art can be used for determining the nuclear morphology. Non-limiting exemplary methods to determine nuclear morphology include nucleic acid staining and nuclear membrane protein immunostaining. In certain embodiments, the nuclear morphology is determined by DAPI counterstaining.

In certain embodiments, measuring the neurite growth comprises measuring neurite length, neurite branching, number of neurite segments, number of neurite nodes, or a combination thereof. In certain embodiments, measuring the neurite growth comprises measuring neurite length and neurite branching.

Any suitable methods known in the art can be used for determining the neurite growth. Non-limiting exemplary methods to determine neurite growth include microtubule-associated protein 2 (MAP2) immunostaining, and class III β-tubulin (TUBB3) immunostaining. In certain embodiments, the neurite growth is determined by MAP2 immunostaining.

In certain embodiments, measuring the membrane excitability comprises measuring percentage of cells expressing an immediate early gene (IEG) product. In certain embodiments, measuring the membrane excitability comprises subtracting the percentage of cells expressing the IEG product with percentage of control cells expressing the IEG product, wherein the control cells are not subject to the contact of potassium chloride.

In certain embodiments, IEG product comprises FOS, EGR1, ARC, NPAS4, and a combination thereof.

### 5.6. Kits

The present disclosure provides kits for promoting in vitro maturation of cells (e.g., immature cells, precursors or progenitors disclosed in Section 5.3 of the present disclosure). In certain embodiments, the kit comprises at least one inhibitor of an epigenetic regulator, and at least one agonist of a calcium channel (e.g., the inhibitors of an epigenetic regulator and the agonists of a calcium channel disclosed in Section 5.2. of the present disclosure). In certain embodiments, the kits comprises at least two inhibitors of an epigenetic regulator and at least two agonists of a calcium channel (e.g., the inhibitors of an epigenetic regulator and the agonists of a calcium channel disclosed in Section 5.2. of the present disclosure). In certain embodiments, the kit comprises an LSD1 inhibitor, a DOT1L inhibitor, a glutamate receptor agonist, and an LTCC agonist (e.g., the LSD1 inhibitors, DOT1L inhibitors, glutamate receptor agonist, and LTCC agonist disclosed in Section 5.2. of the present disclosure). In certain embodiments, the kit comprises GSK2879552, EPZ-5676, NMDA, and Bay K 8644.

In certain embodiments, the kit comprises at least one inhibitor of an epigenetic regulator (e.g., the inhibitors of an epigenetic regulator disclosed in Section 5.2. of the present disclosure). In certain embodiments, the kit comprises a DOT1L inhibitor (e.g., the DOT1L inhibitors disclosed in Section 5.2. of the present disclosure). In certain embodiments, the kit comprises an EZH2 inhibitor (e.g., the EZH2 inhibitors disclosed in Section 5.2. of the present disclosure). In certain embodiments, the kit comprises an EHMT1/2 inhibitor (e.g., the EHMT1/2 inhibitors disclosed in Section 5.2. of the present disclosure). In certain embodiments, the kit comprises GSK343, or a derivative thereof. In certain embodiments, the kit comprises UNC0638, or a derivative thereof. In certain embodiments, the kit comprises EPZ004777, or a derivative thereof.

In certain embodiments, the kit further comprises instructions for promoting in vitro maturation of cells. In certain embodiments, the instructions comprise contacting the cells with the at least one inhibitor of an epigenetic regulator, and at least one agonist of a calcium channel. In certain embodiments, the instructions comprise contacting the cells with the at least one inhibitor of an epigenetic regulator.

In certain embodiments, the instructions comprise contacting the cells with the at least one inhibitor of an epigenetic regulator, and at least one agonist of a calcium channel as described by the methods of the present disclosure (see Section 5.3 of the present disclosure).

In certain embodiments, the instructions comprise contacting the cells with the at least one inhibitor of an epigenetic regulator as described by the methods of the present disclosure (see Section 5.3 of the present disclosure).

In certain embodiments, the present disclosure provides kits comprising an effective amount of a cell population or a composition disclosed herein in unit dosage form (e.g., cell populations and compositions disclosed in Section 5.4 of the present disclosure). In certain embodiments, the kits comprise a sterile container which contains the therapeutic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

### 5.7. Exemplary Embodiments

A1. In certain non-limiting embodiments, the present disclosure provides a composition for promoting in vitro maturation of cells, comprising at least one inhibitor of an epigenetic regulator, and at least one agonist of a calcium channel.

A2. The foregoing composition of A1, wherein the at least one inhibitor of the epigenetic regulator comprises a lysine-specific demethylase 1 (LSD1) inhibitor, a disruptor of telomerase-like 1 (DOT1L) inhibitor, or a combination thereof.

A3. The foregoing composition of A1 or A2, wherein the at least one agonist of the calcium channel comprises a glutamate receptor agonist, an L-type calcium channel (LTCC) agonist, or a combination thereof.

A4. The foregoing composition of A3, wherein the glutamate receptor agonist is selected from the group consisting of NMDA, (RS)-(Tetratazol-5-yl)glycine, ibotenic acid, derivatives thereof, and combinations thereof.

A5. The foregoing composition of any one of A2-A4, wherein the LSD1 inhibitor is selected from the group consisting of GSK2879552, OG-L002, GSK-LSD1, derivatives thereof, and combinations thereof.

A6. The foregoing composition of any one of A2-A5, wherein the DOT1L inhibitor is selected from the group consisting of EPZ-5676, EPZ004777, SYC-522, SGC0946, Dot1L-IN-2, Dot1L-IN-4, Dot1L-IN-5, Dot1L-IN-6, CN-SAH, derivatives thereof, and combinations thereof.

A7. The foregoing composition of any one of A3-A6, wherein the LTCC agonist is selected from the group consisting of Bay K 8644, FPL 64176, derivatives thereof, and combinations thereof.

A8. The foregoing composition of any one of A1-A7, wherein the composition comprises an LSD1 inhibitor, a DOT1L inhibitor, a glutamate receptor agonist, and an LTCC agonist.

A9. The foregoing composition of any one of A1-A8, wherein the composition comprises GSK2879552, EPZ-5676, NMDA, and Bay K 8644.

A10. The foregoing composition of any one of A2-A9, wherein the concentration of the LSD1 inhibitor is between about 0.1 μM and about 10 μM.

A11. The foregoing composition of any one of A2-A10, wherein the concentration of the LSD1 inhibitor is about 1 μM.

A12. The foregoing composition of any one of A2-A11, wherein the concentration of the DOT1L inhibitor is between about 0.1 μM and about 10 μM.

A13. The foregoing composition of any one of A2-A12, wherein the concentration of the DOT1L inhibitor is about 1 μM.

A14. The foregoing composition of any one of A3-A13, wherein the concentration of the glutamate receptor agonist is between about 0.1 μM and about 10 μM.

A15. The foregoing composition of any one of A3-A14, wherein the concentration of the glutamate receptor agonist is about 1 μM.

A16. The foregoing composition of any one of A3-A15, wherein the concentration of the LTCC agonist is between about 0.1 μM and about 10 μM.

A17. The foregoing composition of any one of A3-A16, wherein the concentration of the LTCC agonist is about 1 μM.

B1. In certain non-limiting embodiments, the present disclosure provides a composition for promoting in vitro maturation of cells, comprising at least one inhibitor of an epigenetic regulator.

B2. The foregoing composition of B1, wherein the at least one inhibitor of the epigenetic regulator comprises a disruptor of telomerase-like 1 (DOT1L) inhibitor, an enhancer of zeste homolog 2 (EZH2) inhibitor, an euchromatic histone-lysine-N-methyltransferases 1 and 2 (EHMT1/2) inhibitor, or a combination thereof.

B3. The foregoing composition of B2, wherein the EZH2 inhibitor is selected from the group consisting of 3-deazaneplanocin A (DZNep), GSK343, GSK126, EPZ-6438, EPZ005687, GSK926, EPZ6438, EPZ011989, CPI-1205, CPI-169, ZLD1039, PF-06821497, UNC1999, PR-S1/OR-S2, DS-3201b, A-395, EBI-2511, EED226, EEDi-5285, EI1, EZH2-IN-2, EZH2-IN-3, EZH2-IN-4, EZH2-IN-5, GNA002, GSK503, JQEZ5, MAK683, MS1943, PF-06726304, UNC 1999, UNC6852, UNC6852, AM41-44A, BR-001, CPI-1328, CPI-905, DCE_254, EBI-2511, YM181, YM181, ZLD1039, ZLD10A, derivatives thereof, and combinations thereof.

B4. The foregoing composition of any one of B2-B3, wherein the EHMT1/2 inhibitor is selected from the group consisting of UNC0638, UNC0224, UNC0321, UNC0642, UNC0646, UNC0642, UNC0631, A-366, BIX-01294, BRD4770, BRD9539, CM-272, CM-579, CPUY074020, CSV0C018875, EHMT2-IN-1, EHMT2-IN-2, EML741, derivatives thereof, and combinations thereof.

B5. The foregoing composition of any one of B2-B4, wherein the DOT1L inhibitor is selected from the group consisting of EPZ-5676, EPZ004777, SYC-522, SGC0946, Dot1L-IN-2, Dot1L-IN-4, Dot1L-IN-5, Dot1L-IN-6, CN-SAH, derivatives thereof, and combinations thereof.

B6. The foregoing composition of any one of B1-B5, comprising GSK343, EPZ004777, UNC0638, or a combination thereof.

B7. The foregoing composition of any one of B1-B6, wherein the concentration of the at least one inhibitor of the epigenetic regulator is between about 0.1 μM and about 10 μM.

B8. The foregoing composition of any one of B1-B7, wherein the concentration of the at least one inhibitor of the epigenetic regulator is about 2 μM or about 4 μM.

C1. In certain non-limiting embodiments, the present disclosure provides an in vitro method for promoting the maturation of cells, comprising contacting the cells with at least one inhibitor of an epigenetic regulator, and at least one agonist of a calcium channel.

C2. The foregoing method of C1, wherein the at least one inhibitor of the epigenetic regulator comprises a lysine-specific demethylase 1 (LSD1) inhibitor, a disruptor of telomerase-like 1 (DOT1L) inhibitor, or a combination thereof.

C3. The foregoing method of C1 or C2, wherein the at least one agonist of the calcium channel comprises a glutamate receptor agonist, an L-type calcium channel (LTCC) agonist, or a combination thereof.

C4. The foregoing method of C2 or C3, wherein the LSD1 inhibitor is selected from the group consisting of GSK2879552, OG-L002, GSK-LSD1, derivatives thereof, and combinations thereof.

C5. The foregoing method of any one of C2-C4, wherein the DOT1L inhibitor is selected from the group consisting of EPZ-5676, EPZ004777, SYC-522, SGC0946, Dot1L-IN-2, Dot1L-IN-4, Dot1L-IN-5, Dot1L-IN-6, CN-SAH, derivatives thereof, and combinations thereof.

C6. The foregoing method of any one of C3-C5, wherein the glutamate receptor agonist is selected from the group consisting of NMDA, (RS)-(Tetratazol-5-yl)glycine, ibotenic acid, derivatives thereof, and combinations thereof.

C7. The foregoing method of any one of C3-C6, wherein the LTCC agonist is selected from the group consisting of Bay K 8644, FPL 64176, derivatives thereof, and combinations thereof.

C8. The foregoing method of any one of C1-C7, wherein the method comprises contacting the cells with an LSD1 inhibitor, a DOT1L inhibitor, a glutamate receptor agonist, and an LTCC agonist.

C9. The foregoing method of any one of C1-C8, wherein the method comprises contacting the cells with GSK2879552, EPZ-5676, NMDA, and Bay K 8644.

C10. The foregoing method of any one of C2-C9, wherein the concentration of the LSD1 inhibitor is between about 0.1 μM and about 10 μM.

C11. The foregoing method of any one of C2-C10, wherein the concentration of the LSD1 inhibitor is about 1 μM.

C12. The foregoing method of any one of C2-C11 wherein the concentration of the DOT1L inhibitor is between about 0.1 μM and about 10 μM.

C13. The foregoing method of any one of C2-C12, wherein the concentration of the DOT1L inhibitor is about 1 μM.

C14. The foregoing method of any one of C3-C13, wherein the concentration of the glutamate receptor agonist is between about 0.1 μM and about 10 μM.

C15. The foregoing method of any one of C3-C14, wherein the concentration of the glutamate receptor agonist is about 1 μM.

C16. The foregoing method of any one of C3-C15, wherein the concentration of the LTCC agonist is between about 0.1 μM and about 10 μM.

C17. The foregoing method of any one of C3-C15, wherein the concentration of the LTCC agonist is about 1 μM.

C18. The foregoing method of any one of C1-C17, wherein the cells are contacted with the at least one inhibitor of the epigenetic regulator and the at least one agonist of the calcium channel for at least about 3 days and/or for up to about 30 days.

D1. In certain non-limiting embodiments, the present disclosure provides an in vitro method for promoting the maturation of cells, comprising contacting the cells with at least one inhibitor of an epigenetic regulator.

D2. The foregoing method of D1, wherein the at least one inhibitor of the epigenetic regulator comprises a disruptor of telomerase-like 1 (DOT1L) inhibitor, an enhancer of zeste homolog 2 (EZH2) inhibitor, an euchromatic histone-lysine-N-methyltransferases 1 and 2 (EHMT1/2) inhibitor, or a combination thereof.

D3. The foregoing method of D2, wherein the EZH2 inhibitor is selected from the group consisting of 3-deazaneplanocin A (DZNep), GSK343, GSK126, EPZ-6438, EPZ005687, GSK926, EPZ6438, EPZ011989, CPI-1205, CPI-169, ZLD1039, PF-06821497, UNC1999, PR-S1/OR-S2, DS-3201b, A-395, EBI-2511, EED226, EEDi-5285, EI1, EZH2-IN-2, EZH2-IN-3, EZH2-IN-4, EZH2-IN-5, GNA002, GSK503, JQEZ5, MAK683, MS1943, PF-06726304, UNC 1999, UNC6852, UNC6852, AM41-44A, BR-001, CPI-1328, CPI-905, DCE_254, EBI-2511, YM181, YM181, ZLD1039, ZLD10A, derivatives thereof, and combinations thereof.

D4. The foregoing method of any one of D2-D3, wherein the EHMT1/2 inhibitor is selected from the group consisting of UNC0638 UNC0224, UNC0321, UNC0642, UNC0646, UNC0642, UNC0631, A-366, BIX-01294, BRD4770, BRD9539, CM-272, CM-579, CPUY074020, CSV0C018875, EHMT2-IN-1, EHMT2-IN-2, EML741, derivatives thereof, and combinations thereof.

D5. The foregoing method of any one of D2-D4, wherein the DOT1L inhibitor is selected from the group consisting of EPZ-5676, EPZ004777, SYC-522, SGC0946, Dot1L-IN-2, Dot1L-IN-4, Dot1L-IN-5, Dot1L-IN-6, CN-SAH, derivatives thereof, and combinations thereof.

D6. The foregoing method of any one of D1-D5, comprising contacting the cells with GSK343, EPZ004777, UNC0638, or a combination thereof.

D7. The foregoing method of any one of D1-D6, wherein the concentration of the at least one inhibitor of the epigenetic regulator is between about 0.1 μM and about 10 μM.

D8. The foregoing method of any one of D1-D7, wherein the concentration of the at least one inhibitor of the epigenetic regulator is about 2 μM or about 4 μM.

D9. The foregoing method of any one of D1-D8, wherein the cells are immature neuronal cells, precursors thereof, progenitors thereof, or a combination thereof.

D10. The foregoing method of D9, wherein the neuronal cells are selected from the group consisting of cortical neurons, spinal motor neurons, and combinations thereof.

D11. The foregoing method of D9 or D10, wherein the cells form a brain organoid.

D12. The foregoing method of D11, wherein the brain organoid is a dorsal forebrain organoid.

D13. The foregoing method of any one of D1-D8, wherein the cells are immature non-neuronal cells, precursors thereof, progenitors thereof, or a combination thereof.

D14. The foregoing method of D13, wherein the cells are selected from the group consisting of pancreatic beta cells, melanocytes, and combinations thereof.

D15. The foregoing method of any one of D1-D14, wherein the cells are in vitro differentiated from stem cells.

D16. The foregoing method of D15, wherein the stem cells are selected from the group consisting of embryonic stem cells, induced pluripotent stem cells, parthenogenetic stem cells, primordial germ cell-like pluripotent stem cells, epiblast stem cells, and F-class pluripotent stem cells, embryonic neural stem cells, adult neural stem cells, and long-term self-renewing neural stem cells, and combinations thereof.

E1. In certain non-limiting embodiments, the present disclosure provides an in vitro method for promoting the maturation of cells, comprising contacting the cells with the composition of any one of A1-A17 or B1-B8.

F1. In certain non-limiting embodiments, the present disclosure provides for the use of the composition of any one of A1-A17 or B1-B8 for promoting the maturation of cells.

G1. In certain non-limiting embodiments, the present disclosure provides a kit for promoting in vitro maturation of cells, comprising at least one inhibitor of an epigenetic regulator, and at least one agonist of a calcium channel.

G2. The foregoing kit of G1, wherein the at least one inhibitor of the epigenetic regulator comprises a lysine-specific demethylase 1 (LSD1) inhibitor, a disruptor of telomerase-like 1 (DOT1L) inhibitor, or a combination thereof.

G3. The foregoing kit of G1 or G2, wherein the at least one agonist of the calcium channel comprises a glutamate receptor agonist, an L-type calcium channel (LTCC) agonist, or a combination thereof.

G4. The foregoing kit of G2 or G3, wherein the LSD1 inhibitor is selected from the group consisting of GSK2879552, OG-L002, GSK-LSD1, derivatives thereof, and combinations thereof.

G5. The foregoing kit of any one of G2-G4, wherein the DOT1L inhibitor is selected from the group consisting of EPZ-5676, EPZ004777, SYC-522, SGC0946, Dot1L-IN-2, Dot1L-IN-4, Dot1L-IN-5, Dot1L-IN-6, CN-SAH, derivatives thereof, and combinations thereof.

G6. The foregoing kit of any one of G3-G4, wherein the glutamate receptor agonist is selected from the group consisting of NMDA, (RS)-(Tetratazol-5-yl)glycine, ibotenic acid, derivatives thereof, and combinations thereof.

G7. The foregoing kit of any one of G3-G6, wherein the LTCC agonist is selected from the group consisting of Bay K 8644, FPL 64176, derivatives thereof, and combinations thereof.

G8. The foregoing kit of any one of G1-G7, wherein the kit comprises an LSD1 inhibitor, a DOT1L inhibitor, a glutamate receptor agonist, and an LTCC agonist.

G9. The foregoing kit of any one of G1-G8, wherein the kit comprises GSK2879552, EPZ-5676, NMDA, and Bay K 8644.

H1. In certain non-limiting embodiments, the present disclosure provides a kit for promoting in vitro maturation of cells, comprising at least one inhibitor of an epigenetic regulator.

H2. The foregoing kit of H1, wherein the at least one inhibitor of the epigenetic regulator comprises a disruptor of telomerase-like 1 (DOT1L) inhibitor, an enhancer of zeste homolog 2 (EZH2) inhibitor, an euchromatic histone-lysine-N-methyltransferases 1 and 2 (EHMT1/2) inhibitor, or a combination thereof.

H3. The foregoing kit of H2, wherein the EZH2 inhibitor is selected from the group consisting of 3-deazaneplanocin A (DZNep), GSK343, GSK126, EPZ-6438, EPZ005687, GSK926, EPZ6438, EPZ011989, CPI-1205, CPI-169, ZLD1039, PF-06821497, UNC1999, PR-S1/OR-S2, DS-3201b, A-395, EBI-2511, EED226, EEDi-5285, EI1, EZH2-IN-2, EZH2-IN-3, EZH2-IN-4, EZH2-IN-5, GNA002, GSK503, JQEZ5, MAK683, MS1943, PF-06726304, UNC 1999, UNC6852, UNC6852, AM41-44A, BR-001, CPI-1328, CPI-905, DCE_254, EBI-2511, YM181, YM181, ZLD1039, ZLD10A, derivatives thereof, and combinations thereof.

H4. The foregoing kit of H2 or H3, wherein the EHMT1/2 inhibitor is selected from the group consisting of UNC0638, UNC0224, UNC0321, UNC0642, UNC0646, UNC0642, UNC0631, A-366, BIX-01294, BRD4770, BRD9539, CM-272, CM-579, CPUY074020, CSV0C018875, EHMT2-IN-1, EHMT2-IN-2, EML741, derivatives thereof, and combinations thereof.

H5. The foregoing kit of any one of H2-H4, wherein the DOT1L inhibitor is selected from the group consisting of EPZ-5676, EPZ004777, SYC-522, SGC0946, Dot1L-IN-2, Dot1L-IN-4, Dot1L-IN-5, Dot1L-IN-6, CN-SAH, derivatives thereof, and combinations thereof.

H6. The foregoing kit of any one of H1-H5, comprising GSK343, EPZ004777, UNC0638, or a combination thereof.

H7. The foregoing kit of any one of H1-H6, further comprising instructions for promoting in vitro maturation of cells.

I1. In certain non-limiting embodiments, the present disclosure provides an in vitro method of screening a compound that is suitable for promoting in vitro maturation of cells, comprising:
(a) contacting a population of immature neuronal cells to a test compound;
(b) withdrawing the test compound;
(c) contacting the cells with potassium chloride between about 3 days and about 20 days after the withdrawal of the test compound;
(d) measuring nuclear morphology, neurite growth and membrane excitability of the cells;
(e) performing principal component analysis on the nuclear morphology, neurite growth and membrane excitability measured in step (d); and
(f) identifying a test compound that is suitable for promoting in vitro maturation of neuronal cells based on the principal component analysis performed in (e).

I2. The foregoing method of I1, wherein the cells are contacted with potassium chloride about 7 days after the withdrawal of the test compound.

I3. The foregoing method of I1 or I2, wherein the concentration of potassium chloride is between about 10 mM and about 100 mM.

I4. The foregoing method of any one of I1-I3, wherein the concentration of potassium chloride is about 50 mM.

I5. The foregoing method of any one of I1-I4, wherein measuring the nuclear morphology comprises measuring nuclear area and nuclear roundness.

I6. The foregoing method of any one of I1-I5, wherein the nuclear morphology is determined by DAPI counterstaining.

I7. The foregoing method of any one of I1-I6, wherein measuring the neurite growth comprises measuring neurite length and neurite branching.

I8. The foregoing method of any one of I1-I7, wherein the neurite growth is determined by microtubule-associated protein 2 (MAP2) immunostaining.

I9. The foregoing method of any one of I1-I8, wherein measuring the membrane excitability comprises measuring percentage of cells expressing an immediate early gene (IEG) product.

I10. The foregoing method of I9, wherein measuring the membrane excitability comprises subtracting the percentage of cells expressing the IEG product with percentage of control cells expressing the IEG product, wherein the control cells are not subject to the contact of potassium chloride.

I11. The foregoing method of I10, wherein the IEG product comprises FOS, EGR1, and a combination thereof.

I12. The foregoing method of any one of I1-I11, wherein the neuronal cells are cortical neurons.

### 6. EXAMPLES

The present disclosure will be better understood by reference to the following Example, which is provided as exemplary of the present disclosure, and not by way of limitation.

### Example 1: Combined Small Molecule Treatment Accelerates Timing of Maturation in Human Pluripotent Stem Cell-Derived Neurons

Within a given micro-environment, cell-intrinsic maturation rates appear dominant and seem to be determined by a species-specific molecular clock, which runs especially slow in human neurons (Barry, C. et al. Dev. Biol. 423, 101-110 (2017); Marchetto, M. C. et al. Elife 8, (2019)). For example, the maturation of hPSC-derived cortical neurons transplanted into the developing mouse brain follows human-specific timing, requiring 9 months to achieve postnatal morphologies and spine function (Linaro, D. et al. Neuron 104, 972-986.e6 (2019)). Similarly, the rescue of Parkinsonian rats by transplanting either mouse, pig or human dopamine neurons into an identical host brain environment, results in functional rescue after 4 weeks, 3 months or 5 months respectively, matching the pace of dopamine neuron maturation across those species in vivo (Isacson, O. & Deacon, T. Trends Neurosci. 20, 477-482 (1997)).

The present disclosure identified effectors of intrinsic maturation timing and developed a chemical strategy to accelerate it. A multi-phenotypic, image-based assay is disclosed presently to monitor maturation in nearly pure populations of hPSC-derived deep layer cortical neuron cultures and applied it to screen 2688 bioactive compounds. Among the screening hits, compounds targeting chromatin remodeling and calcium-dependent transcription were combined into a maturation cocktail that was effective across a broad range of maturation phenotypes and capable of driving maturation in both neuronal and non-neuronal lineages.

### Results

### High Content Assay of Neuron Maturity

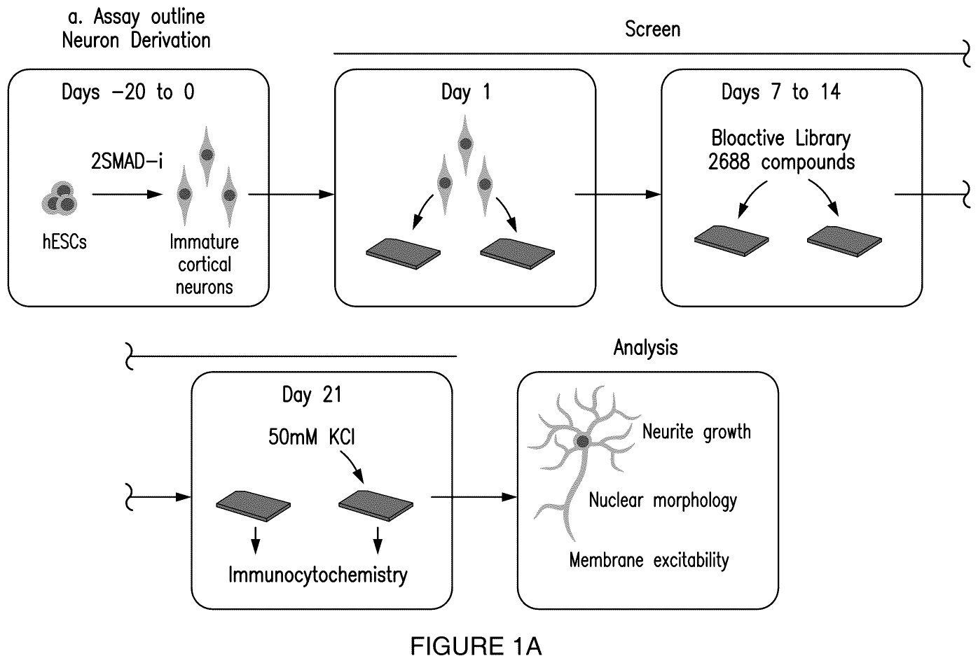

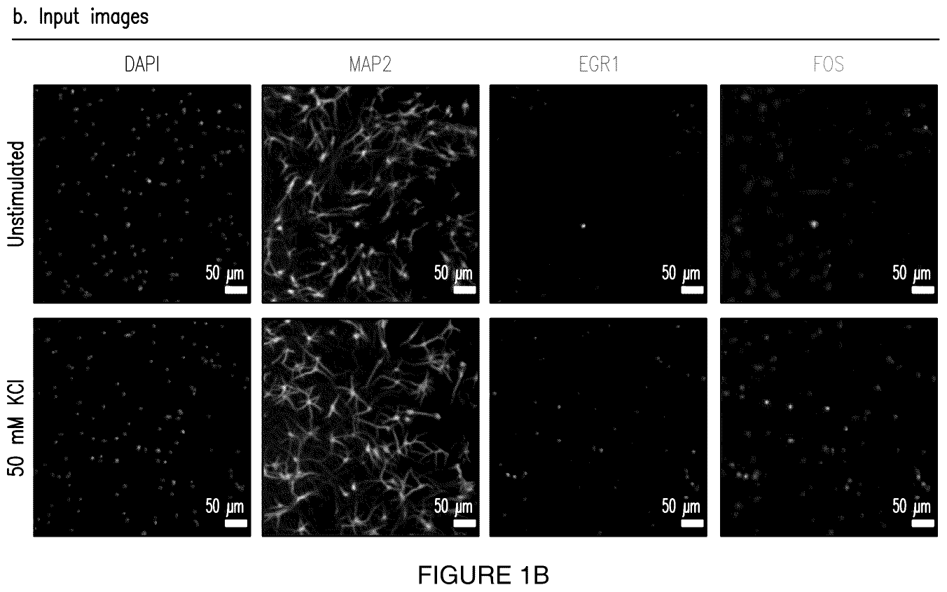

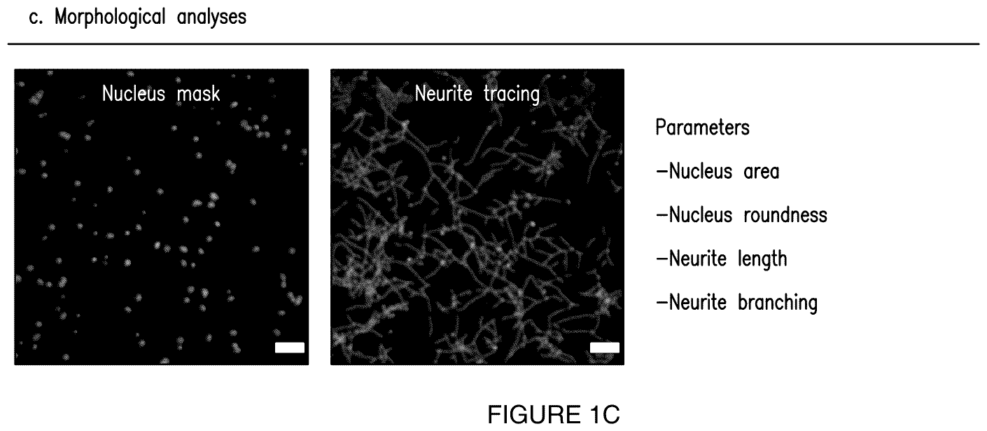

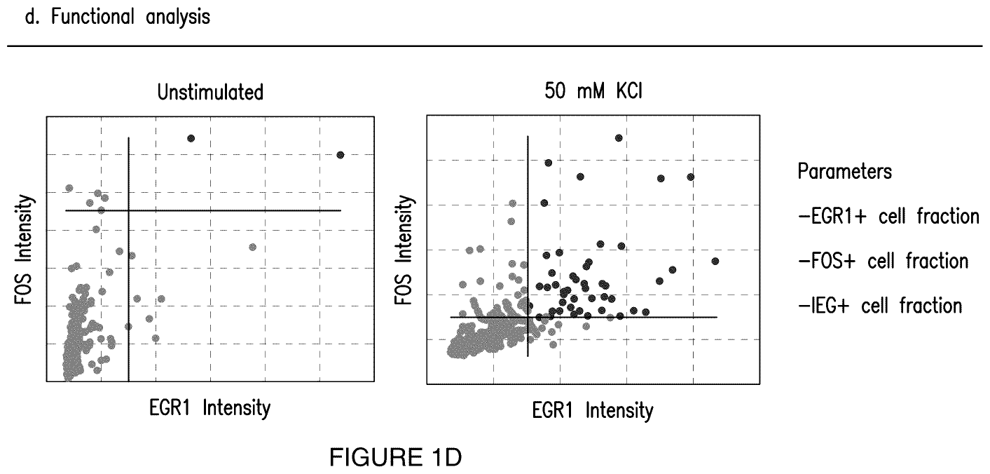

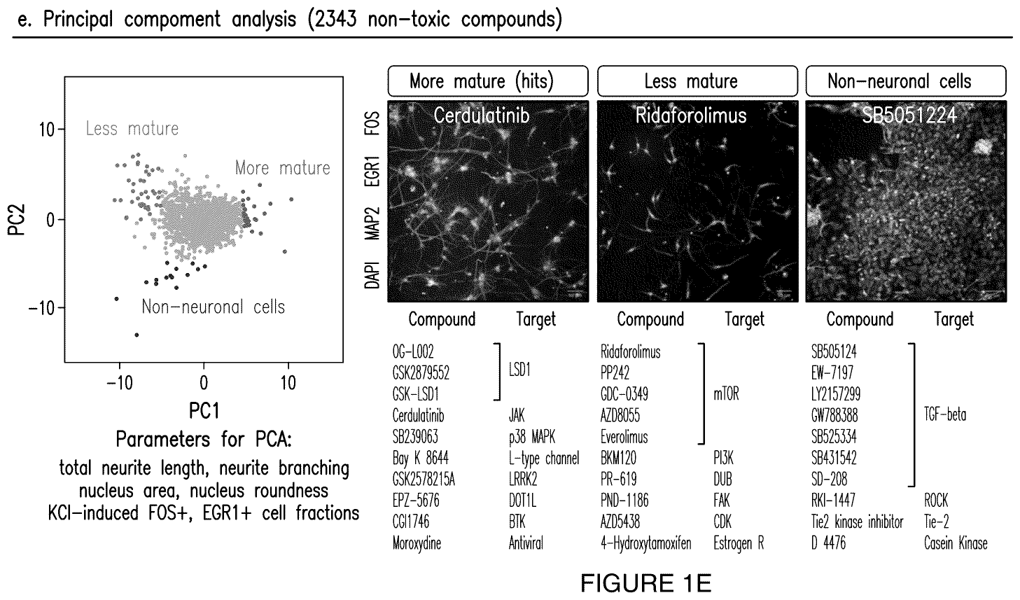

The phenotypic complexity of neurons makes single-readout assays unsuitable to fully capture maturation stages. Therefore, a multi-phenotype approach (via high-content screening, HCS) (Boutros M., Heigwer F. & Laufer C. Cell vol. 163 1314-1325 (2015)) was used to design an assay that simultaneously monitors distinct features of neuronal maturation (FIG. 1A). Dendritic outgrowth is a widely used parameter of neuron maturity (Wu, G. Y. et al., J. Neurosci. 19, 4472-4483 (1999)) and can be monitored through automated tracing of microtubule-associated protein 2 (MAP2) immunostaining (FIGS. 1B and 1C). Changes in nuclear size and morphology are also characteristic of neuron development and maturation (Ito K. & Takizawa T., Frontiers in Genetics vol. 9 (2018)) and can be tracked via DAPI counterstaining (FIGS. 1B and 1C). As an indirect measurement of neuronal function and excitability, the nuclear expression of immediate early gene (IEG) products FOS and EGR1 were quantified following 2 hours of potassium chloride (KCl) stimulation (FIGS. 1B and 1D). IEGs are defined by their rapid induction in the absence of de-novo protein synthesis by stimuli that include sustained membrane depolarization in neurons (Sheng M. & Greenberg M. E., Neuron vol. 4 477-485 (1990)). In contrast to more traditional measures of neuronal activity such as calcium imaging and electrophysiology, IEG immunoreactivity is readily scalable as a readout for thousands of treatment conditions. However, IEGs can be triggered by stimuli other than neuronal activity including growth factor signaling (Greenberg M. E. & Ziff E. B., Nature 311, 433-438 (1984)) and cellular stress responses (Murai J. et al., Cell Rep. 30, 4137-4151.e6 (2020)). Therefore, to avoid direct activation of IEGs, transient compound treatment (day 7-14) was used and all measurements were performed after rinsing of compounds followed by culture in compound-free medium for an additional 7 days (day 14-21) prior to analysis (FIG. 1A). Furthermore, IEGs under both basal and KCl-stimulated conditions were recorded to specifically determine the depolarization-induced signal by subtracting baseline from KCl-induced responses. Measuring maturation readouts only after compound withdrawal enabled the identification of compounds that trigger a long-lasting “memory” of a maturation stimulus even after compound removal.

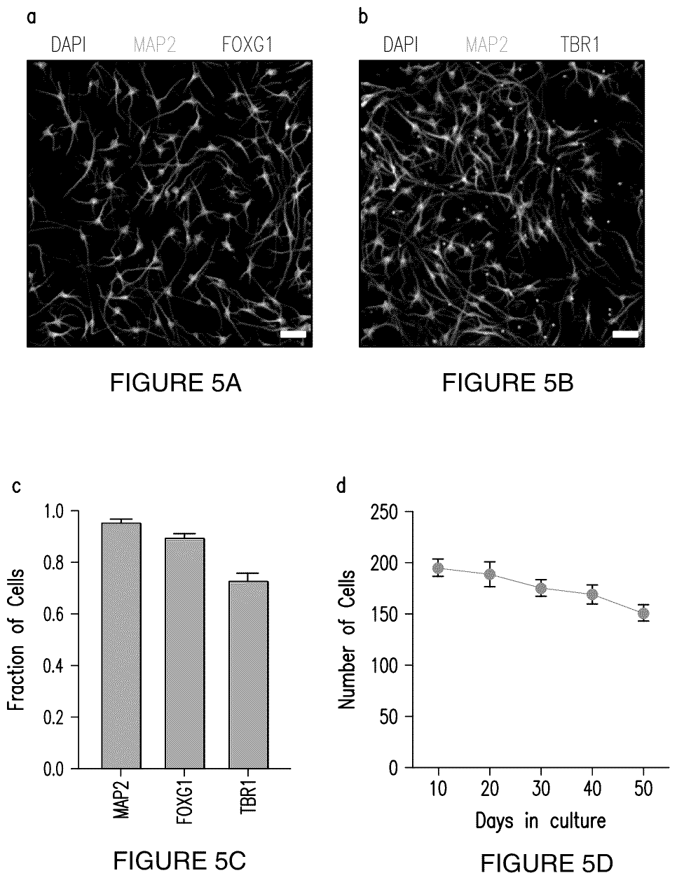

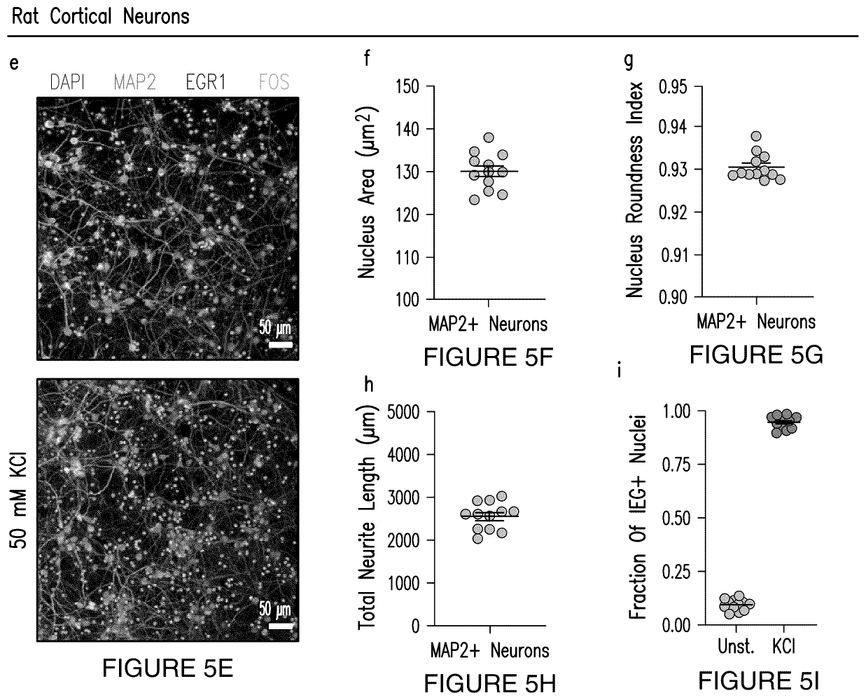

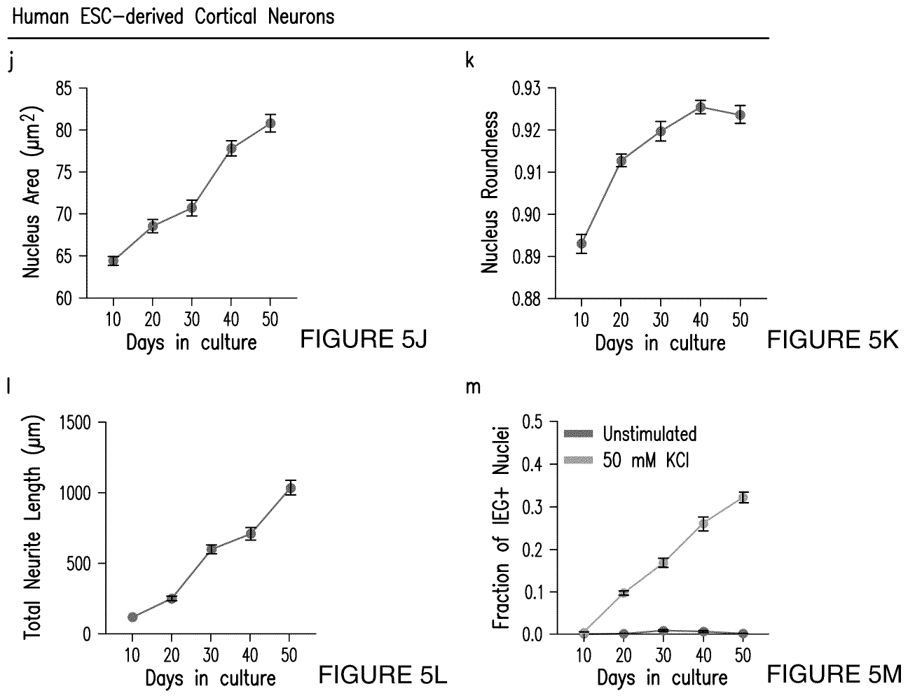

While these readouts are pan-neuronal, and therefore appropriate across different neuronal lineages, cortical neurons were chosen for the screen for both technical and biological reasons. Cortical neurons can be derived at high efficiency in the absence of expensive recombinant proteins, and their even cell distribution free of clusters makes them amenable to high-throughput imaging. They also represent a brain region that undergoes a particularly protracted development, and a region of great importance to human neurological disease. The present cortical neuron differentiation protocol yields highly pure populations of post-mitotic deep-layer TBR1+ cells, which can be readily scaled, cryopreserved and directly thawed for use in large-scale assays (FIGS. 5A-5D).

To benchmark the assay performance in mature cells, primary embryonic rat cortical neurons were employed, which quickly and reliably develop mature-like functionality in vitro (Opitz T. et al., J. Neurophysiol. 88, 2196-2206 (2002)). At 14 days after plating, rat neurons displayed large and round nuclei (130 μm², 0.93 roundness index), extensive neurite growth (>2500 μm/neuron), and near 100% of the neurons showed KCl-induced IEG responses (FIGS. 5E-5I). In contrast, in human PSC-derived cortical neurons, these properties only very gradually increased over a 50-day culture period and never reached the maturity of their rodent counterparts (FIGS. 5J-5M). These results indicate that the present multi-phenotypic assay reliably captures the maturation of developing rat and human PSC-derived human cortical neurons.

### Chemical Screen for Maturation Enhancers

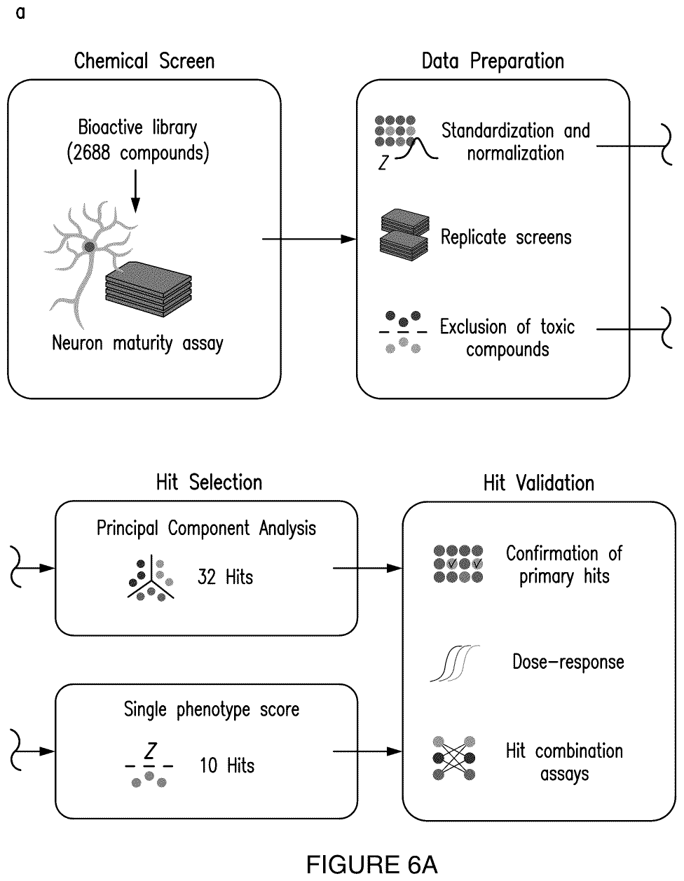

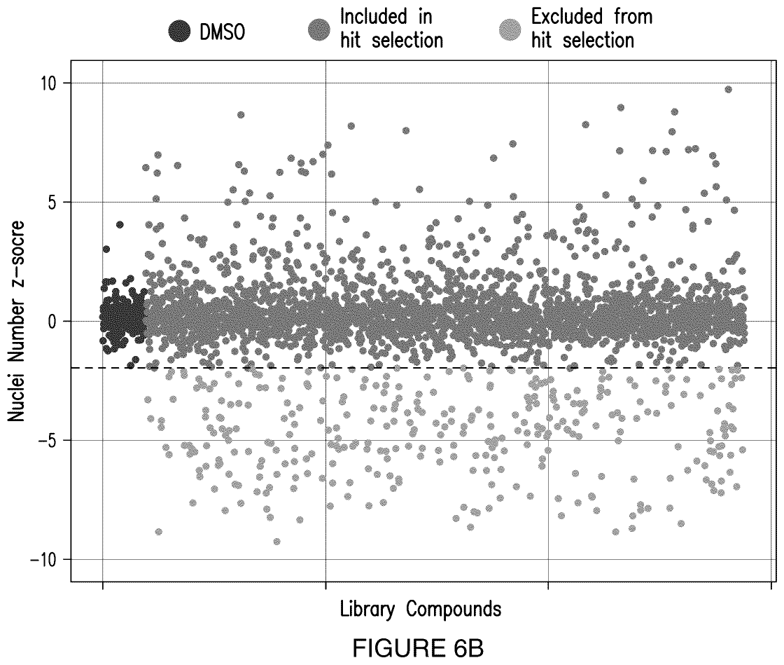

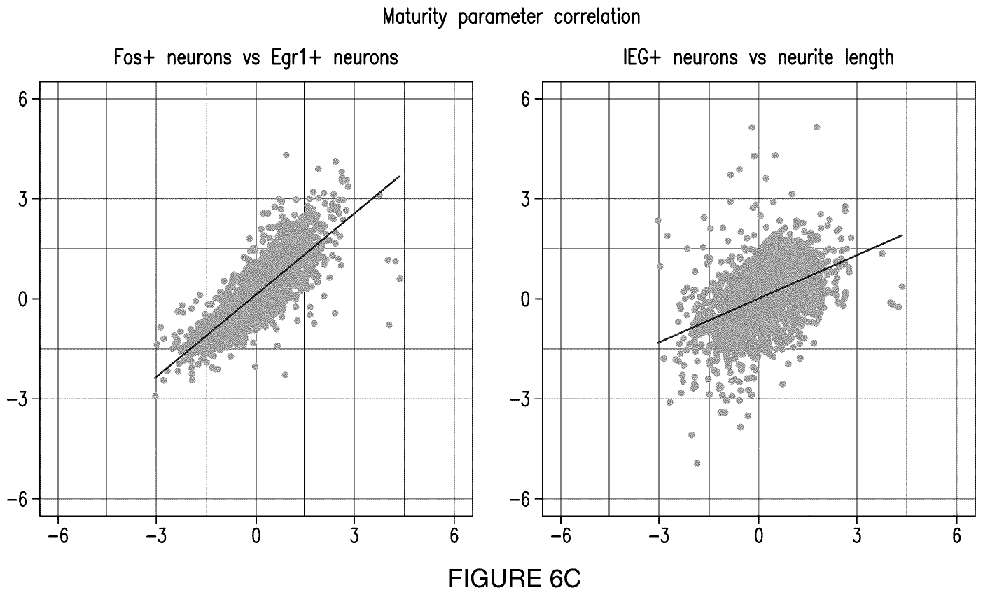

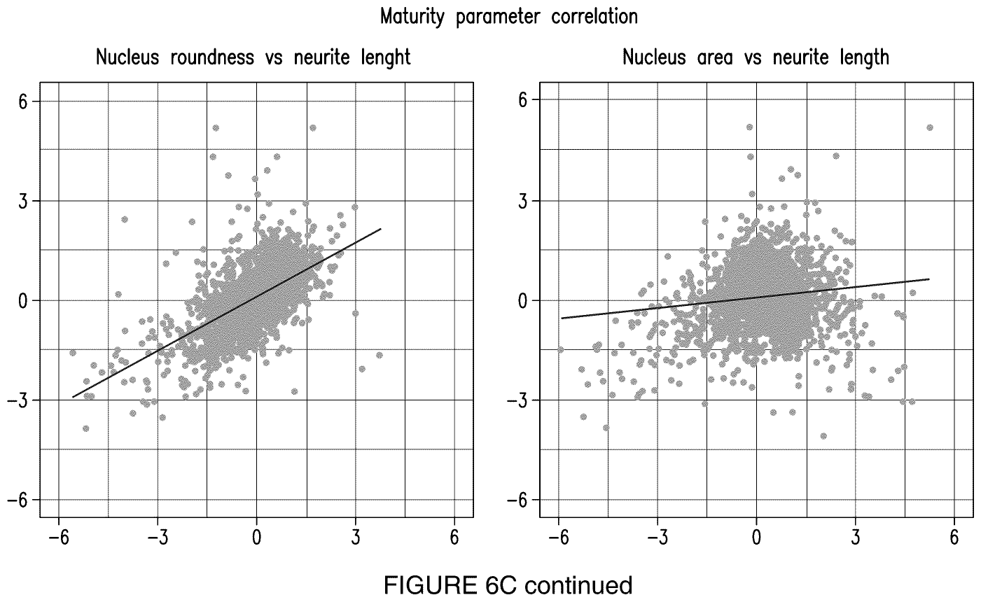

The present maturity assay was then applied to screen a library of 2688 bioactive compounds in hPSC-derived cortical neurons (FIG. 6A). The library was applied at 5 μM and standard scores (z-scores) of duplicate screen runs were averaged for analysis. Viability was determined by quantifying intact nuclei, and 325 toxic compounds with a z-score below −2 were excluded from further analysis (FIG. 6B). For HCS hit selection, principal component analysis (PCA) was applied to 6 maturity z-scores to identify patterns of distribution among compounds, avoiding single threshold hit discrimination (Singh S. et al., Journal of Biomolecular Screening vol. 19 640-650 (2014)) (FIG. 1E, left panel). The 6 parameters were: nuclear size and roundness, total neurite length and branching (number of segments per cell), and fractions of KCl-induced FOS+ and EGR1+ cells. Three phenotypic clusters of compounds were identified by PCA: maturation enhancers (hits); maturation suppressors, consisting mostly of inhibitors of the PI3K/AKT/mTOR axis; and inducers of non-neuronal contaminant proliferation, which were highly enriched in TGF-β signaling inhibitors as well as inhibitors of rho-associated protein kinase (ROCK) and other signaling pathways (FIG. 1E, right panel).

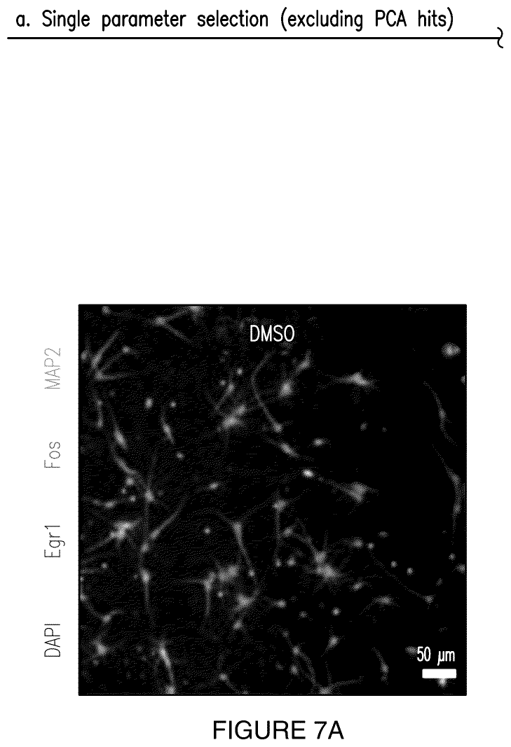

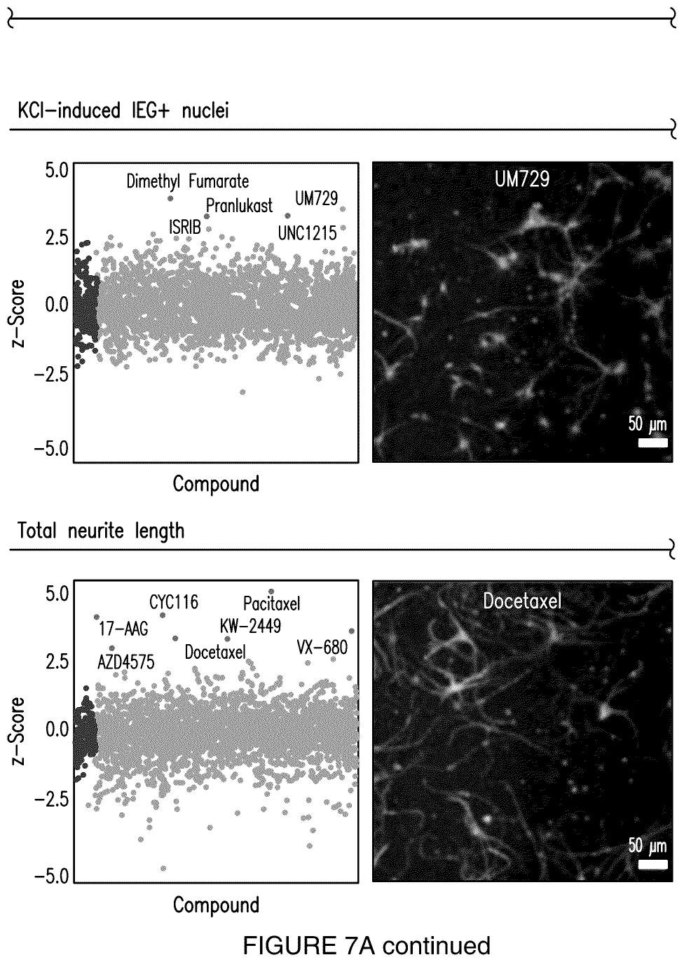

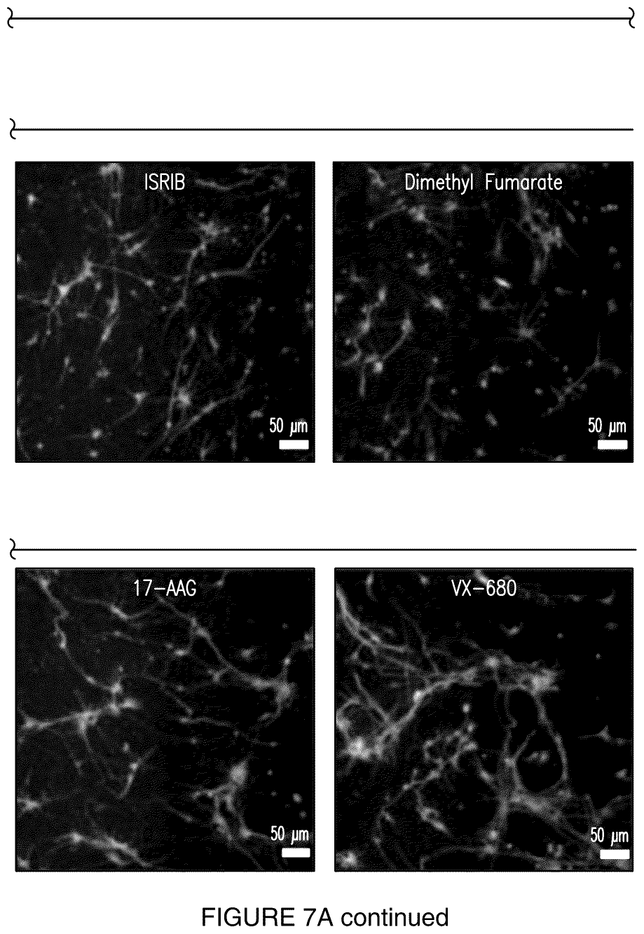

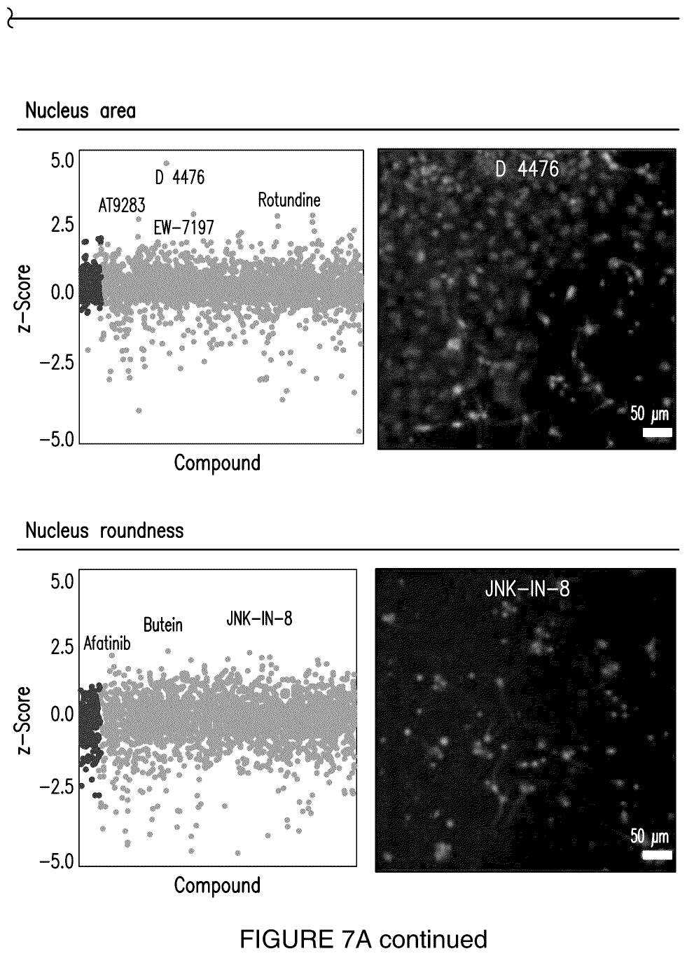

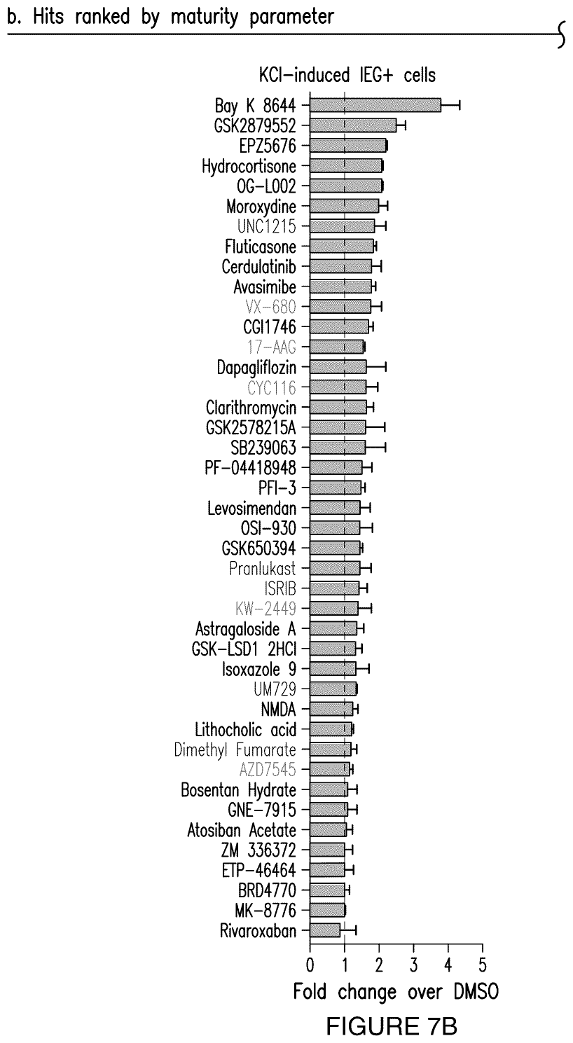

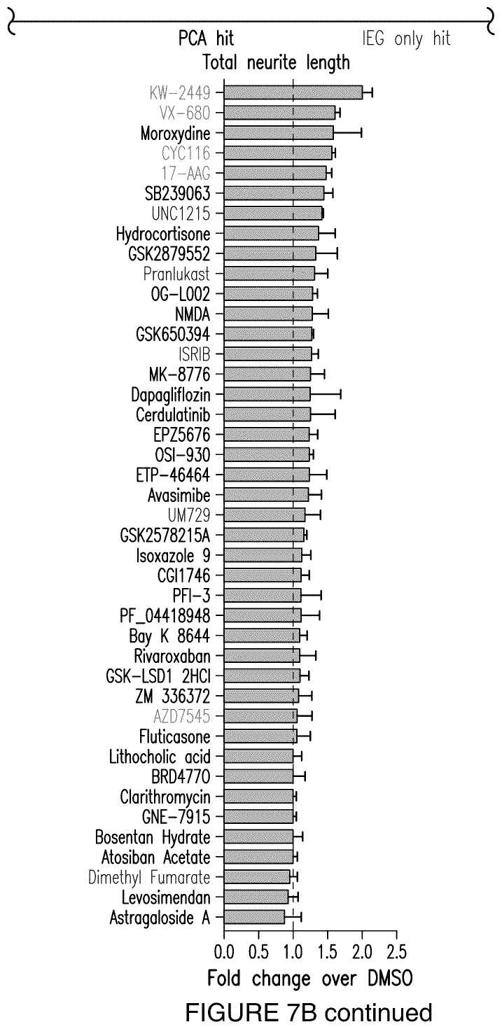

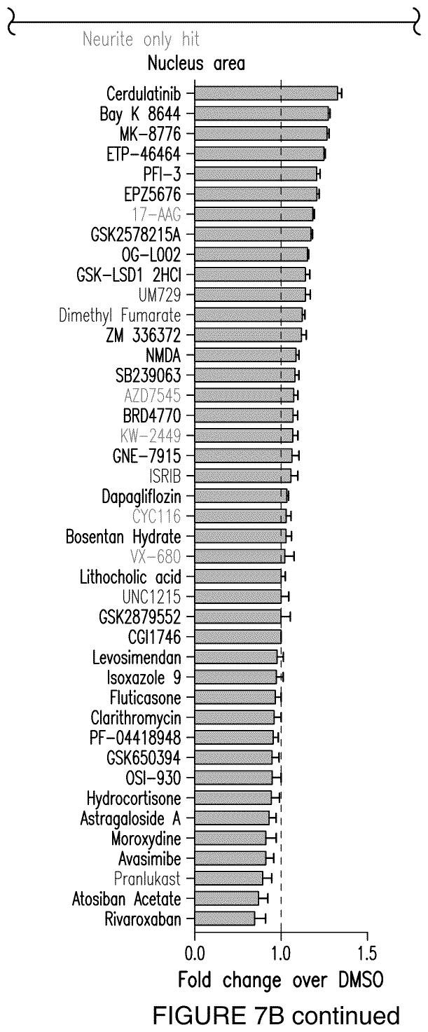

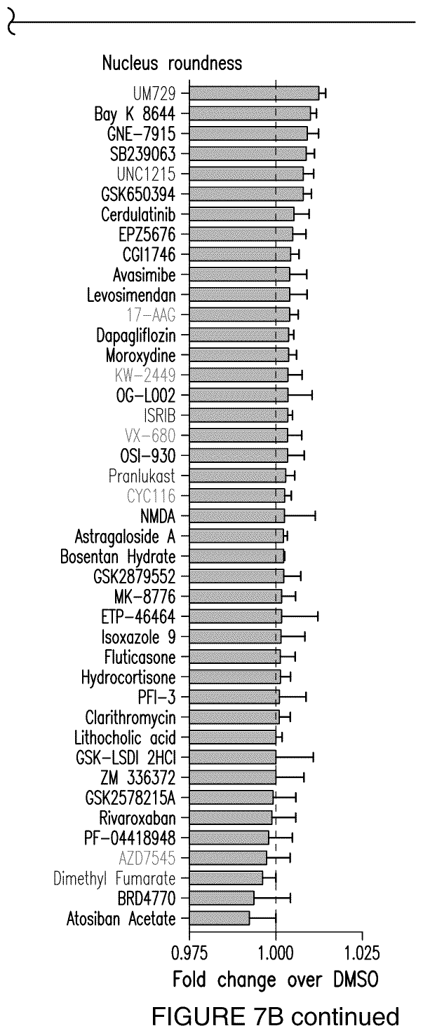

Thirty-two compounds were selected within the mature cluster for validation. While PCA identifies compounds with the greatest overall maturation effect, compounds with strong effects on single parameters could also be of interest. Therefore the top 5 highest scoring compounds were added for each, total neurite length and double FOS/EGR1 positive cells, excluding compounds already selected by PCA (FIG. 7A). Because single-parameter readouts are susceptible to false positives, drugs with known maturation-independent effects, such as microtubule stabilizers docetaxel and paclitaxel, were excluded. Interestingly, neurite-only hits included several inhibitors of Aurora kinase, in agreement with recent phenotypic screens targeting this phenotype (Shlevko E. et al., Cell Rep. 28, 3224-3237.e5 (2019); Blazejewski S. et al., bioRxiv 2020.06.25.162271 (2020)). Using these combined criteria, 42 primary hits were selected, as shown in Table 1.

TABLE 1
Identified 42 primary hits.

| Compound | Target/Description |
|---|---|
| 17-AAG (Tanespimycin) | Hsp-90 inhibitor |
| Astragaloside A | Saponin |
| Atosiban Acetate | Oxytocin inhibitor |
| Avasimibe | ACAT inhibitor |
| AZD7545 | PDHK inhibitor |
| Bay K 8644 | L-type calcium channel agonist |
| Bosentan Hydrate | ET receptor antagonist |
| BRD4770 | EHMT2 inhibitor |
| Cerdulatinib (PRT062070_PRT2070) | JAK inhibitor |
| CGI1746 | BTK inhibitor |
| Clarithromycin | CYP3A4 inhibitor |
| CYC116 | Aurora kinase inhibitor |
| Dapagliflozin | SGLT inhibitor |
| Dimethyl Fumarate | NF-kB inhibitor |
| EPZ5676 | DOT1L inhibitor |
| ETP-46464 | ATR inhibitor |
| Fluticasone propionate | Glucorticoid |
| GNE-7915 | LRRK2 inhibitor |
| GSK-LSD1 | LSD1 inhibitor |
| GSK2578215A | LRRK2 inhibitor |
| GSK2879552 | LSD1 inhibitor |
| GSK650394 | SGK inhibitor |
| Hydrocortisone | Glucorticoid |
| Isoxazole 9 (ISX-9) | Promotes adult neurogenesis |
| ISRIB (trans-isomer) | PERK inhibitor |
| KW-2449 | FLT3 inhibitor |
| Levosimendan | Calcium sensitizer |
| Lithocholic acid | Bile acid |
| MK-8776 (SCH 900776) | ChK inhibitor |
| Moroxydine HCl | Antiviral (biguanidine) |
| NMDA (N-Methyl-D-aspartic acid) | NMDAR agonist |
| OG-L002 | LSD1 inhibitor |
| OSI-930 | c-Kit/c-RAF inhibitor |
| PF-04418948 | EP2 receptor antagonist |
| PFI-3 | SMARCA inhibitor |
| Pranlukast | Antileukotriene |
| Rivaroxaban | Factor Xa blocker |
| SB239063 | p38 MAPK inhibitor |
| UM729 | AhR antagonist enhancer |
| UNC1215 | L3MBTL3 inhibitor |
| VX-680 (Tozasertib_MK-0457) | Aurora kinase inhibitor |
| ZM 336372 | c-RAF inhibitor |

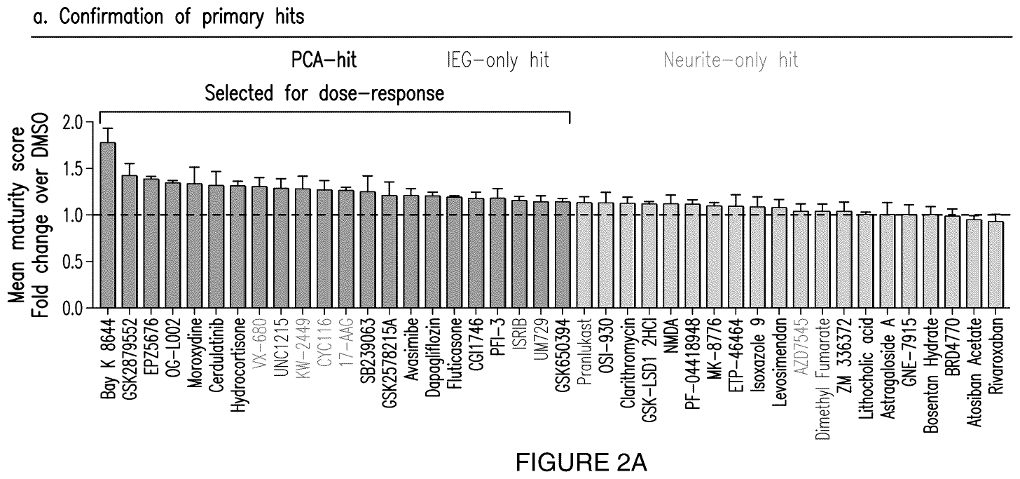

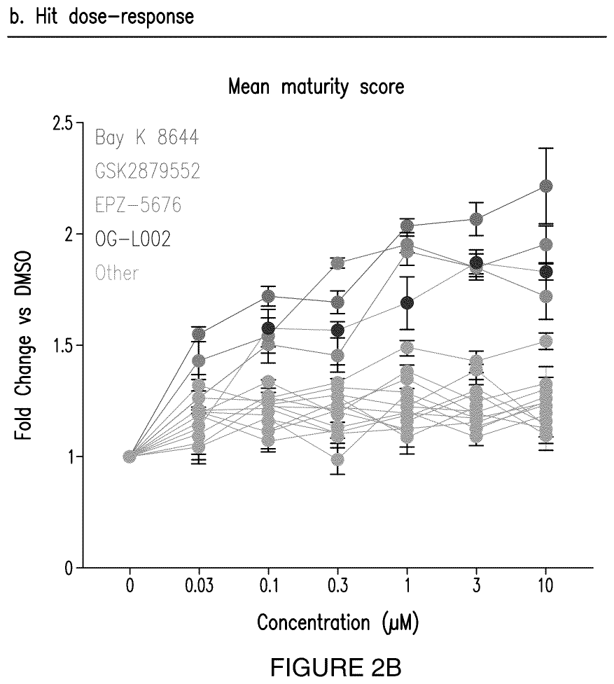

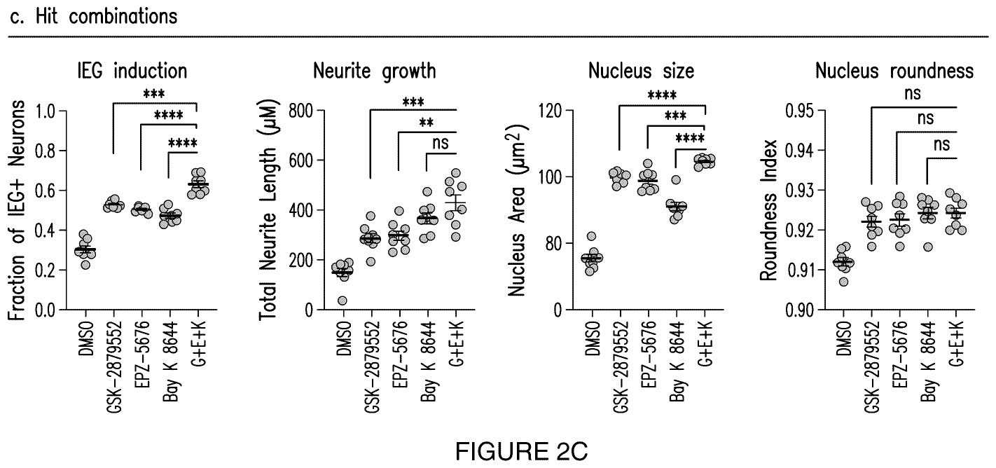

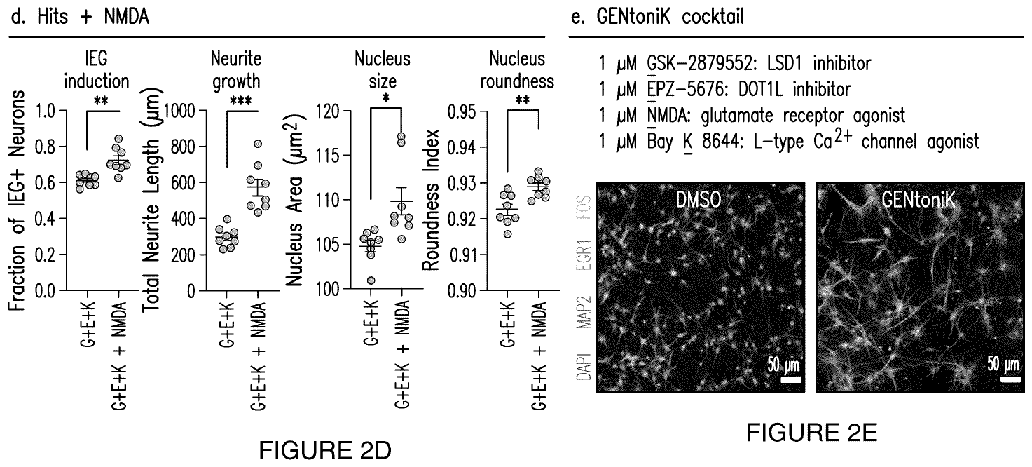

To validate primary hits, the 42 compounds were applied to the maturity assay in triplicates at the screening concentration (5 μM) and ranked by their effect on 4 maturity parameters: nuclear size and roundness, total neurite length, and double KCl-induced FOS/EGR1 cells (FIG. 7B). The 22 compounds with the highest mean normalized score over DMSO across all parameters underwent additional dose-response studies (FIG. 2A) resulting in the identification of 4 compounds with the most pronounced, dose-dependent effects on the mean maturation score (FIG. 2B).

### Small Molecule Cocktail Promoted Neuron Maturity

The 4 confirmed maturation-promoting compounds consisted of two inhibitors of lysine-specific demethylase 1 (LSD1/KDM1A), one inhibitor of disruptor of telomerase-like 1 (DOT1L), and one agonist of L-type calcium channels (LTCC). LSD1 is a histone 3 demethylase at lysine 4 and 9. DOT1L is the sole methyltransferase targeting lysine 79 within the globular domain of histone 3. LTCCs are involved in calcium-dependent transcription and play important roles in neuron development. Transcriptional induction by the LTCC agonist can potentiate the effect of chromatin remodeling by epigenetic regulators such as LSD1 and DOT1L.

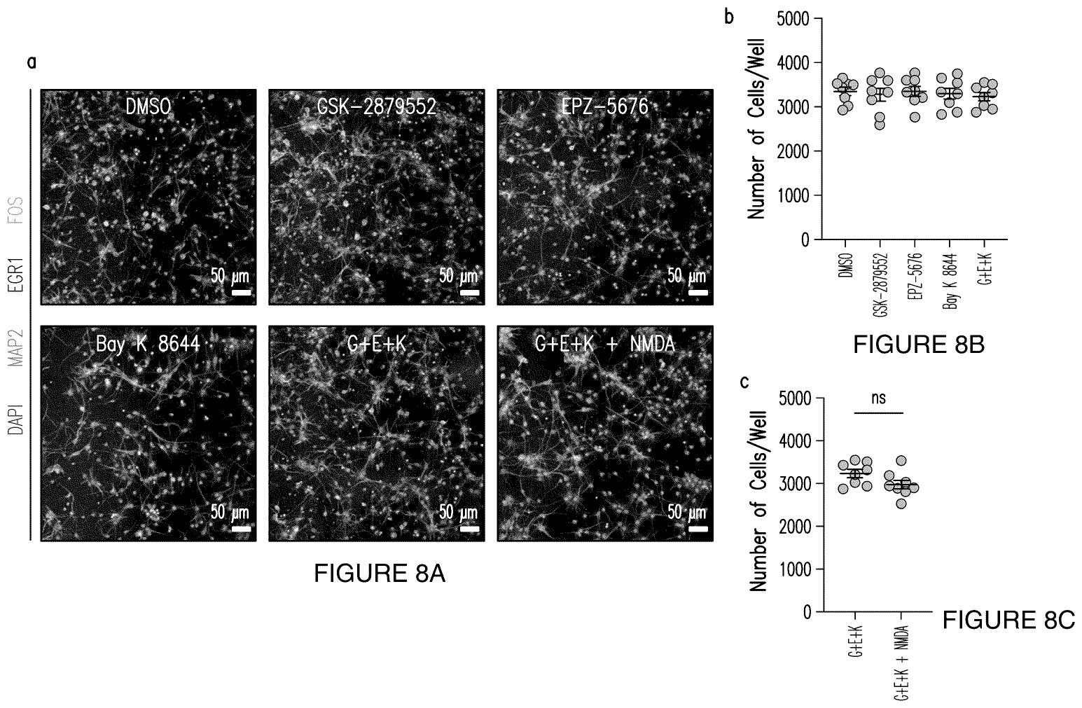

The present disclosure further determined whether a combination of the hits can further enhance neuron maturation. Because two of the confirmed hits target LSD1, it was decided to only pursue one of them (GSK2879552) for combinatorial experiments, as it displayed a stronger combined effect than OG-L002 (FIG. 2B). A combination of the 3 hit compounds significantly increased IEG induction, neurite growth, and nuclear size, but not nuclear roundness, as compared to the results following single compound treatments (FIG. 2C, FIG. 8A). These effects appear to be independent of cell viability, as neither the individual treatments nor combination significantly altered the number of cells with respect to DMSO (FIG. 8B).

In addition to LTCCs, calcium-dependent transcription is initiated through activation of the NMDA glutamate receptors. It was next tested whether the addition of NMDA could further enhance the maturation parameters in the presence of the above 3 hit combination. Significant improvements across all maturity parameters was observed, again without changes in cell survival (FIG. 2D, FIG. 8C), and the resulting 4-drug cocktail (GSK2879552, EPZ-5676, NMDA and Bay K 8644) was nominated as a maturation-promoting strategy, naming it GENtoniK (FIG. 2E).

### GENtoniK Promoted Functional Neuron Maturation

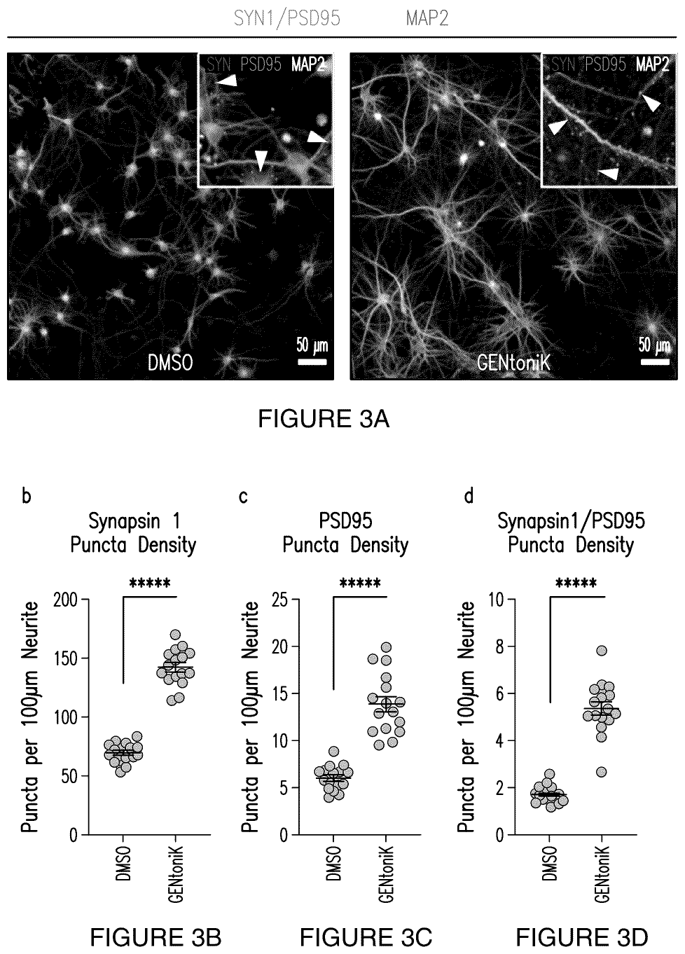

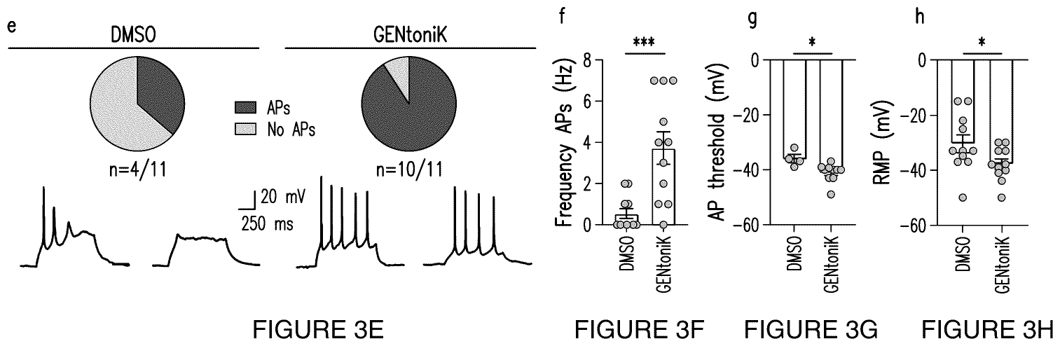

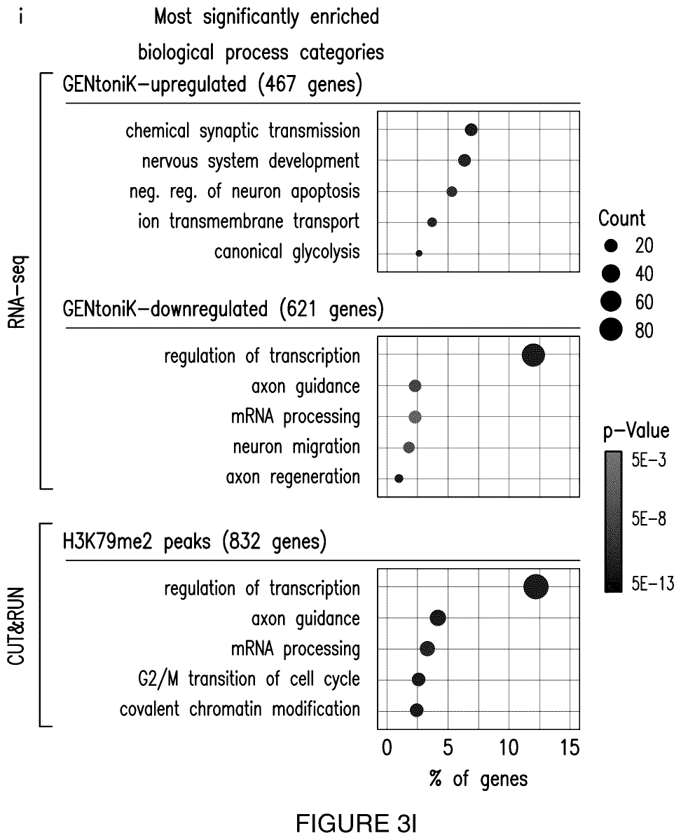

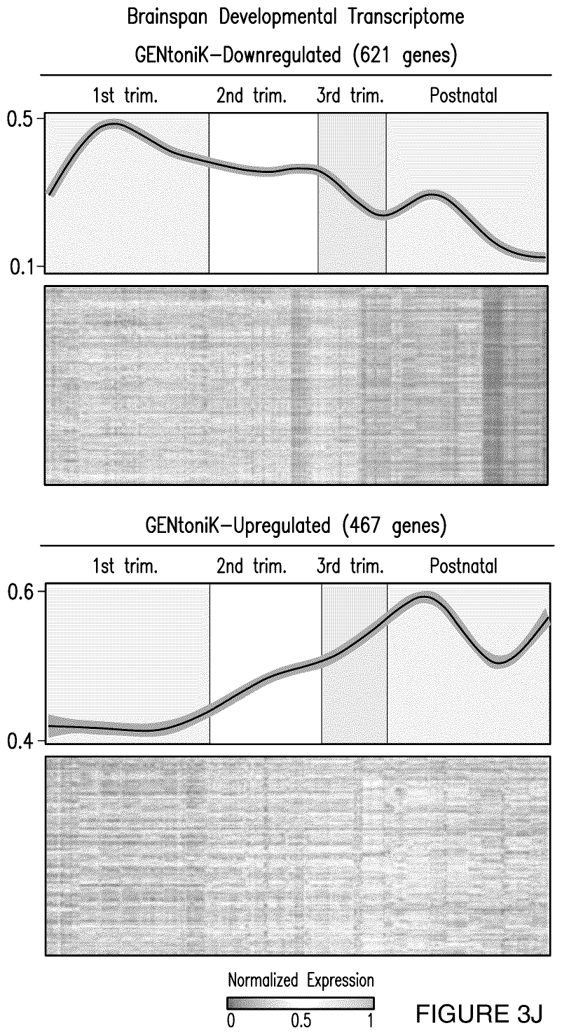

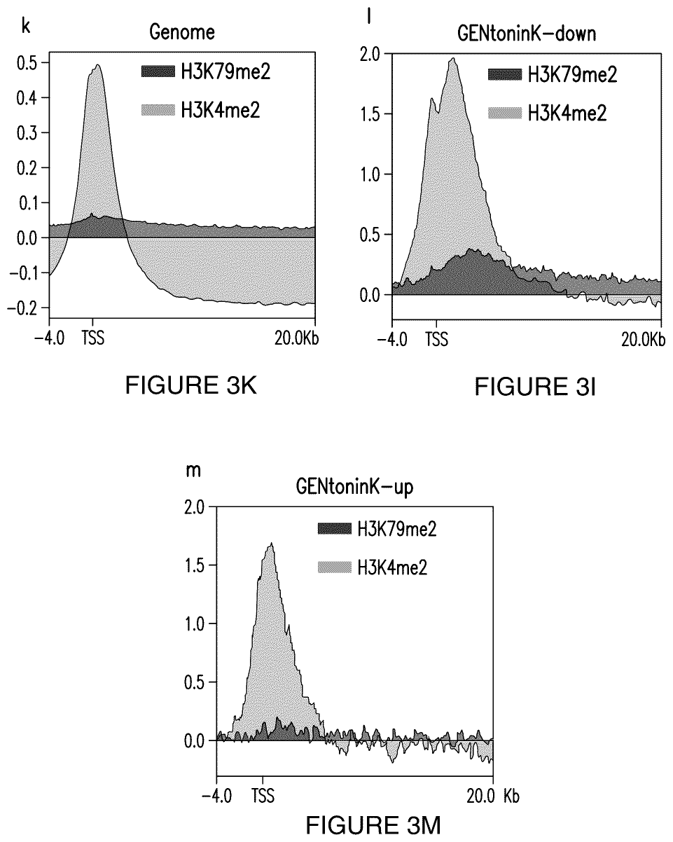

GENtoniK was next validated on additional maturation phenotypes that are orthogonal to those assayed during screening. The formation of chemical synapses is a critical step in neuronal development that also occurs in protracted manner in the human cortex (Liu X. et al., Genome Res. 22, 611-622 (2012)). Immunofluorescent staining was used in day 35 cortical neurons to assess the effect of GENtoniK on synaptogenesis. Density of synaptic assembly was quantified through the apposition of the pre- and post-synaptic markers SYN1 and PSD95 normalized to dendrite length (FIG. 3A). GENtoniK-treated neurons showed increased density of both pre- and post-synaptic markers per neurite length, as well as an increased density of the apposition of synaptic punctae (FIGS. 3B-3D).

Intrinsic electrophysiological features, such as passive membrane properties and the ability to fire action potentials (APs) are also important indicators of functional neuronal maturation (Oswald & Reyes, J. Neurophysiol. 99, 2998-3008 (2008)). To assess the effect of the drug cocktail on membrane properties and excitability, whole-cell patch-clamp recordings were performed in cortical neurons at day 28 from plating. Similar to the IEG studies, treatment was withdrawn 7 days before recordings to ensure that differences were maturation-mediated and not a direct effect of the ion channel activators NMDA and Bay K 8644. Over 90% of GENtoniK-treated neurons displayed evoked APs compared to less than 40% of control neurons (FIG. 3E). Among AP-firing neurons, those treated with GENtoniK displayed higher firing frequencies (FIG. 3F) and lower AP thresholds (FIG. 3G). Resting membrane potential values were significantly more mature in treated neurons (FIG. 3H). These results indicate that GENtoniK significantly promotes synaptic connectivity and excitability.

### GENtoniK Induced Immature to Mature Shift in Transcription

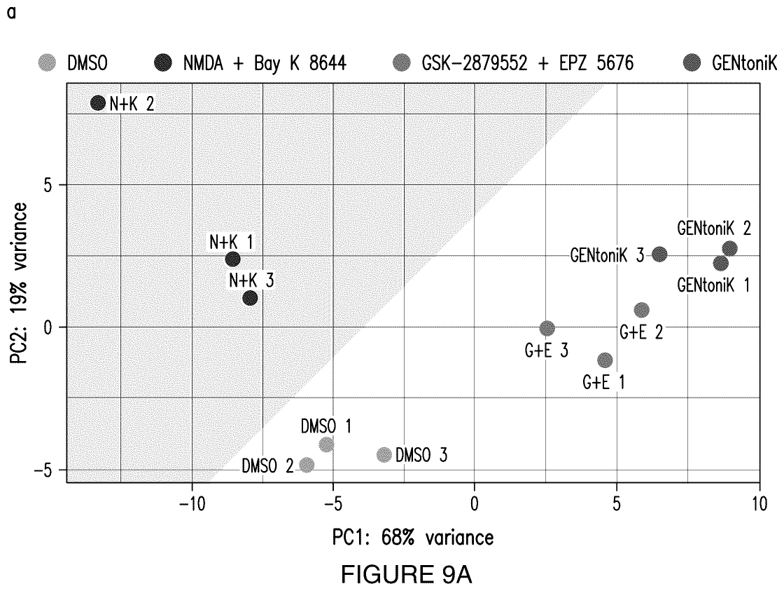

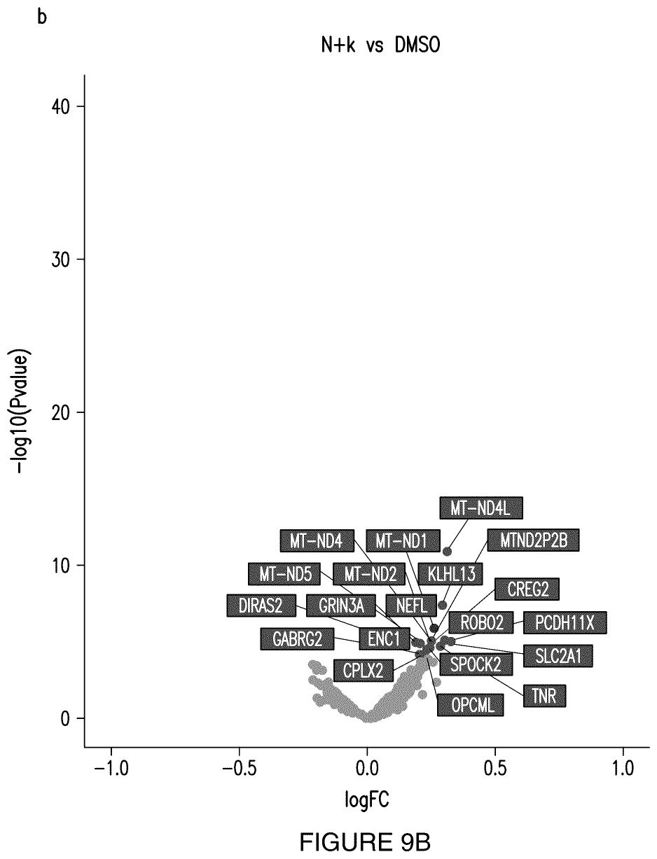

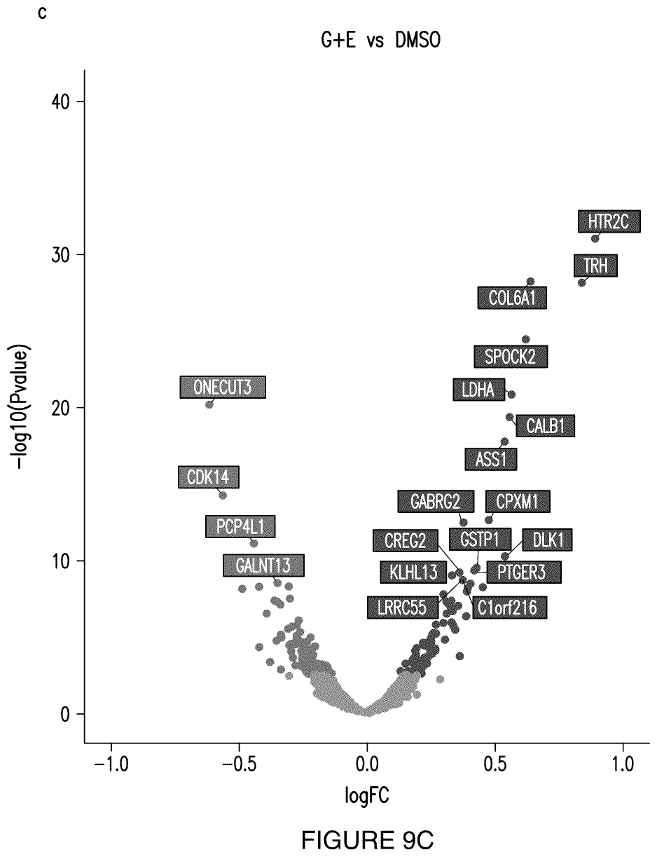

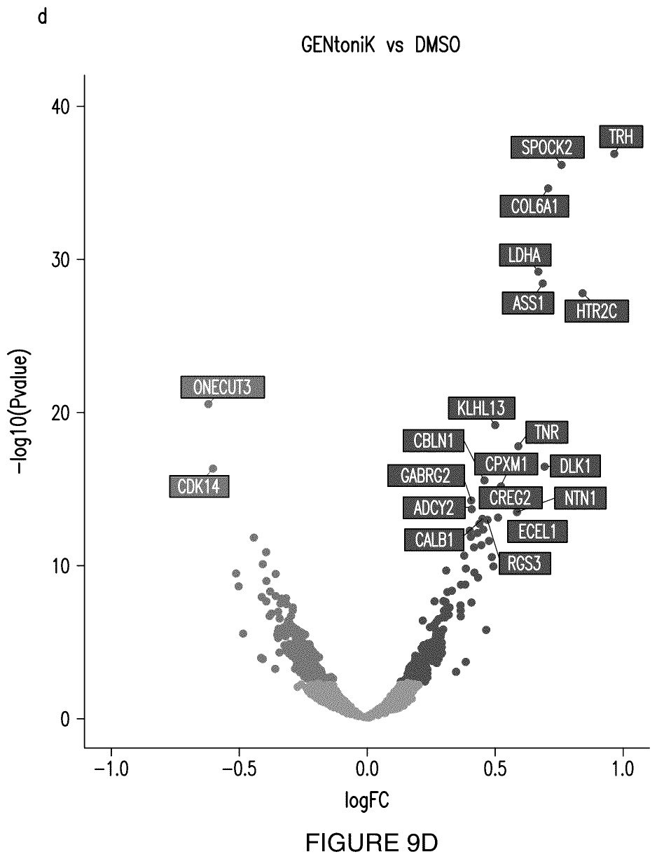

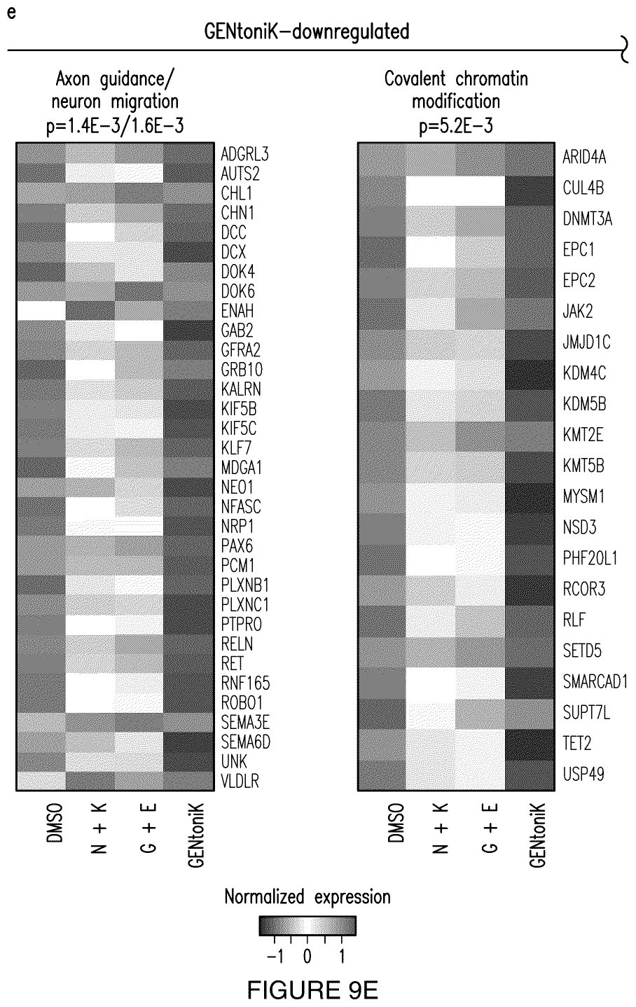

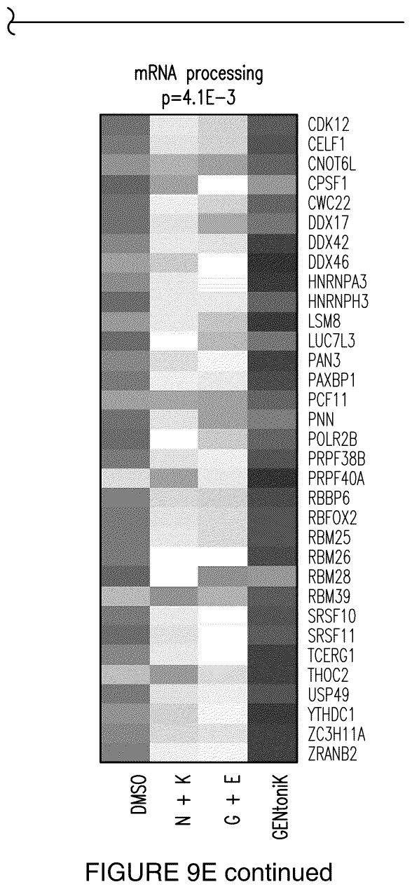

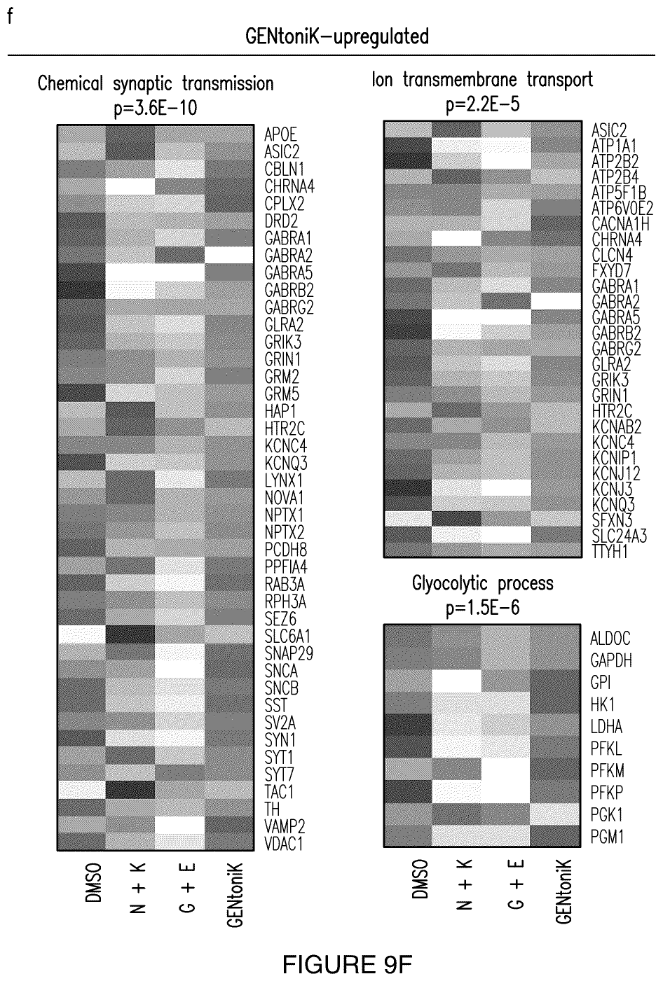

RNA sequencing was conducted to assess global changes in gene expression induced by the small-molecule treatment. In accordance with a dual effect of the cocktail on chromatin state and calcium influx, hPSC-cortical neurons were treated with either the two epigenetic factors, the two calcium channel agonists, or the complete GENtoniK cocktail (FIG. 9A). Genes differentially expressed in GENtoniK were similarly regulated by the epigenetic drugs alone but to a lesser magnitude, which indicated that calcium influx potentiates transcriptional changes facilitated by chromatin remodeling (FIGS. 9B-9D). Although both calcium-channel agonists were identified as maturation enhancers in the present protein-based screen, their combined effect on gene expression was modest 7 days after treatment withdrawal (FIG. 9B).

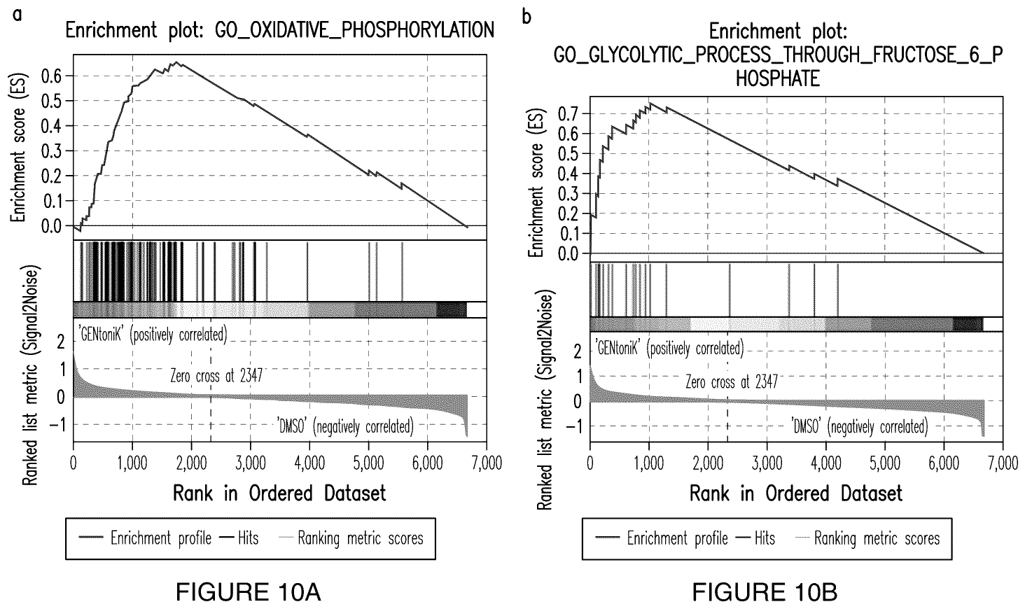

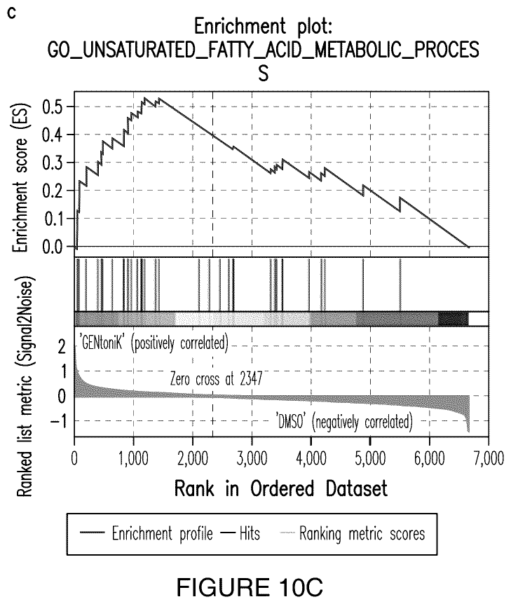

Gene ontology analyses of transcripts downregulated by GENtoniK revealed enrichment in immature, early post-mitotic neuron functions, including migration and axon guidance, as well as transcriptional regulation (FIG. 3I and FIG. 9E). Upregulated genes were enriched in mature neuron functionality, including chemical synaptic transmission and transmembrane ion transport (FIG. 3I and FIG. 9F). While previous studies indicate a switch from glycolytic to oxidative metabolism in maturing neurons (Zheng X. et al., Elife 5, (2016)), it was observed herein enrichment in both glycolysis and oxidative phosphorylation, as well as fatty acid metabolism in treated cells (FIG. 10). To match the transcriptional data with chronological changes of gene expression in vivo, differentially expressed genes were plotted against the BrainSpan Atlas of the Developing Human Brain dataset (Miller J. A. et al., Nature 508, 199-206 (2014)). Genes that are downregulated by GENtoniK were more highly expressed in the early embryo and decreased towards birth (FIG. 3J, left panel). In contrast, genes upregulated by the treatment generally showed an increase in expression through gestation (FIG. 3J, right panel).

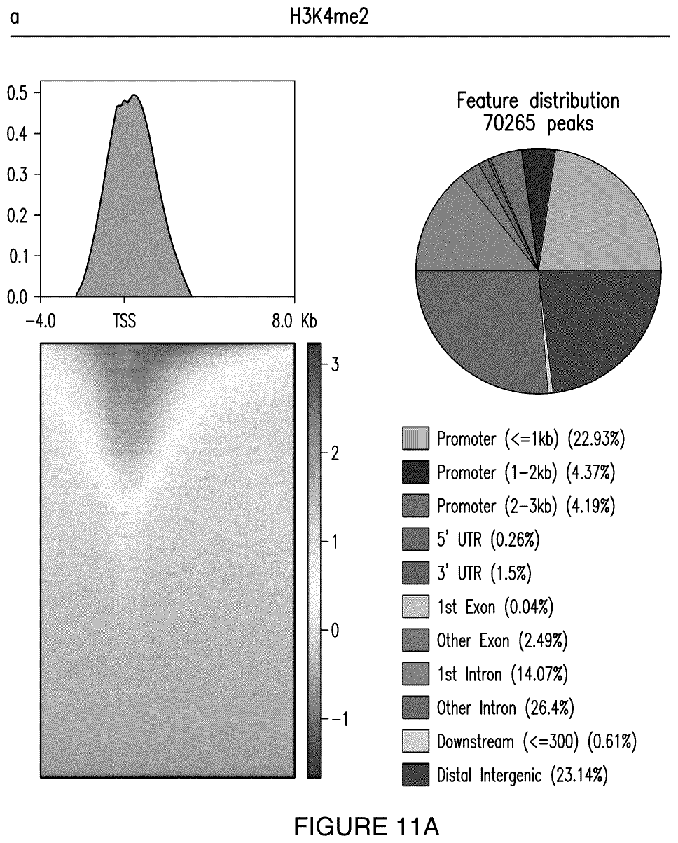

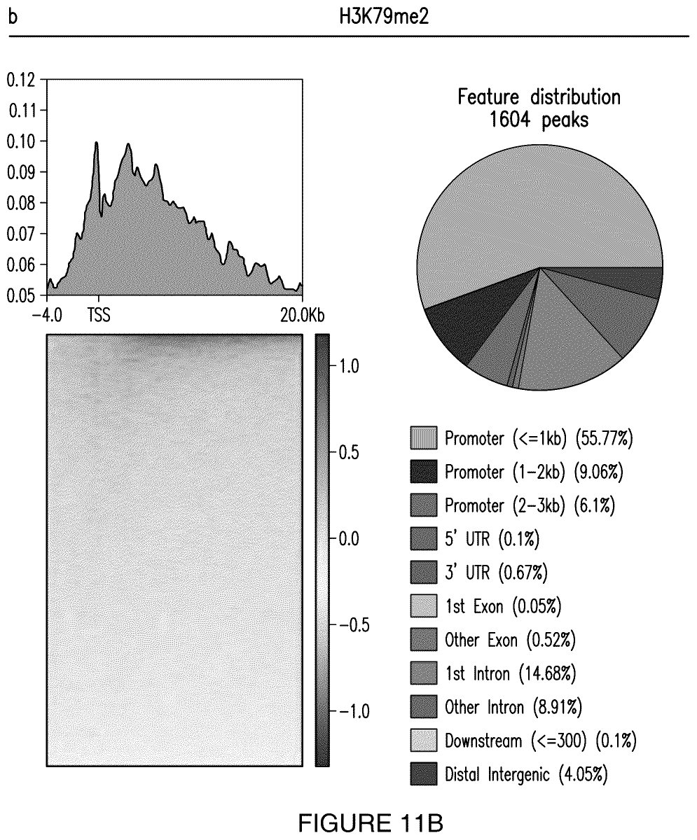

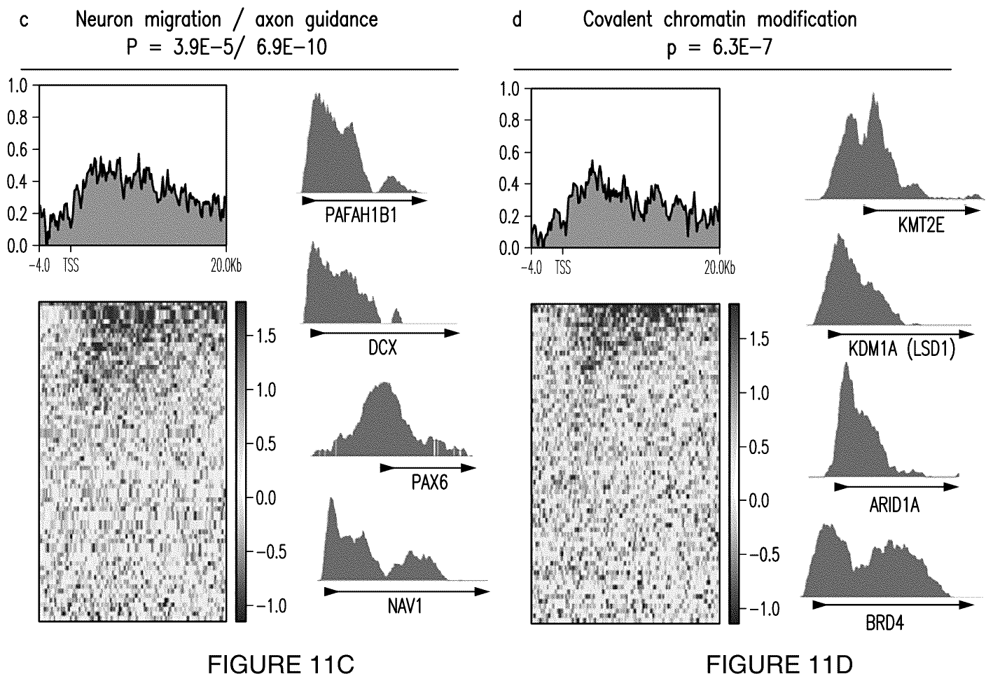

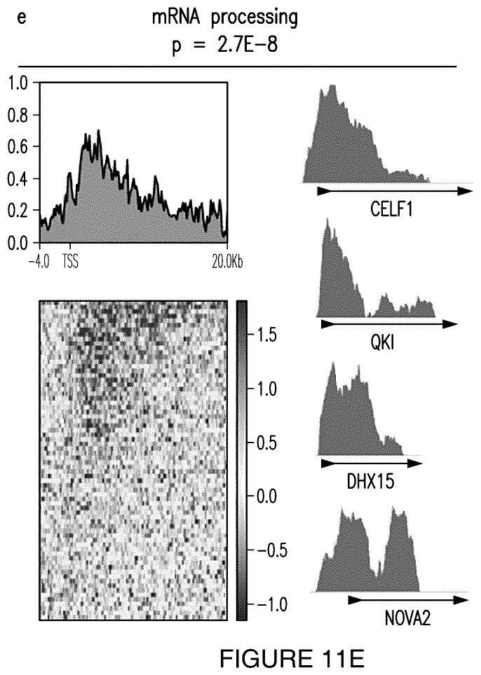

CUT&RUN chromatin profiling was then performed on histone marks downstream of the epigenetic factors targeted by the cocktail (FIG. 3K). Although LSD1 can switch its substrate to H3K9 in the mature neuron-specific variant, the focus herein was on its canonical target H3K4 reasoning that maturation-enhancing inhibition likely targets the immature form. In untreated, day 7 cortical neurons, both H3K4 and H3K79 2-methylation were more highly enriched at GENtoniK-downregulated versus GENtoniK-upregulated genes (FIGS. 3L and 3M). H3K4me2 was widespread in the genome, with highest enrichment in the promoter region and near the transcription start site (FIG. 11A). In contrast, H3K79me2 was enriched at a much smaller subset of genes, where it extended into the transcribed region (FIG. 11B). Interestingly, genes within H3K79 peaks showed near-identical ontology enrichment to those downregulated by GENtoniK by RNA-seq, being overrepresented in neuron migration, chromatin modifying, and RNA processing gene categories (FIG. 3I and FIGS. 11C-11E). Chromatin regulating genes within H3K79me2 peaks include GENtoniK target LSD1 (FIG. 11D), while mRNA processing genes with H3K79me2 peaks, such as NOVA2 and CELF1 (FIG. 11E), have been shown to participate in cortical neuron development (Saito Y. et al., Neuron 101, 707-720.e5 (2019); Popovitchenko T. et al., Nat. Commun. 11, (2020)). These results indicate that H3K79 methylation may play a role in maintaining immature gene expression programs, and that loss of this mark might facilitate neuronal maturation in GENtoniK-treated cells.

### GENtoniK Enhanced Maturation Across Neuronal Culture Systems

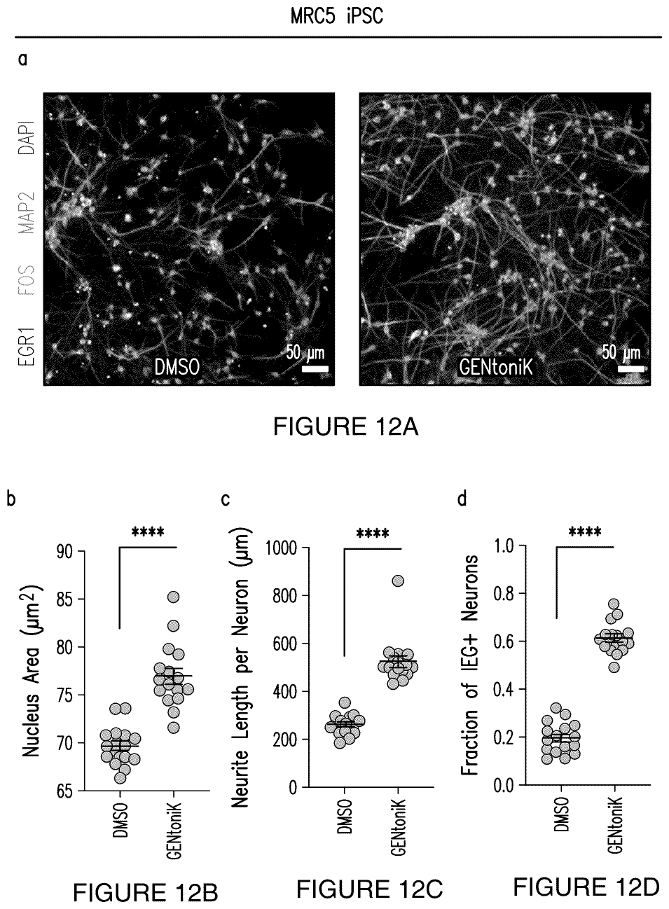

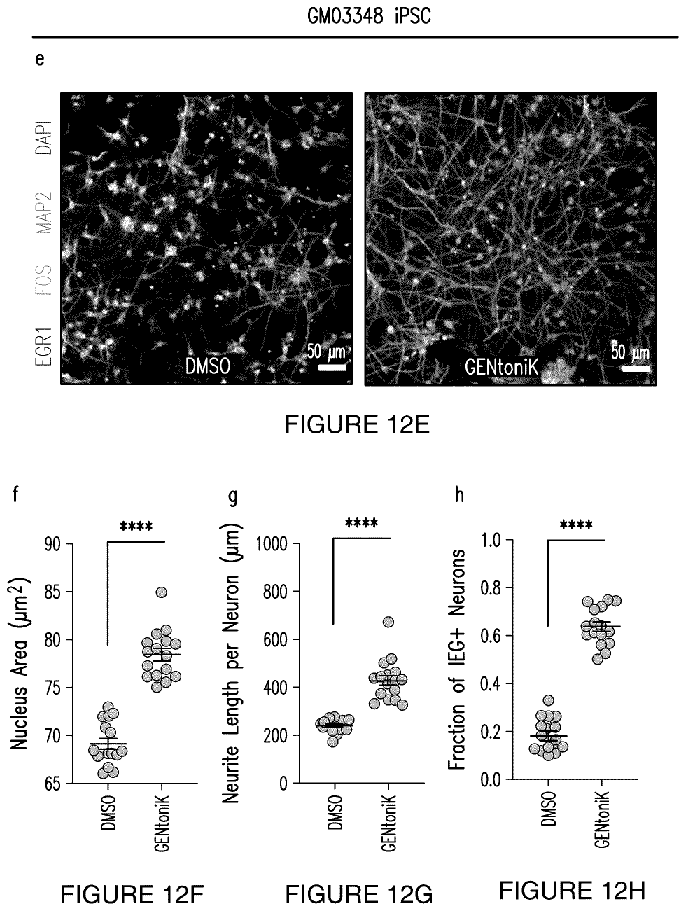

The efficacy of GENtoniK across hPSC-derived neuronal systems was then tested. Because the present screen relied on the female hESC line H9 (WA09), the results in male cortical neurons were first replicated and derived from induced pluripotent stem cell (iPSCs) lines, confirming GENtoniK's effect on maturation across different hPSC lines (hESC versus hiPSC) and across both sexes (FIG. 12).

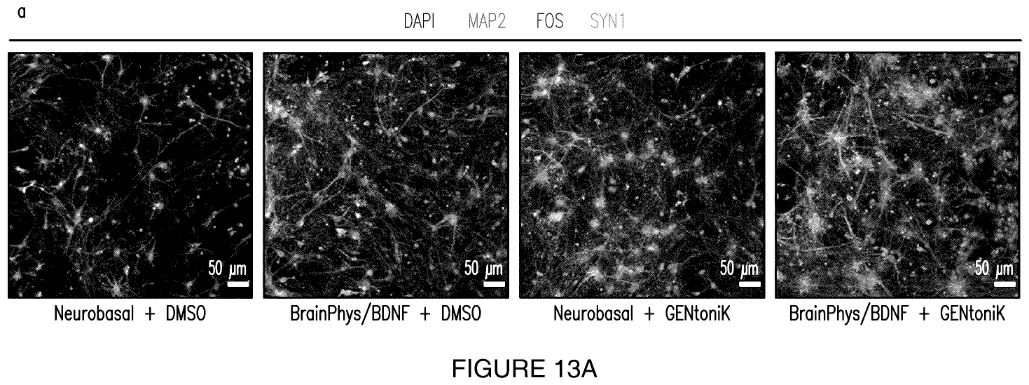

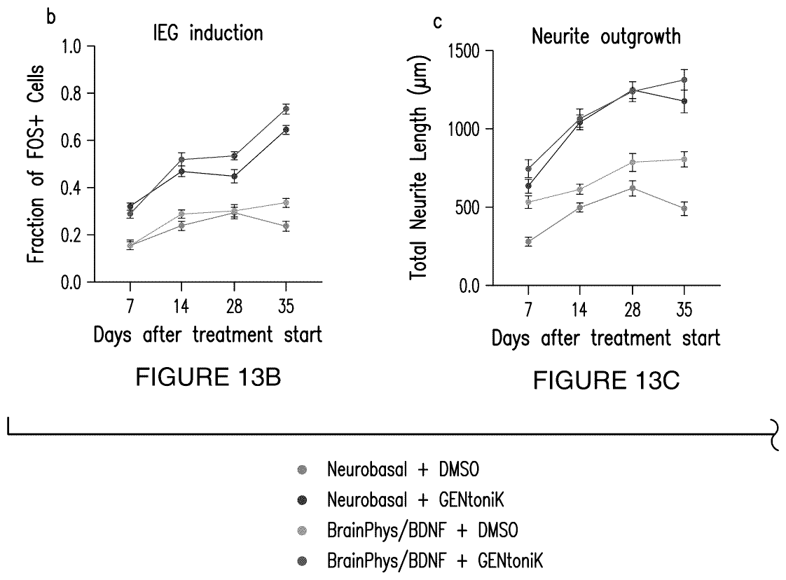

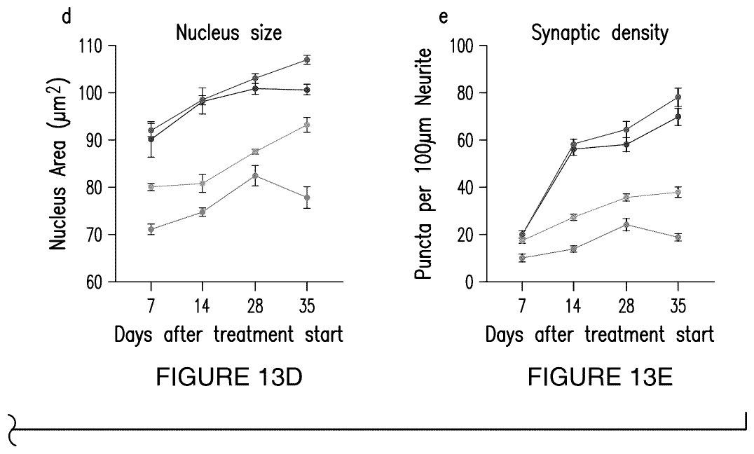

Alternative maturation strategies are routinely employed in neuronal cultures, including the addition of trophic factors such as brain-derived neurotrophic factor (BDNF) and the use of culture media with more physiological levels of glucose and ion concentrations (BrainPhys). Time course experiments were conducted to assess efficacy and compatibility of GENtoniK with existing maturation approaches. GENtoniK in standard Neurobasal medium (without neurotrophic factors) induced neuronal maturation parameters more robustly than the combination of both BrainPhys and BDNF, while treatment with GENtoniK in combination with BrainPhys and neurotrophic factors showed an additional, albeit modest increase in maturation (FIG. 13).

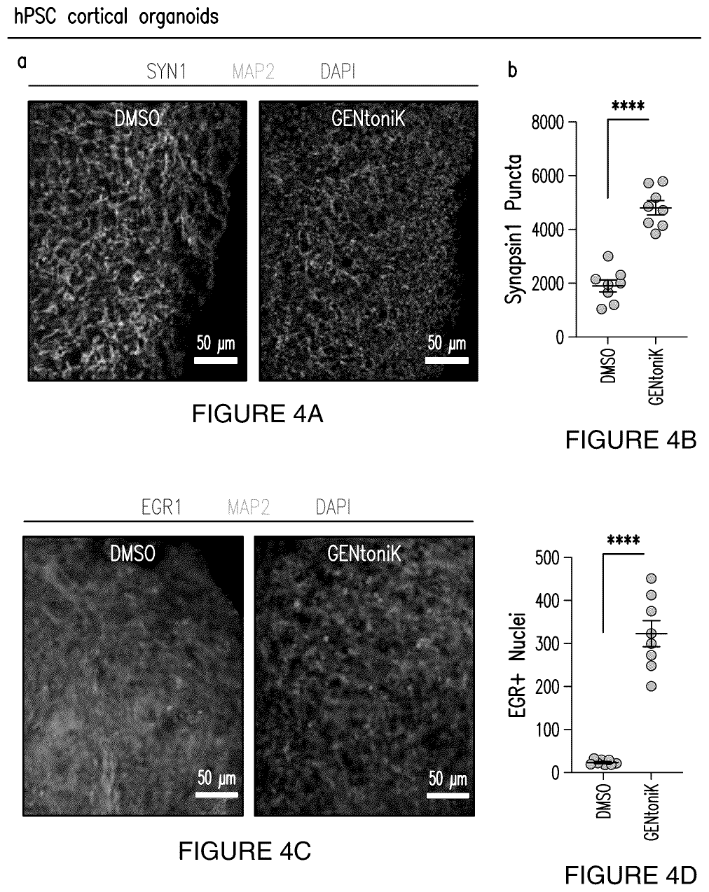

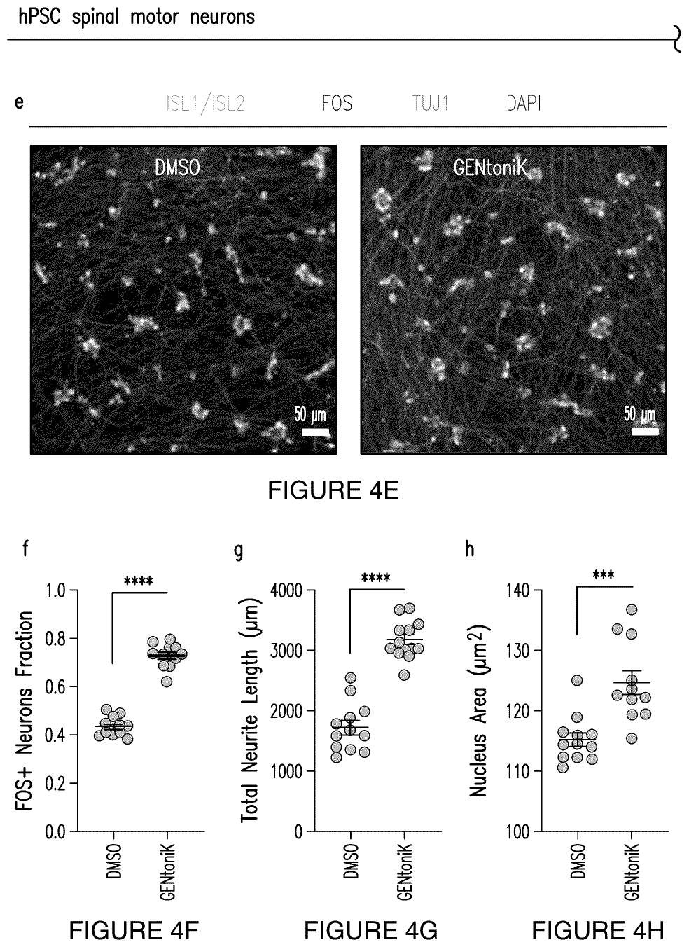

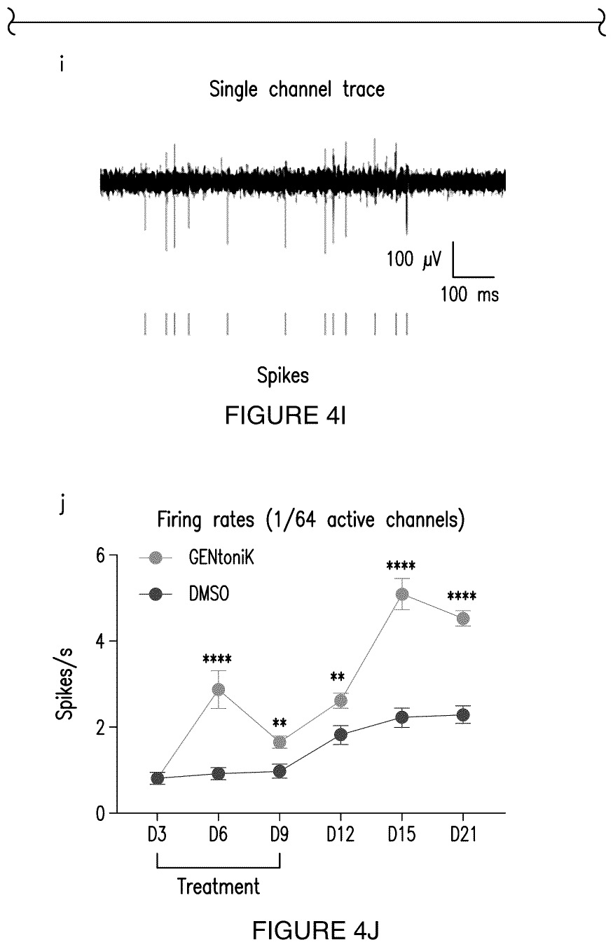

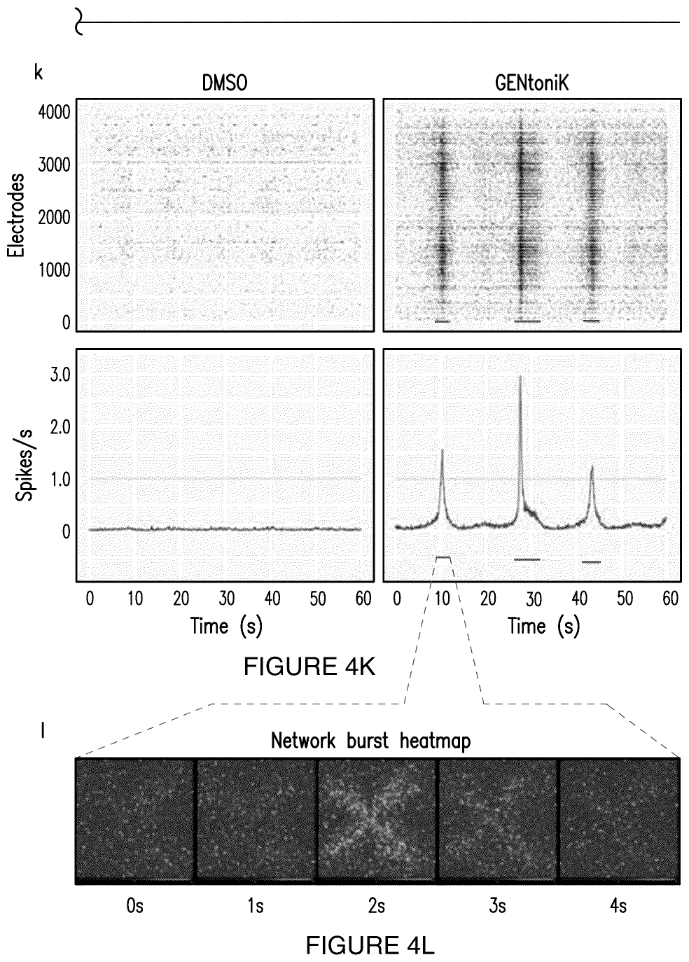

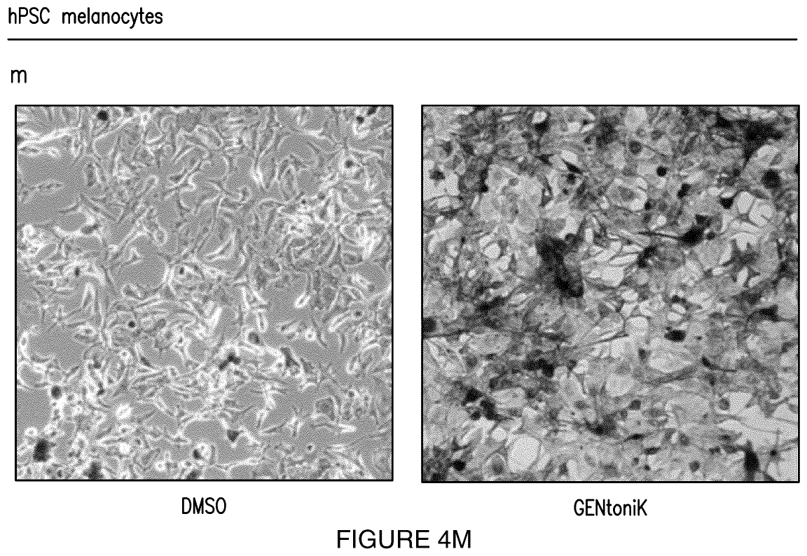

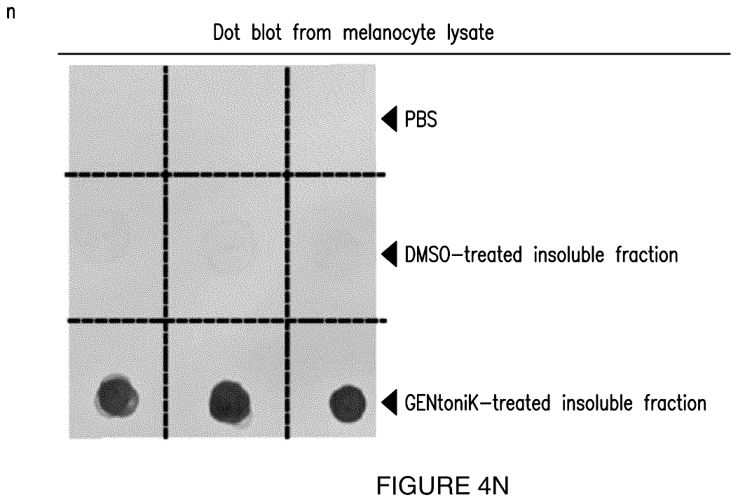

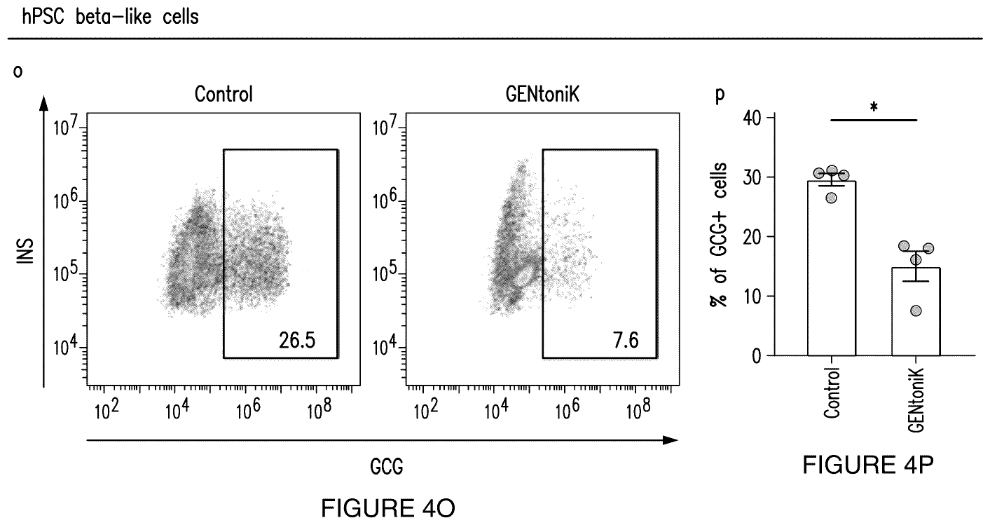

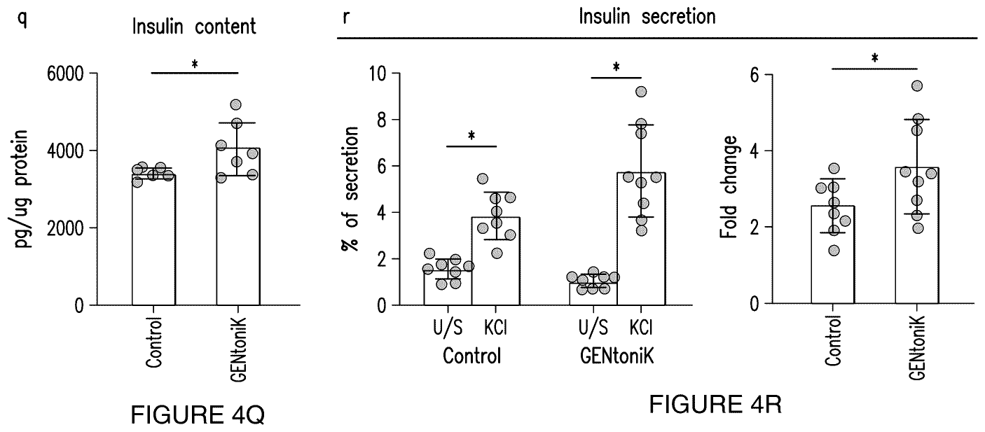

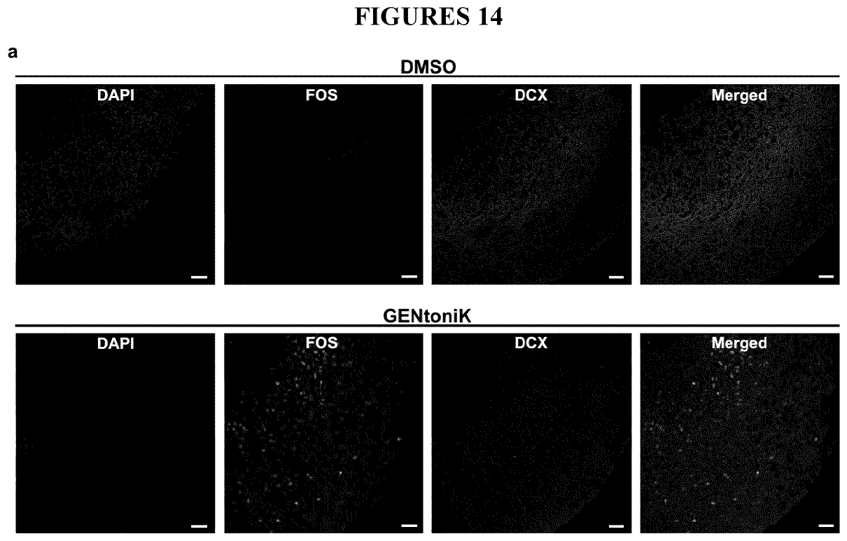

Self-organizing 3D culture systems such as brain organoids have become a widely used model system to study human brain development and disease (Chiaradia & Lancaster, Nature Neuroscience vol. 23 1496-1508 (2020)). However, similar to 2D culture systems, 3D organoids are subject to slow maturation rates (Otani T. et al., Cell Stem Cell 18, 467-480 (2016)). It was observed that forebrain organoids treated with GENtoniK from day 15-50 of derivation, displayed an increased density of SYN1 puncta (FIGS. 4A and 4B), and increased number of cells with nuclear expression of EGR1 and FOS (FIGS. 4C, 4D and FIG. 14) at day 60. For these studies, organoids were not subjected to KCl stimulation before IEG immunostaining, thus indicating higher levels of spontaneous activity following GENtoniK treatment. GENtoniK-treated organoids also displayed lower expression of immature neuron marker DCX (FIG. 14).

It was next addressed whether the treatment can drive the maturation of hPSC-derived neurons outside the cortex or forebrain. ISL1+ spinal motor neurons (SMNs) treated with GENtoniK displayed a highly significant increase across all the maturity parameters tested (FIGS. 4E-4H). It was observed that SMNs exhibit high levels of spontaneous activity when cultured on high-density multielectrode arrays (FIG. 4I). In a time-course experiment, average firing rates were increased modestly in the presence of the drug cocktail (possibly via direct ion channel activation effect). In contrast, a more pronounced effect was observed starting 6 days after treatment withdrawal indicating that the treatment triggered a long-lasting maturation effect (FIG. 4J). Intriguingly, only SMNs pretreated with GENtoniK exhibited highly synchronous bursts of activity in the 0.8-0.6 Hz range (FIGS. 4K, 4L), reminiscent of spontaneous network activity episodes observed in the embryonic spinal cord (Gonzalez-Islas & Wenner, Neuron 49, 563-575 (2006)).

### GENtoniK Enhances Cell Function in Non-Neuronal Lineages

Slow maturation rates of human PSC-derived cells are a common problem across lineages beyond neurons. To assess the potential of GENtoniK in other cell types, neural crest-derived melanocytes which produce the pigment melanin in a maturation-dependent manner were used. The production and secretion of melanin from melanocytes is responsible for human skin and hair color, and hPSCs-melanocytes have been used to model various pigmentation disorders (Mica Y. et al., Cell Rep. 3, 1140-1152 (2013)). Using an established differentiation protocol (Callahan S. J. et al., J. Vis. Exp. 2016, (2016)), treatment of hPSC-derived melanocytes with GENtoniK, starting at day 11, induced a dramatic increase in pigmentation at day 33 of differentiation, compared to untreated melanocytes (FIGS. 4M, 4N).

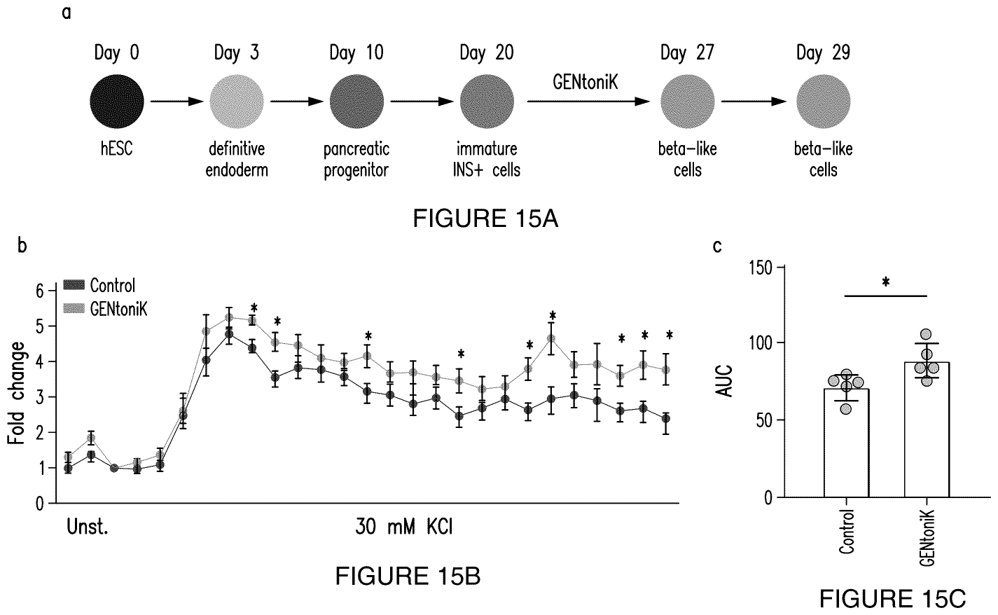

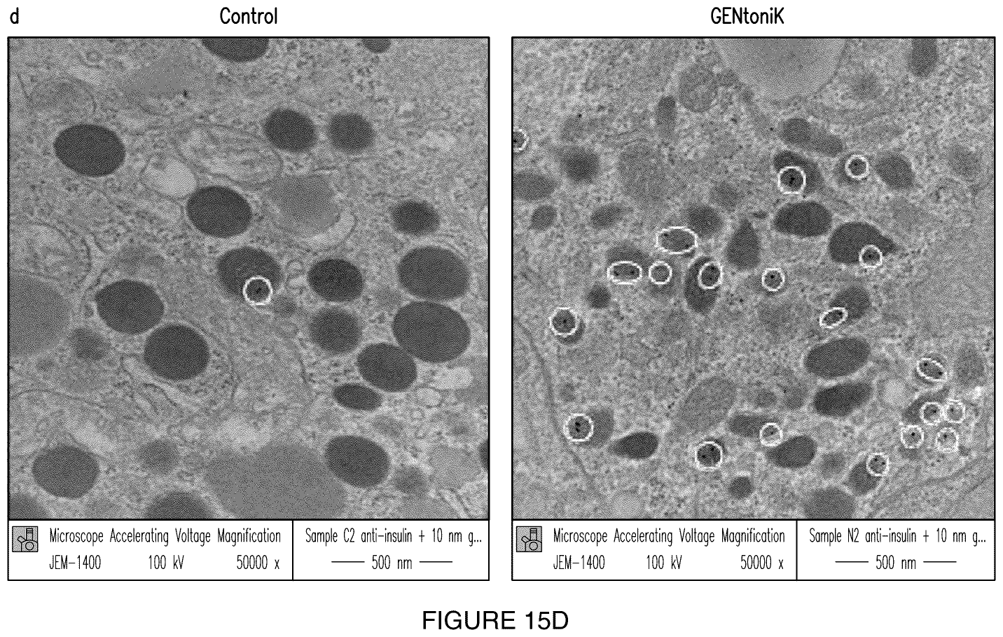

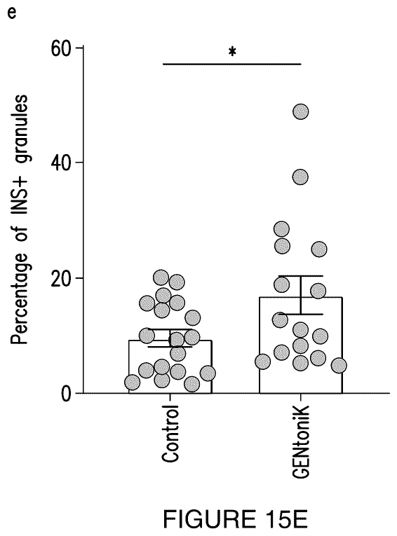

Finally, GENtoniK was tested on a cell type derived from a different germ layer, hPSC-derived insulin-secreting pancreatic beta cells. These cells arise from definitive endoderm (Chen S. et al., Nat. Chem. Biol. 5, 258-265 (2009)) and are of great interest in the development of cell-based treatments for type I diabetes (Mayhew & Wells, Current Opinion in Organ Transplantation vol. 15 54-60 (2010)). Although many protocols have been reported, one major limitation is the generation of a subset of glucagon(GCG)+insulin(INS)+ polyhormonal cells (Teitelman G. et al, Development 118, 1031-1039 (1993)). Flow cytometry analysis revealed that GENtoniK treatment decreased the number of GCG+ cells among INS+ cells (FIGS. 4O, 4P). Importantly, beta-like cells that received GENtoniK treatment from days 20 to 27 of differentiation displayed evidence of improved functional maturation including increased total insulin content, fraction of insulin granules, and KCl-induced insulin secretion at day 29 (FIGS. 4Q-4R; FIG. 15). Therefore, GENtoniK can trigger some aspects of cell function and maturation even in non-neural lineages.

### Discussion

The present disclosure provides a combined chemical strategy to promote the maturation of human stem cell-derived neurons, which was obtained by combining hits from a high-content small molecule screen. Applying a multiparameter readout enabled compounds to be identified that effectively drive neuronal maturation rather than simply promoting individual features such as neurite outgrowth. PCA of the screen results yielded three phenotypic clusters of compounds that either promoted or inhibited neuronal maturation and compounds that promoted the growth of non-neural contaminants. An unexpected finding herein was the identification of TGF-β and ROCK-inhibitors as compounds promoting a “flat cell” non-neuronal fate, which is a known contaminant of neural differentiations and thought to represent a neural crest (Hu & Zhang, Methods Mol. Biol. 636, 123-137 (2010)) or fibroblast-derived (Tiklová K. et al., Nat. Commun. 11, (2020)) mesenchymal cell lineage. Both TGF-β and ROCK-inhibitors are commonly used across many neural differentiation protocols, but the present results indicate that they may promote undesired cell types if used at later differentiation stages.

The present disclosure further discovered the presence of an epigenetic program in immature neurons that prevents rapid maturation of human neurons. GENtoniK acted in a two-pronged manner. The epigenetic probes GSK2879552 and EPZ-5676 induced a shift in chromatin accessibility from an immature (migration, axon guidance) to a mature transcriptional program (synaptic transmission, ion channel subunits). Those changes in chromatin state facilitated NMDA and Bay K 8644-mediated activation of calcium-dependent transcription as an additional driver of maturation.

Several inhibitors of LSD1 were identified herein in the primary screen. The present chromatin profiling data in immature neurons indicated that DOT1L substrate H3K79me2 could be involved in controlling the accessibility of other transcriptional regulators including LSD1, making it an intriguing candidate as a potential master regulator of gene expression during development.

It was demonstrated herein that the same chemical strategy promoted aspects of functional maturation in non-neuronal cells. GENtoniK provided a simple, alternative, and complementary strategy to accelerate the timing of maturation in neuronal and non-neural cell types. Furthermore, the use of GENtoniK facilitated the application of human PSC technology in capturing more mature, adult-like states in modeling human development and disease.

### Materials and Methods

### Cell Culture

Human pluripotent stem cells (hPSCs), both embryonic and induced, were maintained in Essential 8 medium (Thermo) on Vitronectin-coated plates as previously described (Tchieu, J. et al. Cell Stem Cell 21, 399-410.e7 (2017)). Cells were passaged twice per week and collected for differentiations within passages 30 to 50. Mycoplasma testing was conducted every 2 months.

hPSC-derived excitatory cortical neurons were generated using a protocol based on the previously described dual-SMAD inhibition paradigm (Chambers, S. M. et al. Nat. Biotechnol. 27, 275-280 (2009)). Briefly, hESC were dissociated into single cells with Accutase and seeded at 250,000/cm² onto Matrigel-coated plates in Essential 8 medium with 10 μM Y-27632. During days 1 to 10 of the protocol, medium consisted of Essential 6 (Thermo) with 10 μM SB431542 (Tocris) and 100 nM LDN193189 (Stemgent). Wnt inhibitor XAV-939 at 2 μM was included from day 1 to 3 to improve anterior patterning (Tchieu, J. et al. Nat. Biotechnol. 37, 267-275 (2019)). On days 11-20, medium consisted of N2-supplemented DMEM/F12 (Thermo). Cells received daily medium exchanges throughout the differentiation. On day 20 cells were dissociated in Accutase for 30 minutes and cryopreserved in STEM-CELLBANKER solution (Amsbio) at 10 million cells/vial. Neurons were thawed as needed for experiments and plated on poly-L-ornithine and laminin-coated plates (PLO/Lam), in low-glucose (5 mM) Neurobasal-A medium supplemented with 2% B27 and 1% GlutaMAX (Thermo). Neurons received medium exchanges twice per week. During the first 7 days after plating, medium was supplemented with notch-inhibitor DAPT at 10 μM to force lingering progenitors out of the cell cycle (Borghese, L. et al. Stem Cells (2010) doi:10.1002/stem.408).

Primary embryonic rat cortical neurons (Thermo) were thawed following vendor instructions and maintained in the same manner as hPSC-cortical neurons.

Spinal motor neurons derivation was adapted from a previously described protocol (Du, Z. W. et al. Nat. Commun. 6, (2015)) to feeder-free monolayer culture. In brief, Accutase-dissociated hESCs were seeded at 600,000/cm² onto Geltrex-coated plates and underwent dual-SMAD inhibition in the presence of CHIR99021 and Smoothened agonist. On day 11, spinal progenitors were collected and plated on poly-d-lysine, laminin, and fibronectin-coated (PDL/Lam/FN) plates and maintained in N2/B27 medium containing Smoothened agonist, retinoic acid, BDNF, GDNF, CTNF, and DAPT. On day 24, SMNs were re-plated on PDL/Lam/FN and maintained in Neurobasal medium supplemented with 2% B-27, ascorbic acid, retinoic acid, BDNF, GDNF, and CTNF. Treatment with GENtoniK or DMSO was initiated the day after re-plating.

Dorsal forebrain organoid generation was adapted from a previously reported protocol (Cederquist, G. Y. et al. Nat. Biotechnol. 37, 436-444 (2019)). Briefly, 10,000 EDTA-dissociated hPSCs were plated per well of a 96-well V-bottom low-attachment plate (S-bio). Cells were allowed to self-aggregate in hPSC growth medium overnight. From days 1 to 8, medium was changed every two days with Essential 6 supplemented with 10 μM SB431542, 100 nM LDN193189, and 2 μM XAV-939. On day 8, media was switched to organoid growth medium consisting of a 50:50 mixture of Neurobasal and DMEM/F12 with 1% NeuroBrew 21 (Miltenyi), 0.5% N2, 1% GlutaMAX, 0.5% MEM non-essential amino acids solution, 0.1% 2-mercaptoethanol, and 1 μM recombinant human insulin (Sigma). Organoids were collected from the wells on day 14 and transferred to 10 cm dishes at roughly 20 organoids per dish. Dishes were placed on an orbital shaker set to gentle motion to prevent organoid fusion.

Melanocyte differentiation was executed as previously reported (Baggiolini, A. et al. bioRxiv 2020.05.09.081554 (2020)). In brief, the day before differentiation, hPSCs with were plated on Matrigel at 200,000 cells per cm² in E8 medium with 10 μM Y-27632. From days 0 to 11 of the protocol, cells received daily exchanges of Essential 6 containing: 1 ng/ml BMP4, 10 μM SB431542 and 600 nM CHIR99021 (days 0-2); 10 μM SB431542 and 1.5 μM CHIR99021 (days 2-4); 1.5 μM CHIR99021 (days 4-6); and 1.5 μM CHIR99021, 5 ng/ml BMP4 and 100 nM EDN3 (days 6-11). On day 11, melanoblasts were sorted using a BD-FACS Aria6 cell sorter at the Flow Cytometry Core Facility of MSKCC. Cells were dissociated into single cells with Accutase for 20 minutes and then stained with an APC-conjugated antibody against cKIT (Invitrogen). Cells positive for APC (cKIT) were sorted and 4,6-diamidino-2-phenylindole (DAPI) was used to exclude dead cells. Upon FACS sorting, cKIT+ melanoblasts were plated onto dried PO/Lam/FN dishes. Cells were fed with melanocyte medium every 2 to 3 days and passaged using Accutase at a ratio of 1:4 once a week. Melanocyte media consisted of Neurobasal supplemented with: 50 ng/ml SCF, 500 μM cAMP, 10 ng/ml FGF2, 3 μM CHIR99021, 25 ng/ml BMP4, 100 nM EDN3, 1 mM L-glutamine, 0.1 mM MEM NEAA, 2% B27 and +2% N2.

Pancreatic beta cell differentiation was performed using INS^{GFP/W} MEL-1 cells. Cells were cultured on Matrigel-coated 6-well plates in StemFlex medium (Thermo Fisher) and maintained at 37° C. with 5% CO₂. MEL-1 cells were differentiated using a previously reported strategy (Zeng, H. et al. Cell Stem Cell 19, 326-340 (2016)). Briefly, on day 0, cells were exposed to basal medium RPMI 1640 (Corning) supplemented with 1× GlutaMAX (Thermo Fisher), 50 μg/mL Normocin, 100 ng/ml Activin A (R&D systems), and 3 μM of CHIR99021 (Cayman Chemical) for 24 hours. The medium was changed on day 2 to basal RPMI 1640 medium supplemented with 1× GlutaMAX, 50 μg/mL Normocin, 0.2% FBS (Corning), 100 ng/ml Activin A for 2 days. On day 4, the resulting definitive endoderm cells were cultured in MCDB131 medium supplemented with 1.5 g/L sodium bicarbonate, 1× glutamax, 10 mM glucose, 2% BSA, 50 ng/ml FGF7, 0.25 mM ascorbic acid for 2 days. On day 6, the cells were differentiated in MCDB131 medium supplemented with 2.5 g/L sodium bicarbonate, 1× GlutaMAX, 10 mM glucose, 2% BSA, 0.25 mM ascorbic acid, 2 μM retinoic acid, 0.25 μM SANT1, 50 ng/ml FGF7, 200 nM TPB, 200 nM LDN193189 and 0.5×ITS-X supplement for 2 days to pancreatic progenitor stage 1 cells. On day 8, the cells were induced to differentiate to pancreatic progenitor stage 2 cells in MCDB131 medium supplemented with 2.5 g/L sodium bicarbonate, 1× glutamax, 10 mM glucose, 2% BSA, 0.25 mM ascorbic acid, 0.2 μM retinoic acid, 0.25 μM SANT1, 2 ng/ml FGF7, 100 nM TPB, 400 nM LDN193189 and 0.5×ITS-X supplement for 3 days. On day 11, the cells were induced to differentiate to insulin expressing cells in MCDB131 medium supplemented with 1.5 g/L sodium bicarbonate, 1× glutamax, 20 mM glucose, 2% BSA, 0.1 μM retinoic acid, 0.25 μM SANT1, 200 nM LDN193189, 1 μM T3, 10 μM ALKi5, 10 μM zinc sulfate, 10 μg/mL heparin and 0.5×ITS-X for 3 days. On day 14, the cells for static or dynamic KCl stimulated insulin secretion (KSIS) analysis were scraped off from plates and relocated onto 24 mm insert and 3.0 μm polycarbonate membrane, 6-well tissue culture trans-well plate into hemispherical colonies and the cells for insulin content analysis and flow cytometry analysis were kept on original plates. All the cells then were further maturated in MCDB131 medium supplemented with 1.5 g/L sodium bicarbonate, 1× glutamax, 20 mM glucose, 2% BSA, 100 nM LDN193189, 1 μM T3, 10 μM zinc sulfate, 10 μg/mL heparin, 100 nM GS in XX and 0.5×ITS-X for 7 days. Then cells were further matured in MCDB131 medium supplemented with 1.5 g/L sodium bicarbonate, 1× glutamax, 20 mM glucose, 2% BSA, 1 μM T3, 10 μM zinc sulfate, 10 μg/mL heparin, 1 mM acetylcysteine, 10 μM Trolox, 2 μM R428 and 0.5×ITS-X with GENtoniK or control treatment for 7 days.

### Small Molecule Treatment

A bioactive compound library containing 2688 compounds was used for screening at a concentration of 5 μM (Selleck Bioactive Library, Selleck Chemicals). 192 DMSO wells contained within the library were used as negative controls. For confirmation of primary hits, compounds were extracted from the library plates with an Agilent Bravo liquid handling platform and re-subjected to the high-content assay in triplicates at 5 μM. 22 confirmed compounds were purchased from Selleck Chemicals, reconstituted in a suitable solvent and applied for dose-response validation in a concentration log scale (30 nM, 100 nM, 300 nM, 1000 nM, 3000 nM, 10,000 nM). GENtoniK cocktail was defined as a mixture of 4 small molecules: GSK2879552, EPZ-5676, Bay K 8644, and NMDA, applied at a working concentration of 1 μM each. Stocks of individual GENtoniK ingredients were reconstituted in DMSO to 10 mM (GSK2879552, EPZ-5676, Bay K 8644), or in water to 50 mM (NMDA) and stored at −20° C. until the day of experiments. Unless stated otherwise, controls received a corresponding volume of DMSO (3:10,000).

### Immunostaining

Monolayer cultures—Cells were fixed in 4% paraformaldehyde in PBS for 30 minutes, permeabilized for 5 minutes in PBS with 0.1% Triton X-100 and blocked for 30 m in PBS with 5% normal goat serum (NGS). Incubation with primary antibodies was performed overnight at 4° C. at the specified dilution in PBS with 2% NGS. Following 3 washes with PBS, cells were incubated with fluorescently conjugated secondary antibodies (2 μg/ml) for 30 minutes at room temperature. Nuclear staining with DAPI at 1 μg/ml was simultaneous to secondary antibody incubation. For high-content experiments, all steps were assisted by automated liquid handling at the MSKCC Gene Editing and Screening Core Facility. A list of antibodies used in the present disclosure is presented in Table 2.

TABLE 2
Antibody Information

| Antigen | Supplier | Catalog # | Host species | Assay | Dilution |
|---|---|---|---|---|---|
| c-Fos | Abcam | ab208942 | Mouse | ICC | 1:500 |
| c-Fos | Cell Signaling | 2250 | Rabbit | ICC | 1:1000 |
| | Technology | | | | |
| EGR1 | Cell Signaling | 4153 | Rabbit | ICC | 1:500 |
| | Technology | | | | |
| MAP2 | Abcam | ab5392 | Chicken | ICC | 1:5000 |
| Synapsin 1 | Cell Signaling | 5297 | Rabbit | ICC | 1:1000 |
| | Technology | | | | |
| PSD95 | Abcam | ab2723 | Mouse | ICC | 1:200 |
| TUJ1 (β3-tubulin) | Abcam | ab107216 | Chicken | ICC | 1:1000 |
| ISL1/2 | DSHB | 39.4D5 | Mouse | ICC | 1:100 |
| DCX | Cell Signaling | 4604 | Rabbit | ICC | 1:1000 |
| | Technology | | | | |
| TBR1 | Abcam | ab183032 | Rabbit | ICC | 1:100 |
| FOXG1 (BF-1) | Takara Bio | M227 | Mouse | ICC | 1:500 |
| H3K4me2 | Upstate | 07-030 | Rabbit | CUT&RUN | 1:100 |
| H3K79me2 | Active Motif | 39143 | Rabbit | CUT&RUN | 1:100 |
| Mouse IgG | Abcam | ab46540 | Rabbit | CUT&RUN | 1:100 |
| CD117 (c-Kit) | Invitrogen | 17-1179-42 | Mouse | Flow | 1:50 |
| Insulin | Dako | A0564 | Guinea pig | Flow, EM | 1:50 |
| Glucagon | Abcam | ab189279 | Rabbit | Flow | 1:100 |

Forebrain organoids—Organoids were collected in 1.5 ml centrifuge tubes, washed in PBS, and fixed with 4% paraformaldehyde solution in PBS overnight at 4° C. Fixed organoids were rinsed in PBS and equilibrated in a solution of 30% weight/volume sucrose in PBS for 24 hours or until sunk to the bottom of the tube. Organoids were embedded in OCT compound (Fisher) on cryomolds, frozen and sectioned to a thickness of 30 μm in a cryostat. Sections were collected in 1 ml centrifuge tubes (1 per antibody), washed in TBS with 0.3% Triton-X and blocked in the same solution with 10% NGS. Primary antibody incubation was done overnight in TBS with 0.5% Tween-20, and followed by washes, and secondary antibody incubation for 2 hours at RT in the same buffer. Sections were mounted on slides with ProLong medium (Fisher) and imaged on a Zeiss microscope equipped with a 20× high numerical aperture objective and an Apotome optical sectioning system (Zeiss). For quantification of SYN1 puncta, images were batch-analyzed using the Synapse Counter ImageJ plugin (Dzyubenko, E. et al., J. Neurosci. Methods 273, 149-159 (2016)).

### High-Content Imaging

High-content maturity assay—Cortical neurons were seeded PLO/Lam-coated 384-well plates at a density of 5000/well and maintained as described. For bioactive compound screening, compounds were added 7 days after plating to a final concentration of 5 μM in replicate plates. Following 7 days of treatment, cells were rinsed twice and maintained in plain medium for an additional 7 days. Before fixation, one replicate plate was stimulated with 50 mM KCl for 2 hours. Immunostaining for FOS, EGR1, and MAP2 and counterstaining with DAPI was performed as described above. Images (4 fields/well at 20× magnification) were captured through an INCell Analyzer 6000 HCA system (GE Healthcare).

Image analysis and quantification of screen results—Phenotypic analysis of screen images was conducted using the Columbus software (Perkin Elmer). Extracted parameters included total number of nuclei, nuclear area, nuclear roundness index (DAPI); total neurite length per nucleus (MAP2); and fraction of FOS-positive, EGR1-positive and double-IEG positive nuclei (FOS/EGR1). For IEG quantification, ratios of positive nuclei were calculated by applying a threshold of fluorescence intensity within DAPI-positive nuclei. IEG nuclei ratios in unstimulated plates were then subtracted from KCl-stimulated plates to isolate the KCl depolarization-mediated response. Morphological variables (nuclear and neurite) were averaged between unstimulated and KCl plates. Sequential b-score and z-score normalization and principal component analysis were performed in the KNIME analytics platform (Berthold, M. R. et al., 4th International Industrial Simulation Conference 2006, ISC 2006 (2006). doi:10.1145/1656274.1656280) with the High Content Screening Tools extension.

Synaptic marker analysis—hPSC-cortical neurons were thawed and plated on PLO/Lam 96-well plates. Drug treatment was initiated after 7 days and maintained for 21 day. Cells were fixed after an additional 7 days in plain medium. Immunostaining for Synapsin 1, PSD95, and MAP2 was conducted as described above. 10 images per well were captured using the confocal modality of the IN Cell 6000 HCA system. A mask was applied to the area surrounding MAP2-positive processes, and SYN1 and PSD95 puncta were quantified within the defined region. For quantification of pre- and post-synaptic marker apposition, a mask was applied to an area containing and immediately surrounding SYN1 puncta, and PSD95 puncta localized within this region were counted. Synaptic puncta counts per field were normalized to total neurite length.

### Electrophysiology

Whole-cell patch-clamp—hPSC-cortical neurons were plated onto PLO/Lam-coated 35 mm dishes at a density of 75 k/cm². Treatment with GENtoniK or DMSO began 7 days after plating and maintained for 14 days. Recordings were initiated 7 days after treatment withdrawal, within days 28 to 33 from plating. Whole-cell recordings were performed at 23-24° C. while the cells were perfused in freshly made ACSF containing (in mM): 125 NaCl, 2.5 KCl, 1.2 NaH₂PO₄, 1 MgSO₄, 2 CaCl₂, 25 NaHCO₃ and 10 D-glucose. Solutions were pH-corrected to 7.4 and 300-310 mOsm. Neurons were recorded with pipettes of 3-7 MΩ resistance filled with a solution containing (in mM): 130 potassium-gluconate, 4 KCl, 0.3 EGTA, 10 Na₂-phosphocreatine, 10 HEPES, 4 Mg₂-ATP, 0.3 Na₂-GTP and 13 biocytin, pH adjusted to 7.3 with KOH and osmolarity to 285-290 mOsmol/kg. Recordings were performed on a computer-controlled amplifier (MultiClamp 700B Axon Instruments, Foster City, CA) and acquired with an AxoScope 1550B (Axon Instruments) at a sampling rate of 10 kHz and low-pass filtered at 1 kHz.

Multi-electrode array recording—hPSC-derived spinal motor neurons were seeded onto poly-l-lysine-coated complementary metal oxide semiconductor multi-electrode array (CMOS-MEA) probes (3Brain) (Amin, H. et al. Front. Neurosci. 10, (2016)). A 100-μl droplet of medium containing 200,000 neurons was placed on the recording area. After 1 hour incubation, 1.5 ml of medium were added to the probe and replaced every 3 days. Cells received treatment with GENtoniK or DMSO during days 3 to 9 from plating. Recordings were performed every 3 days for 18 days, 24 hours after medium changes. 1 minute of spontaneous activity was sampled from 4096 electrodes using the BioCAM system and analyzed using Brain Wave 4 software. Spikes were detected using a sliding window algorithm on the raw channel traces applying a threshold for detection of 9 standard deviations. Network bursts were detected by applying a hard threshold of 1 spike/second on the entire 4096-channel array.

### Gene Expression and Chromatin Profiling

RNA-seq—RNA was extracted using the Direct-zol RNA miniprep kit (Zymo). Total RNA samples were submitted to GENEWIZ for paired-end sequencing at 30-40 million reads. Analysis was conducted in the Galaxy platform (Afgan, E. et al., Nucleic Acids Res. 46, W537-W544 (2018)). Transcript quantification was performed directly from adapter-trimmed FASTQ files using the Salmon quasi-mapping tool (Patro, R. et al., Nat. Methods 14, 417-419 (2017)) referenced to GENCODE Release 36 (GRCh38.p13) transcripts. DESeq2 (Love, M. I., Anders, S. & Huber, W. Genome Biology (2014)) was used for differential expression analysis from Salmon-generated transcript per million (TPM) values. Differentially expressed genes with a Benjamini-Hochberg adjusted p-value below 0.05 and a baseMean cutoff of 1000 were applied to gene set overrepresentation analysis using the Goseq tool (Young, M. D., Wakefield, M. J., Smyth, G. K. & Oshlack, A. Genome Biol. 11, (2010)). For gene set enrichment, all genes with a baseMean above 1000 were analyzed using the GSEA software (Subramanian, A. et al. Proc. Natl. Acad. Sci. U.S.A. 102, 15545-15550 (2005)).

CUT&RUN—hPSC-derived cortical neurons were collected 7 days after plating for CUT&RUN chromatin profiling using the standard protocol (Meers, M. P., Bryson, T. D., Henikoff, J. G. & Henikoff, S., Elife 8, (2019)). Antibodies against H3K4me2 (Upstate), H3K79me2 (Active Motif) and mouse IgG (Abcam) were used at 1:100 for 100 k cells per antibody. DNA was collected via phenol-chloroform extraction and submitted to the MSKCC Integrated Genomics Operation core for paired-end sequencing at 5 million reads. Analysis was performed in the Galaxy platform. Following alignment to ENSEMBL GRCh38 genome build using Bowtie 2 (Langmead, B. & Salzberg, S. L., Nat. Methods 9, 357-359 (2012)), peaks were called using MACS (Feng, J., Liu, T., Qin, B., Zhang, Y. & Liu, X. S. Nat. Protoc. 7, 1728-1740 (2012)), and visualized with ChIPSeeker (Yu, G., Wang, L. G. & He, Q. Y., Bioinformatics 31, 2382-2383 (2015)) and deepTool2 (Ramírez, F. et al., Nucleic Acids Res. 44, W160-W165 (2016)), using mouse IgG as control for normalization.

### Dot Blot for Melanocyte Pigmentation

hESC-melanocytes were dissociated in Accutase, rinsed, and collected in PBS. A pellet containing 1M cells was lysed in 50 μl RIPA buffer with sonication, and centrifuged at 10,000 RCF for 3 minutes. After discarding the supernatant, the insoluble fraction was resuspended in 80 μl of PBS. 10 μl of this solution was applied to a nitrocellulose membrane, air dried, and imaged with a standard office scanner to assess pigmentation.

### Pancreatic Beta Cell Maturation Assays

Flow cytometry analysis—hESC-derived cells were dissociated using Accutase, fixed and permeabilized using Fixation/Permeabilization Solution Kit (BD Biosciences) according to the manufacturer's instructions. Briefly, cells were first fixed with fixation/permeabilization buffer for 30 mins at 4° C. in dark and then washed twice with washing buffer with 10 mins incubation each time at room temperature. Then, the fixed cells were incubated with primary antibody overnight at 4° C., washed twice with washing buffer with 10 minutes incubation each time at RT. After 30 minutes incubation with fluorescence-conjugated secondary antibody at 4° C., cells were washed twice with washing buffer with 10 minutes incubation each time at room temperature and re-suspended in PBS buffer for analysis. The following primary antibodies were used: anti-Insulin (1:50, Dako) and anti-Glucagon (1:100, Abcam). Samples were analyzed with an Accuri C6 flow cytometry instrument and the data were processed using FlowJo v10 software.

Static and dynamic KSIS—On day 30 cells were starved in 2 mL glucose-free pancreatic beta cells maturation media and followed by 2 mL glucose-free DMEM (with GlutaMAX) for 1 hour and additional 1 hour incubation in KRBH buffer (containing 140 mM NaCl, 3.6 mM KCl, 0.5 mM NaH₂PO₄, 0.2 mM MgSO₄, 1.5 mM CaCl₂, 10 mM Hepes (pH 7.4), 2 mM NaHCO₃ and 0.1% BSA) in a 5% CO₂/37° C. incubator. To perform static KSIS, cells were exposed sequentially to 100 μL of KRBH with 2 mM glucose, or 2 mM glucose with 30 mM KCl; supernatants were collected after 60 minutes and spun down to eliminate the cells and debris. Supernatants were used for ELISA (Insulin Chemiluminescence ELISA Jumbo, Alpco). To measure the total insulin levels in cells in each sample, cells were lysed in RIPA buffer supplemented with 1× protease inhibitor cocktail (ThermoFisher Scientific) with vortexing for 2 minutes at RT and flash freeze the samples in liquid nitrogen and thaw to help the lysis and release the cellular insulin. Lysates were spun down, and supernatant was used for ELISA. Insulin secretion from cells in each condition was normalized to KRBH treatment. To perform dynamic KSIS, cells were embedded in chambers with the order of filter paper-biogel P4 beads-cells-biogel P4 beads order sandwich and then the chambers were installed on the biorep perfusion system (Biorep Technology) and first perfused with Krebs buffer containing 2 mM glucose at a flow rate of 100 μL/min and followed by perfusion with 2 mM glucose+30 mM KCl for 25 minutes. Insulin secretion from cells in each fraction in KCl stimulation were normalized to KRBH treatment.

Insulin content measurement—D30 hESC-derived beta-like cells were dissociated using Accutase and resuspended in DMEM containing 2% FBS and 1 mM EDTA. 80,000 INS-GFP⁺DAPI⁻ cells were FACS sorted by an ARIA2 instrument, washed once with PBS and lysed in 200 μL RIPA buffer supplemented with 1× protease inhibitor cocktail (ThermoFisher Scientific). The insulin content was measured by ELISA.

Immuno-electron microscopy—To analyze granular ultrastructure, control or chemical treated-hPSC-derived beta-like cell clusters were washed with serum-free media and fixed with 2.5% glutaraldehyde, 4% paraformaldehyde, 0.02% picric acid in 0.1 M buffer. After three buffer washes, the cell clusters were fixed again using 1% OsO₄⁻1.5% K-ferricyanide at RT for 60 mins followed by three buffer washes. After dehydration steps of 50%, 70%, 85%, 95%, 100%, 100%, 100% EtOH, the cell clusters were infiltrated with 100% EtOH mixed 1:1 with acetonitrile, followed by acetonitrile, acetonitrile 1:1 with EMbed 812 epoxy resin, resin and finally, embedded in fresh resin which was polymerized at 50° C. for 36 hours. Sections were cut at 65 nm and picked up on nickel grids. Sections were washed with saturated Na-periodate, followed by 50 mM glycine, and blocking buffer. Then, the sections were stained with anti-insulin antibody at original dilution followed by 10 nm gold Goat anti-Guinea pig IgG (Aurion, 1:100). Samples were imaged with a JEOL JEM 1400 TEM with an Olympus-SIS 2K×2K Veleta CCD camera.

### Statistical Analysis

Averages are reported as arithmetic means+/−SEM (standard error of the mean) unless otherwise indicated. Statistical significance was marked by asterisk notation as follows: (ns) p>0.05, (*)p≤0.05, (**)p≤0.01, (***)p≤0.001, (****)p≤0.0001.

Although the present disclosure and certain of its advantages have been described in detail, it should be understood that various changes, substitutions and alterations can be made herein without departing from the spirit and scope of the disclosure. Moreover, the scope of the present application is not intended to be limited to the particular embodiments of the process, machine, manufacture, and composition of matter, and methods described in the specification. As one of ordinary skill in the art will readily appreciate from the disclosure of the present disclosure, processes, machines, manufacture, compositions of matter, or methods, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, or methods.

Various patents, patent applications, publications, product descriptions, protocols, and sequence accession numbers are cited throughout this application, the disclosure of which are incorporated herein by reference in their entireties for all purposes.

### Example 2: An Epigenetic Barrier in Neural Progenitor Cells and Early Neuron Determined the Timing of Human Neuronal Maturation

The development of the Central Nervous System (CNS) follows a coordinated sequence of events in which a myriad of cell identities is specified, differentiated, and assembled giving rise to mature functional neuronal circuits. While fundamental developmental steps are broadly conserved throughout mammalian evolution, the pace at which development proceeds vary considerably among species (Toma, K. et al., Dev Growth Differ 58, 59-72 (2016); Ebisuya, M. & Briscoe, J., Development 145, (2018)), with human CNS running at a very protracted timescale compared to rodents and even primates' counterparts. A challenge for understanding the development of brain circuits is to identify the factors that instruct neurons to accomplish each developmental step at the appropriate stage. Neuronal maturation follows an intrinsic species-specific developmental pace that is extremely protracted in humans and is retained during human pluripotent stem cells (hPSCs) differentiations.

The sequential order as well as the duration and pace of developmental transitions are conserved ex vivo during in vitro Pluripotent Stem Cells (PSC) differentiations (Barry, C. et al., Dev Biol 423, 101-110 (2017)). For instance, PSC from different species differentiated toward neurons of the cerebral cortex, faithfully recapitulate in culture the sequential generation of neuron subtypes and glia, following a “schedule” that largely match the species-specific pace of in vivo natural cortical development (Gaspard. N. et al., Nature 455, 351-357 (2008); Espuny-Camacho, I. et al., Neuron 77, 440-456 (2013); Shi, Y. et al., Nature Neuroscience 15, 477-486, S471 (2012); Anderson, S. et al., Current Opinion in Neurobiolog 27C, 151-157 (2014); Otani, T. et al., Cell Stem Cell 18, 467-480 (2016); Shen, Q. et al., Nature Neuroscience 9, 743-751 (2006)). Species-specific differences in developmental rates are also observed at later stages during the maturation of PSC-derived neurons, with more astonishing (˜10-fold) timing differences among mouse and human neurons compared to the 2/3-fold difference in the rate of early embryogenesis (Shi, Y. et al., Nature Neuroscience 15, 477-486, S471 (2012); Otani, T. et al., Cell Stem Cell 18, 467-480 (2016); Linaro, D. et al., Neuron 104, 972-986 e976 (2019); Rayon, T. et al., Science 369, (2020); Matsuda, M. et al., Science 369, 1450 (2020); Cardoso-Moreira, M. et al., Nature 571, 505-509 (2019)). The processes that control embryonic and fetal nervous system patterning and cell fate specification have been largely studied in vivo and in vitro, leading to the establishment of new paradigms for the induction of human PSC (hPSC) toward a large variety of neuronal and non-neuronal cell types (Tabar, V. & Studer, L., Nat Rev Genet 15, 82-92 (2014)). However, the factors that instruct the acquisition of cell maturity subsequent to cell fate specification remain poorly understood, which is a particular challenge in human development. Neuronal maturation represents one of the most lengthy cell transitions that spans fetal and postnatal development and last weeks, months or years depending on the species (Sousa, A. M. M. et al., Cell 170, 226-247 (2017)). One of the most striking examples is the human cerebral cortex, the regions of the CNS involved in high-order cognition and behaviors that increased in size and complexity during evolution (Sousa, A. M. M. et al., Cell 170, 226-247 (2017); Geschwind, D. H. & Rakic, P., Neuron 80, 633-647 (2013); Silbereis, J. C. et al., Neuron 89, 248-268 (2016)), in which the assembly and refinement of neuronal circuits through synaptic-genesis and pruning takes months-to-years and up-to-decades respectively (Sousa, A. M. M. et al., Cell 170, 226-247 (2017); Silbereis, J. C. et al., Neuron 89, 248-268 (2016)). hPSC-derived cortical neurons follow the clock of human maturation and therefore require extremely protracted timing (in the order of months) to acquire adult-like electrophysiological and synaptic function (Ameele, J. van den et al., Trends Neurosci 37, 334-342 (2014)). The retention of largely immature features and gradual maturation are shared among distinct hPSC-derived neuron types, including midbrain dopaminergic (Kriks, S. et al., Nature 480, 547-551 (2011)), sensory (Chambers, S. M. et al., Nat Biotechnol 30, 715-720 (2012)) and more prominently cortical excitatory and inhibitory identities (Shi, Y. et al., Nature Neuroscience 15, 477-486, S471 (2012); Linaro, D. et al., Neuron 104, 972-986 e976 (2019); Maroof, A. M. et al., Cell Stem Cell 12, 559-572 (2013); Nicholas, C. R. et al., in Cell Stem Cell 12, 573-586 (2013); Marín O., Cell Stem Cell 12, 497-499 (2013)).

Extrinsic environmental factors, such as neuron-glia interactions (Ullian, E. M. et al., Science 291, 657-661 (2001)), network activity (Piatti, V. C. et al., J Neurosci 31, 7715-7728 (2011); West, A. E. & Greenberg, M. E., Cold Spring Harb Perspect Biol 3, (2011)) and secreted molecules (Huang, E. J. & Reichardt, L. F., Annu Rev Neurosci 24, 677-736 (2001)) has been shown to modulate aspects of neuronal functionality, including dendritic spine morphogenesis, neuronal excitability and synaptic connectivity. However, several lines of evidence indicate that the temporal progression toward neuronal maturity is primarily timed through the unfolding of developmental programs that appear to be largely cell intrinsic. hPSC-derived cortical neurons transplanted into the rapidly maturing mouse neocortex develop adult-like morphologies, dendritic spine function as well as intrinsic and extrinsic connectivity in ˜9 months compared to ˜4 weeks for the mouse native and PSC-derived transplanted neurons (Linaro, D. et al., Neuron 104, 972-986 e976 (2019); Falkner, S. et al., Nature 539, 248-253 (2016); Qi, Y. et al., Nat Biotechnol 35, 154-163 (2017)) Species-specific maturation rates and features emerged also between more phylogenetically related species, such as human and chimpanzee iPSC-derived neurons grafted into the mouse brain (Marchetto, M. C. et al., Elife 8, (2019)). Similarly, grafting of hPSC-derived midbrain dopaminergic and cortical neurons in parkinsonian rats and mouse models of cortical stroke respectively, required more than 5 months to induced behavioral and functional recovery (Kriks, S. et al., Nature 480, 547-551 (2011); Tornero, D. et al., Brain 136, 3561-3577 (2013)). This evidence indicates that human neurons yet retain species-specific intrinsic maturation timing in the mouse brain in vivo rather than maturing at the pace of the host specie. Furthermore, intrinsic protracted human neuronal maturation poses a challenge not only for the development of cell replacement strategies for brain repair but also for the study of neurological and psychiatric disorders that typically manifest during postnatal life as alterations in the activity of neural networks (Marin, O., Nat Med 22, 1229-1238 (2016)). Thus, understanding the mechanisms that define and drive the time frame of human neuronal maturation is critical to exploit the full potential of hPSC-derived neurons in modelling and treating brain disorders.

Using a novel platform that synchronized the generation of cortical neurons from hPSC, the present disclosure established morphological, functional, and molecular roadmaps of maturation. The present disclosure found that the temporal unfolding of maturation programs proceeded gradually and was limited by the retention of complex epigenetic signatures. Loss-of-function of multiple epigenetic factors at the neuron stage triggered precocious molecular and functional maturation. Transient pharmacological manipulation of a subset of epigenetic factors, including EZH2, EHMT1/2 and DOT1L, at progenitor cell stage was sufficient to induce comprehensive molecular and functional signatures of maturity in neurons. The present disclosure shows that the rate at which neurons mature was determined well before neurogenesis through an establishment of an “epigenetic barrier” in progenitor cells that gets slowly erased in neurons, allowing the gradual onset of maturation programs.

### Results:

A hPSCs-Based Platform to Study Human Neuronal Maturation in a Dish.

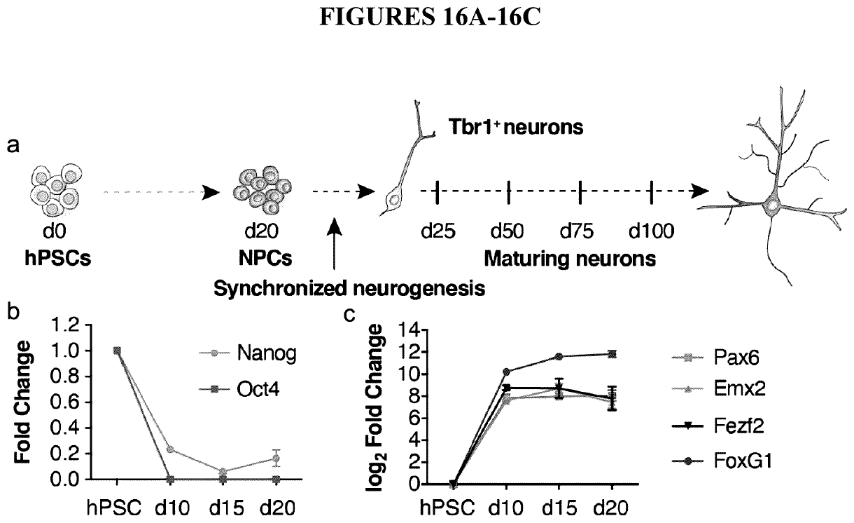

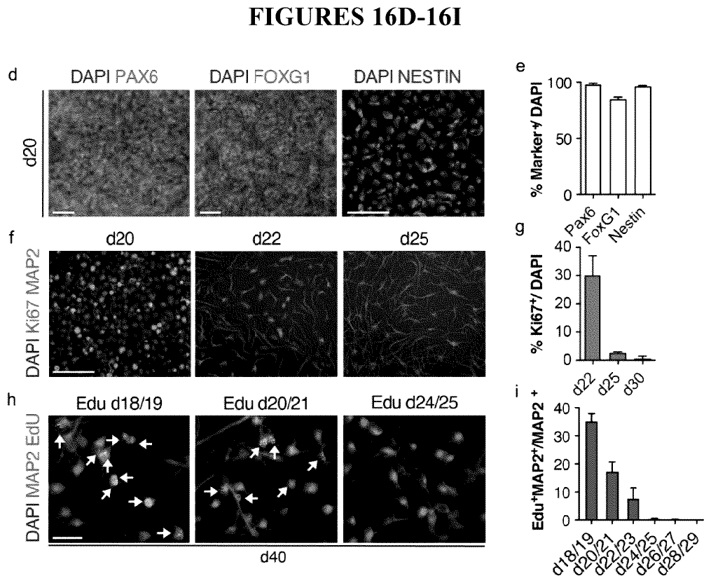

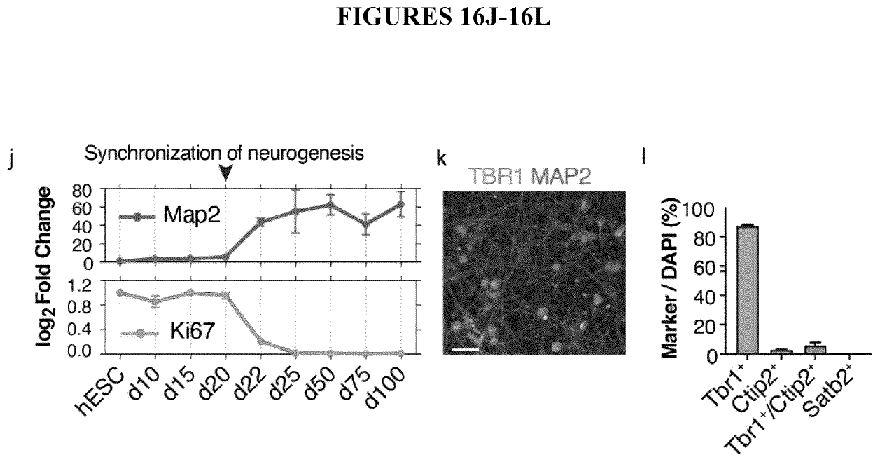

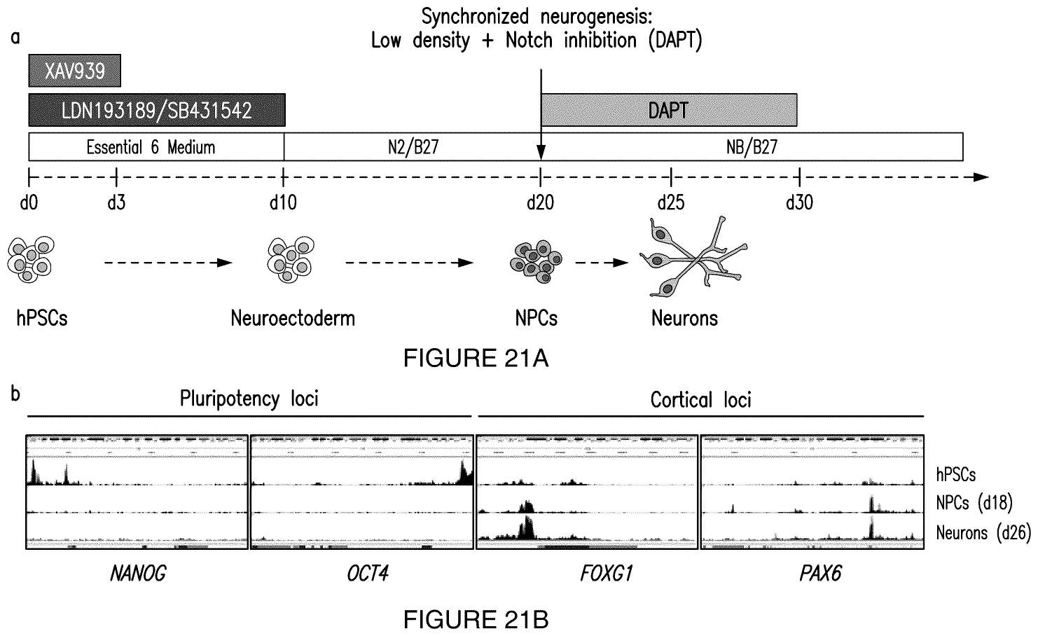

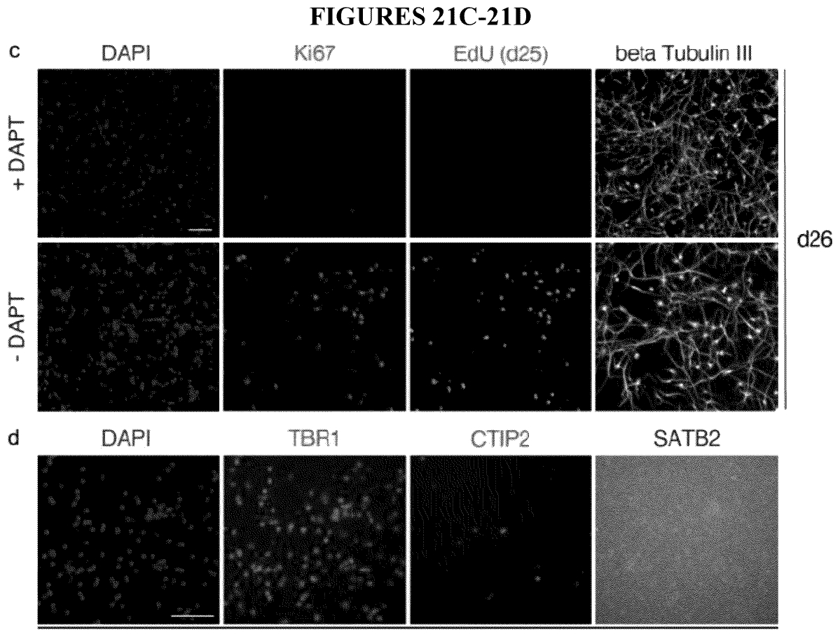

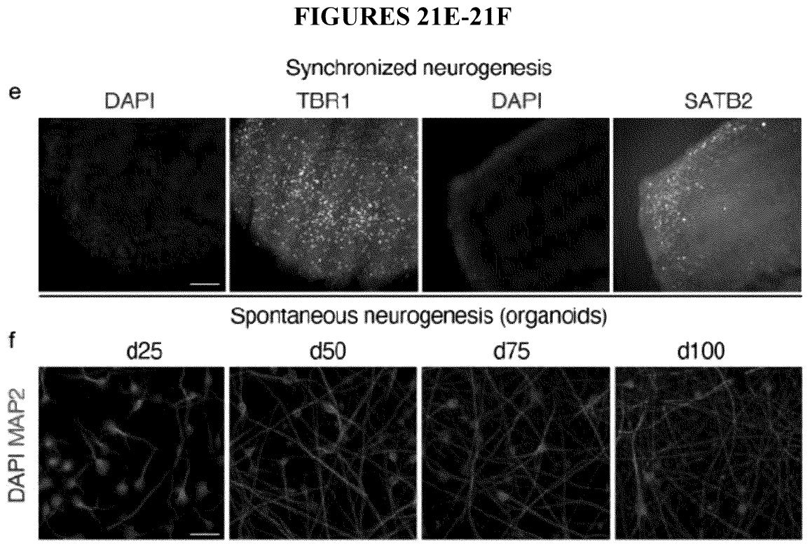

A major limitation for the application of stem cells-based models to study human neuronal maturation is the poor synchronization and the heterogeneity of the cell culture. In current differentiation strategies, different neuronal lineages coexist with precursor cells that yield a constant supply of newly born cell populations that differentiate each at their own pace, representing very different maturation states. To overcome this limitation, a novel platform for the differentiation of human Pluripotent Stem Cells (hPSC) towards homogeneous and synchronized populations of cortical neurons for long-term studies was developed (FIG. 16A, FIGS. 21A-21F). The present disclosure describes induced CNS neuroectodermal patterning by combined inhibition of TGFβ/Activin/Nodal and BMP signaling pathways (i.e. dual-SMAD inhibition, synergistic inhibition of intracellular SMADs, (Chambers, S. M. et al., Nat Biotechnol 30, 715-720 (2012)) and optimized cortical patterning by inhibition of WNT signaling (Maroof, A. M. et al., Cell Stem Cell 12, 559-572 (2013)) (FIG. 21A). These conditions efficiently coaxed hPSC expressing canonical pluripotency markers Oct4 and Nanog towards neural progenitor cells (NPC) that expressed the cortical specific progenitor cell markers FoxG1, Pax6, Emx2 and Fezf2 among others by day (d) 10 of differentiation (FIGS. 16B-16E). Efficient induction of cortical NPC identity was confirmed by robust, stage-dependent changes in chromatin accessibility at pluripotency vs. forebrain-specific genomic loci (FIG. 21B). By d20, the presently disclosed differentiation platform gave rise to a nearly pure homogeneous population of neurogenic cortical precursors (Pax6: 98.3±1.27; FoxG1: 85.12±1.91; Nes: 96.52±0.45; mean %±s.e.m.; FIGS. 16D-16E) that can be further actioned towards the neuronal lineage. To this end, the present disclosure discovered a strategy to trigger synchronous neurogenesis based on optimized density of cell passaging and treatment with the Notch pathway inhibitor DAPT (FIG. 21A, 21C). By d25 of differentiation Ki67⁺ progenitor cells have exited the cell cycle and turned into isochronic MAP2⁺ post-mitotic neurons (FIGS. 16F-16G) that were born few days apart, as confirmed by birth-dating analysis at sequential time windows of EdU labelling (FIG. 16H-16I). In such culture conditions, synchronized neurons were maintained >100 days of differentiation with no major new neurogenic events taking place after d25 (FIG. 16J, FIG. 21F).

There is a strong correlation between the date of birth and the molecular identity of cortical neurons (Gaspard. N. et al., Nature 455, 351-357 (2008); Espuny-Camacho, I. et al., Neuron 77, 440-456 (2013); Molyneaux, B. J. et al., Nature Reviews Neuroscience 8, 427-437 (2007)). Accordingly, the induction of synchronized neurogenesis generated a nearly pure cohort of early born neurons that expressed the lower layer marker Tbr1+ (87.45±0.74; mean %±s.e.m.; FIGS. 16K-16L, FIG. 21D). This contrasted with other cell culture systems in which neurogenesis occurred spontaneously, and gave rise to multiple neuronal identities that coexisted in the same culture (e.g., Tbr1 and Satb2 neurons in brain organoids, FIG. 21E). Thus, induction of synchronized neurogenesis provides an ideal platform to “isolate” a homogeneous population of coetaneous human cortical neurons and investigate their intrinsic functional and molecular maturation at sequential time points over the maturation time course.

hPSCs-Derived Neurons Followed Gradual Functional and Molecular Maturation Programs

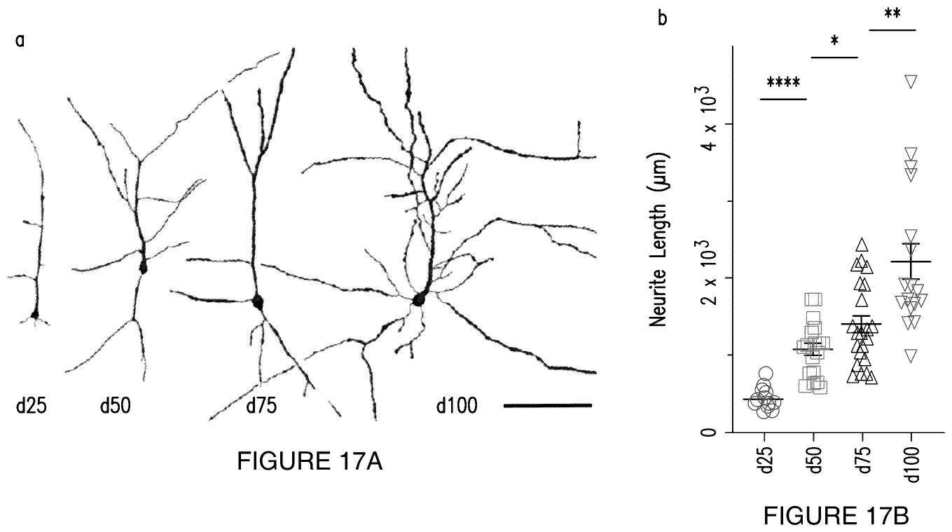

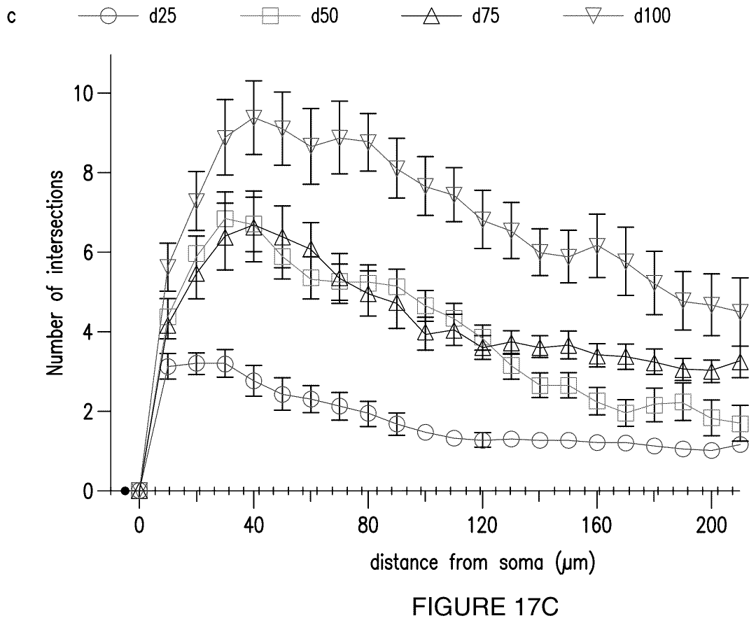

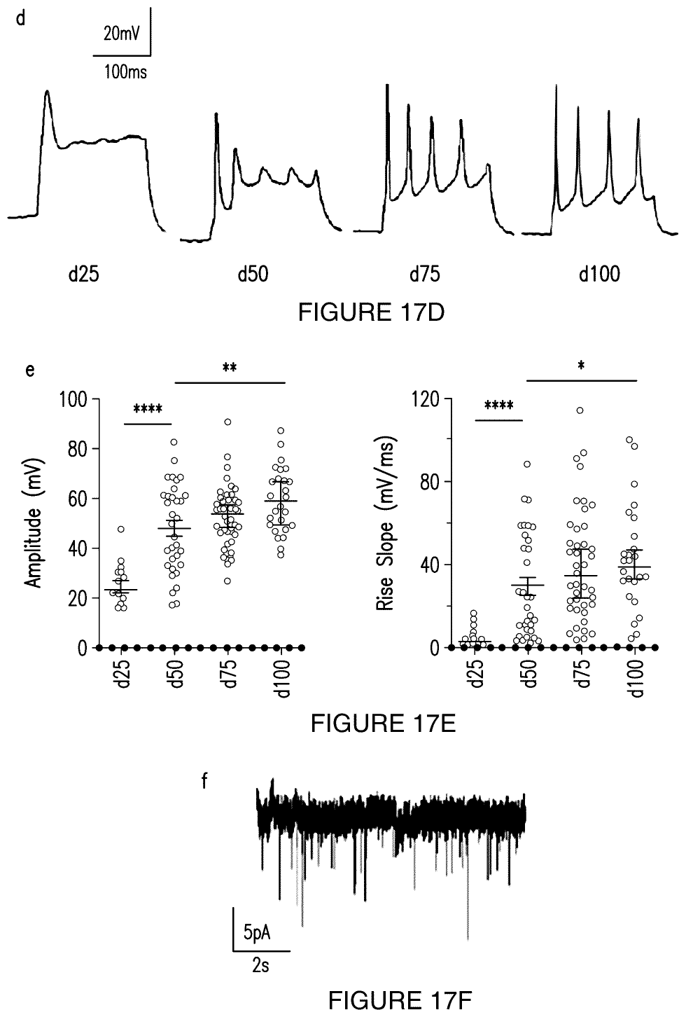

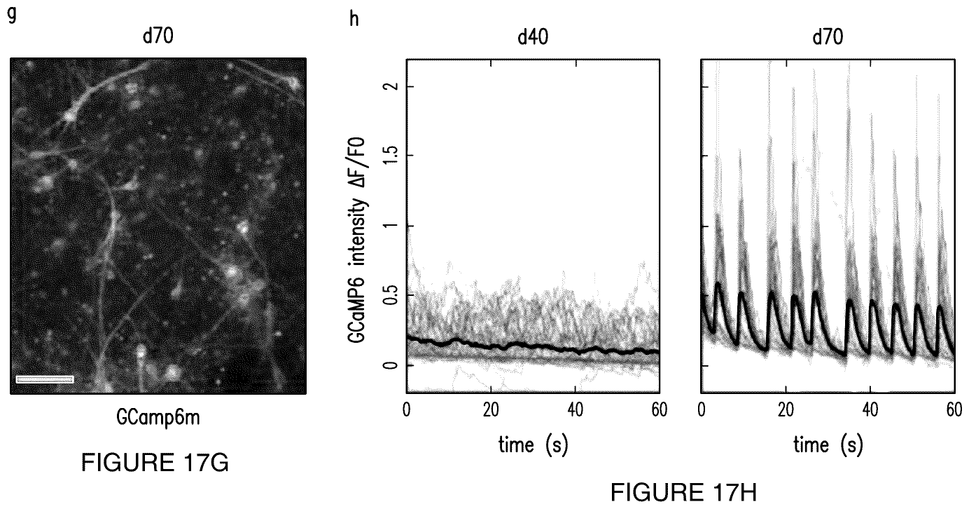

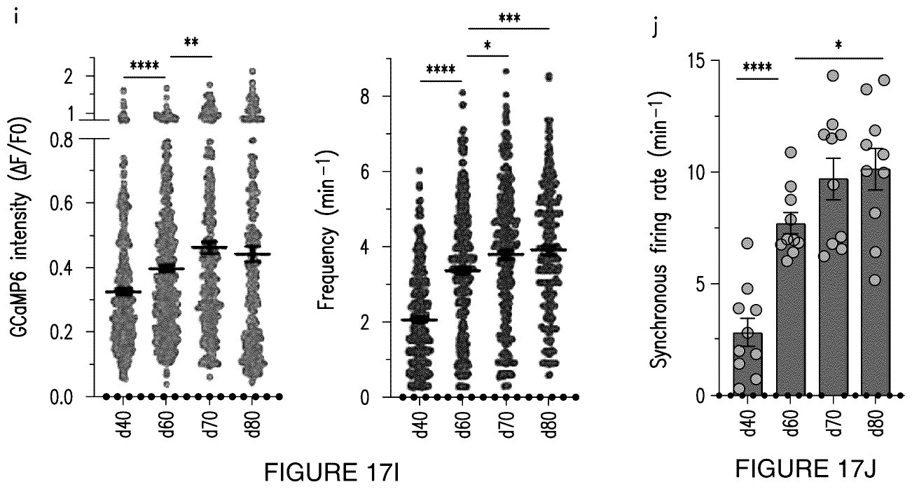

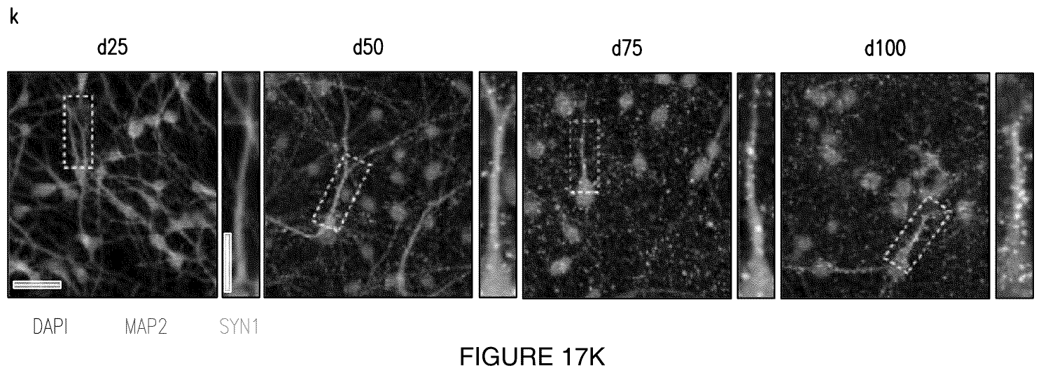

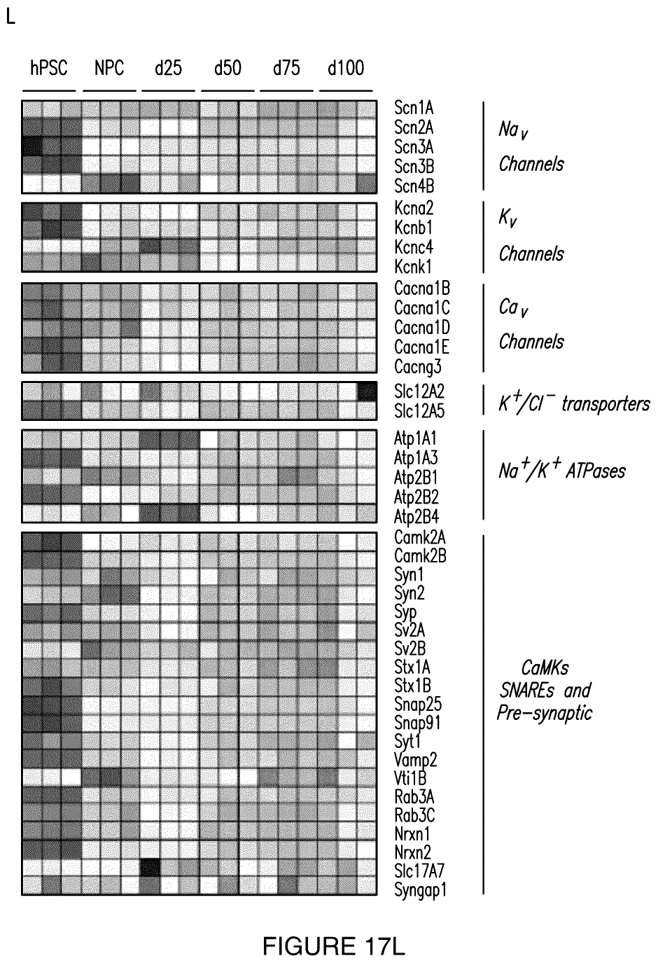

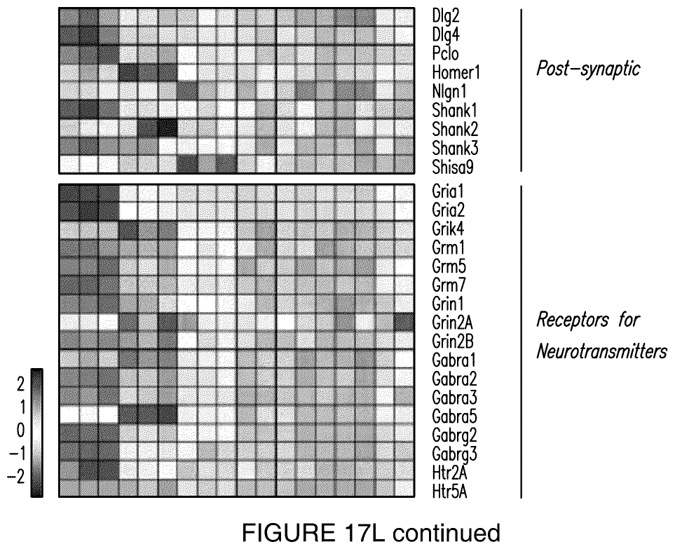

Morphometric development was characterized by infecting NPC at d20 with low-titer lentiviral vector encoding the dTomato fluorescent reporter and digitally reconstructed the morphology of individual neurons at d25, 50, 75 and 100 of differentiation (FIG. 17A). A significant increase in the total length of neurites over time as well as in the complexity of their arborization was measured by Sholl analysis (FIGS. 17A-17C). The growth in size and increased complexity of neuronal morphologies was accompanied by the progressive maturation of intrinsic electrophysiological properties measured by whole-cell patch-clamp recording. Newly born neurons at d25 exhibited immature functional properties such as abortive or low amplitude evoked single action potentials (AP). Over time in vitro neurons progressively acquired more mature intrinsic functional features, including membrane potential hyperpolarization and decreased input resistance; elicited repetitive evoked AP with increased amplitude and faster kinetics (FIGS. 17D-12E; Table 3) and displayed miniature excitatory postsynaptic currents (FIG. 17F). Table 3 shows the quantification of the electrophysiological properties of hPSC-derived neurons at day 25, 50, 75, 100 of differentiation. Results are displayed as mean±s.e.m. RMP, Resting membrane potential; IR, Input resistance, APT, Action potential threshold; APA, Action potential amplitude; APD, Action potential duration at its half amplitude; AHP, After hyperpolarization amplitude; Cm, Membrane capacitance; Rm, Membrane resistance. To measure the functional maturation of individual neurons in large scale and gain insight into dynamics of neuronal activity at network level, live imaging of spontaneous Ca²⁺ transients was performed by infecting developing neurons with lentiviruses encoding the optical calcium sensor GCaMP6m (FIGS. 17G-17H). Consistent with the electrophysiological recordings, a progressive significant temporal increase in the amplitude and frequency of spontaneous Ca²⁺ spikes at single-neuron level was observed (FIG. 17I), paralleled by a switch in network activity from sparse-to-synchronous repetitive firing by day 60 (FIGS. 17H, 17J). Altogether, these results show that isochronic hPSCs-derived cortical neurons followed a coordinated program of morphological and functional maturation, became progressively more excitable, developed synaptic connectivity and engaged in synchronous pattern of network activity. The gradual maturation of functional properties correlated with the establishment of synaptic contacts as suggested by the progressive subcellular localization of Synapsin I in putative presynaptic puncta-like structures (FIG. 17K). To analyze the relationship between the onset of functional properties and the underlying molecular machinery, the expression of a core set of genes involved in neuronal functionality by RNAseq at d25, 50, 75 and 100 of differentiation were tracked (FIG. 17L). The present disclosure shows concerted increased expression of transcripts important for neuronal excitability and maintenance of electrochemical gradients across the plasma membrane, including voltage-gated Na⁺, K⁺ and Ca²⁺ channels, Na⁺/K⁺ and Ca²⁺ ATPase, cation/chloride transporters Kcc2 and Nkcc1 which regulate intracellular chloride homeostasis and the excitatory-to-inhibitory GABA switch during development (Ben-Ari, Y., Nature Reviews Neuroscience 3, 728-739 (2002)). Transcripts important for the assembly of pre and post-synaptic compartments, including members of SNARE, Neuroligin, Neurexin and Shank gene families and receptors for the main neurotransmitters were upregulated as well, including Grin2b/a switch in glutamate receptor subunits, which expression correlates with stage of neuronal maturity (Bar-Shira, O. et al., PLoS Comput Biol 11, e1004559 (2015)).

TABLE 3
Electrophysiological Properties of Cortical Neurons

| | day 25 | day 50 | day 75 | day 100 |
|---|---|---|---|---|
| RMP (mV) | −36.7 ± 2.0 | −50.2 ± 1.2 | −52.3 ± 1.1 | −51.6 ± 1.4 |
| IR (MW) | 1,601.5 ± 100.9 | 731.4 ± 53.3 | 646.0 ± 48.6 | 518.5 ± 65.9 |
| Rheobase (pA) | 98.8 ± 8.5 | 32.7 ± 6.5 | 36.0 ± 4.7 | 29.7 ± 4.5 |
| APT (mV) | −8.8 ± 2.4 | −31.5 ± 1.2 | −30.1 ± 1.0 | −34.5 ± 1.0 |
| APA (mV) | 25.1 ± 1.3 | 48.2 ± 3.1 | 53.1 ± 1.8 | 59.1 ± 2.3 |
| APD (ms) | 23.9 ± 2.1 | 14.1 ± 1.4 | 10.0 ± 1.0 | 9.5 ± 0.7 |
| AHP (ms) | 12.0 ± 1.2 | 6.0 ± 0.7 | 4.0 ± 0.3 | 3.5 ± 0.2 |
| Rise time (ms) | 5.5 ± 0.5 | 2.5 ± 0.3 | 1.7 ± 0.2 | 1.6 ± 0.2 |
| Decay time (ms) | 9.6 ± 0.9 | 7.3 ± 1.0 | 4.3 ± 0.5 | 4.1 ± 0.3 |
| Rise slope (mv/ms) | 4.4 ± 0.8 | 29.7 ± 4.3 | 39.8 ± 4.0 | 42.4 ± 4.3 |
| Decay slope (mv/ms) | −2.9 ± 0.7 | −9.9 ± 1.4 | −14.2 ± 1.3 | −13.7 ± 1.1 |
| Cm (pF) | 21.2 ± 1.6 | 28.3 ± 1.9 | 35.2 ± 2.0 | 31.6 ± 1.9 |
| Rm (MW) | 505.2 ± 61.6 | 192.0 ± 20.0 | 144.8 ± 8.7 | 143.6 ± 13.5 |
| Access resistance (MW) | 39.8 ± 2.6 | 42.9 ± 2.3 | 36.5 ± 1.9 | 39.6 ± 1.7 |
| n | 25 | 33 | 43 | 29 |

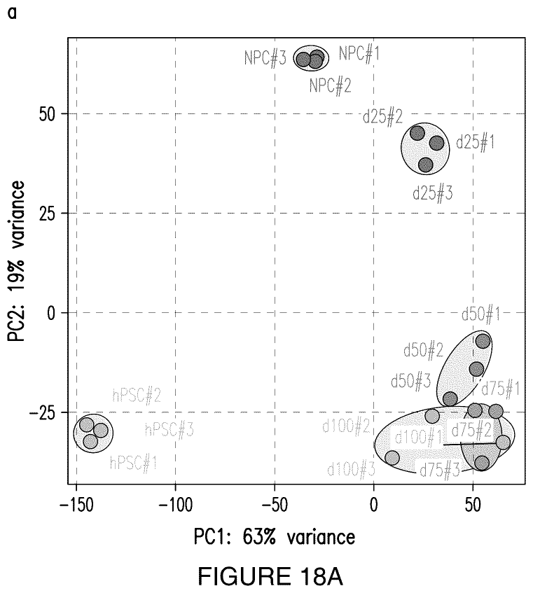

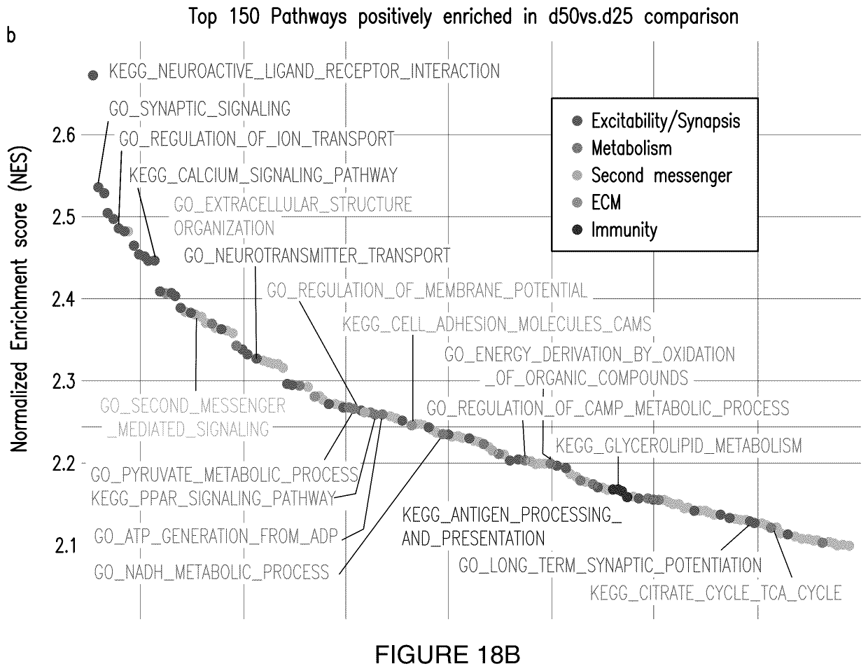

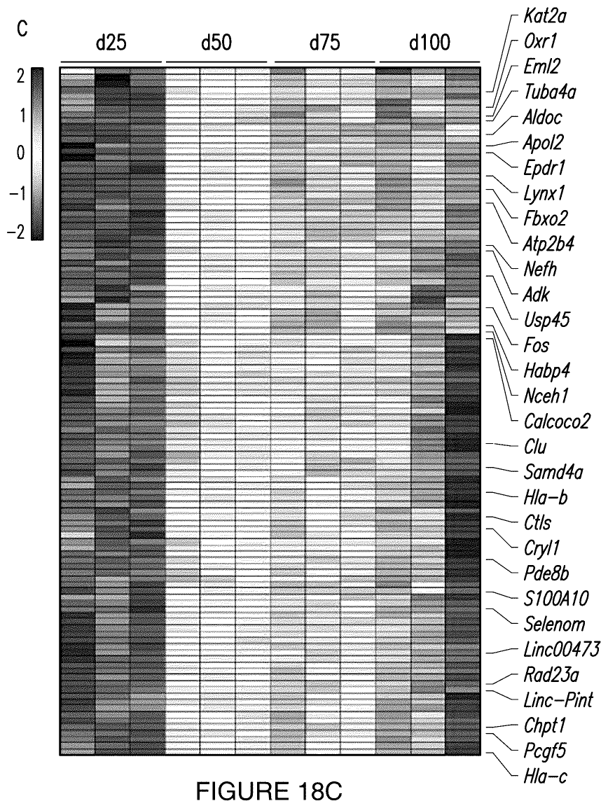

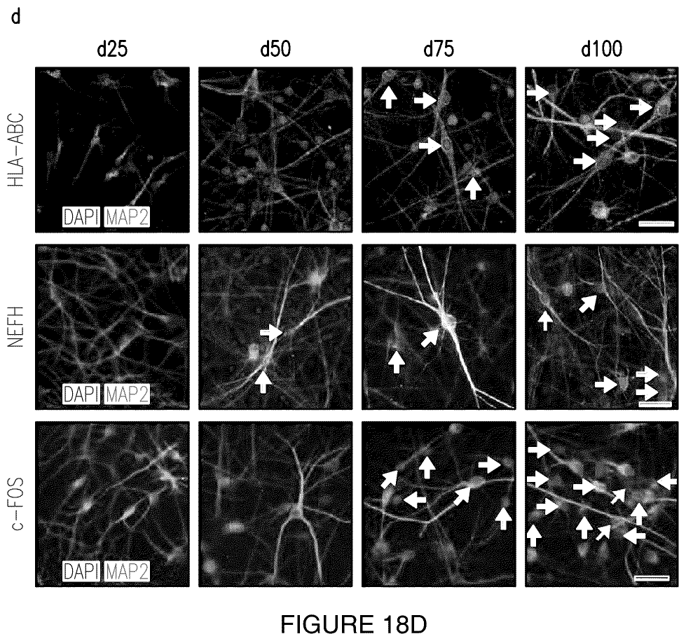

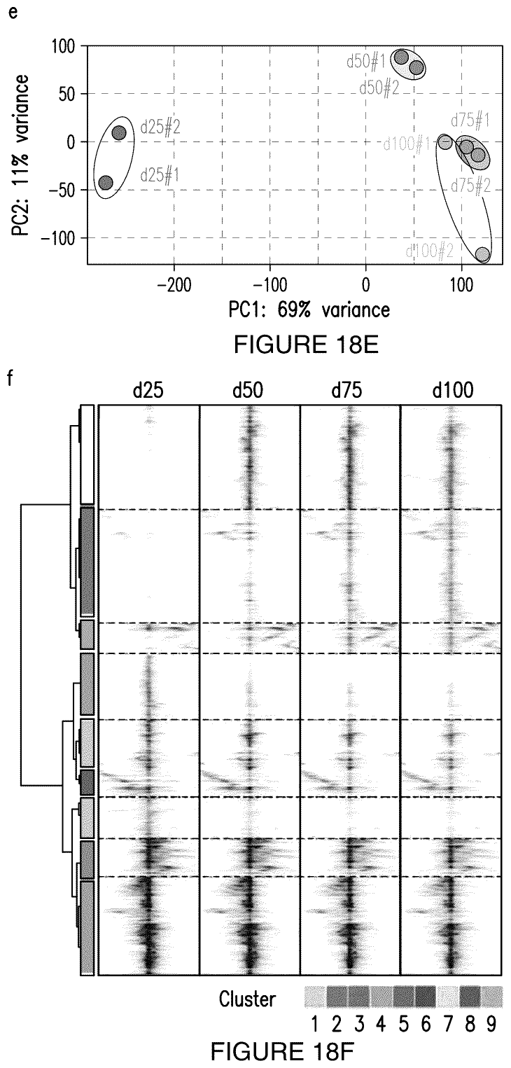

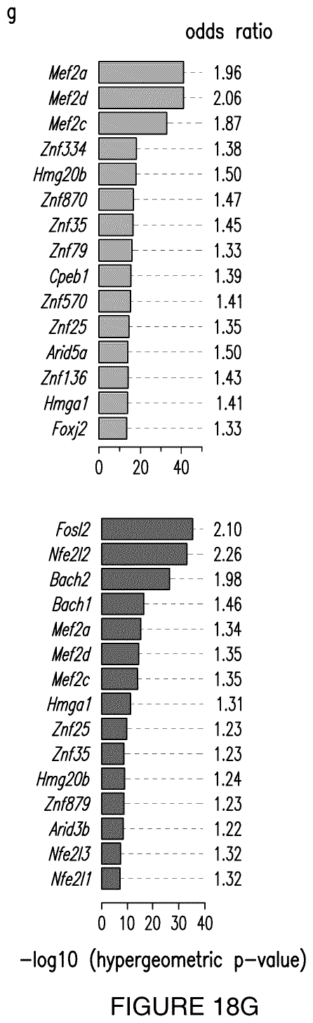

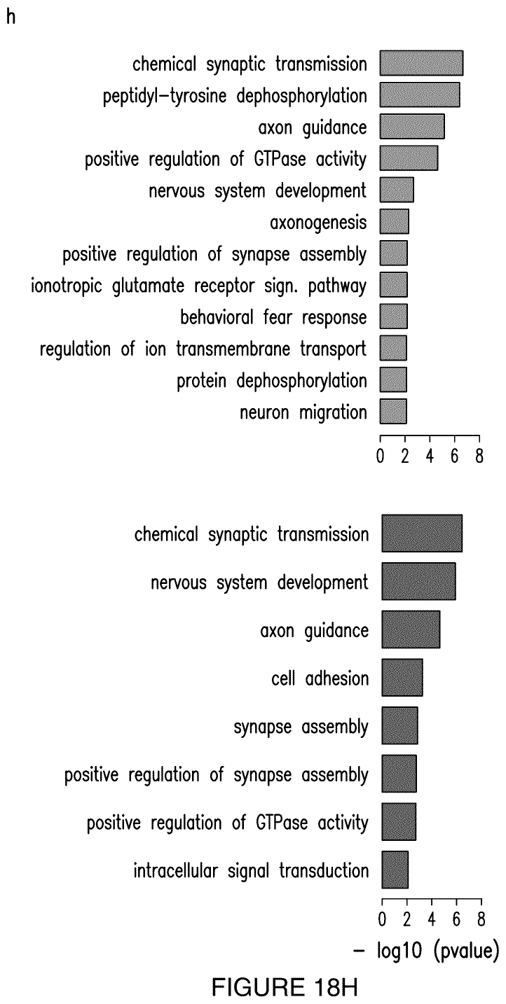

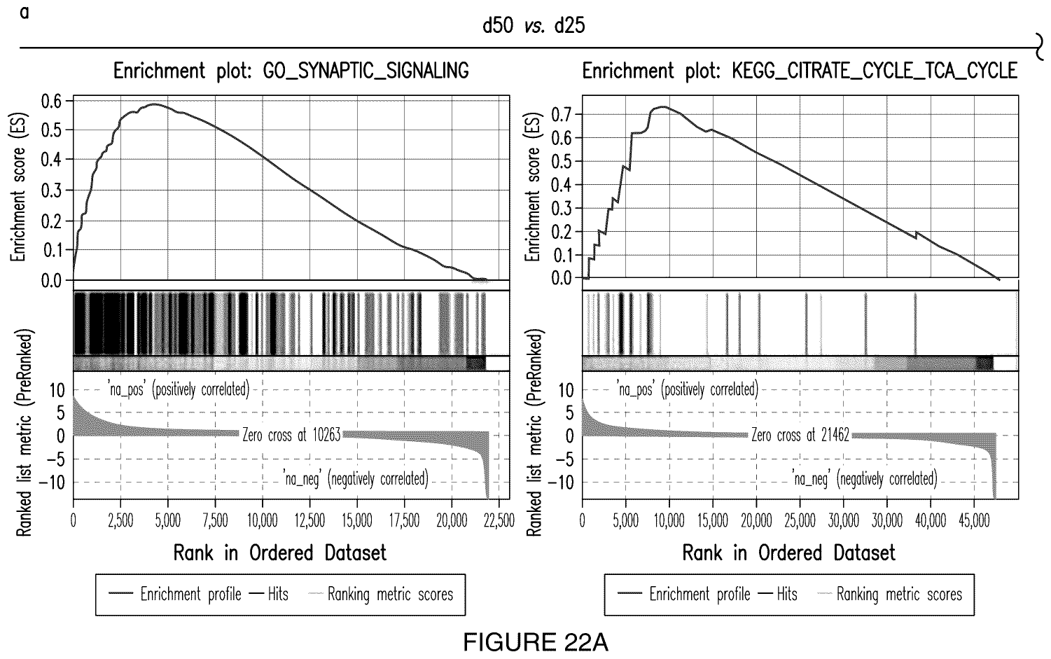

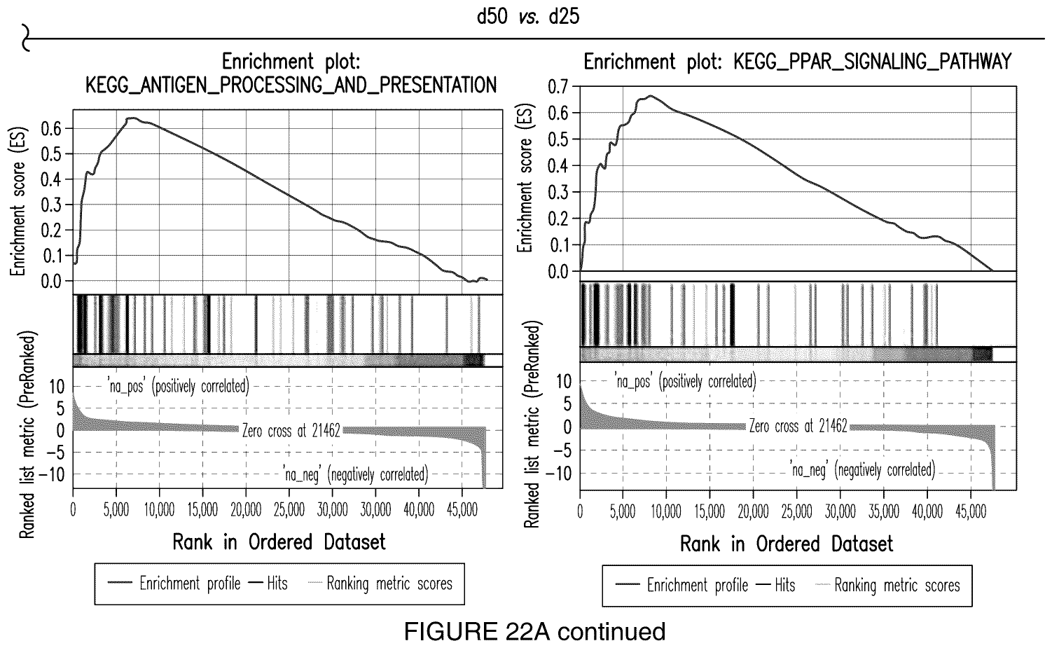

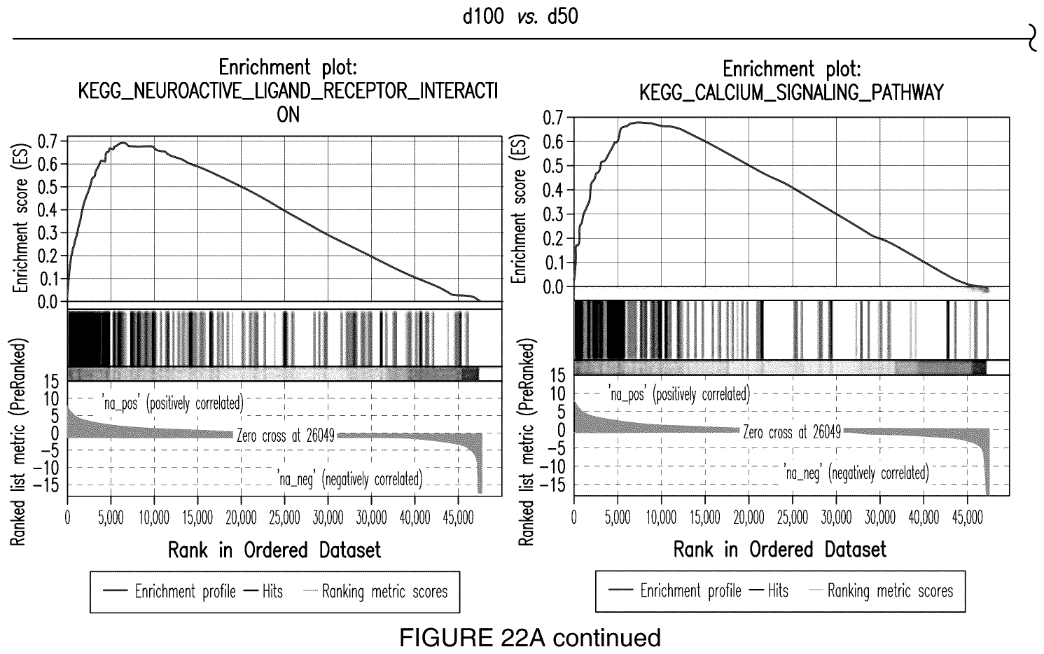

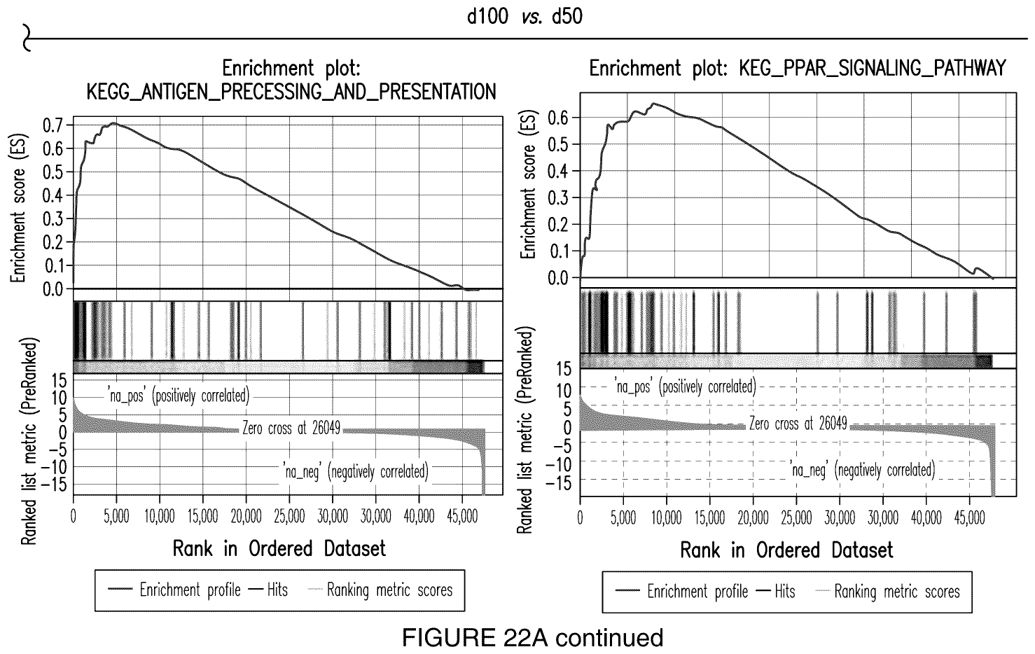

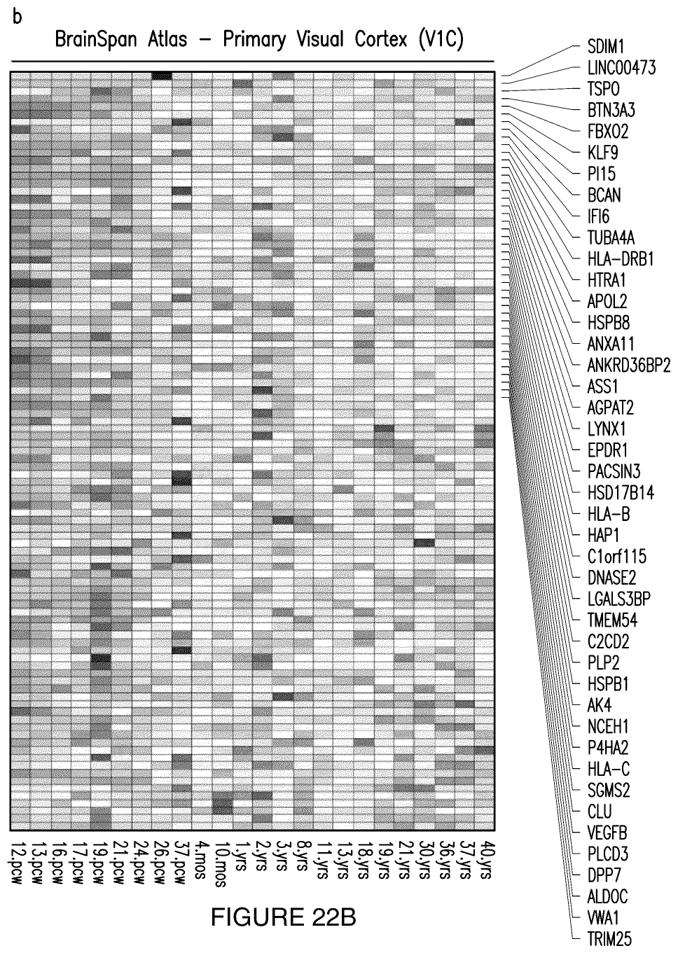

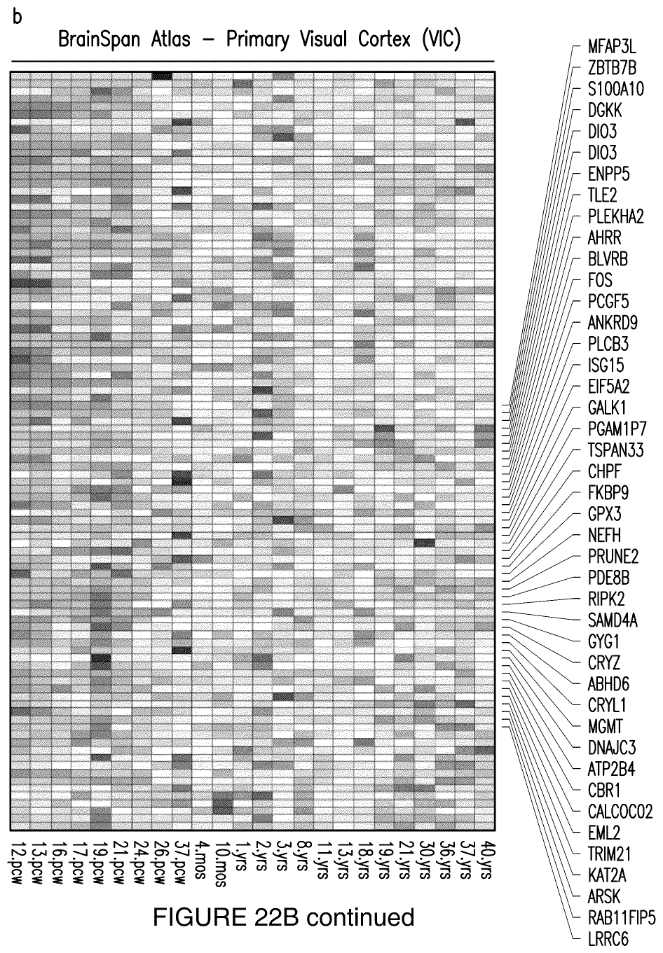

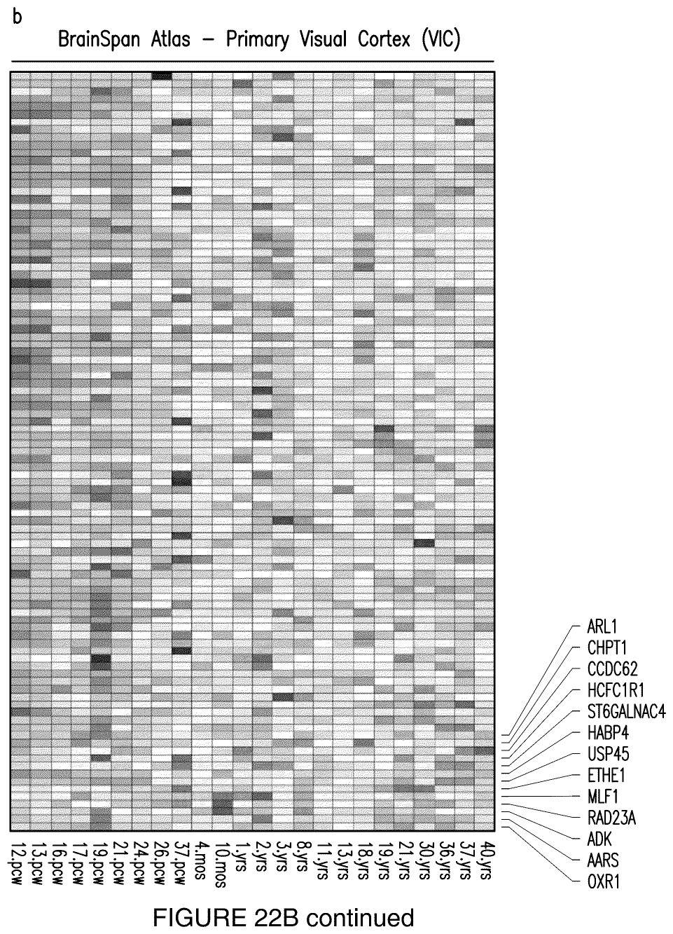

Comprehensive RNAseq analyses was performed to dissect signatures and dynamics of the transcriptional maturation program. Principal Component Analysis (PCA) showed samples distribution according to developmental stages, with hPSC-to-NPC and NPC-to-neurons representing the most distant developmental transitions. At the neuron stage, more pronounced changes occurring between d25 and d50, followed by a more subtle sample-to-sample distance between d50, 75 and 100 neurons, which support a gradual temporal molecular progression towards more mature states (FIG. 18A). The PCA revealed d25, 50 and 100 as 3 putative maturation stages and thus the present disclosure focused on changes occurring among them. Gene Set Enrichment Analysis (GSEA) of enriched Gene Ontologies (GO) in d50 vs. d25 pairwise comparisons revealed a complex signature. GO related to neuronal excitability and synaptic assembly were among the most significant. Metabolic processes, including oxidative phosphorylation, glycerolipid metabolism and PPAR signaling pathways which participate in neuronal maturation (Zheng, X. et al., Elife 5, (2016)) were also enriched at high significance. Enrichment of immunity-related GO, such as antigen processing and presentation, was also observed (FIG. 18B). Similar GO categories were enriched in d100 vs. d50 comparisons (FIG. 22A), demonstrating a lengthy gradual unfolding of the same transcriptional signature over time. Maturation-related transcriptional changes were uncovered, by mapping dynamic trends of gene-expression and unbiasedly selected differentially expressed transcripts that showed monotonic increased expression during maturation (FIG. 18C). Monotonic upregulated transcripts captured multiple dimensions of the maturation program, including component of the cytoskeleton (Tuba4a, Nefh), Ca²⁺ signaling/homeostasis (Atp2b4), ATP biosynthesis (Aldoc), Lipid and cholesterol metabolism (Apol2, Nceh1), protein biosynthesis and degradation (Aars, Fbxo2, Usp45), antioxidant responses (Oxr1), immunological changes (Hla-b/c) and activity-depended transcripts (Fos, Linc00473) among others (FIG. 18C). Immunofluorescence was performed to confirm the stage-specific expression for few monotonically upregulated markers, including Hla-abc, Nefh and c-Fos (FIG. 18D). The progressive upregulation of specific transcripts matched trends of in vivo gene-expression based on BrainSpan Atlas of the Developing Human Brain dataset (Kang, H. J. et al., Nature 478, 483-489 (2011)), with more pronounced changes occurring at late perinatal/early postnatal stages of human cortical development (FIG. 22B).

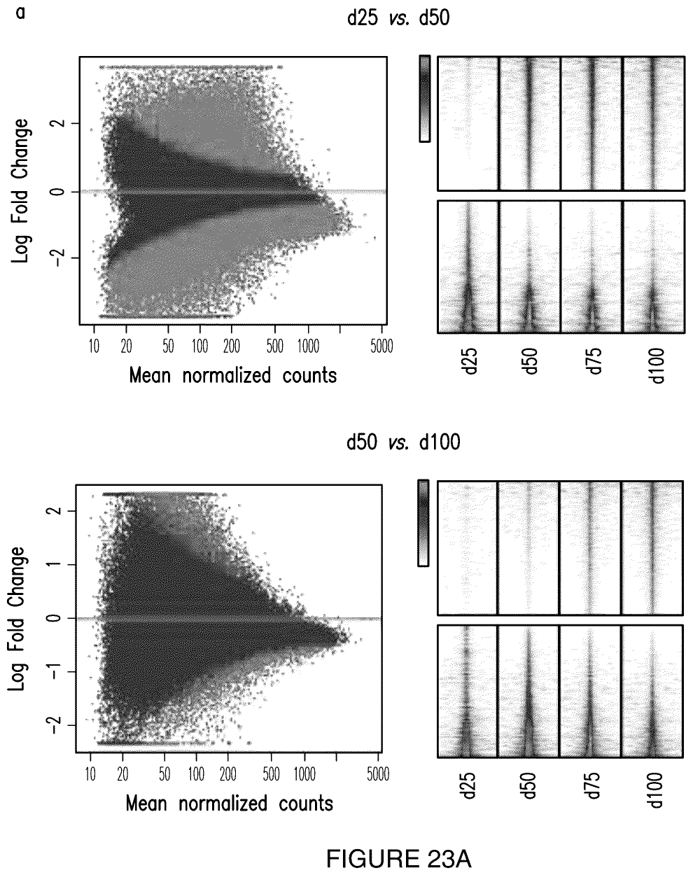

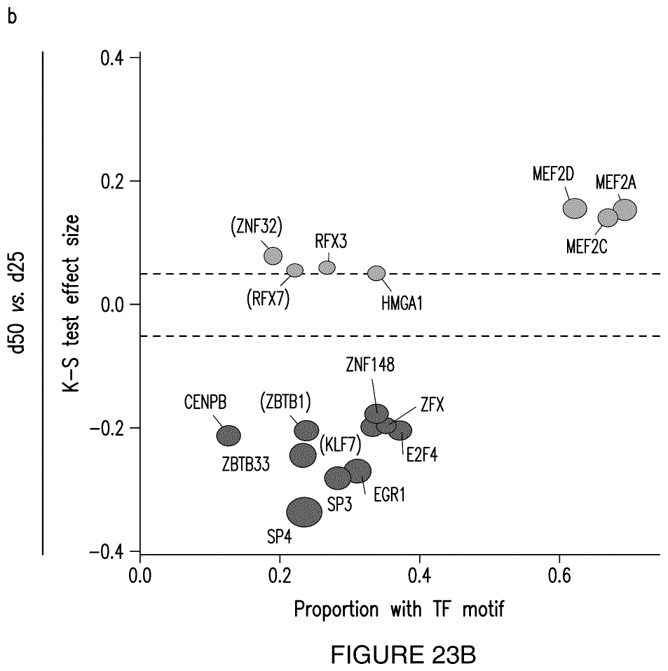

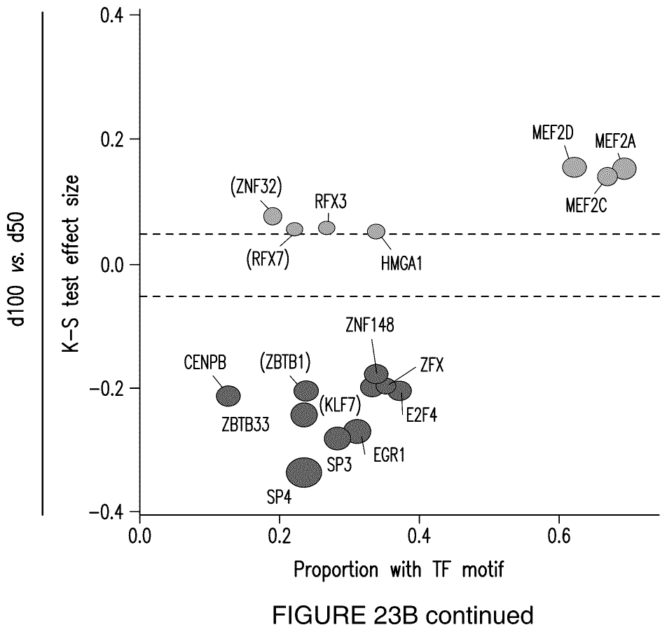

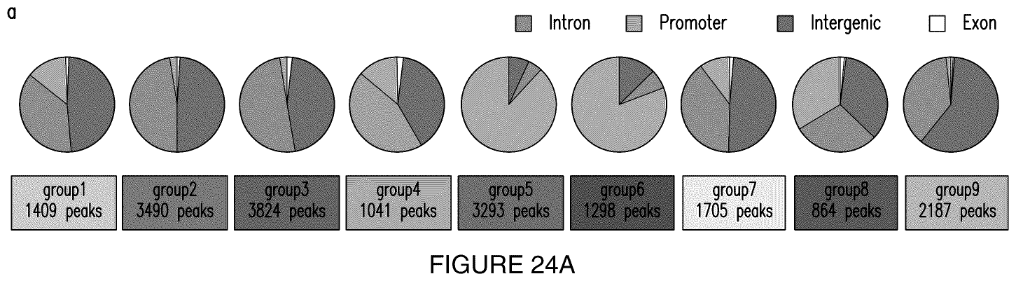

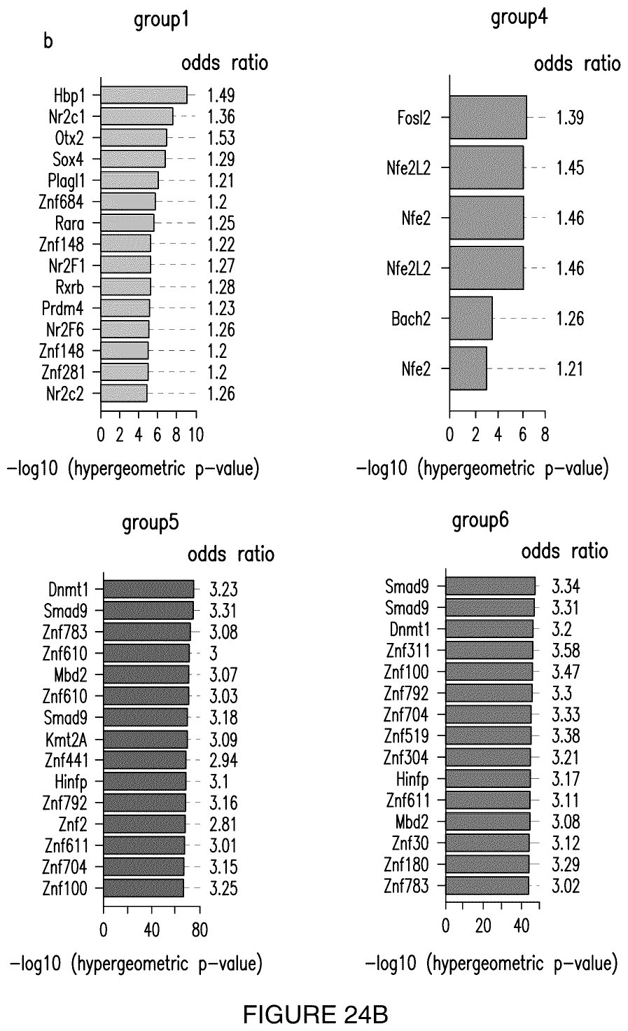

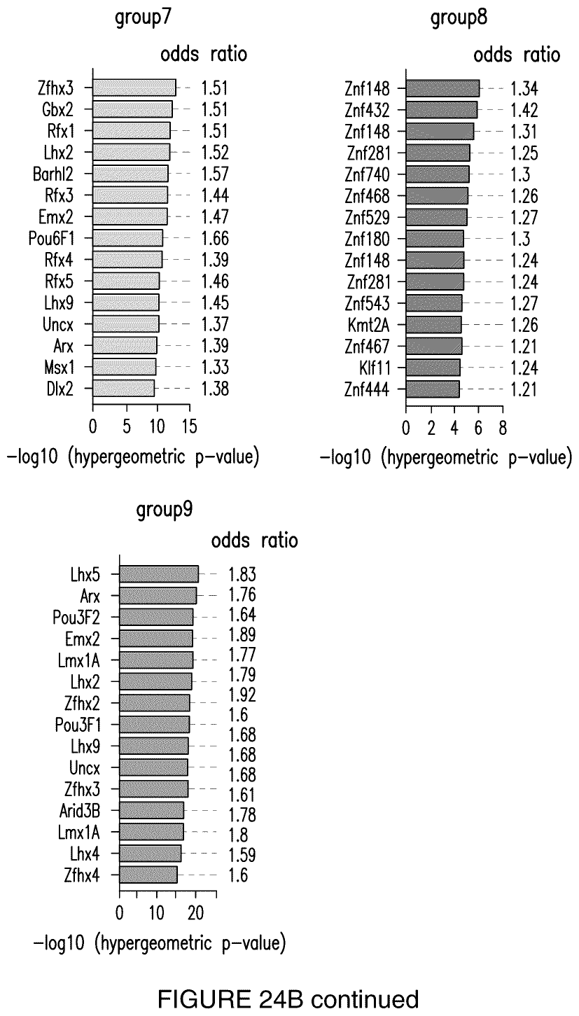

ATACseq was performed to investigate changes in chromatin landscape during neuronal maturation focusing on d25, 50, 75 and 100 stages and including hPSCs and NPCs samples as a reference. Consistent with the RNAseq dataset, PCA analysis revealed sample distribution according to the maturation timeline (FIG. 18E) with a large number of ATACseq peaks changing accessibility between d25 and d50 followed by robust but more subtle differences occurring between d100 vs. d50 (FIG. 23A). Temporal dynamics of chromatin rearrangement during maturation were uncovered from a compiled list of ˜20000 ATACseq peaks whose accessibility changed specifically at neuron stage and comprised differentially accessible peaks from combinations of pairwise comparisons between d25, 50, 75 and 100 samples. Unbiassed clustering of these ATACseq peaks identified 9 groups with different dynamics of chromatin opening/closure (FIG. 18G). Except for group 5, 6 and 8, whose peaks mapped primarily at gene-promoters and showed more subtle temporal trends of accessibility, all the other groups mapped primarily at putative enhancer DNA sequences in intergenic or intronic genomic regions and show marked stage-specific dynamics of chromatin accessibility (FIG. 18G; FIG. 24A). Groups 1 and 9 defined a subset of peaks with increased accessibility in young neurons that get progressively less accessible towards more mature stages. Young neurons specific accessible peaks were in some instances shared with NPCs and hPSCs stages (FIG. 18F). Instead, groups 2, 3 and 4 peaks showed progressive, gradual gain in accessibility towards more mature stages. To infer active regulatory elements including upstream transcriptional regulators, we performed transcription factor (TF) motif enrichment analysis at the group specific peaks. Young neurons specific accessible peaks showed enrichment for TF motifs important for early cortical (Di Bella, D. J. et al., Nature 595, 554-559 (2021)), including Otx2, Sox4, Emx2, Lhx2, Pou3F1 and Pou3F2 among others (FIG. 24B). Instead, group 2 and 3 peaks were highly enriched for TF binding motif belonging to the Myocyte Enhancer Factor gene family (Mef2a, d, c) which regulate synaptic connectivity in an activity-depended manner (Flavell, S. W. et al., Science 311, 1008-1012 (2006); Rajkovich, K. E. et al., Neuron 93, 48-56 (2017)) and basic leucin zipper (bZIP) proteins such as Nfe2l2 and member of the AP-1 complex Fosl2, which participate in maintaining oxidative homeostasis and proteostasis (Pajares, M. et al., Autophagy 12, 1902-1916 (2016)) and in activity-dependent mechanisms of gene-expression and chromatin remodeling respectively (West, A. E. & Greenberg, M. E., Cold Spring Harb Perspect Biol 3, (2011); Malik, A. N. et al., Nat Neurosci 17, 1330-1339 (2014)) (FIG. 18G). The enrichment for Mef2 and AP-1 TF binding motif (including Fos, JunB and JunD) at late-opening peaks was confirmed in d50 vs. d25 and d100 vs. d50 pairwise comparisons of differentially accessible ATACseq peaks (FIG. 23B). It is worth noting that the opening of peaks associated with activity-dependent TF is paralleled by their increased expression (FIGS. 18C-18) and coincided with the onset of synchronous firing of the neuronal network (FIGS. 18G-18J). In addition, GO analysis on putative genes linked with the late-opening group 2 and 3 ATACseq peaks revealed enrichment for synaptic-related categories (FIG. 18H). Altogether, these results indicated that synchronized hPSCs-derived cortical neurons undergo a lengthy functional and molecular program of maturation and show their engagement in activity-dependent mechanisms at late maturation stages.

### Neuronal Maturation Driven by Epigenetic Switch

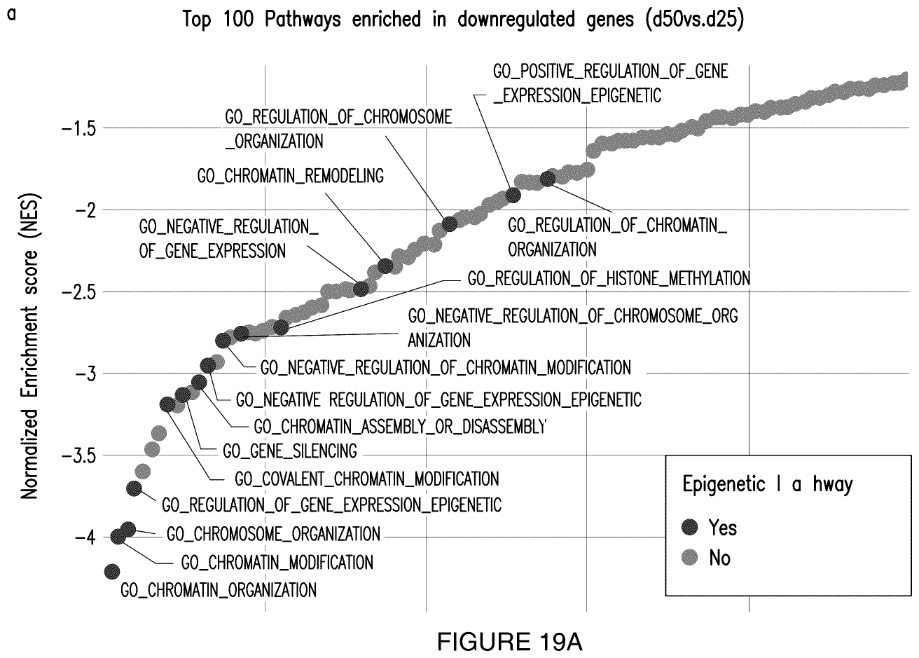

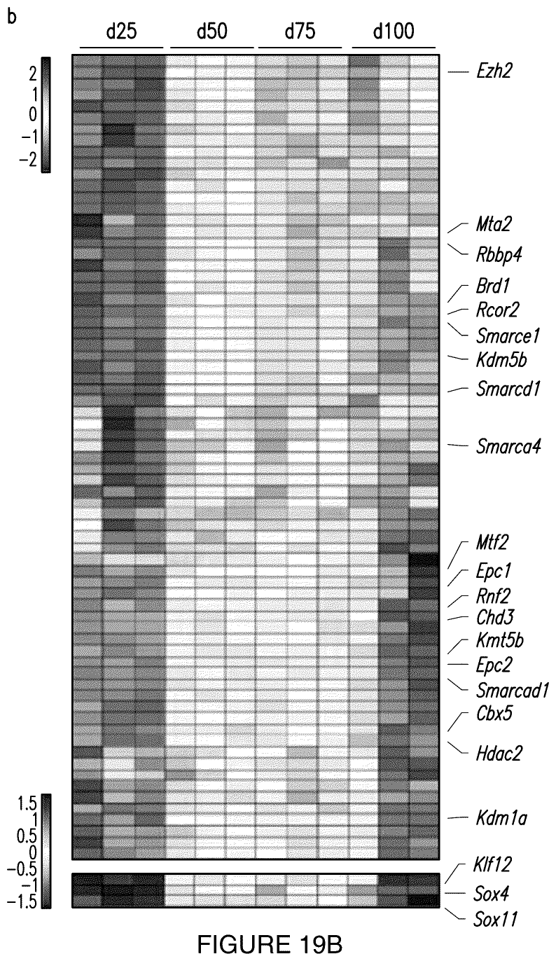

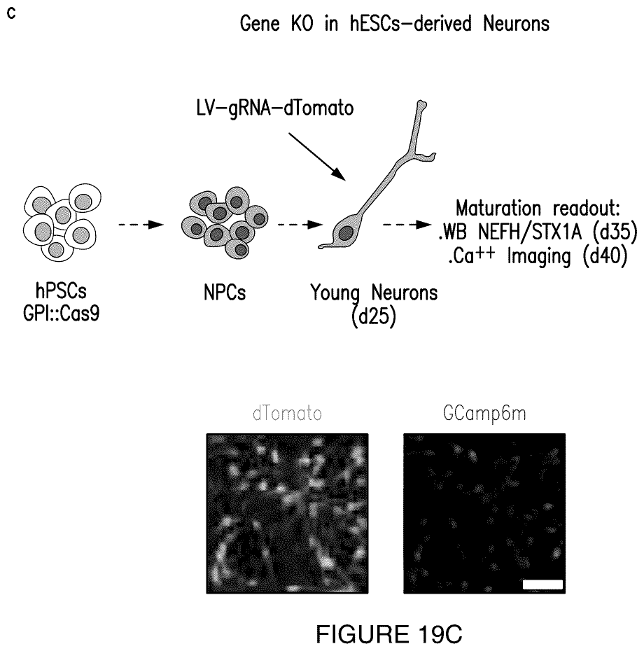

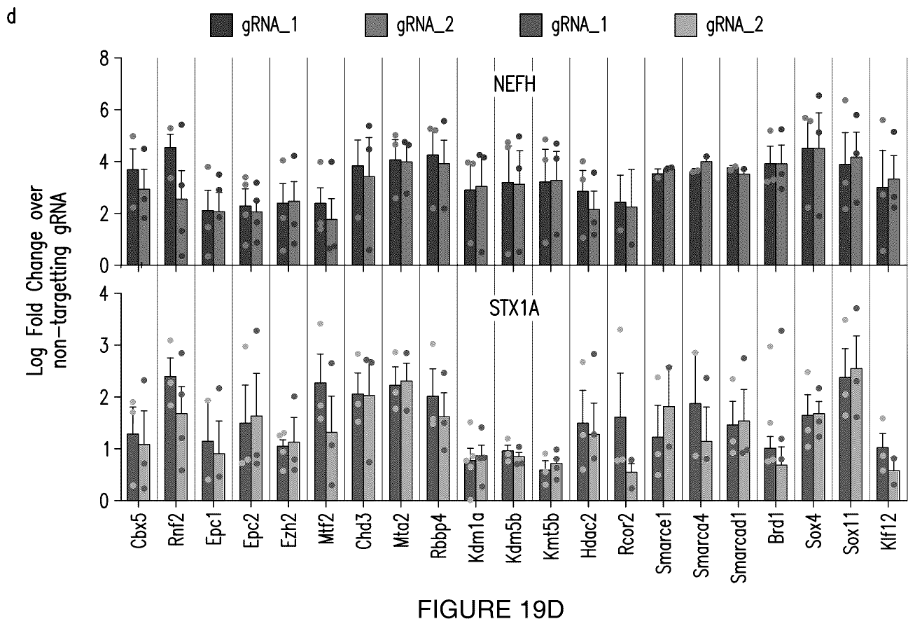

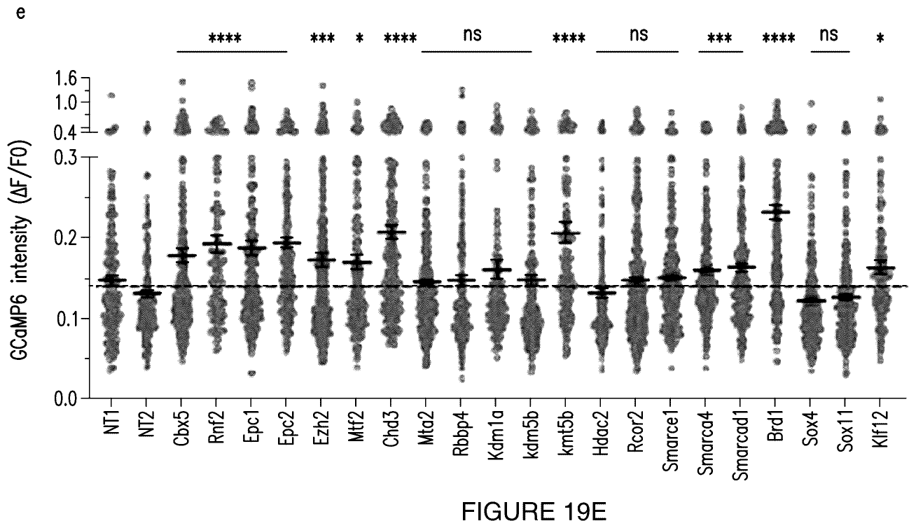

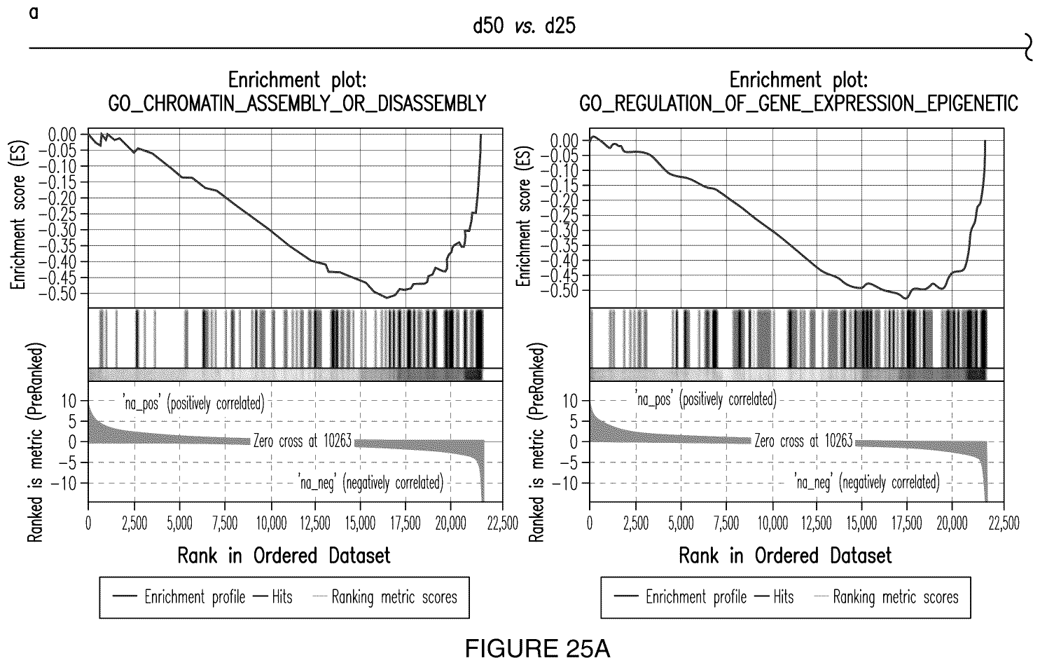

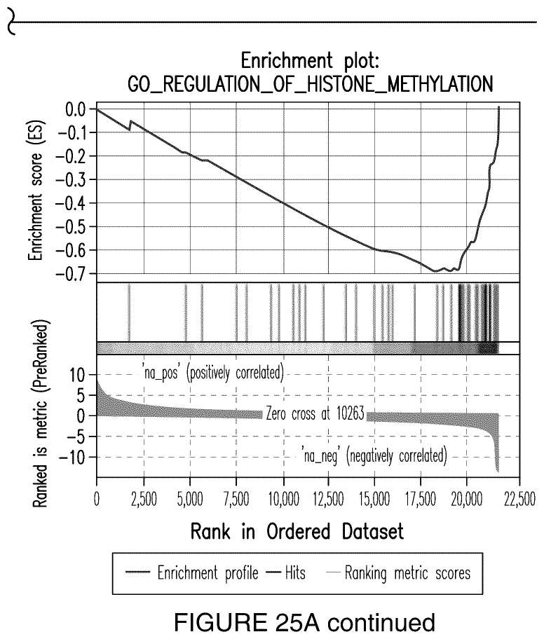

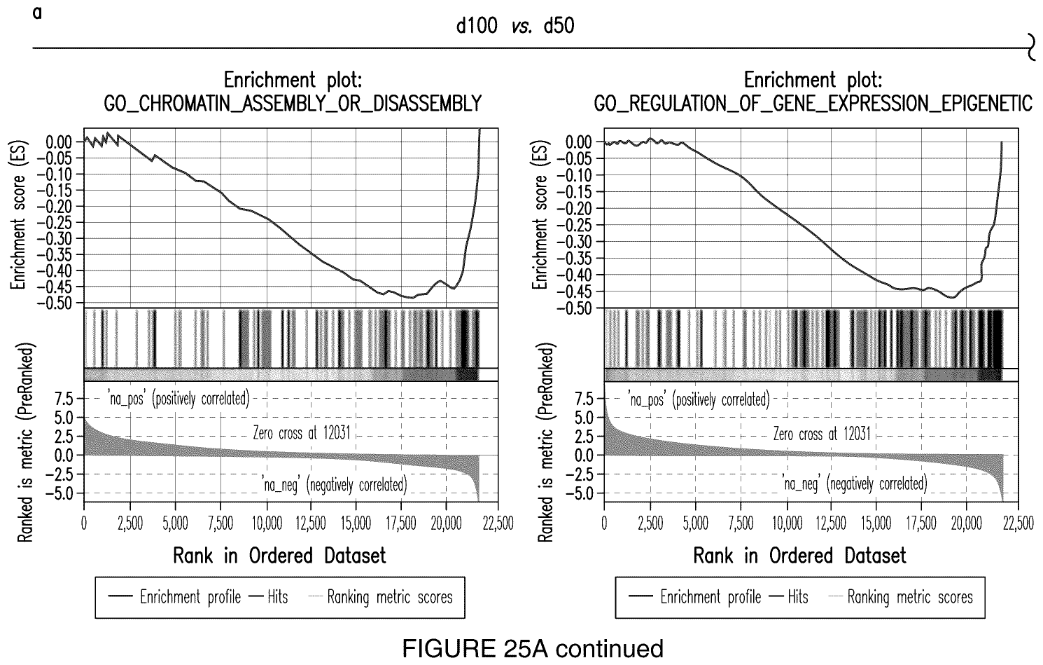

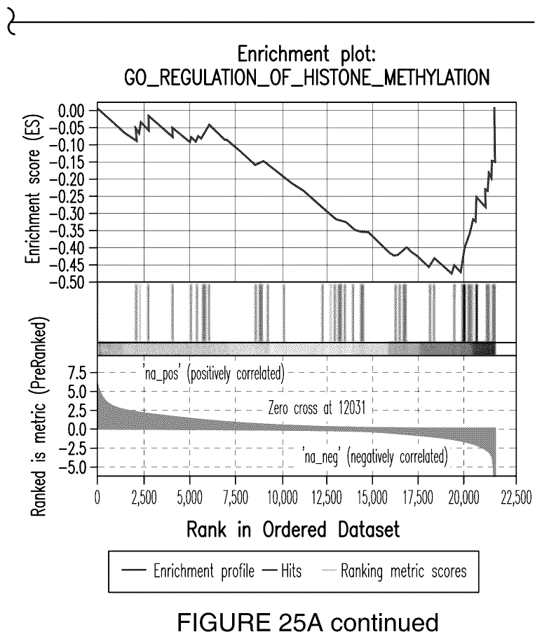

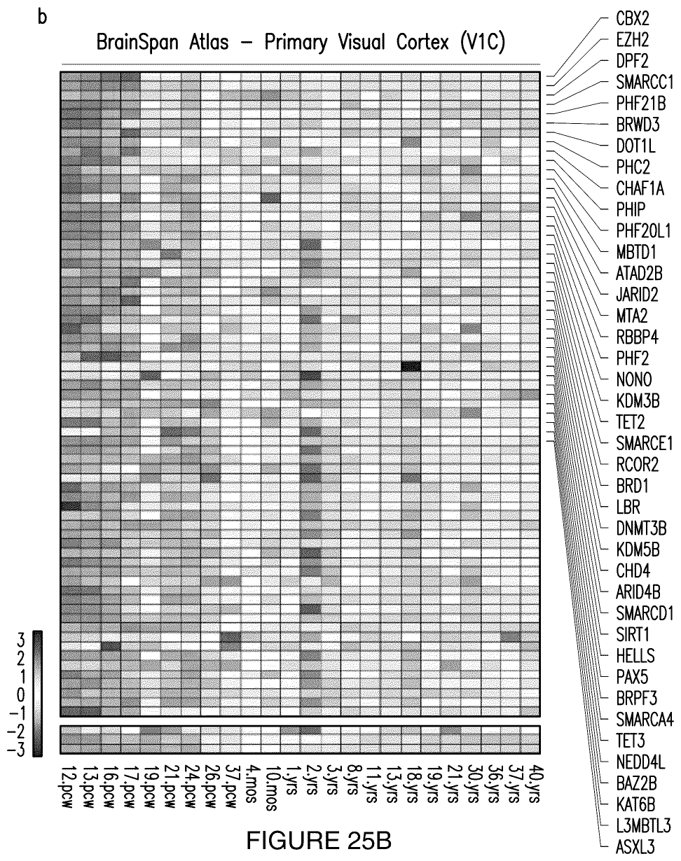

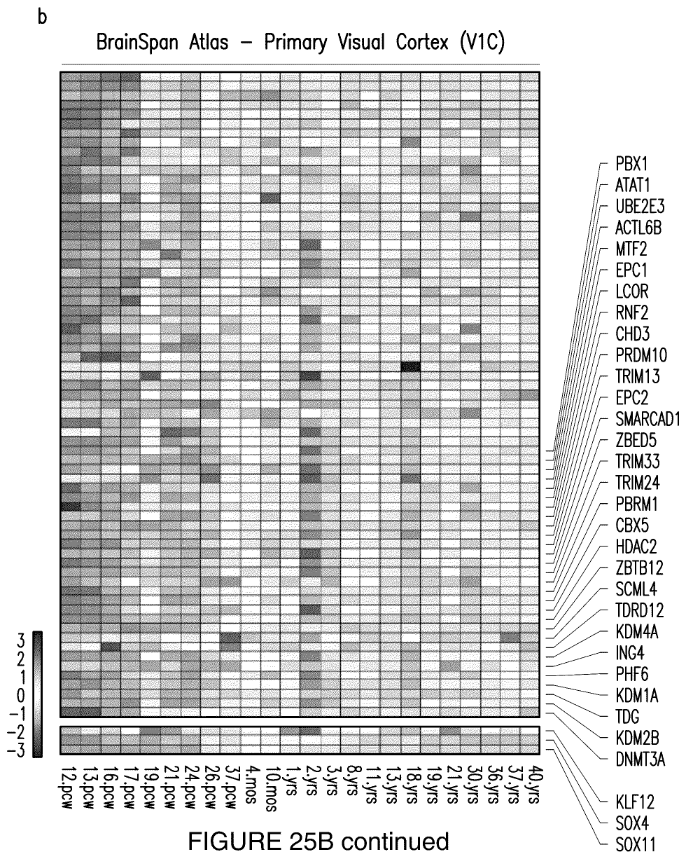

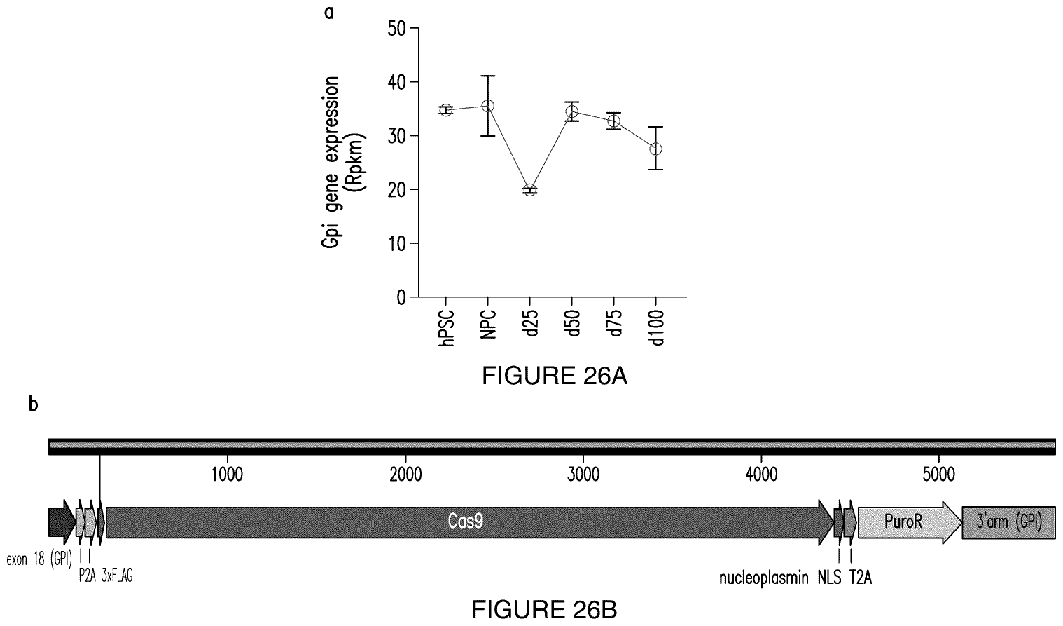

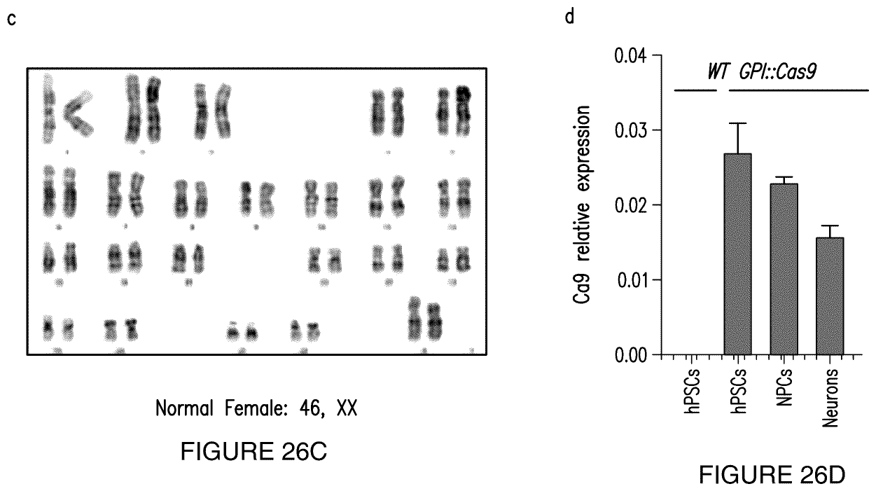

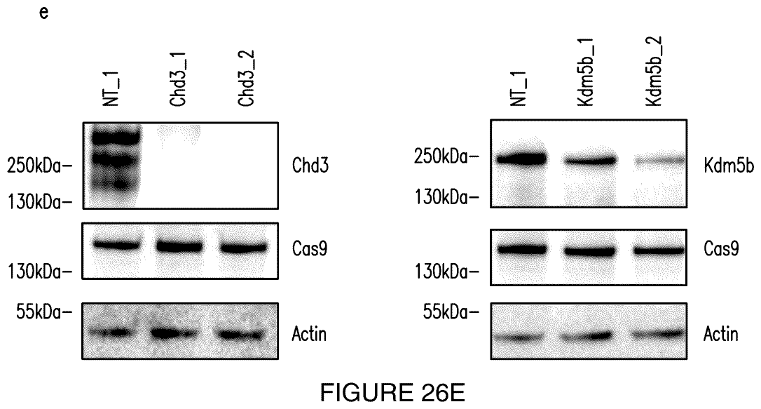

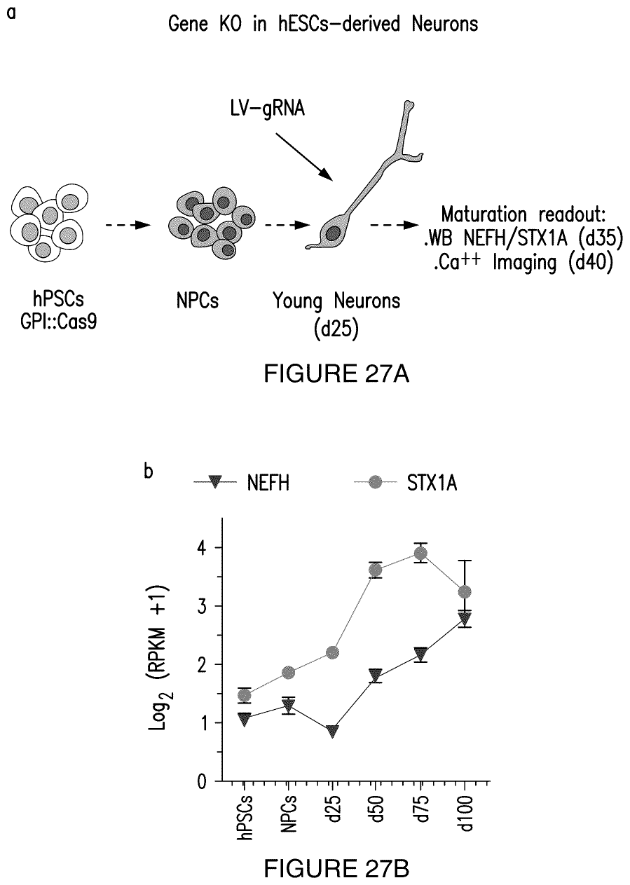

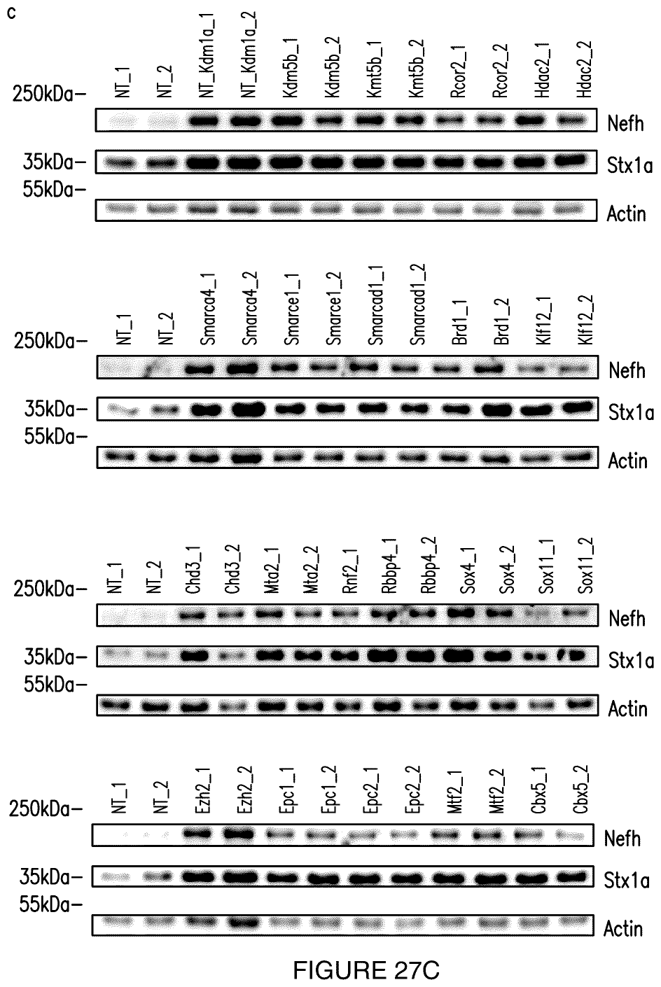

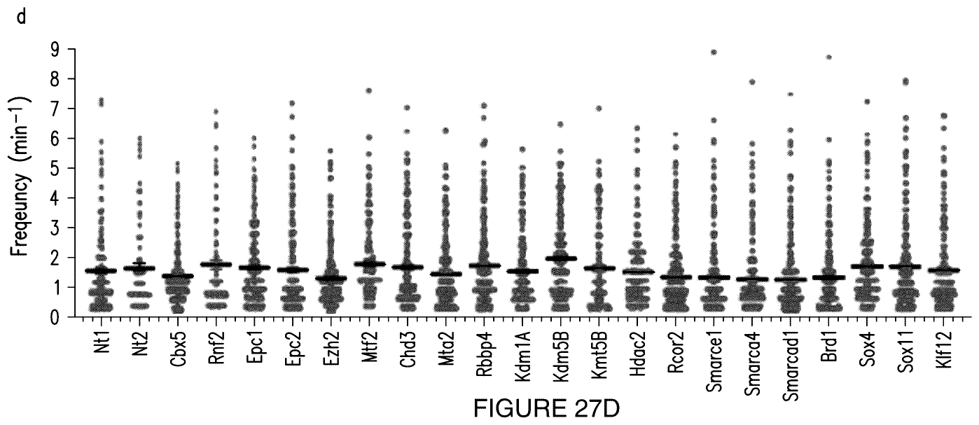

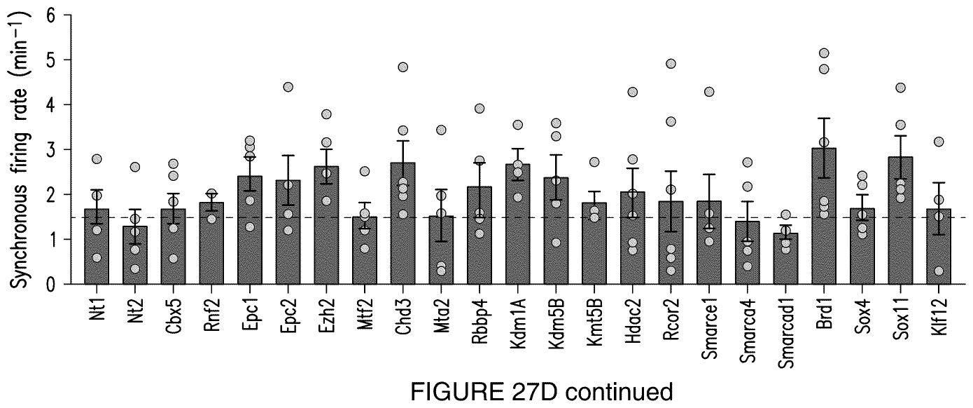

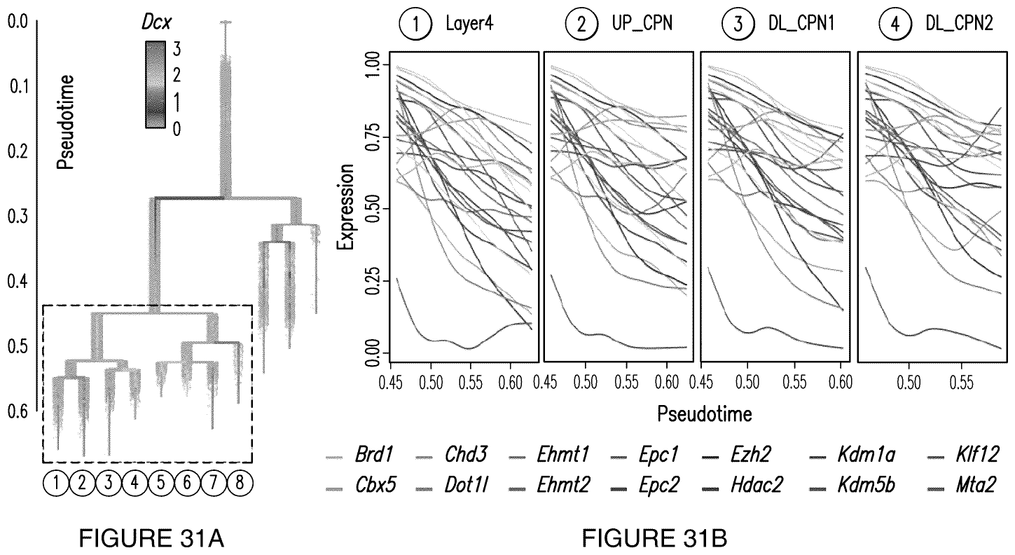

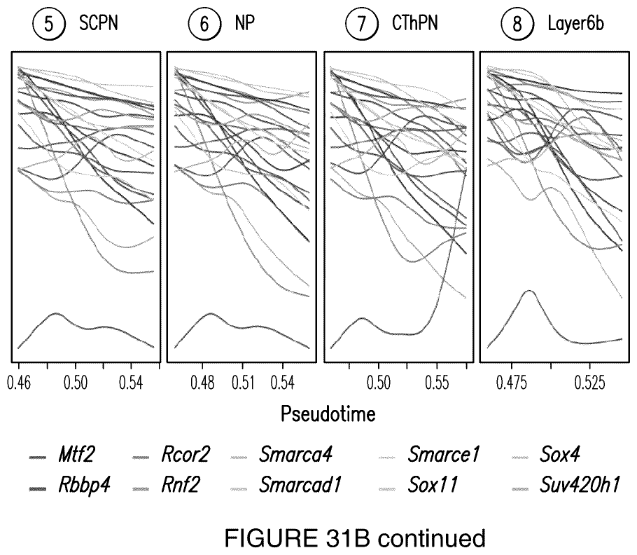

The present disclosure describes a molecular study derived from the analysis of the downregulated genes during maturation. GSEA identified chromatin organization and epigenetic-related pathways as the most significant among negative enriched GO in d50 vs. d25 and d100 vs. d50 comparisons (FIG. 19A, FIG. 25A), showing a gradual downregulation of epigenetic related factors. Indeed, analysis for the dynamic expression of epigenetic factors specifically, identified a core set of transcripts whose levels monotonically decreased during the time course of maturation (FIG. 19B), following a trend that largely recapitulated the expression dynamics of the same set of genes in the cortex in vivo (FIG. 25B). Monotonically downregulated chromatin regulators comprise members of multiple epigenetic complexes including Polycomb repressive complex 1 and 2 (PRC1/2), mammalian SWI/SNF family chromatin remodelers (BAF), MOZ/MORF acetyltransferases, nucleosome remodeling and deacetylase (NuRD) and histone lysine demethylases and methyltransferases. These results indicated an overall inverse correlation between specific epigenetic changes and maturation state, thus, the retention of an epigenetic signature in young neurons limits their progression towards cell maturity. Twenty-one genes were selected that comprise the 18 chromatin regulators and the 3 transcription factors Sox4, Sox11 and Klf12 that also showed monotonic decreased temporal expression and developed CRISPR/Cas9 -based strategy to induce loss-of-function gene perturbations at neuron stage (FIG. 19C). A knock-in hPSCs line for constitutive Cas9 expression driven by the Glucose-6-phosphate isomerase gene was generated (Gpi), which showed sustained expression throughout maturation (FIGS. 26A-26D) and induced gene knock-out upon infection with lentiviral vectors coding gRNAs (FIGS. 26D-26E). Gpi::Cas9 hPSCs and infected synchronized postmitotic cortical neurons at d25 were differentiated with an arrayed library of lentiviral vectors encoding dTomato reporter and gene-specific gRNAs (2 gRNA/gene and 2 non-targeting control gRNAs; Table 4). The present disclosure screened for the ability of each gRNA-induced gene perturbation to trigger preconscious expression of cytoskeleton and pre-synaptic proteins Nefh and Stx1a respectively, which expression captured stage of neuronal maturity (FIGS. 19C-19D; FIG. 27B). Western Blot analysis (WB) at d35 showed widespread increased expression of such markers across the different gene perturbations compared to non-targeting control gRNAs (FIG. 19D; FIG. 27C). The present disclosure investigated the preconscious expression of molecular markers in perturbed neurons triggered maturation-related functional changes through GCaMP6m-based imaging of spontaneous Ca²⁺ spikes at d40 (FIG. 19C). The present disclosure revealed that loss-of-function of ˜half of the perturbed chromatin regulators induced significant robust increase in the amplitude of individual spontaneous Ca²⁺ spikes (FIG. 19E) with a trend towards increased synchronous firing rates compared to non-targeting gRNA control conditions (FIG. 27D). This comprised multiple PRC1/2-related genes (Cbx2, Rnf2, Epc1, Epc2, Ezh2 and Mtf2), the NurD complex member Chd3, the lysine methyltransferase Kmt5b, the BAFs members Smarca4 and SmarcaD1 as well as the bromodomain containing gene Brd1. The present disclosure clearly identified the loss-of-function of few chromatin regulators as key players in the temporal onset of molecular and functional maturation features. Importantly, this epigenetic signature is similarly downregulated during the maturation of multiple cortical neuron subtypes in the developing mouse cortex in vivo (FIGS. 31A and 31B) albeit at a much more rapid pace than in human cells.

TABLE 4
Gene-specific gRNAs

| gRNA ID | gRNA Target sequence | SEQ ID NO |
|---|---|---|
| NT1 | AAAAAGCTTCCGCCTGATGG | 1 |
| NT2 | CATAGGTCCCTAGCAACTCC | 2 |
| BRD1#1 | AAATAGGATTGCGAATCAGG | 3 |
| BRD1#2 | AAACTGCTTCTTCCGCTGAA | 4 |
| CBX5#1 | TACCCAGGGAGCACAATACT | 5 |
| CBX5#2 | TAAATTCAGAAATTAGCTCA | 6 |
| CHD3#1 | GCAACTCTGCTTCTGACCTG | 7 |
| CHD3#2 | GGAGCATGTGTTCTCTGAGG | 8 |
| EPC1#1 | TTTAGGAACATCATCTTCAG | 9 |
| EPC1#2 | GCGGGCTATTTCAGCACAGC | 10 |
| EPC2#1 | TTGACGTTGCTCTCTGCCTC | 11 |
| EPC2#2 | TATTACAATCGCTTGTACAA | 12 |
| EZH2#1 | CGGAAATCTTAAACCAAGAA | 13 |
| EZH2#2 | GCAATGAGCTCACAGAAGTC | 14 |
| HDAC2#1 | TGGGTCATGCGGATTCTATG | 15 |
| HDAC2#2 | ACAGCAAGTTATGGGTCATG | 16 |
| KDM1A#1 | ATACTCATCTTCTGAGAGGT | 17 |
| KDM1A#2 | ACCCCCTCAAGCCCCACCTG | 18 |
| KDM5B#1 | ACTCCCAGTACTTTGCAATC | 19 |
| KDM5B#2 | GCAAAGTACTGGGAGTTACA | 20 |
| KMT5B#1 | TTTCACAGGTAATGGTAACT | 21 |
| KMT5B#2 | AGTCGCTATGTACCATCCTC | 22 |
| MTA2#1 | GCGCCATCAACTGAAGCACC | 23 |
| MTA2#2 | GAAGAGGAATCAAAGCAGCC | 24 |
| MTF2#1 | TGATTGATTCAGATGAAAAA | 25 |
| MTF2#2 | CAGATGAAAAATGGCTCTGT | 26 |
| RBBP4#1 | TCATGTCACCTGGTTACATC | 27 |
| RBBP4#2 | ATTGCCGTTACAGACCAGAA | 28 |
| RNF2#1 | ATCATCACAGCCCTTAGAAG | 29 |
| RNF2#2 | AATTCACTGTGTAGACTTCG | 30 |
| SMARCA4#1 | ATGGTCCCTCTCGCAGCCCA | 31 |
| SMARCA4#2 | CTTTCATCTGGTTGTAGCGC | 32 |
| SMARCE1#1 | CAGCAAATGCCCAGCACACC | 33 |
| SMARCE1#2 | TCCTACCGTGACCCGGCTGT | 34 |
| SMARCAD1#1 | AACTGTATTGGAGCAATTTG | 35 |
| SMARCAD1#2 | GAACTGTATTGGAGCAATTT | 36 |
| RCOR2#1 | TCTGTGGCATAAGCACGATG | 37 |
| RCOR2#2 | GCATTTGCCATGGAAGCCAA | 38 |
| SOX4#1 | GCCCGAGTCCGAGCTCTCGC | 39 |
| SOX4#2 | ATTGTTGGTTTGCTGCACCA | 40 |
| SOX11#1 | GCGGATCATGGTGCAGCAGG | 41 |
| SOX11#2 | ATTCATGGCTTGCAGCCCGG | 42 |
| KLF12#1 | CGTCCACAACTATCCCGATA | 43 |
| KLF12#2 | ACAGATAACGAGTCCTCCGG | 44 |

In summary, the progressive downregulation of few chromatin regulators together with their ability to trigger preconscious maturation under loss-of-function in neurons showed an epigenetic “brake” that prevents maturation and is gradually released allowing the lengthy unfolding of molecular and functional maturation programs.

### An Epigenetic Barrier in NPCs Determined the Rate of Human Neuronal Maturation

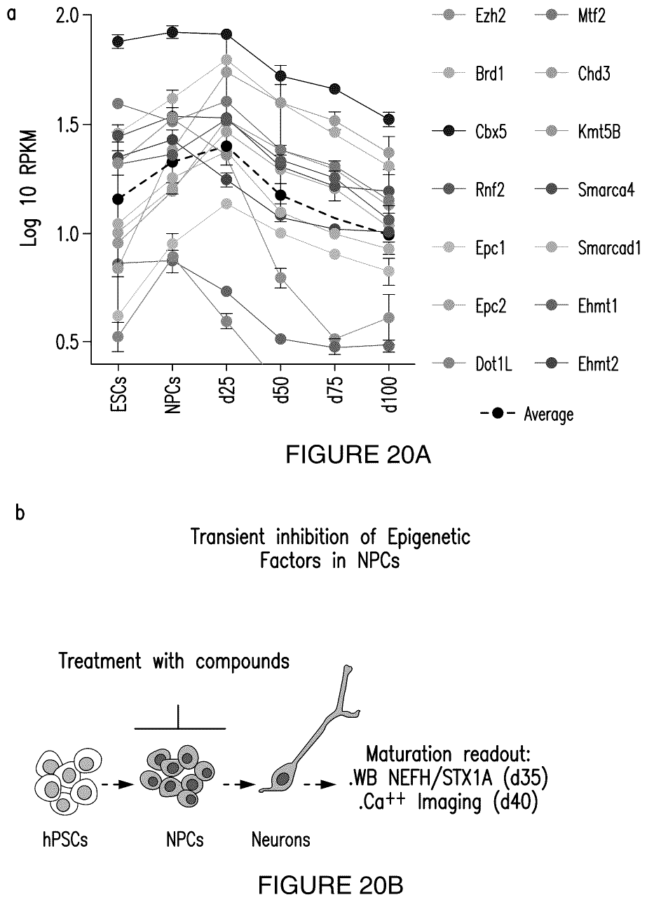

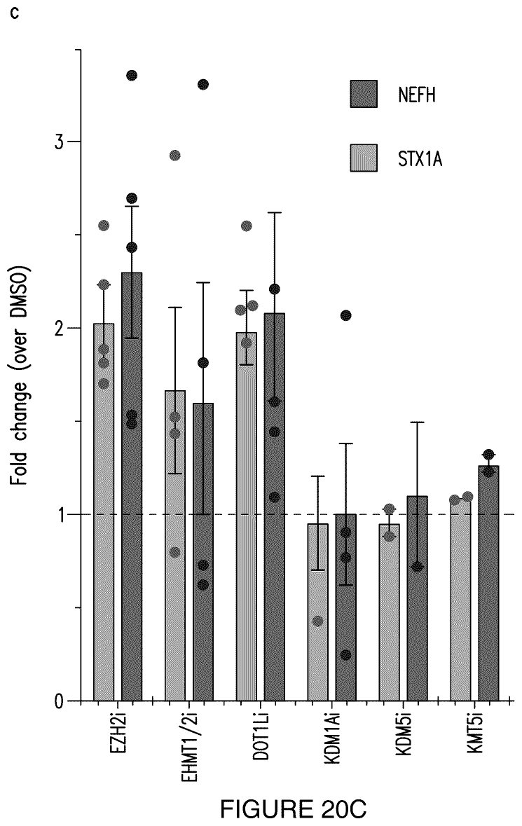

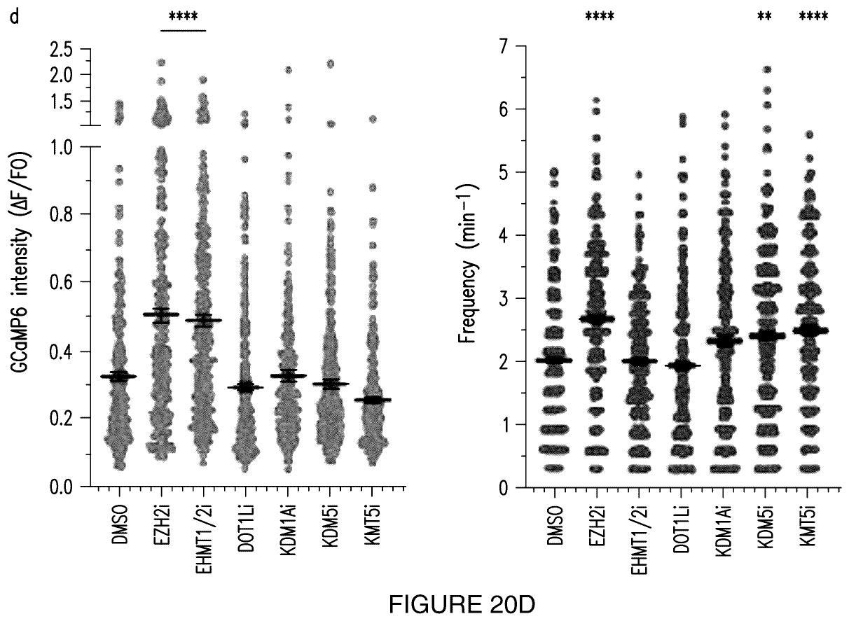

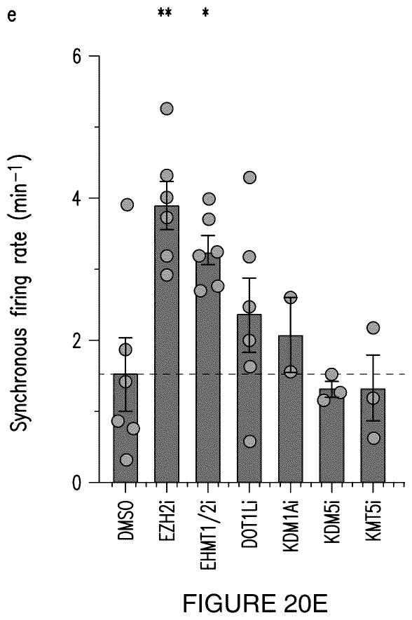

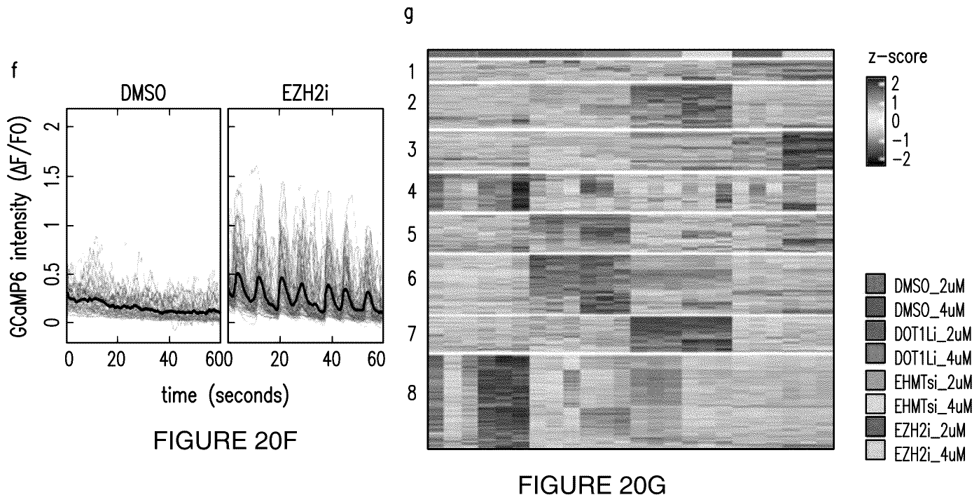

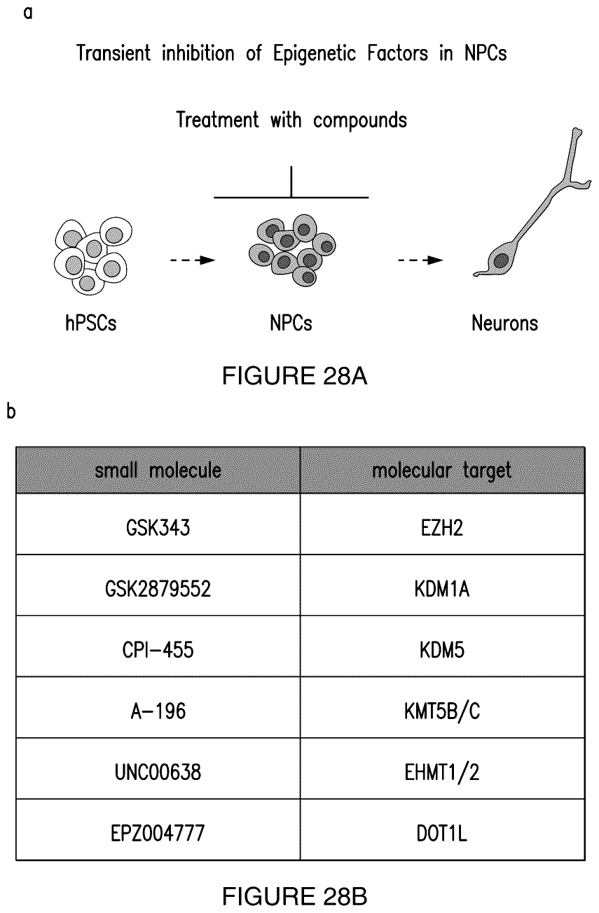

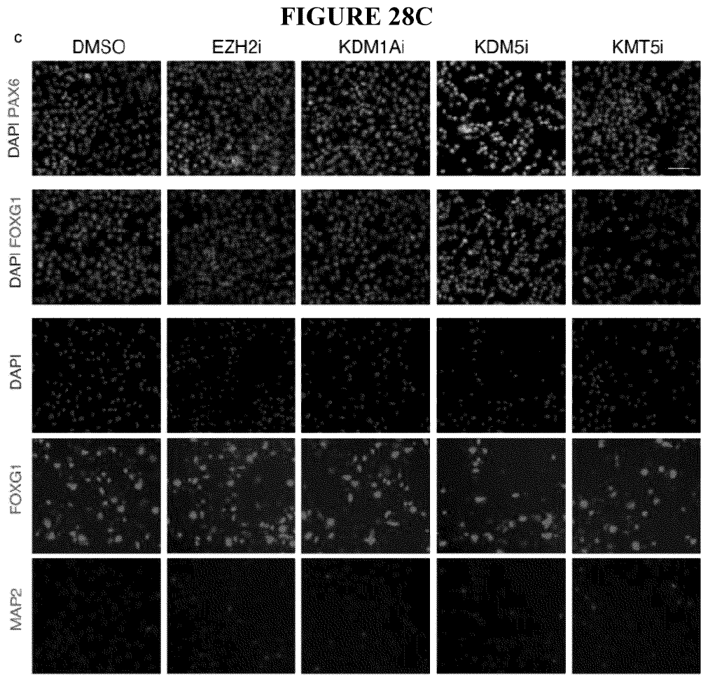

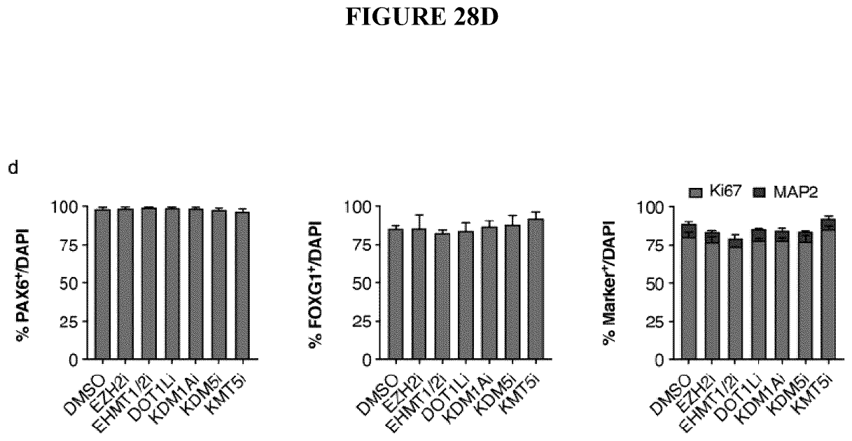

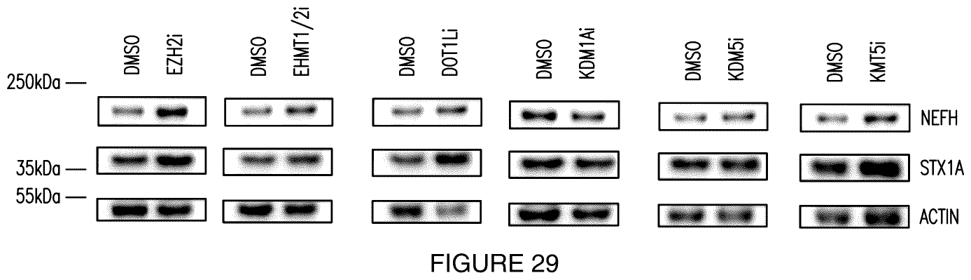

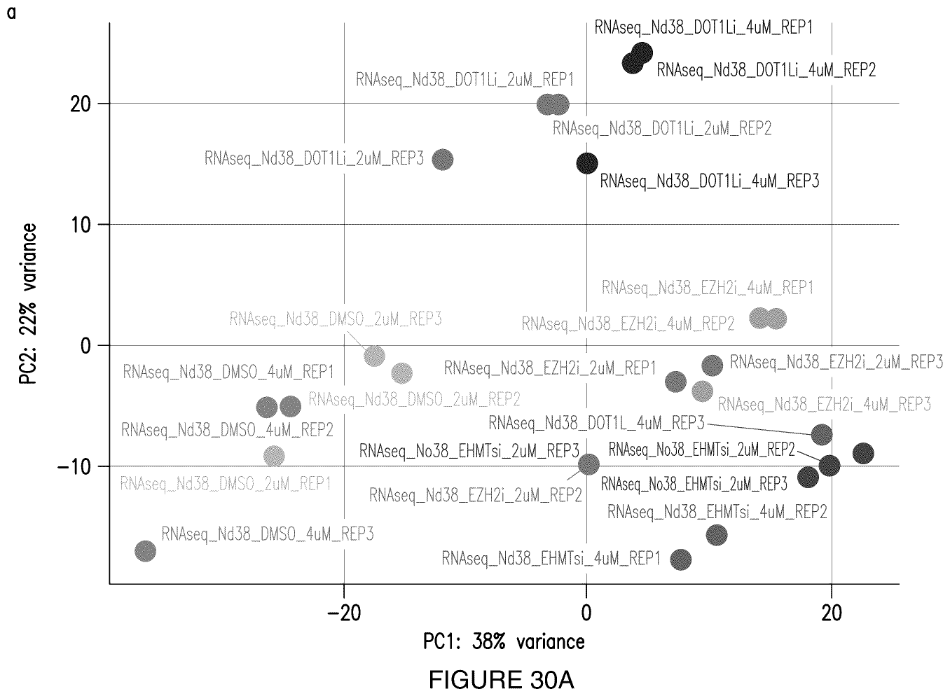

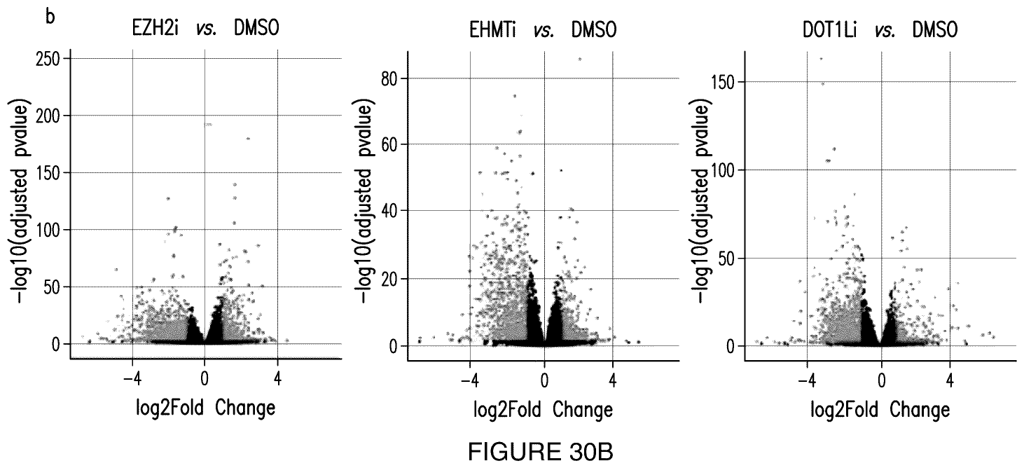

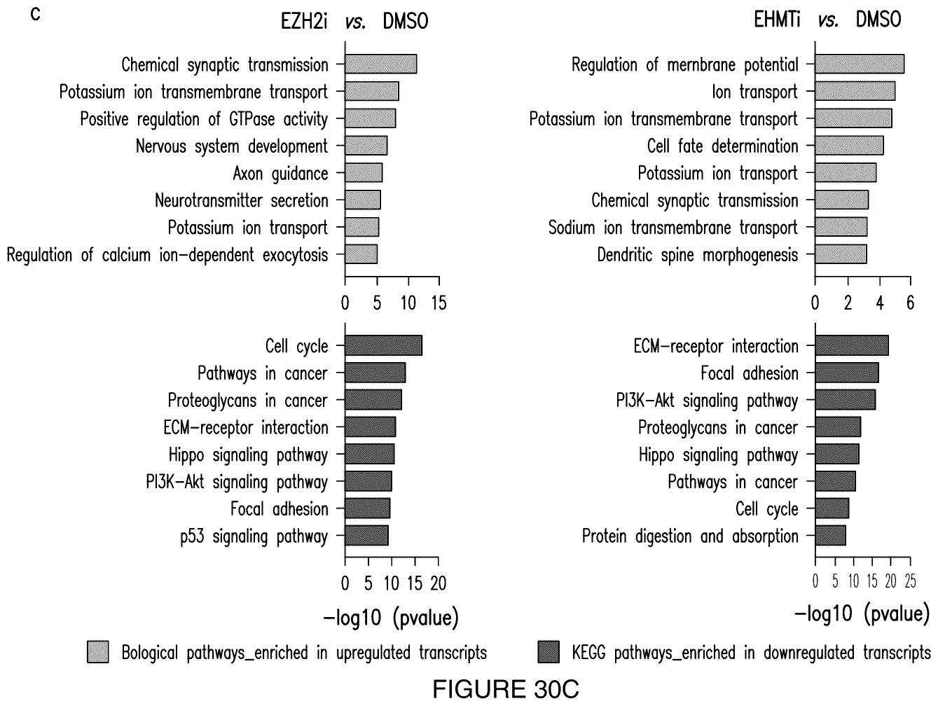

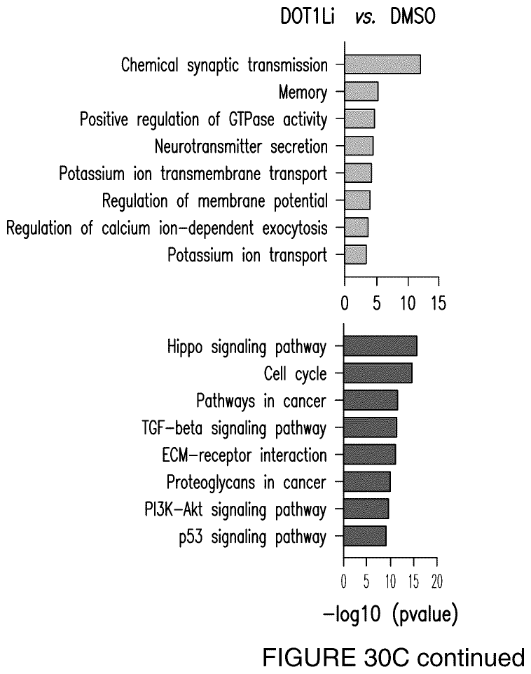

The arrayed genetic screen in hPSC-derived neurons, described in the present disclosure, identified a subset of chromatin regulators (hits) that drove molecular and functional maturation upon loss-of-function at neuron stage. Temporal expression analysis throughout the differentiation revealed that the vast majority of the hits were expressed already in dividing NPC (FIG. 20A), raising the intriguing possibility that a subset of chromatin regulators participate in establishing an “epigenetic barrier” at maturation during hPSC-to-NPC transition, well before the onset of neurogenesis. The enhanced neuronal maturation could be achieved by introducing manipulations specifically at NPC stage. This was shown in the present disclosure, using small molecule inhibitors that targeted some of the epigenetic hits form the loss-of-function screen in neurons (FIG. 28B) and inhibitors of the histone lysine methyltransferases EHMT1/2 and DOT1L, which expression showed also monotonic downregulation during maturation. NPCs were transiently treated with small molecule inhibitors after the induction of cortical CNS patterning from d12 to d20 and small molecules were washed out and windrowed at d20 before the induction of synchronized neurogenesis (FIG. 20B). Neurons derived from treated and DMSO control NPC were grown in the same exact culture conditions. Small molecule treatments under this paradigm did not alter the expression of Pax6 and FoxG1 cortical markers and did not induce preconscious neurogenesis based on the ratio of Ki67⁺ NPC and Map2⁺ neurons at d20 (FIGS. 28C-28D). The extent of maturation achieved by the different manipulations was assessed through WB for maturation markers and Ca²⁺ imaging at d35 and d40 respectively (FIG. 20B). Among the treatments, transient inhibition of EZH2, EHMT1/2 and DOT1L in NPC with GSK343, UNC0638 and EPZ004777 respectively induced robust increased expression of the maturation markers Nefh and Stx1a compared to DMSO control (FIG. 20D; FIG. 29). In addition, transient inhibition of EZH2 in NPC triggered a marked and highly significant increase in all measured functional properties such as amplitude and frequency of individual Ca²⁺ spikes and synchronicity of the neuronal network respect to DMSO control neurons (FIGS. 20D-20F). EHMT1/2 inhibition induced significant changes in the amplitude and synchronicity of Ca²⁺ spikes while neurons derived from NPC treatment with DOT1L inhibitor showed a modest functional enhancement with an increased synchronous firing rate (FIGS. 20D-20F). RNAseq was performed on d38 neurons derived from NPC treated with EZH2, EHMT1/2 and DOT1L inhibitors at two different concentration (2 and 4 uM). PCA showed that all treated samples clustered apart from DMSO controls and distributed according to type of treatment (FIG. 30A) with robust changes in both directions (FIG. 30B). Downregulated genes primarily captured transcripts typically found in progenitor cells (FIG. 30C) and comprised for instances members of the Sox family of TF and Notch pathway-related transcripts among others (FIG. 20g). Upregulated transcripts were instead robustly enriched for maturation related GO such as chemical synaptic transmission and ion transmembrane transport (FIG. 30C) and comprised several maturation markers whose expression monotonically increase during natural maturation, including Hla-bc, Tuba4A, S100A10, Fos, FosB and LINC00473 among others (FIG. 18C; FIG. 20G). Interestingly, while downregulated transcripts were in large part shared among neurons derived from the different NPC manipulations, the induction of maturation related transcripts appeared more diverse; with shared as well as treatment specific signatures (FIG. 20G).

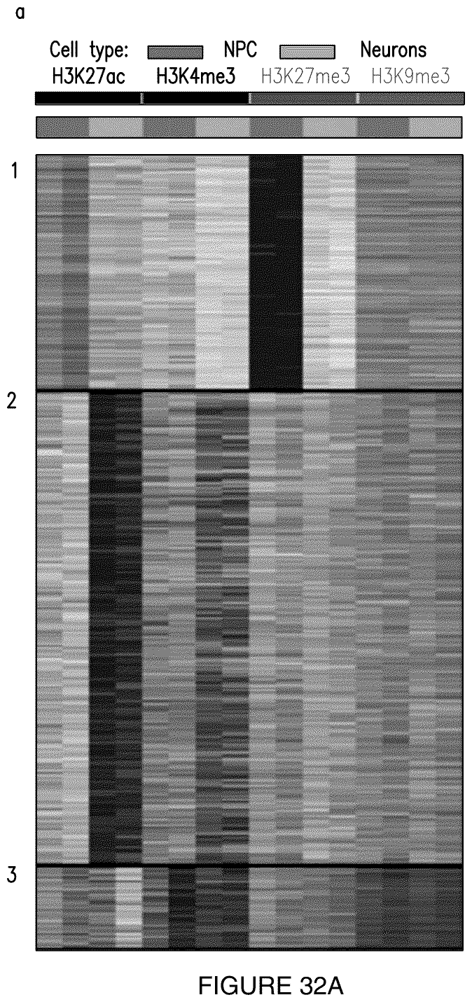

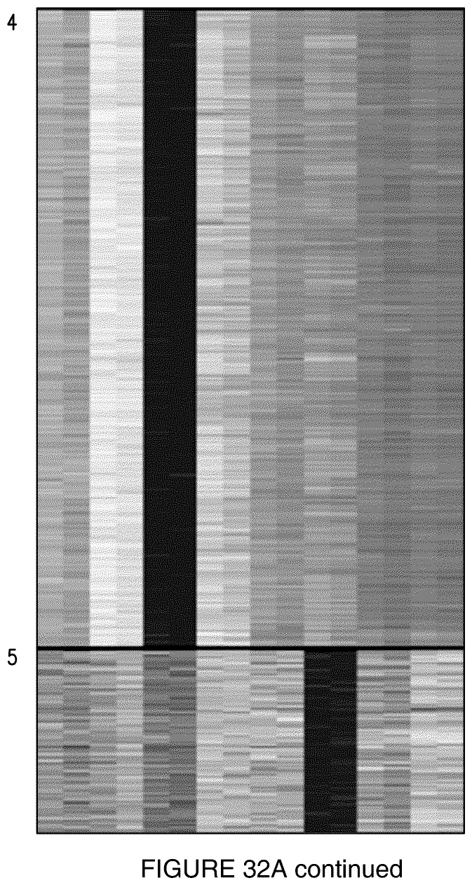

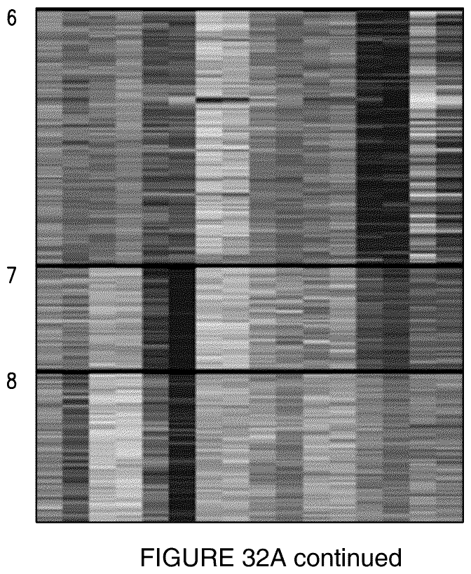

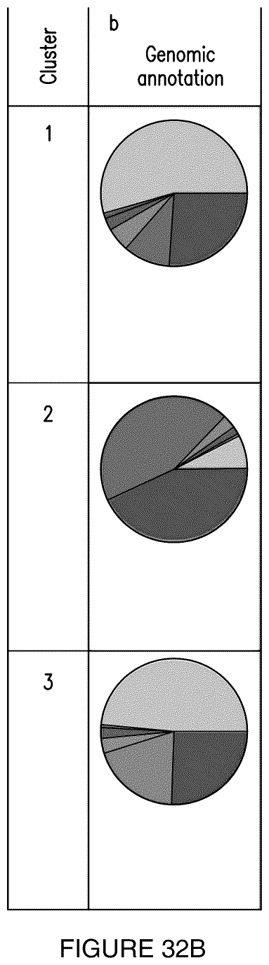

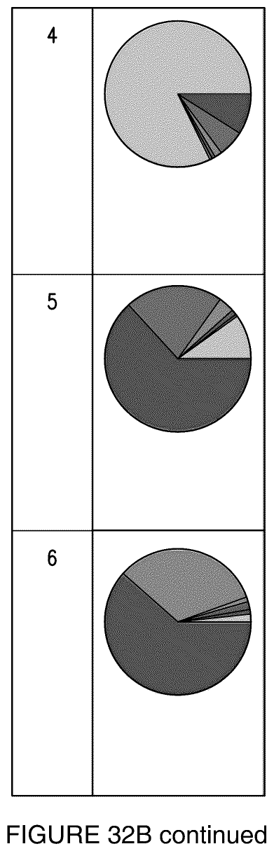

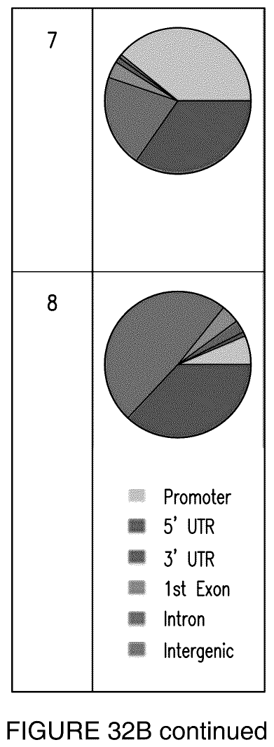

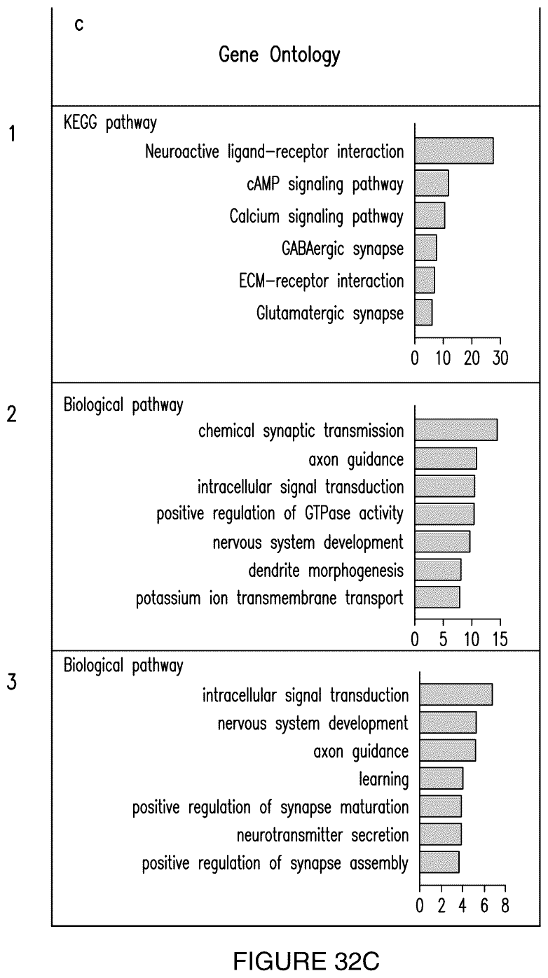

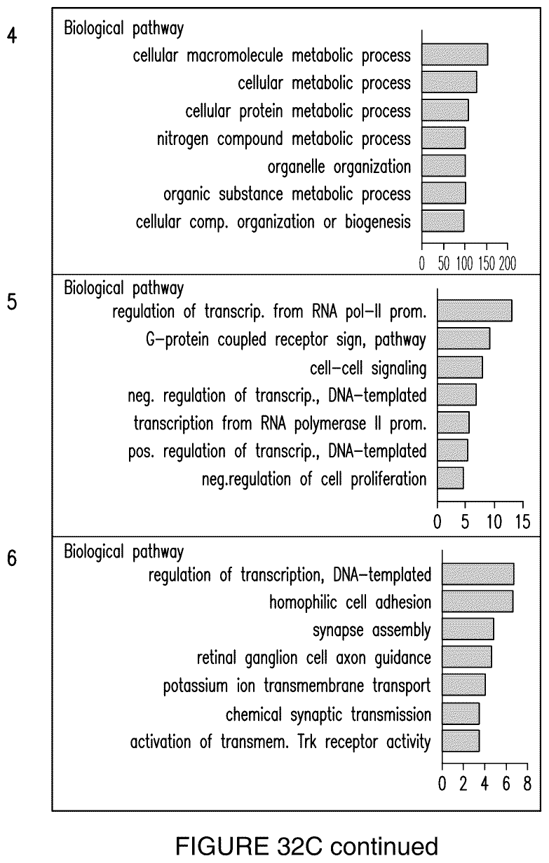

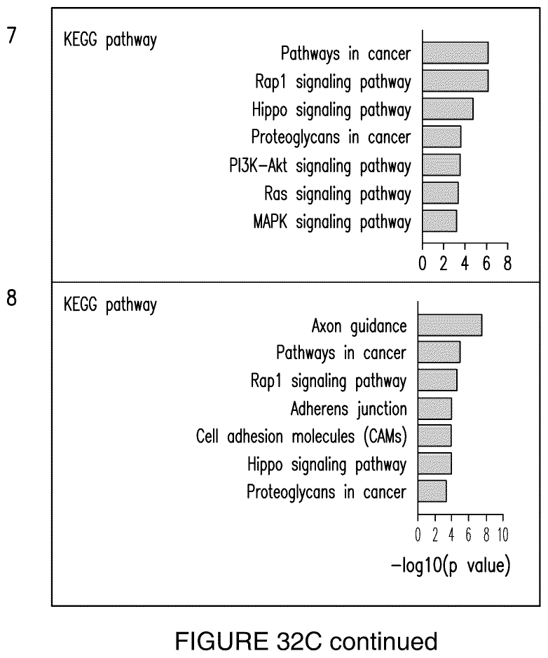

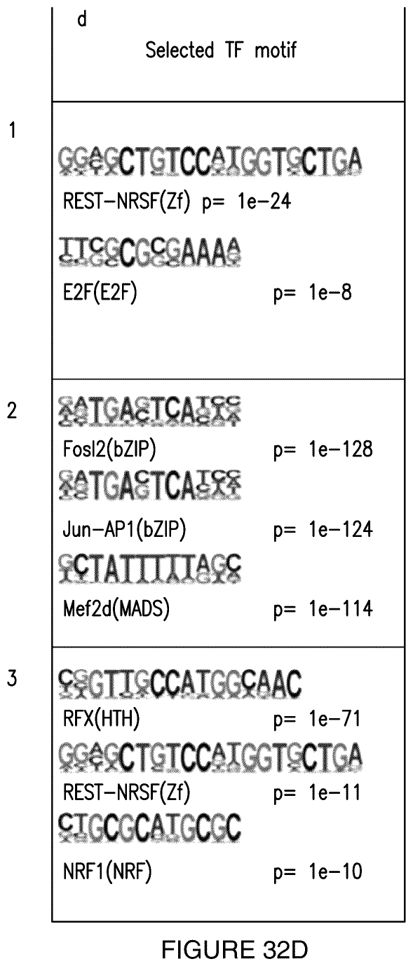

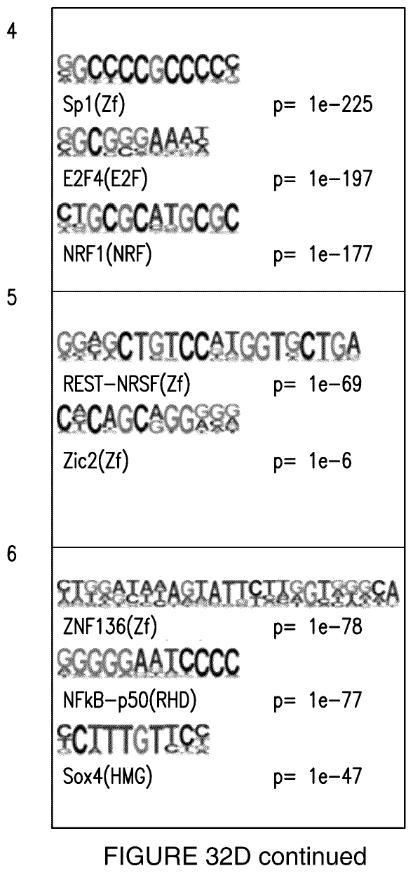

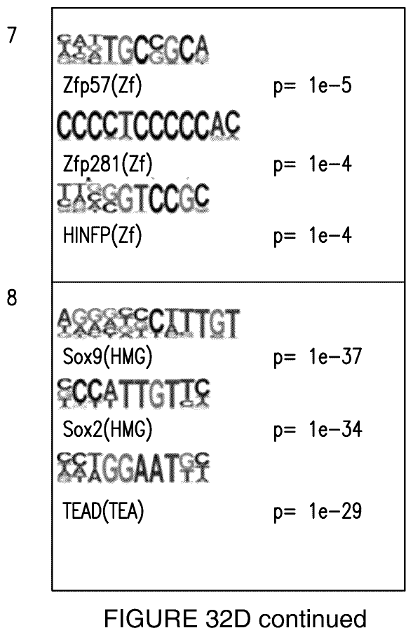

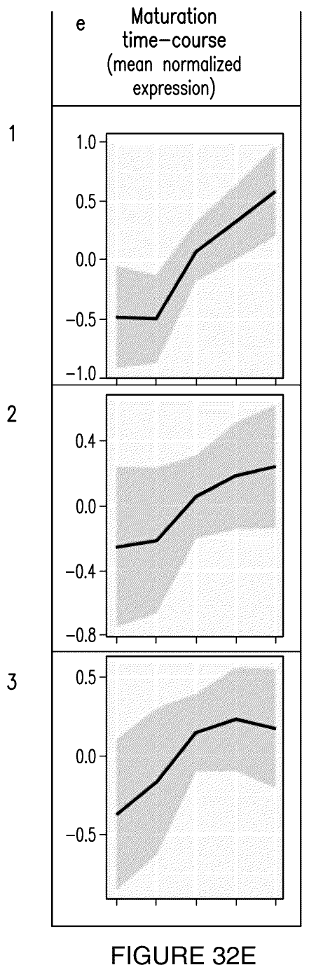

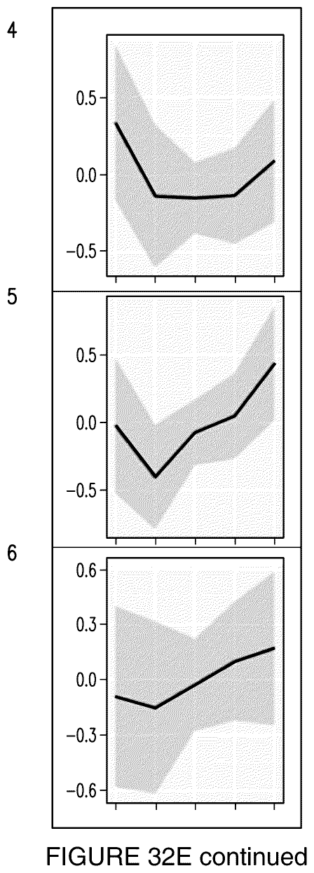

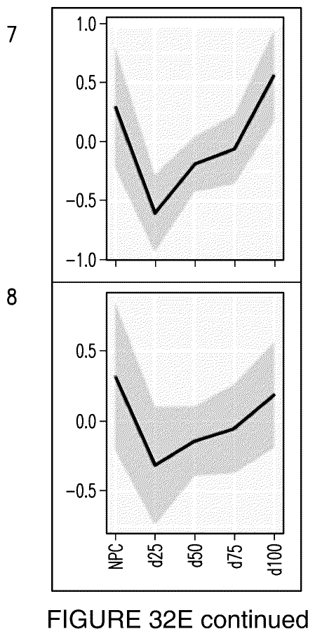

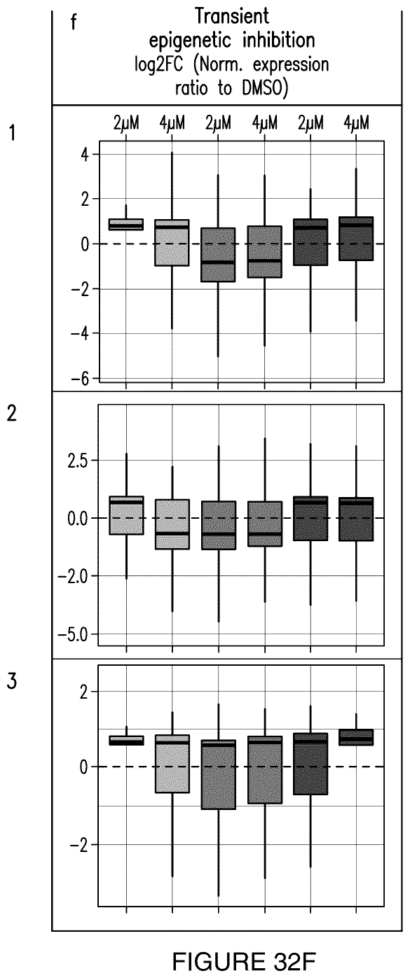

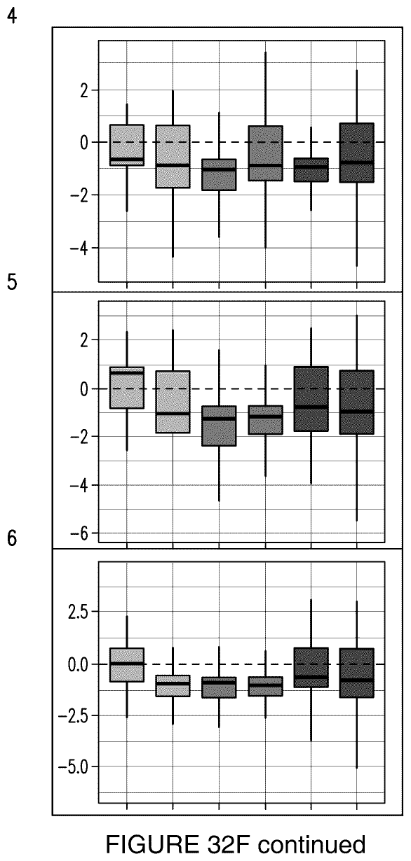

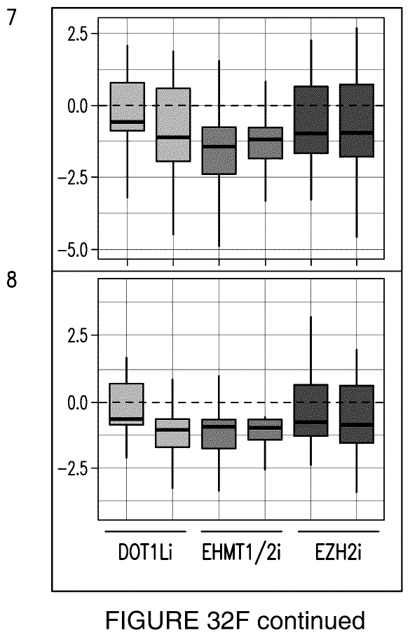

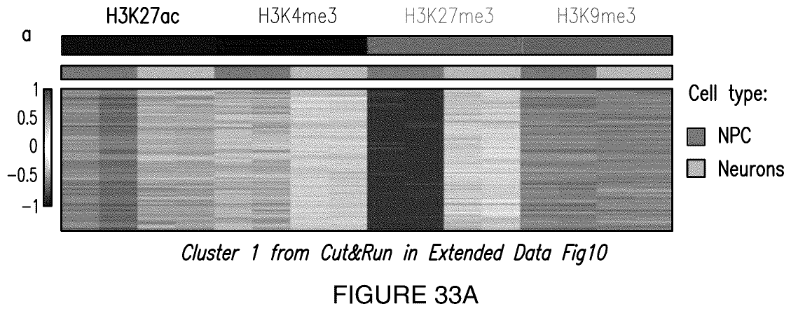

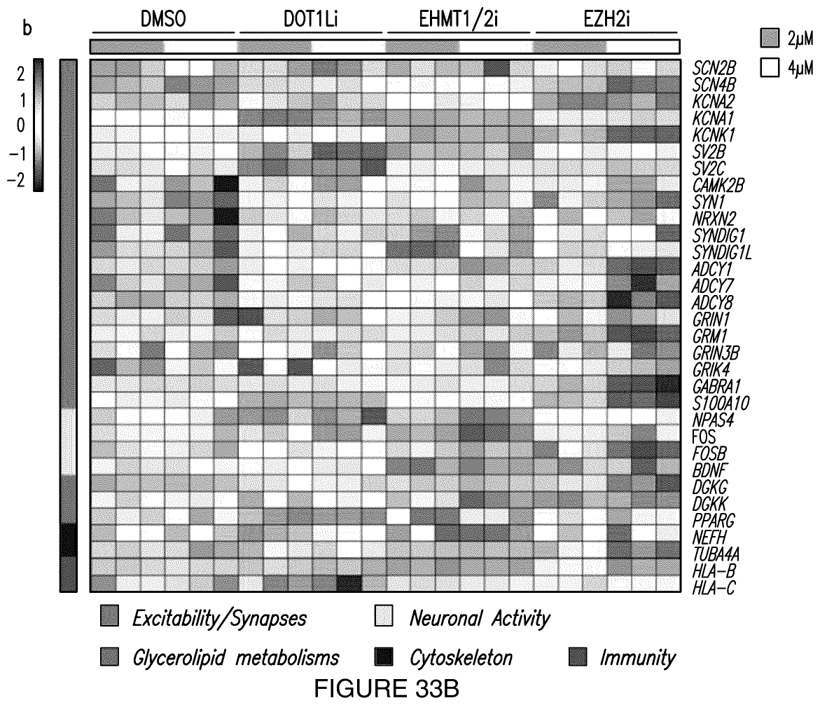

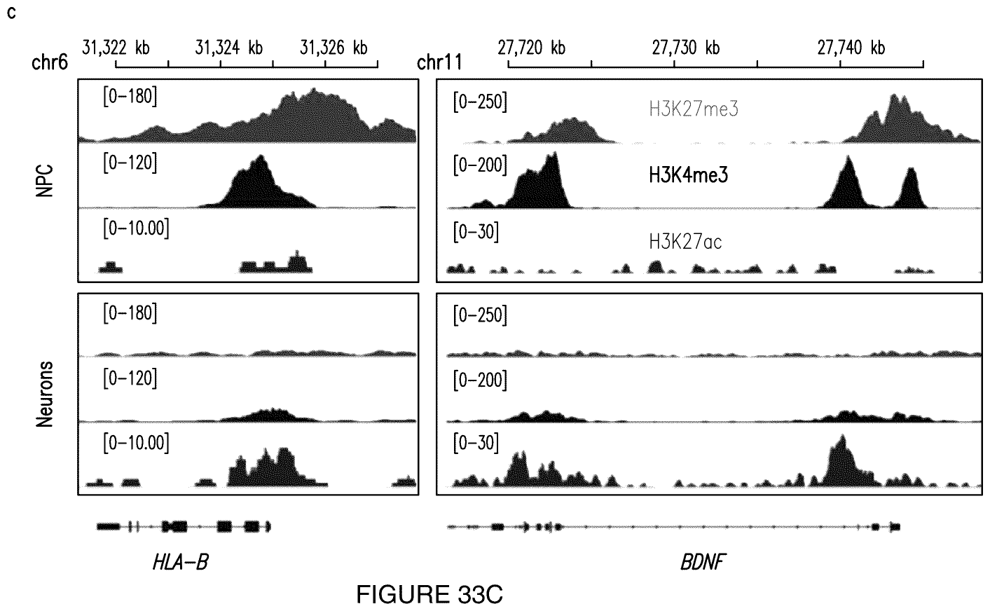

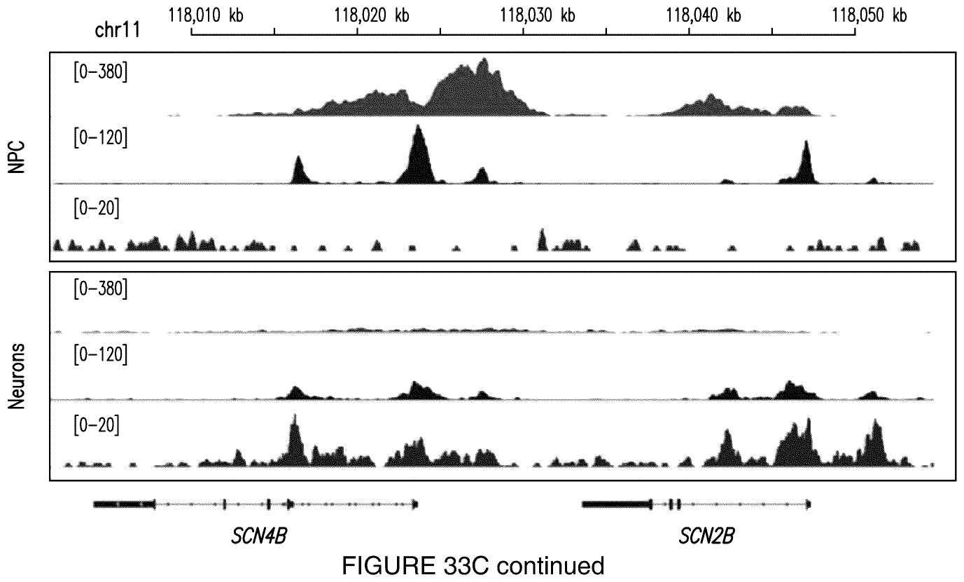

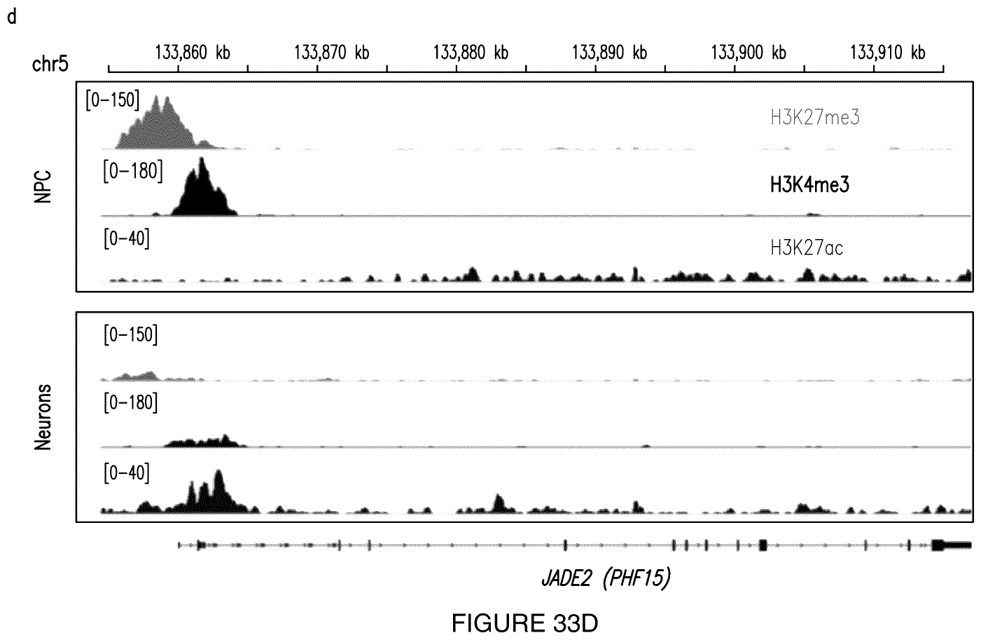

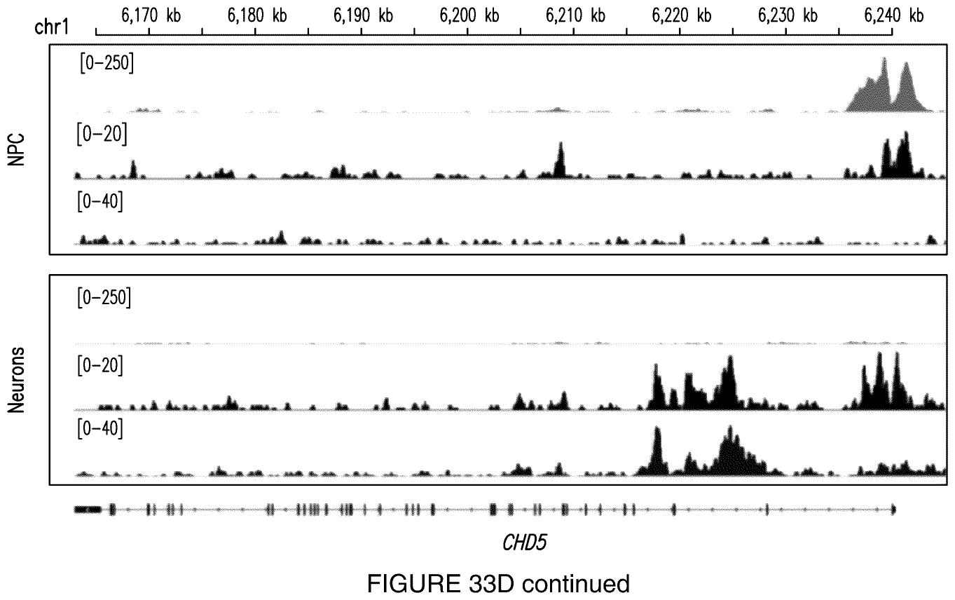

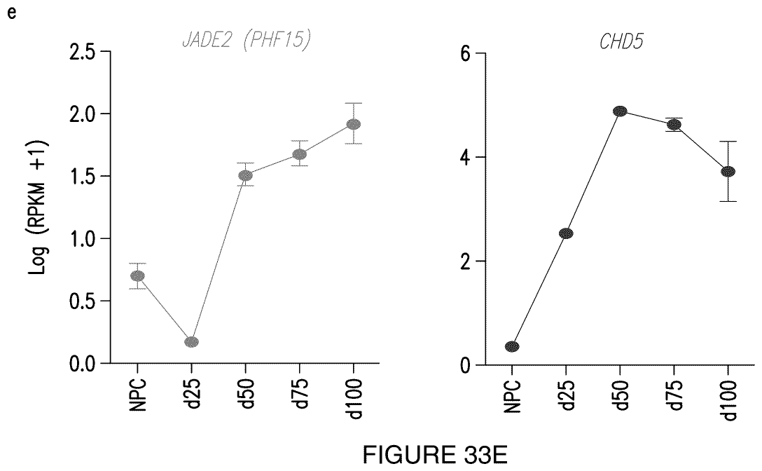

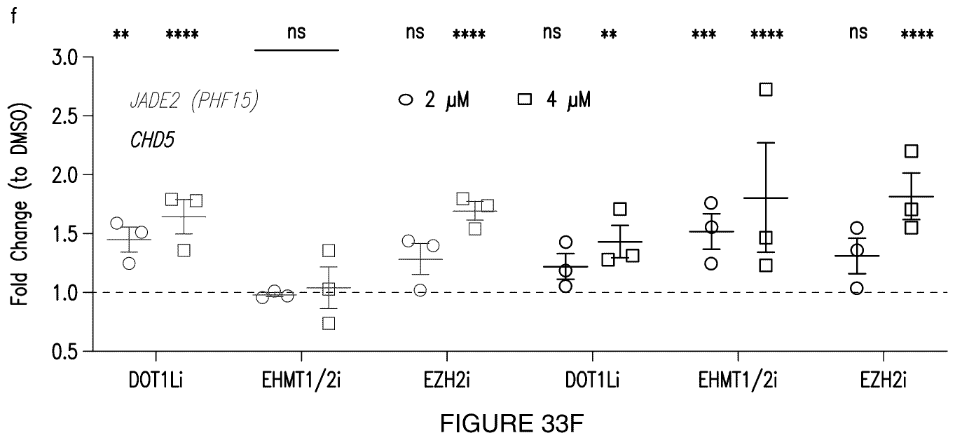

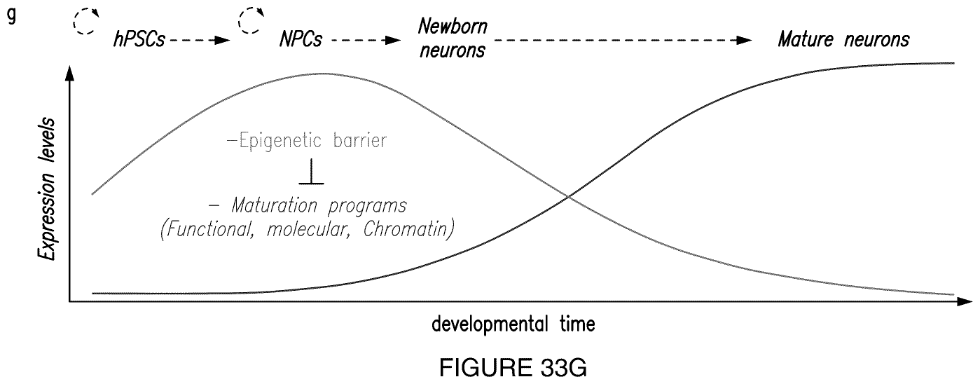

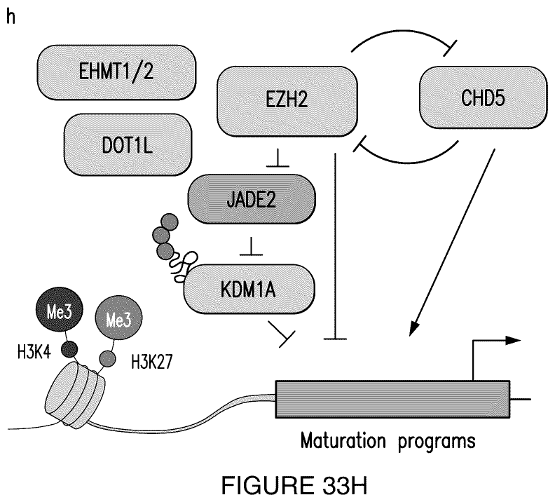

To gain insights into the epigenetic regulation of maturation programs, the present disclosure characterized the dynamics of H3K27ac, H3K4me3, H3K27me3 and H3K9me3 histone post-translational modifications (PTMs) in hPSC-derived cortical NPC and neurons via CUT&RUN experiments. Unsupervised clustering of CUT&RUN peaks with differential binding for histone PTMs in NPC vs. Neurons identified 8 groups of peaks characterized by distinct combinatorial patterns of histone PTMs (FIG. 32A). GO analysis on genes linked to CUT&RUN peaks identified cluster 1, 2 and 3 as highly enriched for chemical synaptic transmission and ion transmembrane transport terms among others, indicating that epigenetic regulation at these genomic loci may play a role in driving maturation-related gene expression (FIG. 32C). The present disclosure then intersected the genes linked to each CUT&RUN cluster with all differentially expressed genes by RNAseq in NPC, d25, d50, d75 and d100 neurons, irrespective of the directionality of the changes. This analysis identified a correlation between the patterns of histone PTMs in clusters 1, 2 and 3 and maturation-dependent changes in gene expression (FIG. 32E). Furthermore, maturation-related genes belonging to cluster 1, 2 and 3 were among the statistically upregulated transcripts in neurons derived from NPC transiently treated with DOT1L and EZH2 inhibitors versus those derived from DMSO treated control NPC (FIG. 32F). Cluster 2 defined a subset of peaks with increased dual binding for H3K27ac and H3K4me3 histone PTMs at neuron stage, marking active chromatin domains at putative enhancer sequences, enriched for activity-dependent TF motif such as AP1 and MEF gene families (FIGS. 32B and 32D) suggesting that activity-dependent mechanisms contribute to driving neuronal maturation. In contrast, cluster 1 was dominated by the dual presence of the EZH2 dependent H3K27me3 repressive mark and the active H3K4me3 PTM at NPC stage. Such poised or bivalent state was resolved toward active chromatin state at neuron stage via loss of the repressive H3K27me3 mark and acquisition of modest levels of the active H3K27ac PTM (FIGS. 32A and 33A). Cluster 3 showed a similar pattern with a partial bivalent state in NPC and a more pronounced acetylation of H3K27 in neurons (FIG. 32A). These results indicate a key role for the EZH2 dependent deposition of the H3K27me3 repressive mark in maintaining maturation programs in a poised state, a finding further supported by the increased expression of various maturation genes (with bivalent chromatin state in NPC; FIGS. 33A and 33B) upon transient treatment with EZH2 inhibitors. Those transcripts match the unperturbed, chronological maturation signature (FIGS. 17L and 18B-18D) and are involved in synaptic assembly and functionality, activity-dependent mechanisms (FOS, FOSB, NPAS4, BDNF), glycerolipid metabolism and PPAR signaling (DGKK, DGKG, PPARG), maturation of the cytoskeleton (NEFH, TUBA4A) and immunological programs (HLA-B and C) (FIGS. 33B and 33C). Interestingly, among the bivalent genes at NPC stage, several chromatin regulators that show a gradual increase in expression during cortical neuron maturation were induced dose-dependently in NPC upon transient epigenetic inhibition (FIGS. 33D-33F). These include JADE2 (also known as PHF15), a ubiquitin ligase that target for degradation KDM1A, whose loss-of-function triggered increased expression of maturation markers (FIG. 19D) and CHD5, which facilitates the expression of neuron specific gene programs. These results indicate that the epigenetic barrier identified in our study controls the temporal onset of maturation via a dual mechanism; by directly maintaining maturation genes in a poised state and indirectly by modulating the expression of competing epigenetic regulators promoting maturation (FIGS. 33G and 33H).

### Discussion

The present disclosure discovered an approach to measure and override the intrinsic human maturation clock. To this end, the present disclosure describes a novel platform for the synchronized generation of cortical neurons from hPSC and established roadmaps for morphological, functional, and molecular maturation. The present disclosure uncovered the unfolding of molecular and functional maturation programs proceeded gradually and was limited by the retention of an epigenetic signature in neurons that prevent the progression toward maturity. In addition, the present disclosure shows that the rate at which neurons mature was determined well before neurogenesis through the establishment of an “epigenetic barrier” in progenitor cells that get slowly erased at neuron stage. The present disclosure also shows that manipulation of epigenetic regulators exclusively in progenitor cells was sufficient to accelerate the maturation of hPSC-derived neurons.

Altogether, these results demonstrated that enhancement of maturation state can also be achieved through inhibition of chromatin regulators at NPC stage and identified EZH2, EHMT1/2 and DOT1L as upstream factors. In addition, these results supported the existence of multiple epigenetic barriers in NPC that get inherited in newborn neurons and retained for protracted periods of time, contributing to the lengthy maturation of human neurons, and ultimately setting the rate of their maturation.

### Material and Methods

### Cell Culture

Human pluripotent stem cells (hPSCs) WA09 (H9; 46XX) and derivate GPI::Cas9 were maintained with Essential 8 media (Life Technologies #A1517001) in feeder-free conditions onto Vitronectin (VTN-N, Thermo Fisher #A14700) coated dishes. hPSCs were passaged as clumps every 4-5 days with EDTA (0.5M EDTA/PBS) and routinely tested for mycoplasma contamination. GPI::Cas9 knock-in hPSCs line was generated using CRISPR/Cas9-mediated homologous recombination by transfecting H9 hPSCs with the Cas9-T2A-Puro targeting cassette downstream of the GPI gene. Selected clones were validated by genomic PCR and Cas9 mRNA and protein expression by qRT-PCR and Western Blot respectively and screened for Karyotype banding.

Synchronized generation of cortical neurons—hPSCs (passage 40-50) were differentiated toward cortical excitatory neurons using an optimized protocol based on dual-SMAD inhibition and WNT inhibition as following. hPSCs were dissociated at single cells using Accutase and plated at 300,000 cells/cm² onto Matrigel (#354234, Corning) coated wells in Essential 8 media supplemented with 10 μM Y-27632. On day 0-2, cells were fed daily by complete medium exchange with Essential 6 medium (E6, #A1516401, Thermo Fisher Scientific) in the presence of 100 nM LDN193189 (#72142, Stem Cell Technologies), 10 μM SB431542 (#1614, Tocris) and 2 μM XAV939 (#3748, Tocris) to induce anterior neuroectodermal patterning. On day 3-9 cells were fed daily with Essential 6 medium (E6, #A1516401, Thermo Fisher Scientific) in the presence of 100 nM LDN193189 (#72142, Stem Cell Technologies), 10 μM SB431542. On day 10-20 cells were fed daily with N2/B27 media (1:1 NB:DMEM/F12 basal media supplemented with 1×N2 and B27 minus vitamin A to generate a neurogenic population of cortical neuronal progenitor cells (NPCs). N2 and B27 supplements were from Thermo. At day 20, NPCs were either cryopreserved in STEM-CELLBANKER solution (Amsbio) or induced for synchronized neurogenesis as following: NPCs were dissociated at single cells following 45 min incubation with Accutase and seeded at 150,000 cells/cm² onto poly-L-ornithine and Laminin/Fibronectin coated plates in NB/B27 medium (1×B27 minus vitamin A, 1% L-glutamine and 1% Pen/Strep in Neurobasal medium) in presence of 10 μM Notch pathway inhibitor DAPT for 10 days (until day 30). For long term culture, neurons were maintained in NB/B27 supplemented with BDNF (#450-10, PreproTech), GDNF (#248-BD-025, R&D biosystems), cAMP (#D0627, Sigma) and AA (#4034-100, Sigma). From day 20 onwards, cells were fed every 4/5 days.

EdU labelling and small molecule treatments—For birth dating experiments of hPSC-derived cortical neurons, 3 μM EdU (5-ethynyl-2′-deoxyuridine, A10044 Invitrogen) was added to the culture for 48 h in the following time window: day 18/19, day 20/21, day 22/23, day 24/25, day 26,27, day 28/29. After treatment, EdU was washed out and neurons were fixed at day 40 of differentiation and processed for immunostaining. Treatment of cortical neuronal progenitor cells (NPCs) with small molecules inhibitors of chromatin regulator was performed from day 12 to 20 of differentiation (FIG. 20B). List of small molecules that are relative intracellular targets are reported in FIG. 28B. Small molecules were dissolved in DMSO and added to the N2/B27 media at 2 or 4 μM depending on the experiment. Small molecules were washed out before the induction of synchronized neurogenesis and neurons derived from all the treatments were maintained in the same conditions.

### Morphological Reconstructions

hPSCs derived neurons were infected with low titer lentiviruses expressing dTomato reporter at day 20 and fixed at day 25, 50, 75 and 100. The dTomato reporter signal was amplified by immunofluorescence staining and individual neurons were imaged at 10×. Neuronal morphology was reconstructed using the filament tracing function of Imaris software. Measurements were performed in the Imaris platform and extracted for quantifications and statistics.

### Immunofluorescence

Cultured cells were fixed with 4% PFA in PBS for 20 min at RT, washed three times with PBS, permeabilized for 30 min in 0.5% Triton X-100 in PBS and then blocked in a solution containing 5% Normal goat serum, 2% BSA and 0.25% Triton X-100 for 1 h at RT. Primary antibodies were incubated overnight at 4° C. The following primary antibodies were used: rabbit anti-Pax6 (901301, Biolegend); rabbit anti-FoxG1 (M227, Clonetech); mouse anti-Nestin (M015012, Neuromics); mouse anti-MAP2 (M1406, Sigma); chicken anti-MAP2 (ab5392, Abcam); rabbit anti-Class III β-tubulin TUJI (MRB-435P, Covance); mouse anti-Ki67 (M7240, Dako); rabbit anti-Ki67 (RM-9106, Thermo Scientific); rabbit anti-Tbr1 (ab183032, Abcam); rat anti-Ctip2 (ab18465, Abcam); mouse anti-Satb2 (ab51502, Abcam); rabbit anti-Synapsin I (S193, Sigma); mouse anti-Neurofilament H (non-phosphorylated) (SMI32; Enzo Life science); mouse anti c-Fos (ab208942, Abcam); mouse anti-HLA Class I ABC (ab70328, abcam); goat anti-RFP (200-101-379, Rockland); rabbit anti-DsRed (632496, Clontech). EdU⁺ cells were detected using the Click-iT EdU Imaging kit (Molecular Probes) with Alexa Fluor 488. Secondary antibodies conjugated to either Alexa 488, Alexa 555 or Alexa 647 (Thermo) were incubated for 45 min. Cell nuclei were stained with 5 μM 4′-6-diamidino-2-phenylindole (DAPI) in PBS.

### Electrophysiological Recording

Neurons were plated in 35 mm dishes and whole-cell patch clamp recordings were performed at day 25, 50, 75 and 100 of differentiation as previously described (Maroof et al., Cell Stem Cell 12, 559-572 (2013)). Briefly, neurons were visualized using a Zeiss microscope (Axioscope) with a 4× objective and a 40× water immersion. Recordings were performed at 23-24° C. and neurons were perfused with freshly prepared ACSF extracellular solution saturated with 95% O₂-5% CO₂ (in mM: 126 NaCl, 26 NaHCO₃, 3.6 KCl, 1.2 NaH₂PO₄, 1.5 MgCl₂, 2.5 CaCl₂, and 10 glucose). Pipette solution for all recordings contained (in mM): 140 CsCl, 10 NaCl, 10 HEPES, 0.5 EGTA, 3 Mg-ATP, 0.2 Na-GTP, and 10 Na₂-phosphocreatine, pH adjusted to 7.3 with CsOH. 20 μM (−)-Bicuculline methochloride (Tocris), 1 μM strychnine HCl (Sigma). 0.5 μM tetrodotoxin (TTX) (Alomone Labs) were added to the ACSF for mEPSC recordings to block GABA_{A} receptors, glycine receptors and Na⁺ channels respectively. Input resistance was measured from a voltage response elicited by intracellular injection of a current pulse (−100 pA, 200 ms). Membrane voltage was low-pass filtered at 5 kHz and digitized at 10 kHz using a Multiclamp 700B amplifier connected to a DigiData 1322A interface (Axon Instruments) using Clampex 10.2 software (Molecular Devices, Foster City, CA). Liquid junction potentials were calculated and corrected off-line. Action potentials (AP) were generated in current clamp for currents injected in 10 pA intervals from 0 to 250 pA. Recordings were analyzed for: resting membrane potential, input resistance, rheobase, threshold, as well as AP amplitude, overshoot, duration, half-width, rise and decay. Neurons were held at −80 mV and continuous recordings of mEPSCs were made using Axoscope software (Molecular Devices, Union City, CA). Data processing and analysis were performed using MiniAnalysis (Synaptosoft, Decatur, GA) and Clampfit 10 (Molecular Devices). Events were detected by setting the threshold value, followed by visual confirmation of mEPSC detection.

### Calcium Imaging and Analysis

hPSC-derived cortical neurons were infected with lentiviruses encoding GC GCaMP6m and cultured on μ-plate 96 Well Black (Ibidi). Ca²⁺ was performed as previously described. Briefly, on the day of the imaging, cells were gently washed twice in modified Tyrode solution (25 mM HEPES (Invitrogen), 140 mM NaCl, 5 mM KCl, 1 mM MgCl₂, 10 mM glucose, 2 mM CaCl₂, 10 μM glycine, 0.1% BSA pH 7.4, pre-warmed to 37° C.) and equilibrated in imaging buffer for 1-2 min (25 mM HEPES, 140 mM NaCl, 8 mM KCl, 1 mM MgCl₂, 10 mM glucose, 4 mM CaCl₂, 10 μM glycine, 0.1% BSA pH 7.4, pre-warmed to 37° C.). GCaMP6m fluorescence was recorded on Celldiscover7 (ZEISS) inverted epi-fluorescence microscope with the 488 nm filter under environmental control (37° C.; 95% O₂-5% CO₂) at the Bio-Imaging Resource Center (BIRC) at Rockefeller University. Neuronal cultures were imaged for ˜3 min at a frame rate of 4-6 frames/second (800 frames/time lapse) using a 10× or 20× objectives. Analysis was performed as previously described. Briefly, the live-imaging image stack was converted to TIFF format and loaded into optimized scripts in MATLAB. Region of Interest (ROI) were placed on the neuron somas to calculate the raw GCaMP6m intensity of each neuron over time. The signal intensity of each raw trace was normalized to the baseline (ΛF/F0) for spike detection. Single-neuron amplitude was calculated from the normalized GCaMp6m intensity for all the detected spikes in each trace (mean ΛF/F0 of detected spikes for each neuron). Single-neuron frequency was calculated as the number of detected spikes in each trace per minute of recording. Network activity was assessed by calculating the synchronous firing rate, defined as the number of detected synchronous Ca²⁺ spikes from all ROI in one Field of View (FOV) per minute of recording.

Image analysis and Quantification

Morphological reconstruction of neurons was performed using Imaris Software. Ca²⁺ imaging analysis was performed using MATLAB software. Quantification of immunofluorescence images was performed in ImageJ or using the Operetta High content imaging system coupled with Harmony software (PerkinElmer).

### Protein Extraction and Western Blots

Cells were harvested and lysed in RIPA buffer (Sigma) with 1:100 Halt™ Protease and Phosphatase Inhibitor Cocktail (Thermo Fisher Scientific) and then sonicated for 3×30 sec at 4° C. Protein lysates were centrifugated for 15 min at >15000 rpm at 4° C. and supernatant was collected and quantified by Precision Red Advanced Protein Assay (Cytoskeleton). 5-10 ug of protein were boiled in NuPAGE LDS sample buffer (Invitrogen) at 95° C. for 5 min and separated using NuPAGE 4%-12% Bis-Tris Protein Gel (Invitrogen) in NuPAGE MES SDS Running Buffer (Invitrogen). Proteins were electrophoretically transferred to nitrocellulose membranes (Thermo Fisher Scientific) with NuPAGE Transfer Buffer (Invitrogen). Blots were blocked for 60 min at RT in TBS-T+5% nonfat milk (Cell Signaling) and incubated overnight in the same solution with the respective primary antibodies at 4° C. The following primary antibodies were used: mouse anti-Neurofilament H (non phosphorylated) (SMI32; Enzo Life science); mouse anti-Syntaxin 1A (110 111; SYSY); mouse anti-actin (MAB1501; Millipore); mouse anti-Cas9 (1497; Cell Signaling Technology); rabbit anti-Chd3 (ab109195, Abcam); rabbit anti-KDM5B (ab181089, abcam). The following secondary antibodies were incubated for 1 hour at RT: anti-mouse IgG HRP-linked (7076; Cell Signaling Technology) and anti-rabbit IgG HRP-linked (7074; Cell Signaling Technology) Blots were revealed using SuperSignal™ West Femto Chemiluminescent Substrate (Thermo Fischer Scientific). Chemiluminescence was imaged and analyzed using Image lab software (Biorad).

RNA Isolation and qRT-PCR

Samples were collected in Trizol and total RNA was isolated by chloroform phase separation using Phase Lock Gel-Heavy tubes, precipitated with EtOH and purified using RNeasy Mini Kit (Qiagen) with on-column DNA digestion step. cDNA was generated using the iScript Reverse Transcription Supermix (Bio-Rad) for RT-qPCR and qPCR reactions were performed using SsoFast EvaGreen®Supermix (Bio-Rad) using Quantitect Primer assays (QIAGEN). Results were normalized to the housekeeping gene GAPDH.

### DNA Construct and Lentivirus Production

Cas9-T2A-PuroR cassette flanked by 5′ and 3′ homology arms for the GPI locus was generated by NEBuilder® HiFi DNA Assembly Cloning Kit of PCR amplified fragments according to manufacturer's instruction. EF1alpha-GCaMP6m lentiviral vector was generated by PCR amplification of GCaMP6m from pGP-CMV-GCaMP6m (Addgene #40754) using with Q5 High Fidelity master mix (NEB) and subcloned into pWPXLd (Addgene #12258) into BamHI and EcoRI restriction site using standard cloning methods. For the simultaneous expression of gene-specific gRNA under transcriptional control of U6 promoter and dTomato fluorescent reporter driven by EF1alpha promoter, the SGL40.EFs.dTomato vector (Addgene #89398) was modified by inserting a P2A-Basticidin cassette downstream of dTomato sequence to generate the SGL40.EFs.dTomato-Blast backbone. gRNA sequences specific to each gene were designed using SYNTEGO CRISPR design tool (https://www.synthego.com/products/bioinformatics/crispr-design-tool) and validated using CRISPOR tools (http://crispor.tefor.net). DNA oligos (IDT) were annealed and subcloned into BsmBI restriction sites of SGL40.EFs.dTomato-Blast lentiviral backbone by standard cloning methods. Lentiviruses were produced by transfection of HEK293T cells using the Xtreme Gene 9 DNA transfection reagent (Sigma) with the respective lentiviral vectors along with the packaging vectors psPAX2 (Addgene, 12260) and pMD2.G (Addgene, 12259). Arrayed CRISPR gRNA lentiviral libraries were produced simultaneously and viruses were harvested 48 h post transfection, filtered with 0.22 μm filters and store in aliquots at −80° C. The sequence of each gRNA used is reported in Table 4.

### RNAseq and Analysis

Total RNA was extracted as described above. Sample for TruSeq stranded ribo-depleted paired-end total RNAseq at 40-50 million reads were submitted at the Epigenomic Core at Weill Cornell Medical College (WCMC). Samples for paired-end poly-A enriched RNAseq at 20-30 million reads were submitted to the Memorial Sloan Kettering Cancer Center (MSKCC) Genomic Core. Quality control of sequenced reads was performed by FastQC. Adaptor-trimmed reads were mapped to the hg19 human genome using STAR. The htseq-count function of the HTSeq Python package was used to count uniquely aligned reads at all exons of a gene. The count values were transformed to reads per kilobase per million (RPKM) to make them comparable across replicates. A threshold of 1 RPKM was used to consider a gene to be present in a sample and genes that were present in at least one sample were used for subsequent analyses. Variance stabilizing transformation (VST) of RNAseq counts was used for the Principal Component Analysis (PCA) Plots and for heatmaps of gene expression. Differential gene expression across time-points was computed using DESeq2. For downstream analysis of trends of gene expression, transcripts were first grouped into “monotonically upregulated” and “monotonically downregulated” based on the characteristics of their expression from day 25 to day 100. The three transitions where differential expression was evaluated and used to categorize genes were: day-25 vs day-50, day-50 vs day-75 and day-75 vs day-100. The present disclosure further split the genes into “strict” and “relaxed” categories based on the consistency of the transition. After fitting the RNA-seq counts to a generalized linear model (GLM) using DESeq2, genes were assigned to a group using the statistical significance in the following manner: (a) strict: all transitions satisfy the statistical significance criteria and (b) relaxed: day 25 vs day 100 transition satisfy the significance criteria and intermediate transitions may not. For all comparisons a significance threshold of FDR≤5% was used. For genes with three statistically significant comparisons, the average expression value per condition was calculated from the expression level normalized by the library size. Monotonically upregulated (strict): (d50 vs. d25: FDR≤5%) AND (d100 vs. d25: FDR≤5%) AND (d100 vs. d50: FDR≤5%) AND (d50 vs. d25: log FC>0) AND (d75 vs. d50: log FC>0) AND (d100 vs. d25 log FC>d50 vs. d25 log FC). Monotonically downregulated (strict): (d50 vs. d25: FDR≤5%) AND (d100 vs. d25: FDR≤5%) AND (d100 vs. d50: FDR≤5%) AND (d50 vs. d25: log FC<0) AND (d75 vs. d50: log FC<0) AND (d100 vs. d25 log FC<d50 vs. d25 log FC). Monotonically upregulated (relaxed): (d100 vs. d25: FDR≤5%) AND (d50 vs. d25: log FC>0) AND ((d100 vs. d25: log FC>=d50 vs. d25: log FC) OR (d75 vs. d50: log FC>0)). Monotonically downregulated (relaxed): (d100 vs. d25: FDR≤5%) AND (d50 vs. d25: log FC<0) AND ((d100 vs. d25: log FC<=d50 vs. d25: log FC) OR (d75 vs. d50: log FC<0)). GSEA was performed on day 50 vs. day 25 and day 100 vs. day 50 pairwise comparisons to test enrichment in KEGG pathways or gene sets from MSigDB using the following parameters: FDR≤5%, minimum gene-set size=15, maximum gene-set size=500, number of permutations=1000. GO analysis was performed using DAVID. Single-cell RNAseq analysis for mouse cortical development in FIGS. 31A-31B derived from the published dataset by Di Bella et al (Nature 595, 554-559, (2021)). Data was processed using the same pipeline as in the original publication and developmental trajectories were inferred using URD algorithm (Farrell, J. A. et al. Science 360, (2018)).

### ATACseq and Analysis

ATACseq libraries were prepared at the Epigenetic Innovation Lab at MSKCC starting from ˜50,000 live cells plated on 96-wells. Size-selected libraries were submitted to the MSKCC Genomic core for paired-end sequencing at 40-60 million reads. Quality control of sequenced reads was performed by FastQC (https://www.bioinformatics.babraham.ac.uk/projects/fastqc/) and adaptor filtration was performed by Trimmomatic version 0.36. The filtered reads were aligned to the hg19 reference genome. Macs2 was used for removing duplicate reads and calling peaks. Differentially accessible peaks in the atlas were called by DESeq2. To define dynamic trends of chromatin accessibility during neuronal maturation as shown in FIG. 18G, agglomerative hierarchical clustering using Ward's methods of merged differentially accessible peaks in pairwise comparisons between d25, d50, d75 and d100 samples was applied. HOMER findMotifsGenome.pl was used to investigate the motif enrichment in pairwise comparisons and unbiasedly clustered groups of peaks. Motif enrichment was also assessed by Kolmogorov-Smirnov and hypergeometric tests as previously described (Lee at al., 2019). ATAC-seq peaks in the atlas were associated with TF motifs in the updated CIS-BP database using FIMO of MEME suite. Hypergeometric test was used to compare the proportion of peaks containing a transcription factor motif in each group (foreground ratio) with that in the entire atlas (background ratio). Odds ratio represents the normalized enrichment of peaks associated with transcription factor motifs in the group compared to the background (foreground ratio/background ratio).

### Cut&Run and Analysis

Cut&Run was performed from 50,000 cells per condition as previously described using the following antibodies: rabbit anti-H3K4me3 (aab8580, abcam); rabbit anti-H3K9mc3 (ab8898, abcam); rabbit anti-H3K27me3 (9733, Cell Signaling Technologies); rabbit anti-H3K27ac (309034, Active Motif), normal rabbit IgG (2729, Cell Signaling Technologies). In brief, cells were harvested and bound to concanavalin A-coated magnetic beads after an 8 min incubation at RT on a rotator. Cell membranes were permeabilized with digitonin and the different antibodies were incubated overnight at 4° C. on a rotator. Beads were washed and incubated with pA-MN. Ca²⁺-induced digestion occurred on ice for 30 min and stopped by chelation. DNA was finally isolated using an extraction method with phenol and chloroform. Sequencing reads were trimmed and filtered for quality and adapter content using version 0.4.5 of TrimGalore (https://www.bioinformatics.babraham.ac.uk/projects/trim_galore) and running version 1.15 of cutadapt and version 0.11.5 of FastQC. Reads were aligned to human assembly hg19 with version 2.3.4.1 of bowtie2 (http://bowtie-bio.sourceforge.net/bowtie2/index.shtml) and MarkDuplicates of Picard Tools version 2.16.0 was used for deduplication. Enriched regions were discovered using MACS2 with a p-value setting of 0.001 and a matched IgG as the control. The BEDTools suite (http://bedtools.readthedocs.io) was used to create normalized read density profiles. A global peak atlas was created by first removing blacklisted regions (https://www.encodeproject.org/annotations/ENCSR636HFF) then merging all peaks within 500 bp and counting reads with version 1.6.1 of featureCounts (http://subread.sourceforge.net). Reads were normalized by sequencing depth (to 10 million mapped fragments) and DESeq2 was used to calculate differential enrichment for all pairwise contrasts. Clustering was performed on the superset of differential peaks using k-means clustering by increasing k until redundant clusters arose. Gene annotations were created by assigning all intragenic peaks to that gene, and otherwise using linear genomic distance to transcription start site. The annotations in each cluster were used to intersect with the RNA-seq time series by plotting the average expression z-score of all peak-associated genes which are differentially expressed across any stage. Motif signatures and enriched pathways were obtained using Homer v4.11 (http://homer.ucsd.edu).

### Statistical Analysis

Statistics were performed in PRISM (GraphPad) and R software. Data are represented as arithmetical means+/−standard error of the mean (s.e.m.) unless otherwise indicated.

### Example 3

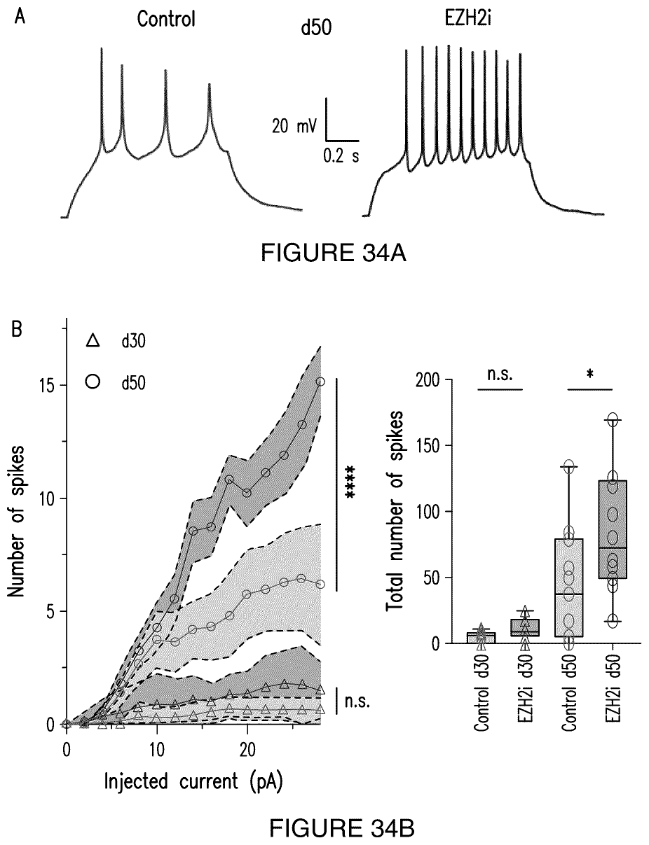

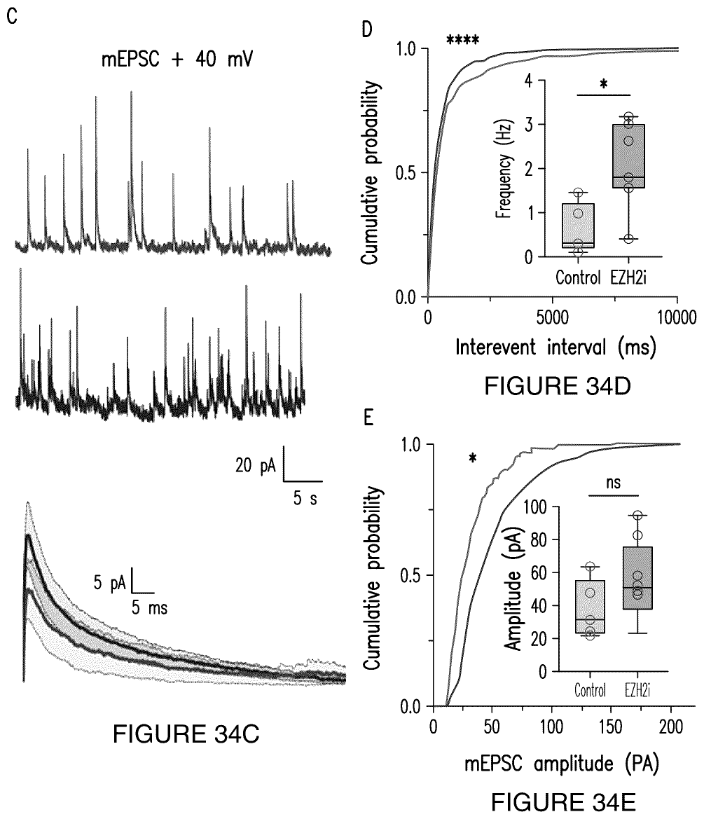

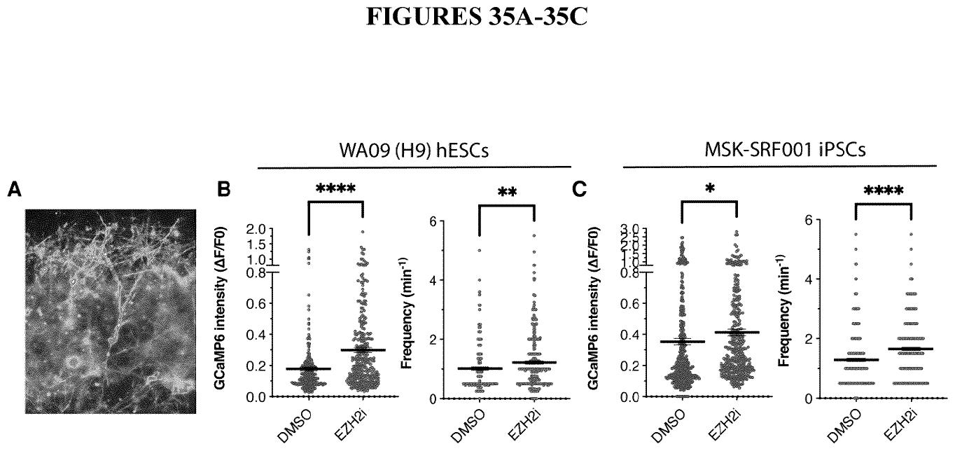

The present example describes a molecular study detailing the effects of EZH2 transient inhibitors used at progenitor cell stage on hPSC-derived cortical neurons and hPSC-derived brain cortical organoids. As shown in FIGS. 34A-34E, EZH2 transient inhibition significantly increased frequency and amplitude of firing of hPSC-derived cortical neurons. Further, significant increased frequency and amplitude of spontaneous individual calcium spikes was observed in the hPSC-derived brain cortical organoids (FIGS. 35A-35C).

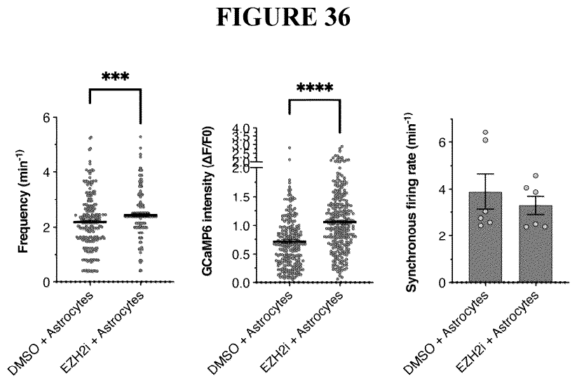

Next, it was determined whether EZH2 transient inhibitors used at progenitor cell stage could affect hPSC-derived cortical neurons co-cultured with rat astrocytes. Consistently with the other tested models, EZH2 transient inhibition significantly increased frequency and amplitude of individual calcium spikes without altering the synchronicity of firing (FIG. 36).

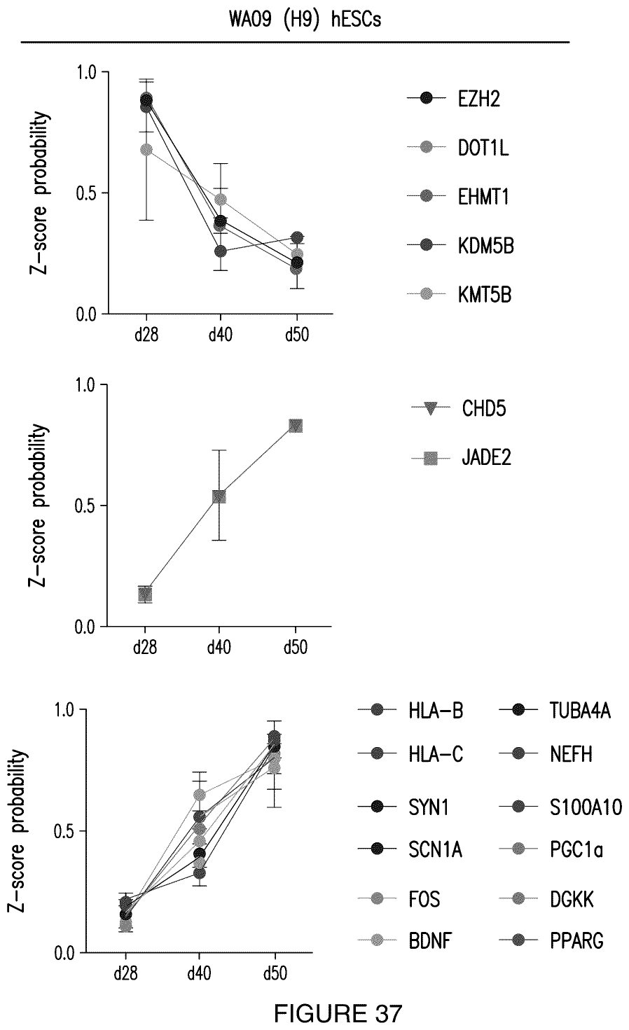

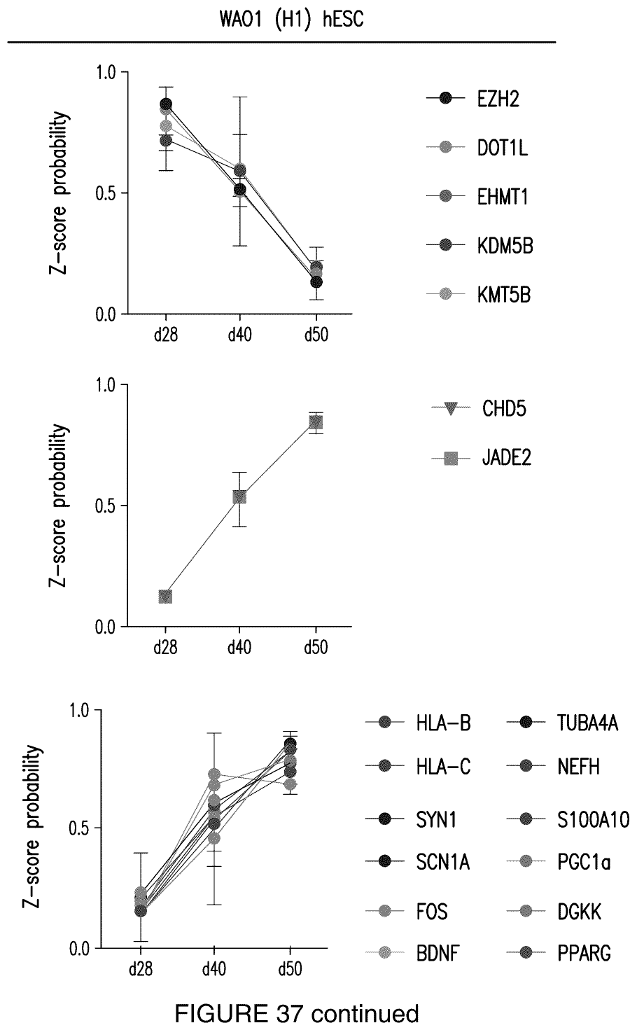

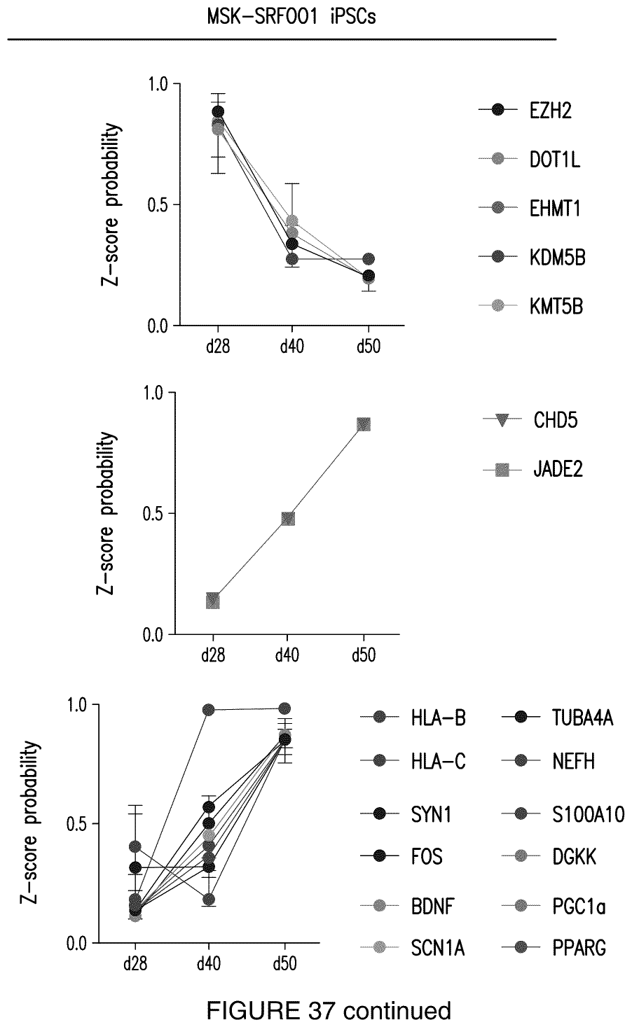

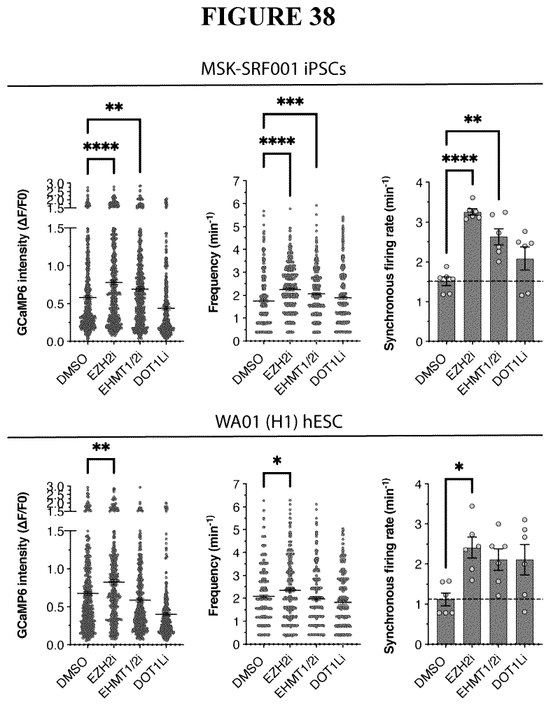

It was next determined whether EZH2 transient inhibitors could modulate maturation of neurons derived from different hPSC lines. Gene profile analysis showed that expression of EZH2, DOT1L, EHMT1, KDM5B, and KMT5B was downregulated over time (FIG. 37). FIG. 37 shows selected examples for the natural expression of maturation markers and epigenetic factors across neurons derived from multiple human Pluripotent Stem Cell lines, confirming that the gradual downregulation of epigenetic factors during neuronal maturation is observed independently of the cell line used for the differentiation. Finally, functional analysis of hESC-derived and iPSC-derived cortical neurons showed that amplitude, frequency and synchronicity of spontaneous individual calcium spikes were significantly increased in neurons derived from progenitor cells treated with epigenetic inhibitors (FIG. 38).

Overall, these data confirm that the epigenetic barrier at maturation is established before the onset of neurogenesis during hPSC-to-NPC transition and in turn gets inherited in newborn neurons.

Although the presently disclosed subject matter and its advantages have been described in detail, it should be understood that various changes, substitutions and alterations can be made herein without departing from the spirit and scope of the invention. Moreover, the scope of the present application is not intended to be limited to the particular embodiments of the process, machine, manufacture, and composition of matter, means, methods and steps described in the specification. As one of ordinary skill in the art will readily appreciate from the invention of the presently disclosed subject matter, processes, machines, manufacture, compositions of matter, means, methods, or steps, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, means, methods, or steps.

Various patents, patent applications, publications, product descriptions, protocols, and sequence accession numbers are cited throughout this application. the inventions of which are incorporated herein by reference in their entireties for all purposes.

What is claimed is:

1. A composition for promoting in vitro maturation of cells, comprising at least one inhibitor of an epigenetic regulator, and at least one agonist of a calcium channel.

2. The composition of claim 1, wherein the at least one inhibitor of the epigenetic regulator comprises a lysine-specific demethylase 1 (LSD1) inhibitor, a disruptor of telomerase-like 1 (DOT1L) inhibitor, or a combination thereof.

3. The composition of claim 1 or 2, wherein the at least one agonist of the calcium channel comprises a glutamate receptor agonist, an L-type calcium channel (LTCC) agonist, or a combination thereof.

4. The composition of claim 2 or 3, wherein the LSD1 inhibitor is selected from the group consisting of GSK2879552, OG-L002, GSK-LSD1, derivatives thereof, and combinations thereof.

5. The composition of any one of claims 2-4, wherein the DOT1L inhibitor is selected from the group consisting of EPZ-5676, EPZ004777, SYC-522, SGC0946, Dot1L-IN-2, Dot1L-IN-4, Dot1L-IN-5, Dot1L-IN-6, CN-SAH, derivatives thereof, and combinations thereof.

6. The composition of any one of claims 3-5, wherein the glutamate receptor agonist is selected from the group consisting of NMDA, (RS)-(Tetratazol-5-yl)glycine, ibotenic acid, derivatives thereof, and combinations thereof.

7. The composition of any one of claims 3-6, wherein the LTCC agonist is selected from the group consisting of Bay K 8644, FPL 64176, derivatives thereof, and combinations thereof.

8. The composition of any one of claims 1-7, wherein the composition comprises an LSD1 inhibitor, a DOT1L inhibitor, a glutamate receptor agonist, and an LTCC agonist.

9. The composition of any one of claims 1-8, wherein the composition comprises GSK2879552, EPZ-5676, NMDA, and Bay K 8644.

10. The composition of any one of claims 2-9, wherein the concentration of the LSD1 inhibitor is between about 0.1 μM and about 10 μM.

11. The composition of any one of claims 2-10, wherein the concentration of the LSD1 inhibitor is about 1 μM.

12. The composition of any one of claims 2-11, wherein the concentration of the DOT1L inhibitor is between about 0.1 μM and about 10 μM.

13. The composition of any one of claims 2-12, wherein the concentration of the DOT1L inhibitor is about 1 μM.

14. The composition of any one of claims 3-13, wherein the concentration of the glutamate receptor agonist is between about 0.1 μM and about 10 μM.

15. The composition of any one of claims 3-14, wherein the concentration of the glutamate receptor agonist is about 1 μM.

16. The composition of any one of claims 3-15, wherein the concentration of the LTCC agonist is between about 0.1 μM and about 10 μM.

17. The composition of any one of claims 3-16, wherein the concentration of the LTCC agonist is about 1 μM.

18. A composition for promoting in vitro maturation of cells, comprising at least one inhibitor of an epigenetic regulator.

19. The composition of claim 18, wherein the at least one inhibitor of the epigenetic regulator comprises a disruptor of telomerase-like 1 (DOT1L) inhibitor, an enhancer of zeste homolog 2 (EZH2) inhibitor, an euchromatic histone-lysine-N-methyltransferases 1 and 2 (EHMT1/2) inhibitor, or a combination thereof.

20. The composition of claim 19, wherein the EZH2 inhibitor is selected from the group consisting of 3-deazaneplanocin A (DZNep), GSK343, GSK126, EPZ-6438, EPZ005687, GSK926, EPZ6438, EPZ011989, CPI-1205, CPI-169, ZLD1039, PF-06821497, UNC1999, PR-S1/OR-S2, DS-3201b, A-395, EBI-2511, EED226, EEDi-5285, EI1, EZH2-IN-2, EZH2-IN-3, EZH2-IN-4, EZH2-IN-5, GNA002, GSK503, JQEZ5, MAK683, MS1943, PF-06726304, UNC 1999, UNC6852, UNC6852, AM41-44A, BR-001, CPI-1328, CPI-905, DCE_254, EBI-2511, YM181, YM181, ZLD1039, ZLD10A, derivatives thereof, and combinations thereof.

21. The composition of claim 19 or 20, wherein the EHMT1/2 inhibitor is selected from the group consisting of UNC0638, UNC0224, UNC0321, UNC0642, UNC0646, UNC0642, UNC0631, A-366, BIX-01294, BRD4770, BRD9539, CM-272, CM-579, CPUY074020, CSV0C018875, EHMT2-IN-1, EHMT2-IN-2, EML741, derivatives thereof, and combinations thereof.

22. The composition of any one of claims 19-21, wherein the DOT1L inhibitor is selected from the group consisting of EPZ-5676, EPZ004777, SYC-522, SGC0946, Dot1L-IN-2, Dot1L-IN-4, Dot1L-IN-5, Dot1L-IN-6, CN-SAH, derivatives thereof, and combinations thereof.

23. The composition of any one of claims 18-22, comprising GSK343, EPZ004777, UNC0638, or a combination thereof.

24. The composition of any one of claims 18-23, wherein the concentration of the at least one inhibitor of the epigenetic regulator is between about 0.1 μM and about 10 μM.

25. The composition of any one of claims 18-24, wherein the concentration of the at least one inhibitor of the epigenetic regulator is about 2 μM or about 4 μM.

26. An in vitro method for promoting the maturation of cells, comprising contacting the cells with at least one inhibitor of an epigenetic regulator, and at least one agonist of a calcium channel.

27. The method of claim 26, wherein the at least one inhibitor of the epigenetic regulator comprises a lysine-specific demethylase 1 (LSD1) inhibitor, a disruptor of telomerase-like 1 (DOT1L) inhibitor, or a combination thereof.

28. The method of claim 26 or 27, wherein the at least one agonist of the calcium channel comprises a glutamate receptor agonist, an L-type calcium channel (LTCC) agonist, or a combination thereof.

29. The method of claim 27 or 28, wherein the LSD1 inhibitor is selected from the group consisting of GSK2879552, OG-L002, GSK-LSD1, derivatives thereof, and combinations thereof.

30. The method of any one of claims 27-29, wherein the DOT1L inhibitor is selected from the group consisting of EPZ-5676, EPZ004777, SYC-522, SGC0946, Dot1L-IN-2, Dot1L-IN-4, Dot1L-IN-5, Dot1L-IN-6, CN-SAH, derivatives thereof, and combinations thereof.

31. The method of any one of claims 28-30, wherein the glutamate receptor agonist is selected from the group consisting of NMDA, (RS)-(Tetratazol-5-yl)glycine, ibotenic acid, derivatives thereof, and combinations thereof.

32. The method of any one of claims 28-31, wherein the LTCC agonist is selected from the group consisting of Bay K 8644, FPL 64176, derivatives thereof, and combinations thereof.

33. The method of any one of claims 27-32, wherein the method comprises contacting the cells with an LSD1 inhibitor, a DOT1L inhibitor, a glutamate receptor agonist, and an LTCC agonist.

34. The method of any one of claims 26-33, wherein the method comprises contacting the cells with GSK2879552, EPZ-5676, NMDA, and Bay K 8644.

35. The method of any one of claims 27-34, wherein the concentration of the LSD1 inhibitor is between about 0.1 μM and about 10 μM.

36. The method of any one of claims 27-35, wherein the concentration of the LSD1 inhibitor is about 1 μM.

37. The method of any one of claims 27-36, wherein the concentration of the DOT1L inhibitor is between about 0.1 μM and about 10 μM.

38. The method of any one of claims 27-37, wherein the concentration of the DOT1L inhibitor is about 1 μM.

39. The method of any one of claims 28-38, wherein the concentration of the glutamate receptor agonist is between about 0.1 μM and about 10 μM.

40. The method of any one of claims 28-39, wherein the concentration of the glutamate receptor agonist is about 1 μM.

41. The method of any one of claims 28-40, wherein the concentration of the LTCC agonist is between about 0.1 μM and about 10 μM.

42. The method of any one of claims 28-41, wherein the concentration of the LTCC agonist is about 1 μM.

43. The method of any one of claims 26-42, wherein the cells are contacted with the at least one inhibitor of the epigenetic regulator and the at least one agonist of the calcium channel for at least about 3 days and/or for up to about 30 days.

44. An in vitro method for promoting the maturation of cells, comprising contacting the cells with at least one inhibitor of an epigenetic regulator.

45. The method of claim 44, wherein the at least one inhibitor of the epigenetic regulator comprises a disruptor of telomerase-like 1 (DOT1L) inhibitor, an enhancer of zeste homolog 2 (EZH2) inhibitor, an euchromatic histone-lysine-N-methyltransferases 1 and 2 (EHMT1/2) inhibitor, or a combination thereof.

46. The method of claim 45, wherein the EZH2 inhibitor is selected from the group consisting of 3-deazaneplanocin A (DZNep), GSK343, GSK126, EPZ-6438, EPZ005687, GSK926, EPZ6438, EPZ011989, CPI-1205, CPI-169, ZLD1039, PF-06821497, UNC1999, PR-S1/OR-S2, DS-3201b, A-395, EBI-2511, EED226, EEDi-5285, EI1, EZH2-IN-2, EZH2-IN-3, EZH2-IN-4, EZH2-IN-5, GNA002, GSK503, JQEZ5, MAK683, MS1943, PF-06726304, UNC 1999, UNC6852, UNC6852, AM41-44A, BR-001, CPI-1328, CPI-905, DCE_254, EBI-2511, YM181, YM181, ZLD1039, ZLD10A, derivatives thereof, and combinations thereof.

47. The method of claim 45 or 46, wherein the EHMT1/2 inhibitor is selected from the group consisting of UNC0638 UNC0224, UNC0321, UNC0642, UNC0646, UNC0642, UNC0631, A-366, BIX-01294, BRD4770, BRD9539, CM-272, CM-579, CPUY074020, CSV0C018875, EHMT2-IN-1, EHMT2-IN-2, EML741, derivatives thereof, and combinations thereof.

48. The method of any one of claims 45-47, wherein the DOT1L inhibitor is selected from the group consisting of EPZ-5676, EPZ004777, SYC-522, SGC0946, Dot1L-IN-2, Dot1L-IN-4, Dot1L-IN-5, Dot1L-IN-6, CN-SAH, derivatives thereof, and combinations thereof.

49. The method of any one of claims 44-48, comprising contacting the cells with GSK343, EPZ004777, UNC0638, or a combination thereof.

50. The method of any one of claims 44-49, wherein the concentration of the at least one inhibitor of the epigenetic regulator is between about 0.1 μM and about 10 μM.

51. The method of any one of claims 44-50, wherein the concentration of the at least one inhibitor of the epigenetic regulator is about 2 μM or about 4 μM.

52. The method of any one of claims 26-51, wherein the cells are immature neuronal cells, precursors thereof, progenitors thereof, or a combination thereof.

53. The method of claim 52, wherein the neuronal cells are selected from the group consisting of cortical neurons, spinal motor neurons, and combinations thereof.

54. The method of claim 52 or 53, wherein the cells form a brain organoid.

55. The method of claim 54, wherein the brain organoid is a dorsal forebrain organoid.

56. The method of any one of claims 26-51, wherein the cells are immature non-neuronal cells, precursors thereof, progenitors thereof, or a combination thereof.

57. The method of claim 56, wherein the cells are selected from the group consisting of pancreatic beta cells, melanocytes, and combinations thereof.

58. The method of any one of claims 26-57, wherein the cells are in vitro differentiated from stem cells.

59. The method of claim 58, wherein the stem cells are selected from the group consisting of embryonic stem cells, induced pluripotent stem cells, parthenogenetic stem cells, primordial germ cell-like pluripotent stem cells, epiblast stem cells, and F-class pluripotent stem cells, embryonic neural stem cells, adult neural stem cells, and long-term self-renewing neural stem cells, and combinations thereof.

60. An in vitro method for promoting the maturation of cells, comprising contacting the cells with the composition of any one of claims 1-25.

61. Use of the composition of any one of claims 1-25 for promoting the maturation of cells.

62. A kit for promoting in vitro maturation of cells, comprising at least one inhibitor of an epigenetic regulator, and at least one agonist of a calcium channel.

63. The kit of claim 62, wherein the at least one inhibitor of the epigenetic regulator comprises a lysine-specific demethylase 1 (LSD1) inhibitor, a disruptor of telomerase-like 1 (DOT1L) inhibitor, or a combination thereof.

64. The kit of claim 62 or 63, wherein the at least one agonist of the calcium channel comprises a glutamate receptor agonist, an L-type calcium channel (LTCC) agonist, or a combination thereof.

65. The kit of claim 63 or 64, wherein the LSD1 inhibitor is selected from the group consisting of GSK2879552, OG-L002, GSK-LSD1, derivatives thereof, and combinations thereof.

66. The kit of any one of claims 63-65, wherein the DOT1L inhibitor is selected from the group consisting of EPZ-5676, EPZ004777, SYC-522, SGC0946, Dot1L-IN-2, Dot1L-IN-4, Dot1L-IN-5, Dot1L-IN-6, CN-SAH, derivatives thereof, and combinations thereof.

67. The kit of any one of claims 64-66, wherein the glutamate receptor agonist is selected from the group consisting of NMDA, (RS)-(Tetratazol-5-yl)glycine, ibotenic acid, derivatives thereof, and combinations thereof.

68. The kit of any one of claims 64-67, wherein the LTCC agonist is selected from the group consisting of Bay K 8644, FPL 64176, derivatives thereof, and combinations thereof.

69. The kit of any one of claims 62-68, wherein the kit comprises an LSD1 inhibitor, a DOT1L inhibitor, a glutamate receptor agonist, and an LTCC agonist.

70. The kit of any one of claims 62-69, wherein the kit comprises GSK2879552, EPZ-5676, NMDA, and Bay K 8644.

71. A kit for promoting in vitro maturation of cells, comprising at least one inhibitor of an epigenetic regulator.

72. The kit of claim 71, wherein the at least one inhibitor of the epigenetic regulator comprises a disruptor of telomerase-like 1 (DOT1L) inhibitor, an enhancer of zeste homolog 2 (EZH2) inhibitor, an euchromatic histone-lysine-N-methyltransferases 1 and 2 (EHMT1/2) inhibitor, or a combination thereof.

73. The kit of claim 72, wherein the EZH2 inhibitor is selected from the group consisting of 3-deazaneplanocin A (DZNep), GSK343, GSK126, EPZ-6438, EPZ005687, GSK926, EPZ6438, EPZ011989, CPI-1205, CPI-169, ZLD1039, PF-06821497, UNC1999, PR-S1/OR-S2, DS-3201b, A-395, EBI-2511, EED226, EEDi-5285, EI1, EZH2-IN-2, EZH2-IN-3, EZH2-IN-4, EZH2-IN-5, GNA002, GSK503, JQEZ5, MAK683, MS1943, PF-06726304, UNC 1999, UNC6852, UNC6852, AM41-44A, BR-001, CPI-1328, CPI-905, DCE_254, EBI-2511, YM181, YM181, ZLD1039, ZLD10A, derivatives thereof, and combinations thereof.

74. The kit of claim 72 or 73, wherein the EHMT1/2 inhibitor is selected from the group consisting of UNC0638, UNC0224, UNC0321, UNC0642, UNC0646, UNC0642, UNC0631, A-366, BIX-01294, BRD4770, BRD9539, CM-272, CM-579, CPUY074020, CSV0C018875, EHMT2-IN-1, EHMT2-IN-2, EML741, derivatives thereof, and combinations thereof.

75. The kit of any one of claims 72-74, wherein the DOT1L inhibitor is selected from the group consisting of EPZ-5676, EPZ004777, SYC-522, SGC0946, Dot1L-IN-2, Dot1L-IN-4, Dot1L-IN-5, Dot1L-IN-6, CN-SAH, derivatives thereof, and combinations thereof.

76. The kit of any one of claims 71-75, comprising GSK343, EPZ004777, UNC0638, or a combination thereof.

77. The kit of any one of claims 71-76, further comprising instructions for promoting in vitro maturation of cells.

78. An in vitro method of screening a compound that is suitable for promoting in vitro maturation of cells, comprising:
(a) contacting a population of immature neuronal cells to a test compound;
(b) withdrawing the test compound;
(c) contacting the cells with potassium chloride between about 3 days and about 20 days after the withdrawal of the test compound;
(d) measuring nuclear morphology, neurite growth and membrane excitability of the cells;
(e) performing principal component analysis on the nuclear morphology, neurite growth and membrane excitability measured in step (d); and
(f) identifying a test compound that is suitable for promoting in vitro maturation of neuronal cells based on the principal component analysis performed in (e).

79. The method of claim 78, wherein the cells are contacted with potassium chloride about 7 days after the withdrawal of the test compound.

80. The method of claim 78 or 79, wherein the concentration of potassium chloride is between about 10 mM and about 100 mM.

81. The method of any one of claims 78-80, wherein the concentration of potassium chloride is about 50 mM.

82. The method of any one of claim 78-81, wherein measuring the nuclear morphology comprises measuring nuclear area and nuclear roundness.

83. The method of any one of claim 78-82, wherein the nuclear morphology is determined by DAPI counterstaining.

84. The method of any one of claim 78-83, wherein measuring the neurite growth comprises measuring neurite length and neurite branching.

85. The method of any one of claim 78-84, wherein the neurite growth is determined by microtubule-associated protein 2 (MAP2) immunostaining.

86. The method of any one of claim 78-85, wherein measuring the membrane excitability comprises measuring percentage of cells expressing an immediate early gene (IEG) product.

87. The method of claim 86, wherein measuring the membrane excitability comprises subtracting the percentage of cells expressing the IEG product with percentage of control cells expressing the IEG product, wherein the control cells are not subject to the contact of potassium chloride.

88. The method of claim 87, wherein the IEG product comprises FOS, EGR1, and a combination thereof.

89. The method of any one of claims 78-88, wherein the neuronal cells are cortical neurons.
